(19) United States
(12) Patent Application Publication
(10) Pub. No.: US 2026/0226026 A1
(43) Pub. Date: Aug. 6, 2026

(54) COMPOUND AS CTPS1 INHIBITOR

(71) Applicants: Hangzhou Innogate Pharma Co., Ltd., Hangzhou, Zhejiang (CN); Innorace Biopharma Co., Ltd., Hangzhou, Zhejiang (CN)

(72) Inventors: Hancheng ZHANG, Hangzhou, Zhejiang (CN); Congcong CAI, Hangzhou, Zhejiang (CN); Shiya ZHU, Hangzhou, Zhejiang (CN)

(21) Appl. No.: 19/149,677

(22) PCT Filed: Jan. 19, 2024
(86) PCT No.: PCT/CN2024/073373

§ 371 Date: Jul. 21, 2025

(30) Foreign Application Priority Data
Jan. 19, 2023 (CN) 202310060850X
Aug. 11, 2023 (CN) 2023110170718

(51) Int. Cl. C07D 401/14; A61K 31/497; A61K 31/506; A61K 31/5377; C07D 403/12; C07D 405/14; C07D 409/14; C07D 417/14

(52) U.S. Cl. CPC C07D 401/14; A61K 31/497; A61K 31/506; A61K 31/5377; C07D 403/12; C07D 405/14; C07D 409/14; C07D 417/14

(57) ABSTRACT

Provided in the present invention is a class of compounds. Specifically, provided in the present invention is a compound having a struc-ture as shown in formula (1), or an optical isomer, pharmaceutically accept-able salt, prodrug, deuterated derivative, hydrate and solvate thereof. The compound can effectively inhibit CTPS 1, and is used for treating or prevent-ing diseases or disorders associated with the activity or expression quantity of CTPS1.

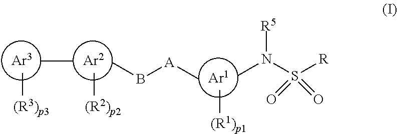

### TECHNICAL FIELD

The present invention relates to the field of medicinal chemistry. Specifically, the present invention relates to a class of novel compounds, their synthesis methods, and their application as CTPS1 (Cytidine triphosphate synthase 1) inhibitors in the preparation of medicaments for treating tumors and related diseases.

### BACKGROUND OF INVENTION

Nucleotides are compounds composed of a purine or pyrimidine base, a ribose or deoxyribose sugar, and phosphate groups. They are essential components in cellular metabolic processes, particularly in the synthesis of deoxyribonucleic acid (DNA) and ribonucleic acid (RNA). Cytidine triphosphate (CTP) serves not only as a precursor for DNA and RNA synthesis but also as a substrate for the biosynthesis of phospholipids. Among the four canonical nucleotides (UTP, ATP, GTP, and CTP), CTP is present at the lowest concentration, making it a critical rate-limiting molecule in nucleic acid synthesis and other CTP-dependent processes.

CTP is derived from two main pathways: the salvage pathway and the de novo synthesis pathway. CTP synthase 1 (CTPS1) and CTP synthase 2 (CTPS2) are the terminal and rate-limiting enzymes in the de novo synthesis of CTP. These enzymes catalyze the conversion of uridine triphosphate (UTP) and glutamine into cytidine triphosphate (CTP) and L-glutamate. Both CTPS1 and CTPS2 contain two distinct domains: an N-terminal synthetase domain and a C-terminal glutaminase domain. The N-terminal synthetase domain transfers a phosphate group from adenosine triphosphate (ATP) to the 4-position of UTP, forming an activated intermediate, 4-phosphoryl-UTP. The glutaminase domain, via a conserved active-site cysteine and a covalent thioester intermediate, generates ammonia from glutamine, which is then used by the synthetase domain to convert 4-phosphoryl-UTP into CTP.

Although the two human CTPS isoforms, CTPS1 and CTPS2, share approximately 75% sequence homology, they exhibit distinct physiological characteristics. CTPS2 is broadly expressed across various tissue types, whereas CTPS1 is generally expressed at low levels but is markedly upregulated in activated T cells. Loss-of-function mutations in CTPS1 due to deleterious homozygous variants result in severe immunodeficiency, and the functionally intact CTPS2 isoform is unable to compensate for the loss of CTPS1 in affected individuals. In these patients, T cells fail to undergo effective activation and proliferation upon antigen stimulation. Notably, these homozygous mutations are not associated with other clinical abnormalities. T cells isolated from such CTPS1-deficient individuals can regain proliferative capacity upon supplementation with exogenous CTP, highlighting CTPS1 as the essential source of CTP for lymphocyte expansion.

Given the pivotal role of lymphocyte populations such as T and B cells in a wide array of autoimmune disorders and other diseases, CTPS1 has emerged as a promising therapeutic target for a novel class of immunosuppressants. Inhibitors of CTPS1 could attenuate the proliferation of autoreactive immune cells and hematologic malignancies, offering broad therapeutic potential.

### SUMMARY OF THE INVENTION

The object of the present invention is to provide a novel class of CTPS1 inhibitors.

In a first aspect, the present invention provides a compound having a structure represented by Formula (I), or an optical isomer, pharmaceutically acceptable salt, prodrug, deuterated derivative, hydrate, or solvate thereof:

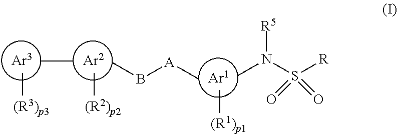

wherein in formula (I):
A is selected from Formula (Ia), Formula (Ib), or Formula (Ic):

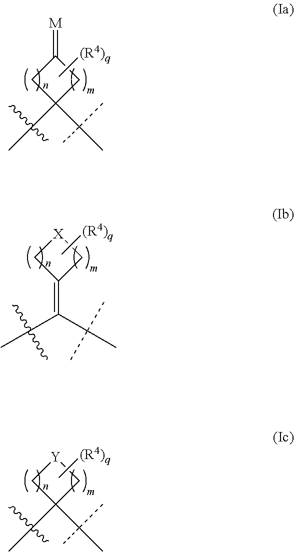

wherein in Formula (Ia), Formula (Ib), and Formula (Ic), “ [symbol] ” represents the point which attach to Ar¹ in the compound of Formula (I); “ [symbol] ” represents the point which attach to B in the compound of Formula (I);
B is —NHC(═O)— or —C(═O)NH—;
Ar¹, Ar² and Ar³ are each independently selected from aryl and heteroaryl;
R is selected from C₁₋₆ alkyl, C₃₋₆ cycloalkyl, 3- to 6-membered heterocyclyl, C₃₋₆ cycloalkyl-C₁₋₄ alkyl, 3- to 6-membered heterocyclyl-C₁₋₄ alkyl, C₃₋₆ cycloalkyl-C₂₋₄alkynyl, 3- to 6-membered heterocyclyl-C₂₋₄alkynyl, and C₃₋₆ cycloalkyl-C(═O)—C₁₋₂ alkyl;
R¹ is selected from hydrogen, halogen, C₁₋₄ alkyl, C₁₋₄ haloalkyl, C₁₋₄ alkoxy and CN;
R² is selected from hydrogen, halogen, C₁₋₄ alkyl, C₁₋₄ haloalkyl, C₁₋₄ alkoxy, C₁₋₄ haloalkoxy, and CN;
R³ is selected from hydrogen, halogen, C₁₋₄ alkyl, C₁₋₄ haloalkyl, C₂₋₄ alkenyl, C₂₋₄ alkynyl, C₃₋₆ cycloalkyl, C₃₋₆ cycloalkyl-C₁₋₄ alkyl, C₃₋₆ cycloalkyl-C₂₋₄alkynyl, 3- to 6-membered heterocyclyl, 3- to 6-membered heterocyclyl-C₂₋₄alkynyl, OR^{a}, SR^{a}, NR^{c}R^{c}, CN, or a group of Formula (Ie) (as shown below); wherein R^{a} is selected from hydrogen, C₁₋₄ alkyl, C₁₋₄ haloalkyl, C₃₋₆ cycloalkyl, or C₃₋₆ cycloalkyl-C₁₋₄ alkyl; each R^{c} is independently selected from hydrogen, C₁₋₄ alkyl, and C₁₋₄ haloalkyl;
R⁴ is selected from hydrogen, halogen, C₁₋₄ alkyl, C₁₋₄ haloalkyl, hydroxy, C₁₋₄ alkoxy, and CN;
R⁵ is selected from hydrogen, C₁₋₄ alkyl, C₃₋₆ cycloalkyl, 3- to 6-membered heterocyclyl, aryl, and heteroaryl;
M is selected from CR^{h}Rⁱ; wherein R^{h} and Rⁱ are each independently selected from hydrogen, halogen, C₁₋₄ alkyl, C₃₋₆ cycloalkyl, 3- to 8-membered heterocyclyl, aryl, heteroaryl, CN, C(O)R^{g}, C(O)OR^{f}, or C(O)NR^{d}R^{d}; wherein said alkyl, cycloalkyl, heterocyclyl, aryl, and heteroaryl are each optionally substituted by one or more substituents independently selected from the group consisting of: halogen, C₁₋₄ alkyl, C₂₋₄ alkenyl, C₂₋₄ alkynyl, C₃₋₆ cycloalkyl, 3- to 6-membered heterocyclyl, aryl, heteroaryl, CN, OR^{f}, SR^{f}, NR^{d}R^{d}, C(O)R^{g}, C(O)OR^{f}, OC(O)R^{g}, C(O)NR^{d}R^{d}, NR^{d}C(O)R^{g}, NR^{d}C(O)NR^{d}R^{d}, OC(O)NR^{d}R^{d}, NR^{d}(O)OR^{f}, OC(O)OR^{f}, S(O)₂NR^{d}R^{d}, NR^{d}S(O)₂R^{g}, and NR^{d}S(O)₂NR^{d}R^{d}; or R^{h} and Rⁱ together with the carbon atom to which they are attached form a 3- to 8-membered cyclic structure, said cyclic structure optionally containing 0, 1, or 2 heteroatoms independently selected from N, O and S, and optionally substituted by one or more substituents independently selected from the group consisting of halogen, C₁₋₄ alkyl, CN, OR^{f}, SR^{f}, NR^{d}R^{d}, and =M, wherein M is as defined above; wherein each R^{d} is independently selected from hydrogen, C₁₋₄ alkyl, C₁₋₄ haloalkyl, C₁₋₄ alkoxyC₂₋₄alkyl, C₃₋₆ cycloalkyl, and 3- to 6-membered heterocyclyl; each Rf is independently selected from hydrogen, C₁₋₄ alkyl, C₁₋₄ haloalkyl, C₃₋₆ cycloalkyl, and 3- to 6-membered heterocyclyl; each R^{g} is independently selected from hydrogen, C₁₋₄ alkyl, C₁₋₄ haloalkyl, C₂₋₄ alkenyl, C₂₋₄ alkynyl, C₃₋₆ cycloalkyl, 3- to 6-membered heterocyclyl, aryl, and heteroaryl;
X and Y are each independently selected from O, CR^{e}R^{e} and NR^{b}; wherein each R^{e} is independently selected from hydrogen, halogen, C₁₋₄ alkyl, C₃₋₆ cycloalkyl, 3- to 6-membered heterocyclyl, aryl, heteroaryl, CN, OR, SR, NR^{d}R^{d}, C(O)R^{g}, C(O)OR^{f}, OC(O)R^{g}, C(O)NR^{d}R^{d}, NR^{d}C(O)R^{g}, NR^{d}C(O)NR^{d}R^{d}, OC(O)NR^{d}R^{d}, NR^{d}C(O)OR^{f}, OC(O)OR^{f}, S(O)₂NR^{d}R^{d}, NR^{d}S(O)₂R^{g} and NR^{d}S(O)₂NR^{d}R^{d}; R^{b} is selected from hydrogen, C₁₋₄ alkyl, C₃₋₆ cycloalkyl, 3- to 6-membered heterocyclyl, aryl, heteroaryl, C(O)R^{g}, C(O)OR^{f}, C(O)NR^{d}R^{d}, S(O)₂NR^{d}R^{d} and S(O)₂R^{g}; the alkyl in R^{e} or R^{b} is optionally substituted by one or more substituents selected from the group consisting of halogen, C₃₋₆ cycloalkyl, 3- to 6-membered heterocyclyl, aryl, heteroaryl, CN, OR, SR, NR^{d}R^{d} C(O)R^{g}, C(O)O^{f}, OC(O)R^{g}, C(O)NR^{d}R^{d}, NR^{d}C(O)R^{g}, NR^{d}C(O)NR^{d}R^{d}, OC(O)NR^{d}R^{d}, NR^{d}C(O)OR^{f}, OC(O)OR^{f}, S(O)₂NR^{d}R^{d}, NR^{d}S(O)₂R^{g}, and NR^{d}S(O)₂NR^{d}R^{d}; R^{d}, Rf R^{g} are defined as above;
provided that when A is formula (Ic) and Y is NR^{b}, then NR^{b} is selected from aryl, heteroaryl, C(O)R^{g′}, or Formula (Id),

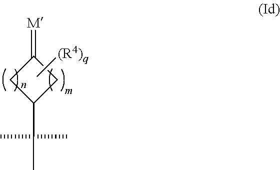

wherein “ [symbol] ” represents the point which attach to N in Formula (Id); R^{g′} is selected from C₂₋₄ alkenyl, C₂₋₄ alkynyl, C₃₋₆ cycloalkyl, or 3- to 6-membered heterocyclyl; M′ is selected from O or CR^{h}Rⁱ; R⁴, R^{h} and Rⁱ are as defined above;
with the proviso that, when A is selected from formula (Ic), Y is selected from O, CR^{e}R^{e}, or NR^{b}, and R^{b} is other than aryl, heteroaryl, C(O)R^{g′}, and Formula (Id), then R is selected from C₃₋₆ cycloalkyl-C₂₋₄ alkynyl, 3- to 6-membered heterocyclyl C₂₋₄ alkynyl, or C₃₋₆ cycloalkyl C(═O)C₁₋₂ alkyl; or R³ is selected from C₃₋₆ cycloalkyl-C₂₋₄ alkynyl, 3- to 6-membered heterocyclyl C₂₋₄ alkynyl, or formula (Ie);

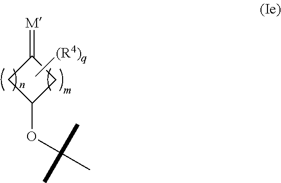

wherein “ [symbol] ” represents the point which attach to Ar³ in Formula (Ie); and R^{e}, R^{b}, R^{g′}, M′, and R⁴ are as defined above;
wherein p1, p2 and p3 are each independently selected from 0, 1, 2 and 3;
each m and each n are independently selected from 0, 1, 2, 3, 4, 5 and 6; provided that m and n are not both 0;
each q is independently selected from 0, 1, 2, 3 and 4;
wherein each of the aforementioned alkyl, alkenyl, alkynyl, cycloalkyl, heterocyclyl, cyclic structure, aryl, and heteroaryl groups is optionally and independently substituted by 1 to 3 substituents independently selected from the group consisting of: halogen, C₁₋₄ alkyl, C₁₋₄ haloalkyl, C₂₋₄ alkenyl, C₂₋₄ alkynyl, C₃₋₈ cycloalkyl, 3- to 8-membered heterocyclyl, aryl, heteroaryl, CN, NO₂, OR^{f}, SR^{f}, NR^{d}R^{d}, C(O)R^{g}, C(O)OR^{f}, C(O)NR^{d}R^{d}, NR^{d}C(O)R^{g}, NR^{d}S(O)₂R^{g} and S(O)₂R^{g}, provided that the resulting chemical structure is stable and chemically meaningful; wherein R^{d}, R^{f}, and R^{g} are as defined above;
unless otherwise specified, the aforementioned aryl is an aromatic group containing 6 to 12 carbon atoms; heteroaryl is a 5- to 15-membered heteroaromatic group; and cyclic structure is a saturated or unsaturated cyclic group which optionally containing heteroatoms.

In another preferred embodiment, the formula (I) is Formula (IIa) or Formula (IIb):

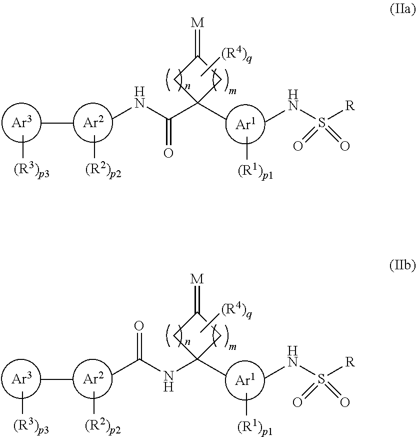

wherein the definitions of the groups in Formula (IIa) and Formula (IIb) are as defined in the first aspect of the present invention.

In another preferred embodiment, the Formula (I) is Formula (III):

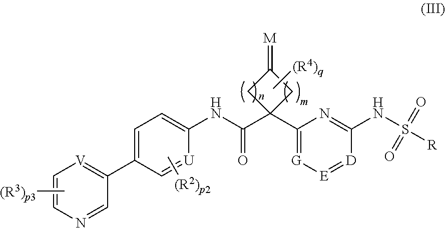

D, E, and G are each independently selected from N or CR¹, provided that no more than two of D, E, and G are N;
U is N or CR²;
V is N or CR³;
R¹, R², R³, and the definitions of the remaining groups in Formula (III) are as defined in the first aspect of the present invention.

In another preferred embodiment, the formula (I) is formula (IVa), formula (IVb), or formula (IVc):

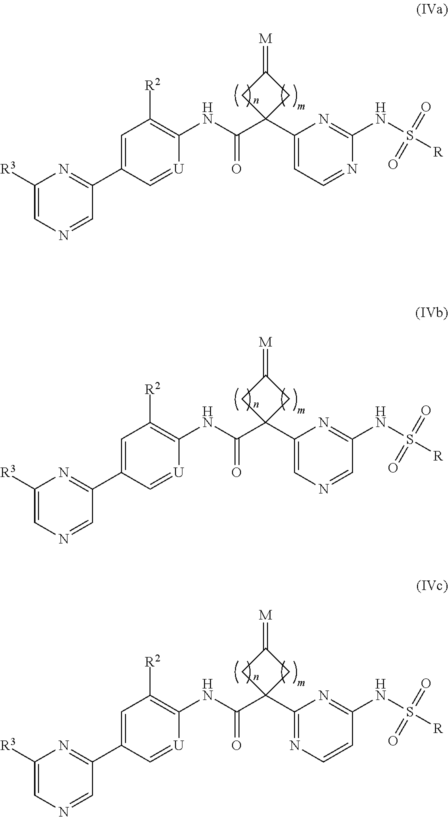

U is N or CH;
the remaining groups in Formula (IVa), Formula (IVb), and Formula (IVc) are as defined in the first aspect of the present invention.

In another preferred embodiment, the formula (I) is formula (V):

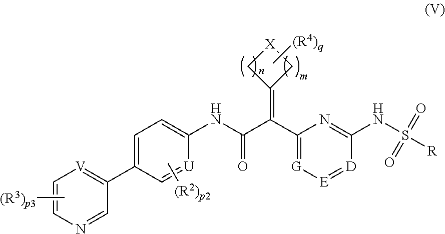

D, E and G are each independently selected from N or CR¹, provided that no more than two of D, E, and G are N;
U is N or CR²;
V is N or CR³;
R¹, R², R³, and the remaining groups in Formula (V) are as defined in the first aspect of the present invention.

In another preferred embodiment, the Formula (I) is formula (VIa), formula (VIb), or formula (VIc):

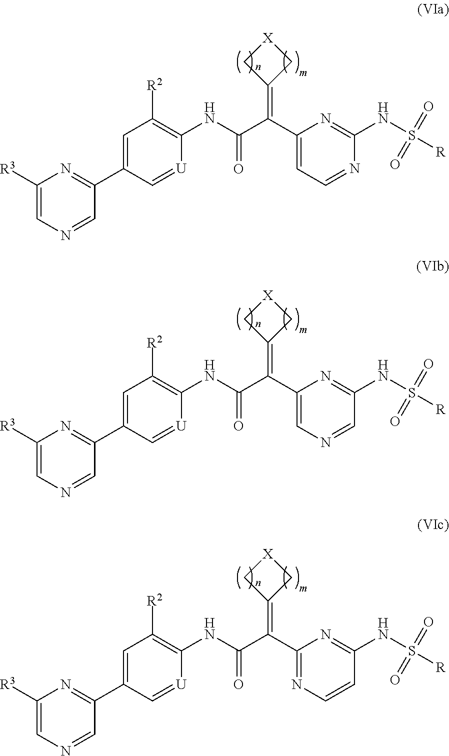

U is N or CH;
the remaining groups in Formula (VIa), Formula (VIb), and Formula (VIc) are as defined in the first aspect of the present invention.

In another preferred embodiment, the formula (I) is formula (VII):

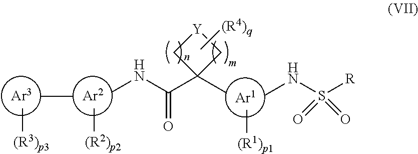

the remaining groups in formula (VII) and provisos are as defined in the first aspect of the present invention.

In another preferred embodiment, the formula (I) is formula (VIII):

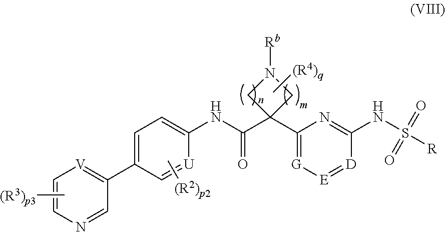

D, E and G are each independently selected from N or CR¹, provided that no more than two of D, E, and G are N;
U is N or CR²;
V is N or CR³;
R¹, R², R³, and the remaining groups in Formula (VIII) are as defined in the first aspect of the present invention.

In another preferred embodiment, the formula (I) is formula (IXa), formula (IXb), formula (IXc):

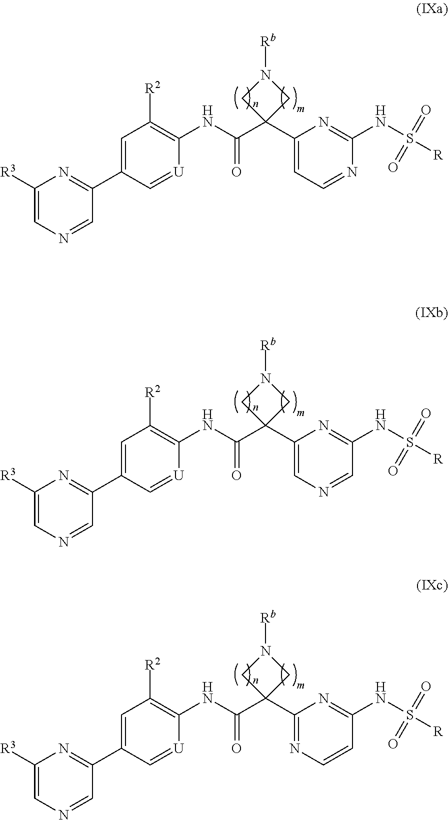

U is N or CH;
the remaining groups in Formula (IXa), Formula (IXb), and Formula (IXc) and the provisos are as defined in the first aspect of the present invention.

In another preferred embodiment, the Formula (I) is Formula (Xa) or Formula (Xb):

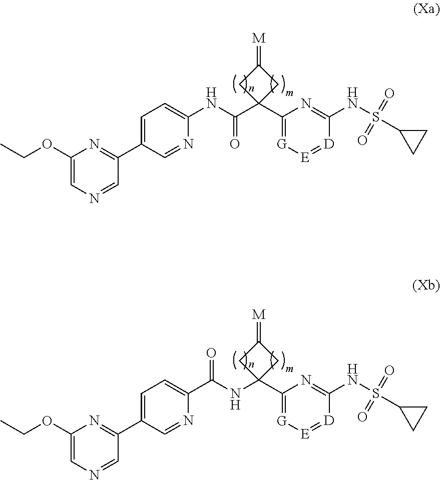

D, E, and G are each independently selected from N or CH, provided that no more than two of D, E, and G are N;
M is selected from CR^{h}Rⁱ; wherein R^{h} and Rⁱ are each independently selected from hydrogen, halogen, C₁₋₄ alkyl, C₃₋₆ cycloalkyl, 3- to 8-membered heterocyclyl, aryl, heteroaryl, CN, C(O)R^{g}, C(O)OR^{f}, or C(O)NR^{d}R^{d}; wherein said alkyl, cycloalkyl, heterocyclyl, aryl, and heteroaryl are each optionally substituted by one or more substituents independently selected from the group consisting of halogen, C₁₋₄ alkyl, C₂₋₄ alkenyl, C₂₋₄ alkynyl, C₃₋₆ cycloalkyl, 3- to 6-membered heterocyclyl, aryl, heteroaryl, CN, OR^{f}, SR^{f}, NR^{d}R^{d}, C(O)R^{g}, C(O)OR^{f}, OC(O)R^{g}, C(O)NR^{d}R^{d}, NR^{d}C(O)R^{g}, NR^{d}C(O)NR^{d}R^{d}, OC(O)NR^{d}R^{d}, NR^{d}(O)OR^{f}, OC(O)OR^{f}, S(O)₂NR^{d}R^{d}, NR^{d}S(O)₂R^{g} and NR^{d}S(O)₂NR^{d}R^{d}; or
R^{h} and Rⁱ, together with the carbon atom to which they are attached, form a 3- to 8-membered cyclic structure, said cyclic structure optionally containing 0, 1 or 2 heteroatoms independently selected from N, O and S, and optionally substituted by one or more substituents independently selected from the group consisting of: halogen, C₁₋₄ alkyl, CN, OR^{f}, SR^{f}, NR^{d}R^{d} and =M, wherein M is as defined above;
each R^{d} is independently selected from hydrogen, C₁₋₄ alkyl, C₁₋₄ haloalkyl, C₁₋₄ alkoxy-C₂₋₄ alkyl, C₃₋₆ cycloalkyl, or 3- to 6-membered heterocyclyl; each Rf is independently selected from hydrogen, C₁₋₄ alkyl, C₁₋₄ haloalkyl, C₃₋₆ cycloalkyl, or 3- to 6-membered heterocyclyl; each R^{g} is independently selected from hydrogen, C₁₋₄ alkyl, C₁₋₄ haloalkyl, C₂₋₄ alkenyl, C₂₋₄ alkynyl, C₃₋₆ cycloalkyl, 3- to 6-membered heterocyclyl, aryl, or heteroaryl;
m and n are each independently selected from 0, 1, 2, 3, 4, 5, or 6.

In another preferred embodiment, the formula (I) is formula (XIa), formula (XIb), formula (XIc), formula (XId), formula (XIe), or formula (XIf):

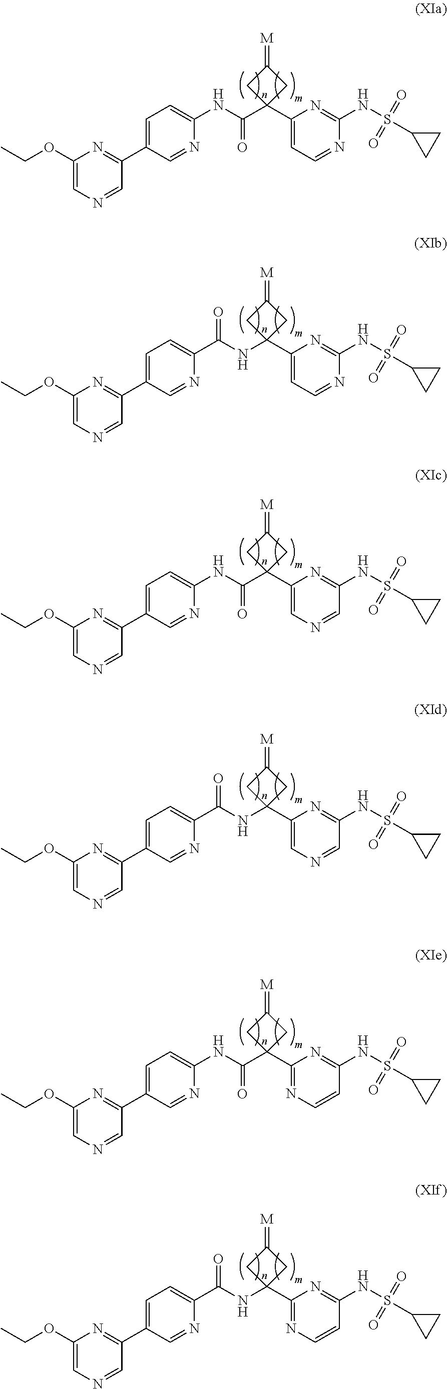

wherein M is selected from CR^{h}Rⁱ; wherein R^{h} and Rⁱ are each independently selected from hydrogen, halogen, C₁₋₄ alkyl, C₃₋₆ cycloalkyl, 3- to 8-membered heterocyclyl, aryl, heteroaryl, CN, C(O)R^{g}, C(O)OR^{f}, or C(O)NR^{d}R^{d};
wherein said alkyl, cycloalkyl, heterocyclyl, aryl, and heteroaryl are each optionally substituted by one or more substituents selected from the group consisting of: halogen, C₁₋₄ alkyl, C₂₋₄ alkenyl, C₂₋₄ alkynyl, C₃₋₆ cycloalkyl, 3- to 6-membered heterocyclyl, aryl, heteroaryl, CN, OR^{f}, SR^{f}, NR^{d}R^{d}, C(O)R^{g}, C(O)OR^{f}, OC(O)R^{g}, C(O)NR^{d}R^{d}, NR^{d}C(O)R^{g}, NR^{d}C(O)NR^{d}R^{d}, OC(O)NR^{d}R^{d}, NR^{d}C(O)OR^{f}, OC(O)OR^{f}, S(O)₂NR^{d}R^{d}, NR^{d}S(O)₂R^{g}, and NR^{d}S(O)₂NR^{d}R^{d};
or R^{h} and Rⁱ together with the carbon atom to which they are attached form a 3- to 8-membered cyclic structure, said cyclic structure optionally containing 0, 1 or 2 heteroatoms selected from N, O, and S, and optionally substituted by one or more substituents selected from the group consisting of halogen, C₁₋₄ alkyl, CN, OR^{f}, SR^{f}, NR^{d}R^{d} and =M, wherein M is as defined above; wherein each R^{d} is independently selected from hydrogen, C₁₋₄ alkyl, C₁₋₄ haloalkyl, C₁₋₄ alkoxy-C₂₋₄ alkyl, C₃₋₆ cycloalkyl, or 3- to 6-membered heterocyclyl; each Rf is independently selected from hydrogen, C₁₋₄ alkyl, C₁₋₄ haloalkyl, C₃₋₆ cycloalkyl, or 3- to 6-membered heterocyclyl; each R^{g} is independently selected from hydrogen, C₁₋₄ alkyl, C₁₋₄ haloalkyl, C₂₋₄ alkenyl, C₂₋₄ alkynyl, C₃₋₆ cycloalkyl, 3- to 6-membered heterocyclyl, aryl, or heteroaryl;
m and n are each independently selected from 0, 1, 2, 3, 4, 5 and 6.

In another preferred embodiment, the M is CR^{h}Rⁱ; wherein R^{h} and Rⁱ are each independently selected from hydrogen, halogen, C₁₋₄ alkyl, C₃₋₆ cycloalkyl, 3- to 6-membered heterocyclyl, aryl, heteroaryl, CN, C(O)R^{g}, C(O)OR^{f}, and C(O)NR^{d}R^{d}; wherein said alkyl, cycloalkyl, heterocyclyl, aryl, and heteroaryl are each optionally substituted by one or more substituents selected from the group consisting of halogen, C₁₋₄ alkyl, C₂₋₄ alkenyl, C₂₋₄ alkynyl, C₃₋₆ cycloalkyl, 3- to 6-membered heterocyclyl, CN, OR^{f}, NR^{d}C(O)R^{g}, and NR^{d}S(O)₂R^{g};
wherein R^{d}, R^{f}, and R^{g} are as defined as any of the above;
and wherein in and n are each independently 1 or 2.

In another preferred embodiment, the formula (I) is formula (XIIa) or formula (XIIb):

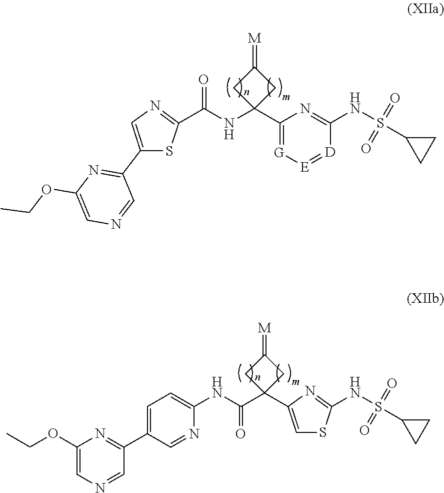

D, E, and G are each independently selected from N or CH, provided that no more than two of D, E, and G are N;
M, in and n are as defined in any of the above.

In another preferred embodiment, the compound is selected from the group consisting of

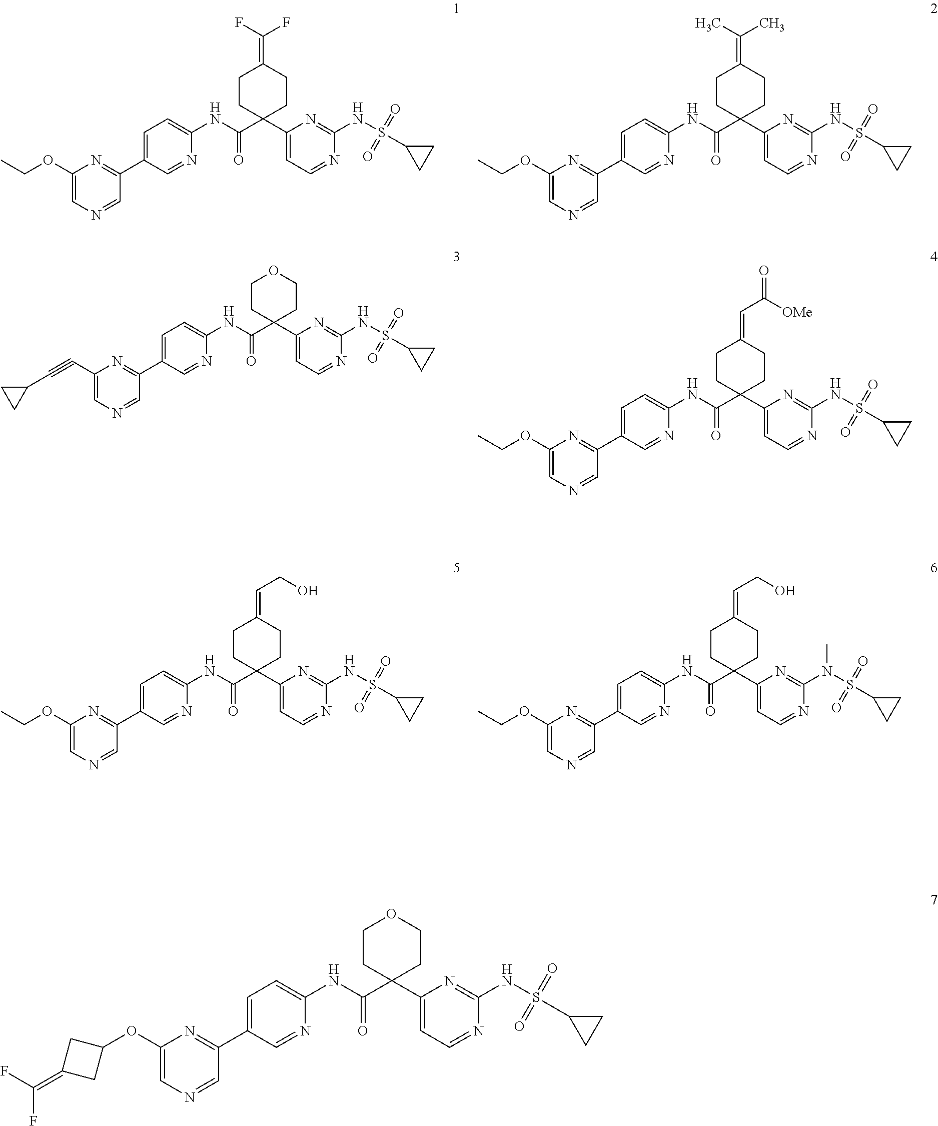

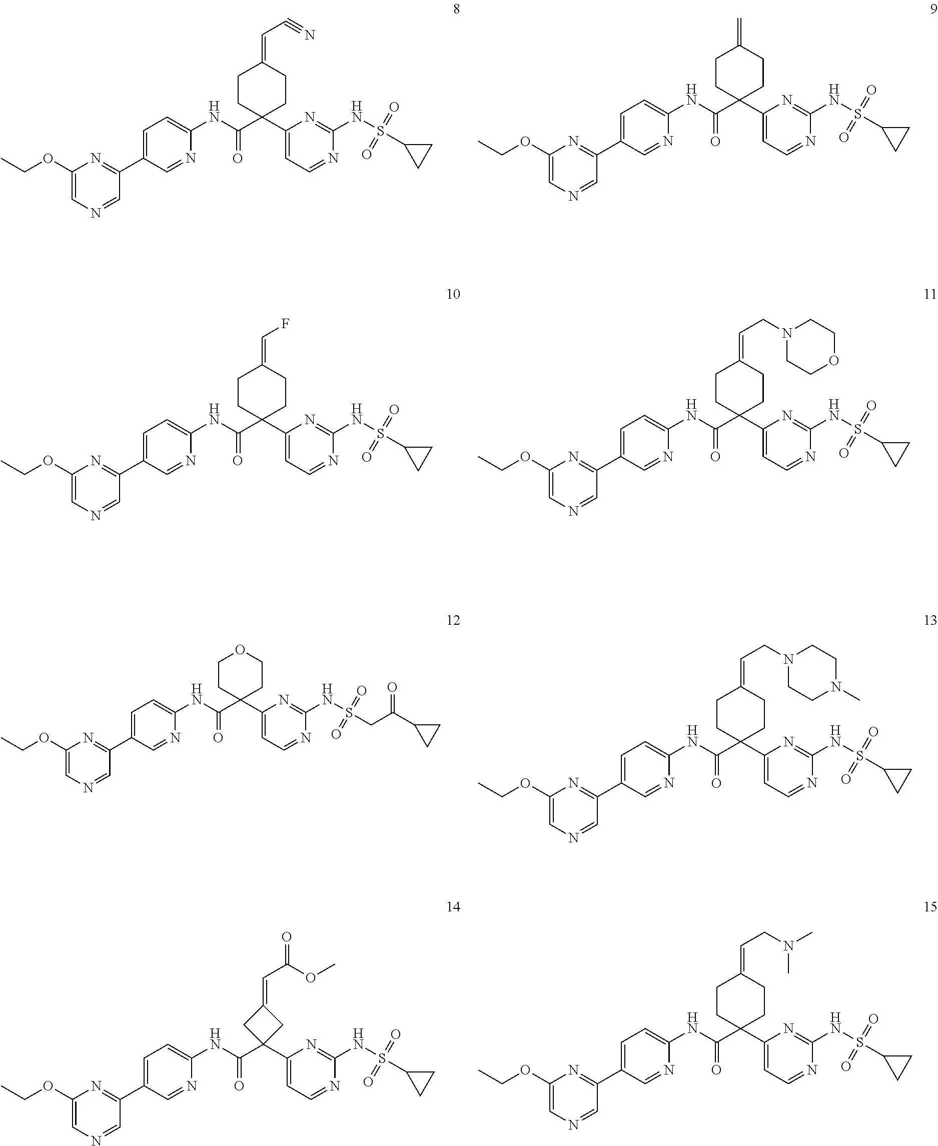

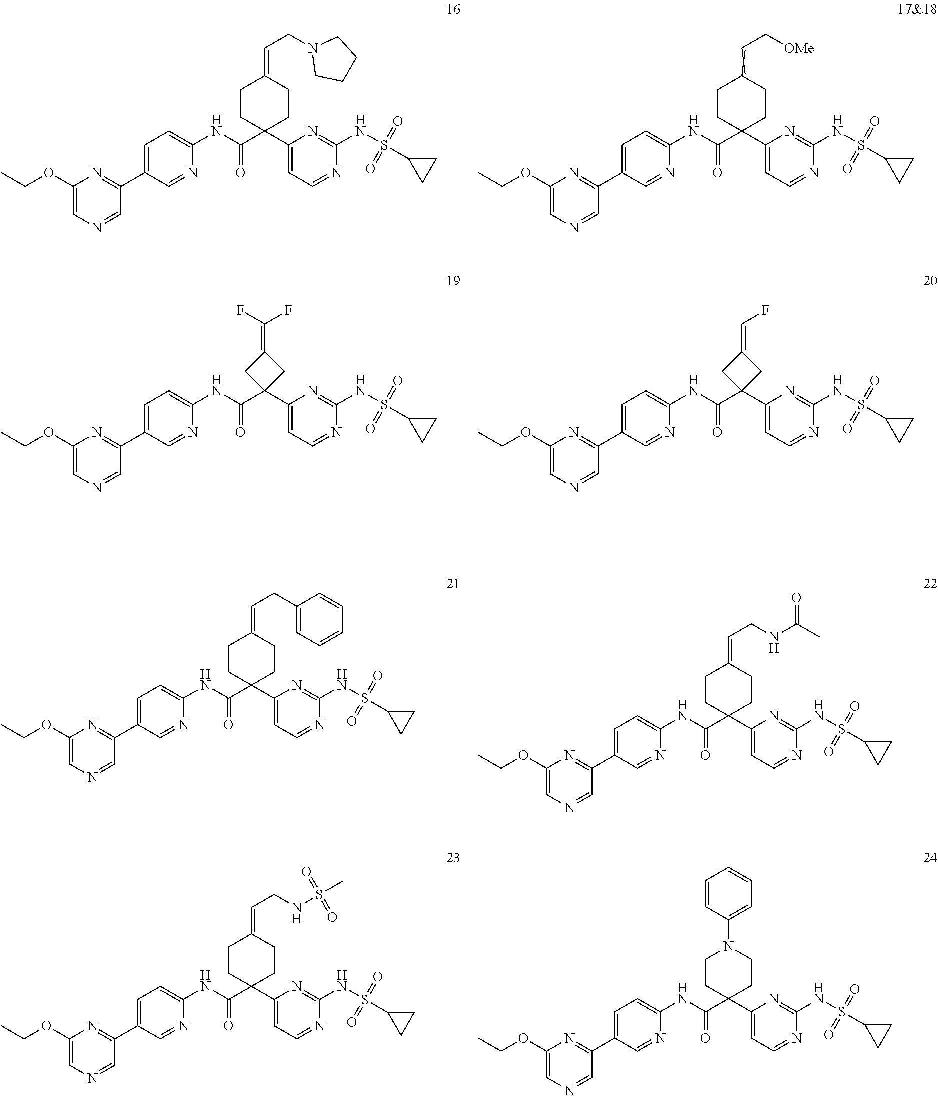

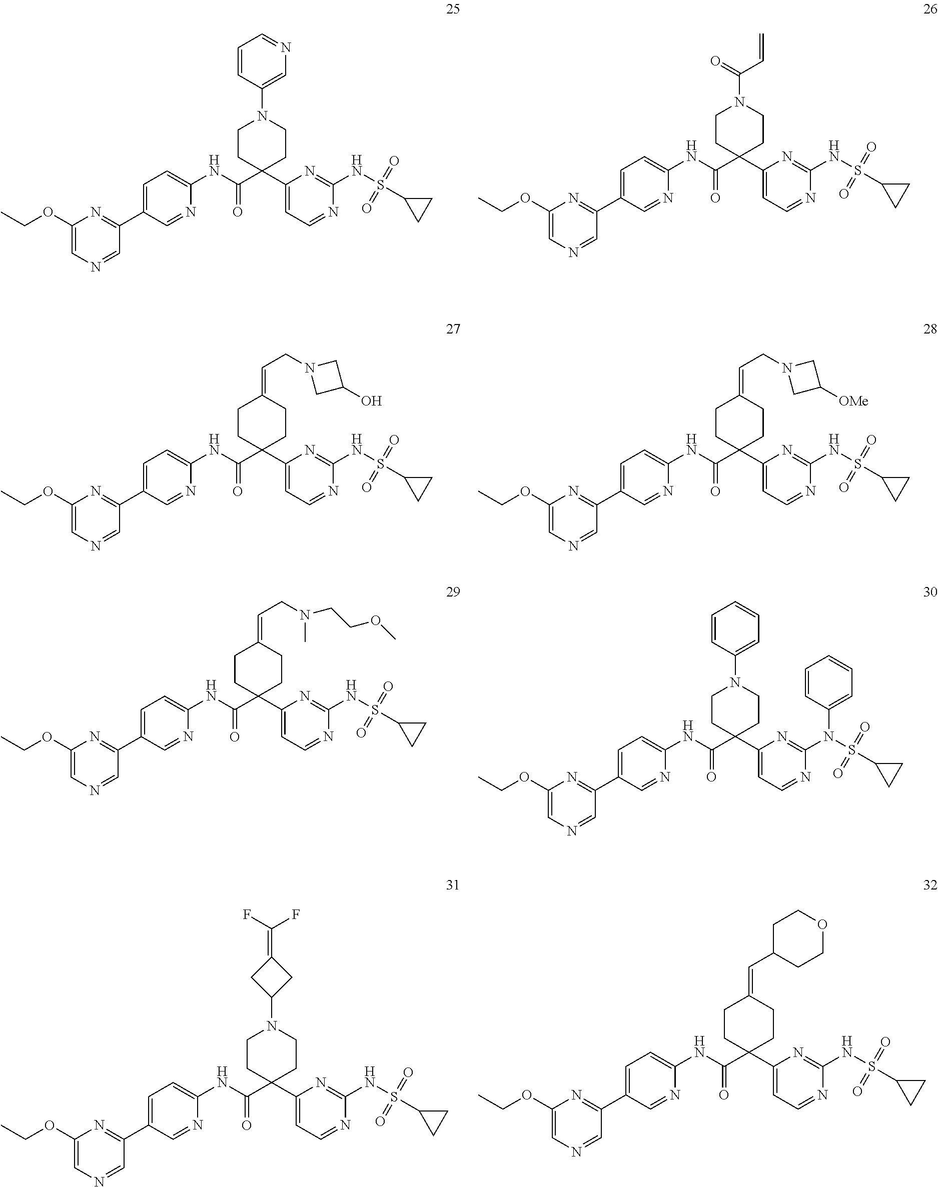

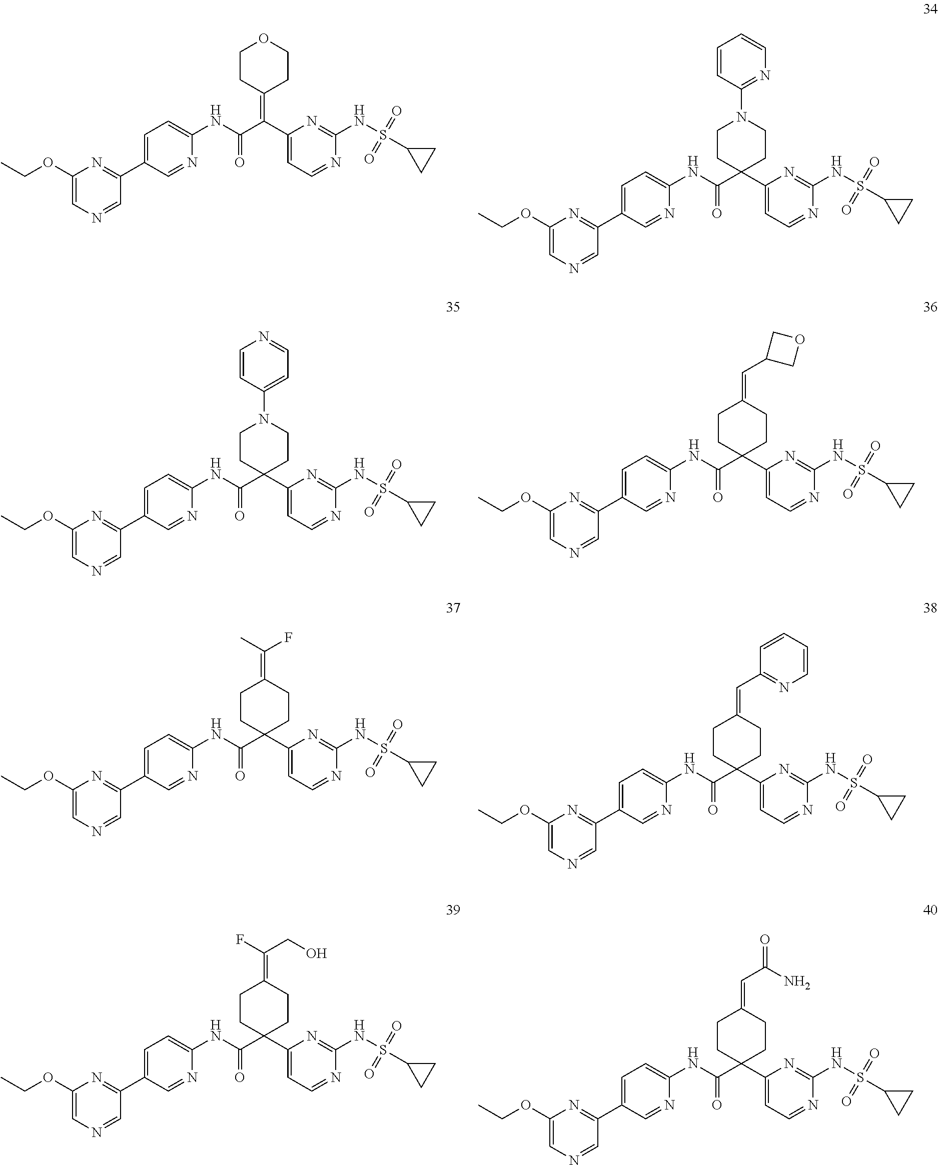

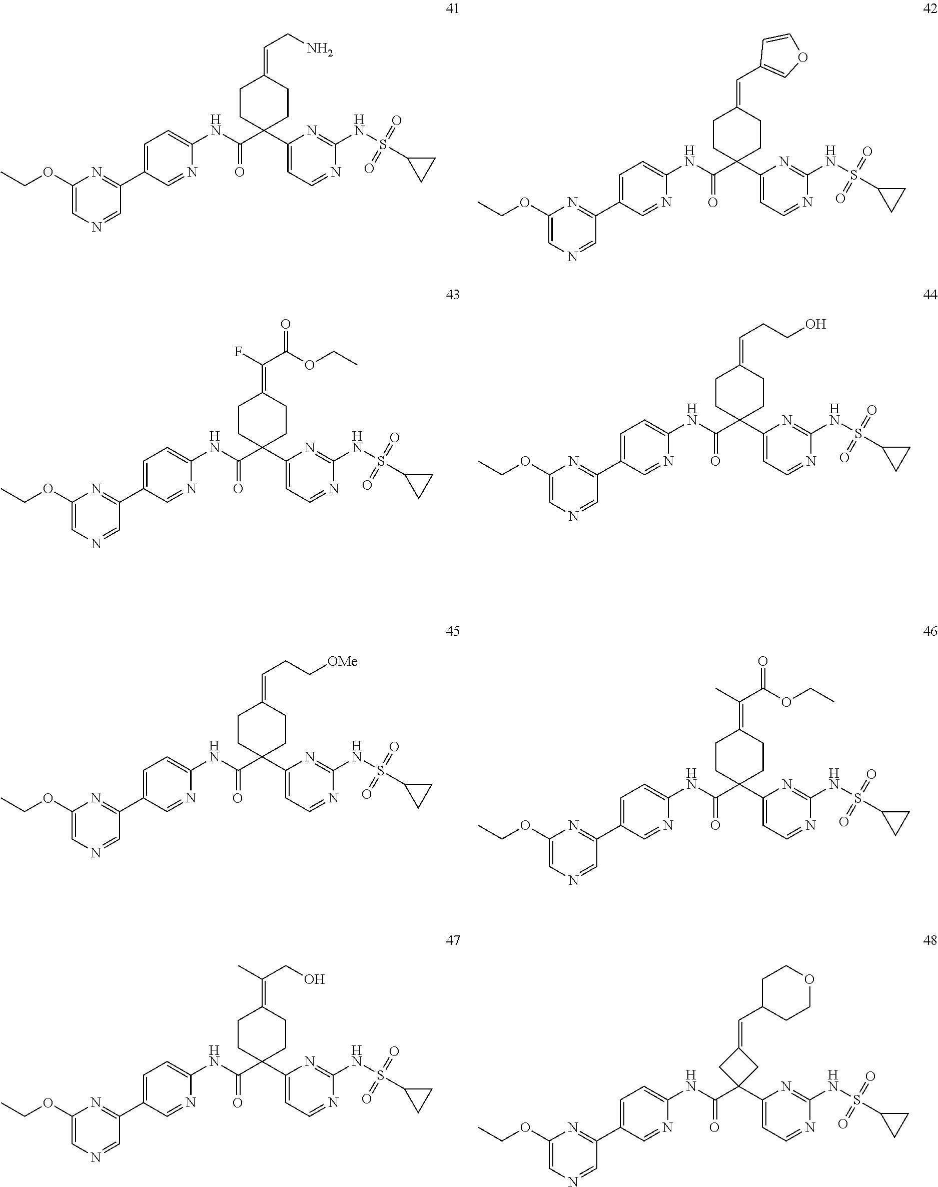

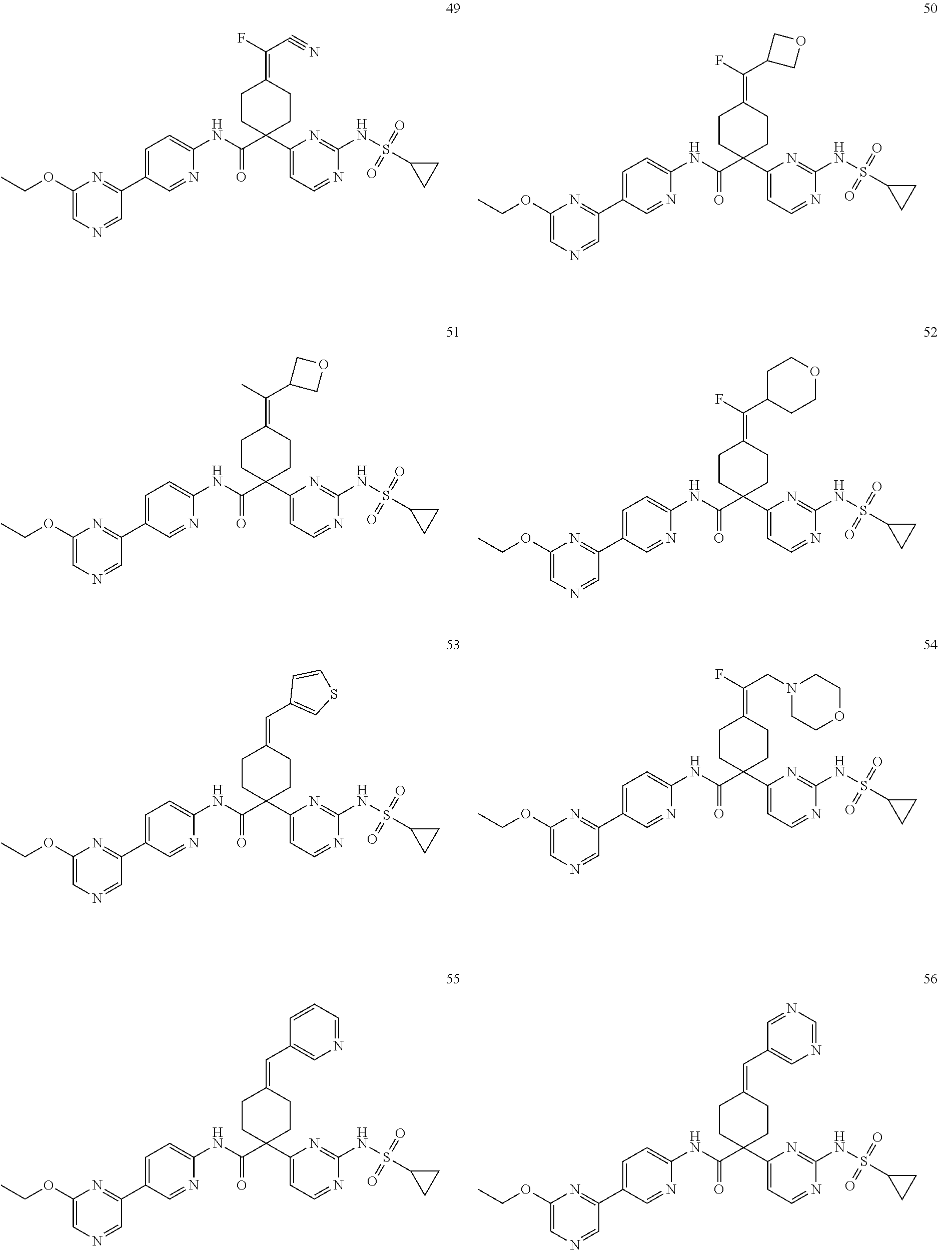

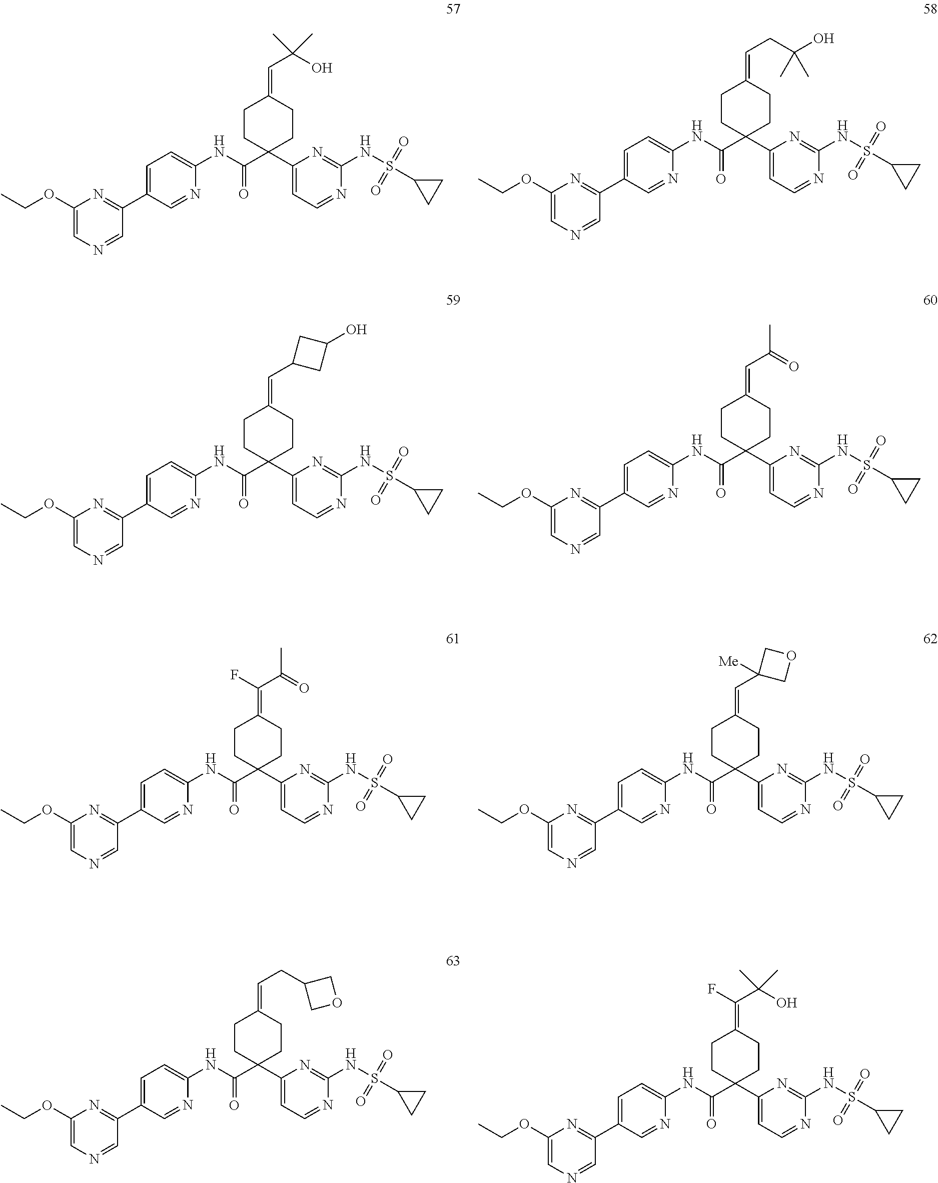

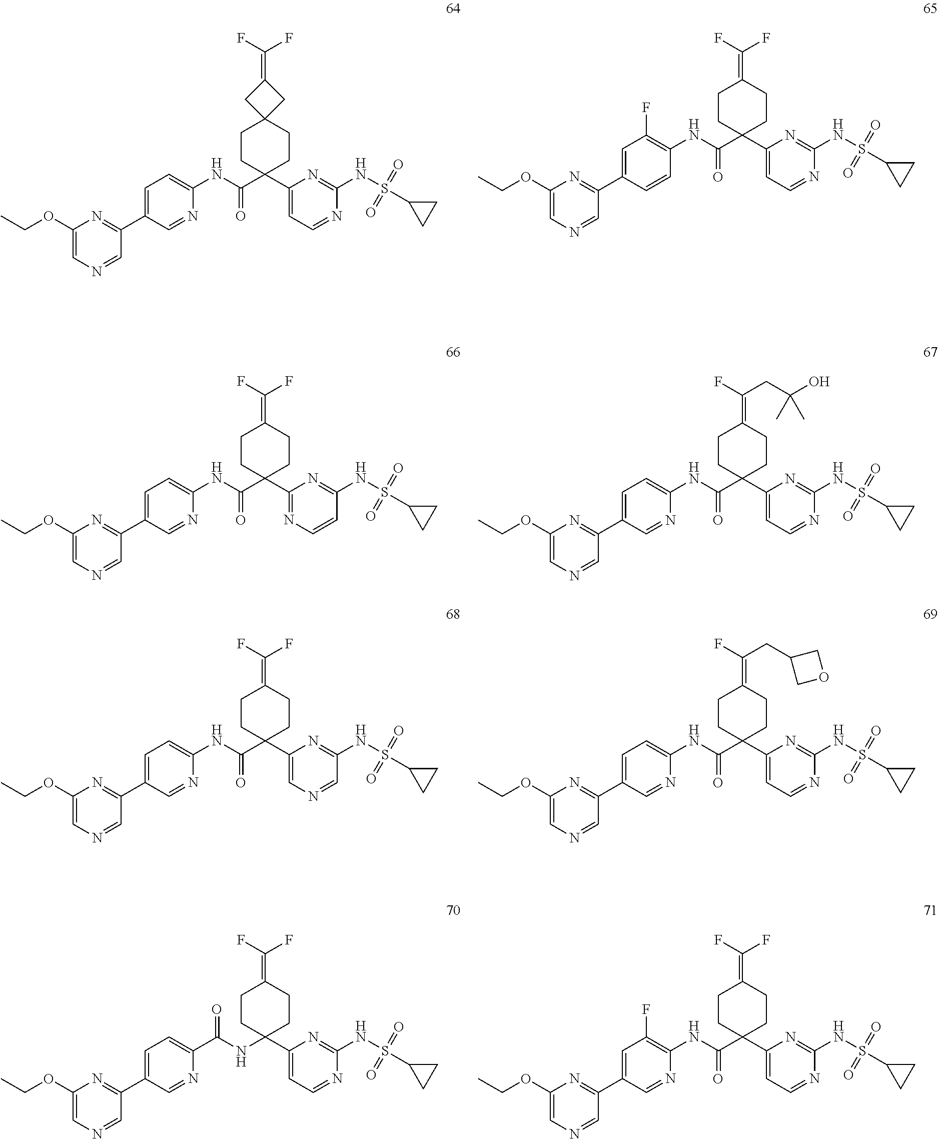

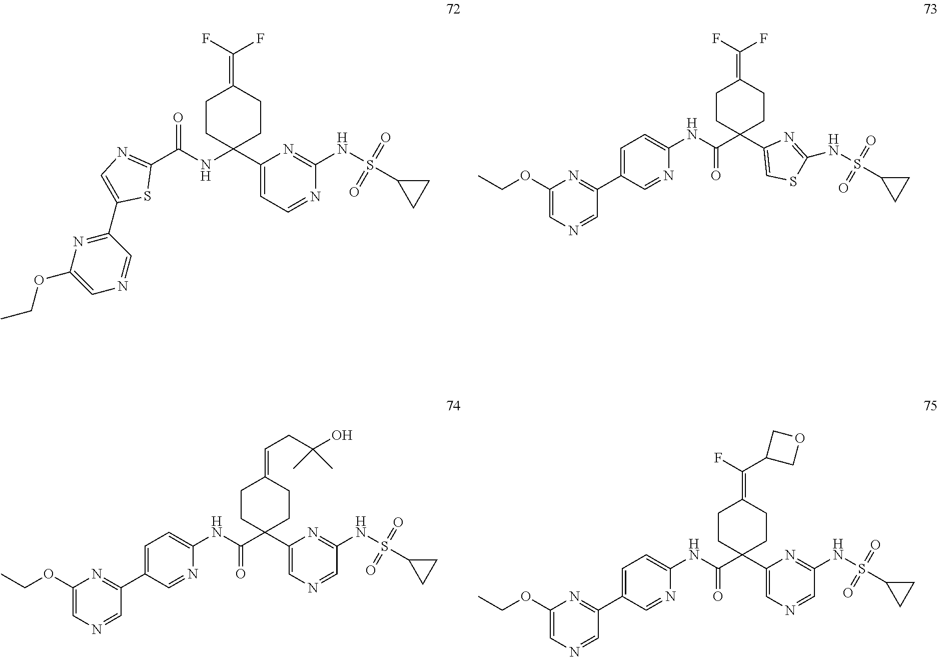 [SMILES: CCOc1cncc(-c2cnc(C(=O)NC3(c4ccnc(NS(=O)(=O)C5CC5)n4)CCC(=C(F)F)CC3)s2)n1 ; CCOc1cncc(-c2ccc(NC(=O)C3(c4csc(NS(=O)(=O)C5CC5)n4)CCC(=C(F)F)CC3)nc2)n1 ; CCOc1cncc(-c2ccc(NC(=O)C3(c4cncc(NS(=O)(=O)C5CC5)n4)CCC(=CCC(C)(C)O)CC3)nc2)n1 ; CCOc1cncc(-c2ccc(NC(=O)C3(c4cncc(NS(=O)(=O)C5CC5)n4)CCC(=C(F)C4COC4)CC3)nc2)n1]

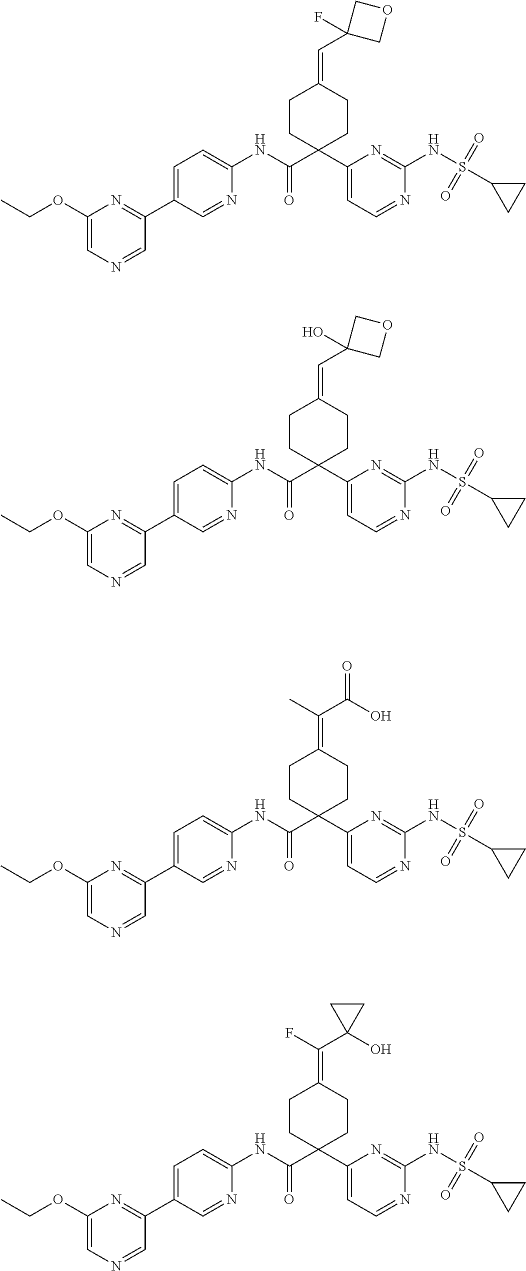 [SMILES: CCOc1cncc(-c2ccc(NC(=O)C3(c4ccnc(NS(=O)(=O)C5CC5)n4)CCC(=CC4(F)COC4)CC3)nc2)n1 ; CCOc1cncc(-c2ccc(NC(=O)C3(c4ccnc(NS(=O)(=O)C5CC5)n4)CCC(=CC4(O)COC4)CC3)nc2)n1 ; CCOc1cncc(-c2ccc(NC(=O)C3(c4ccnc(NS(=O)(=O)C5CC5)n4)CCC(=C(C)C(=O)O)CC3)nc2)n1 ; CCOc1cncc(-c2ccc(NC(=O)C3(c4ccnc(NS(=O)(=O)C5CC5)n4)CCC(=C(F)C4(O)CC4)CC3)nc2)n1]

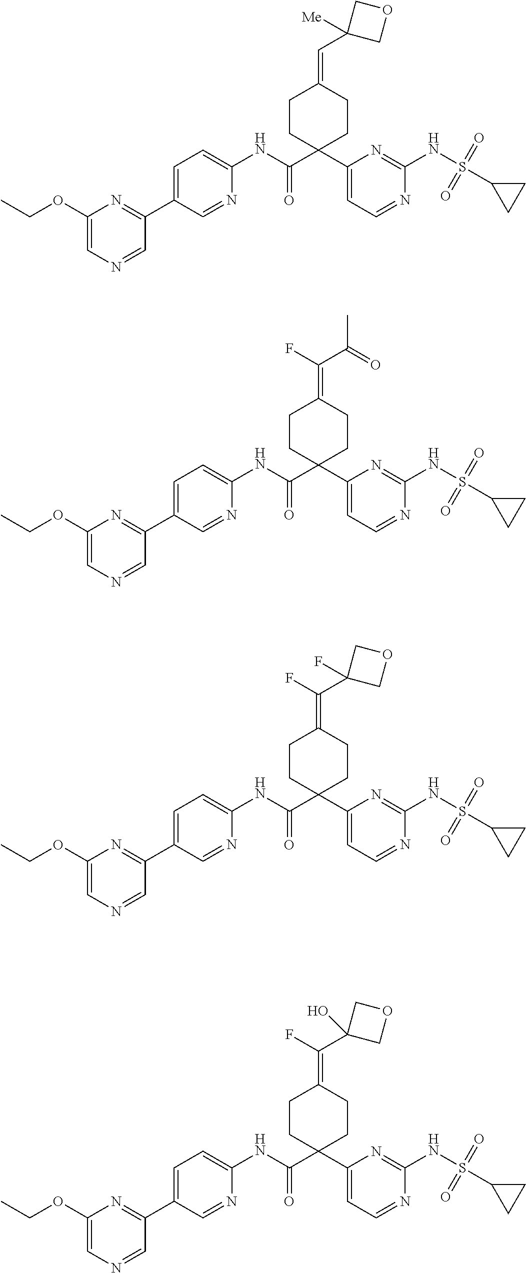 [SMILES: CCOc1cncc(-c2ccc(NC(=O)C3(c4ccnc(NS(=O)(=O)C5CC5)n4)CCC(=CC4(C)COC4)CC3)nc2)n1 ; CCOc1cncc(-c2ccc(NC(=O)C3(c4ccnc(NS(=O)(=O)C5CC5)n4)CCC(=C(F)C(C)=O)CC3)nc2)n1 ; CCOc1cncc(-c2ccc(NC(=O)C3(c4ccnc(NS(=O)(=O)C5CC5)n4)CCC(=C(F)C4(F)COC4)CC3)nc2)n1 ; CCOc1cncc(-c2ccc(NC(=O)C3(c4ccnc(NS(=O)(=O)C5CC5)n4)CCC(=C(F)C4(O)COC4)CC3)nc2)n1]

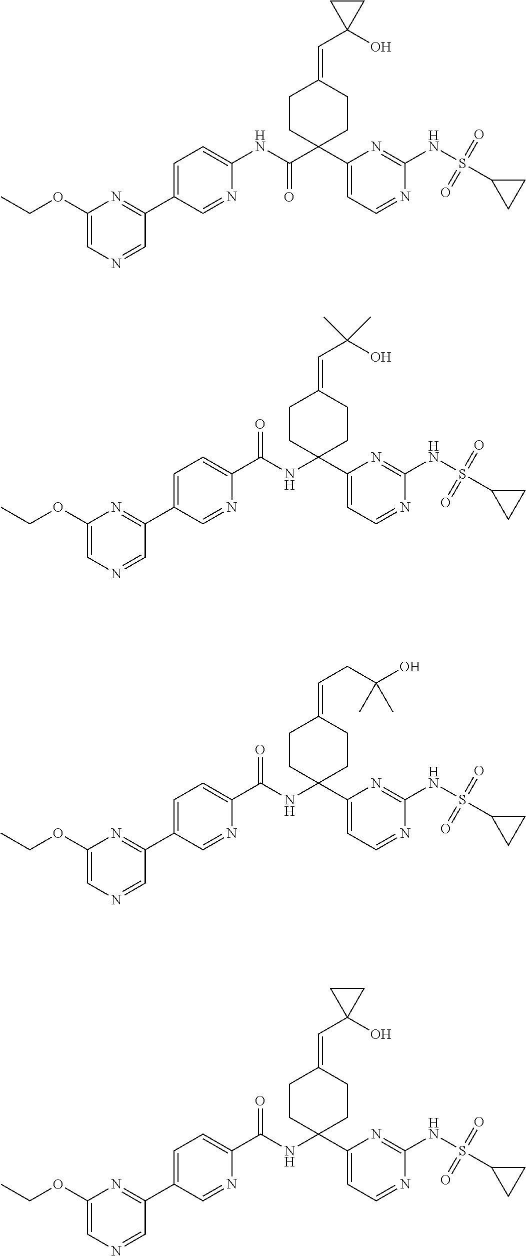 [SMILES: CCOc1cncc(-c2ccc(NC(=O)C3(c4ccnc(NS(=O)(=O)C5CC5)n4)CCC(=CC4(O)CC4)CC3)nc2)n1 ; CCOc1cncc(-c2ccc(C(=O)NC3(c4ccnc(NS(=O)(=O)C5CC5)n4)CCC(=CC(C)(C)O)CC3)nc2)n1 ; CCOc1cncc(-c2ccc(C(=O)NC3(c4ccnc(NS(=O)(=O)C5CC5)n4)CCC(=CCC(C)(C)O)CC3)nc2)n1 ; CCOc1cncc(-c2ccc(C(=O)NC3(c4ccnc(NS(=O)(=O)C5CC5)n4)CCC(=CC4(O)CC4)CC3)nc2)n1]

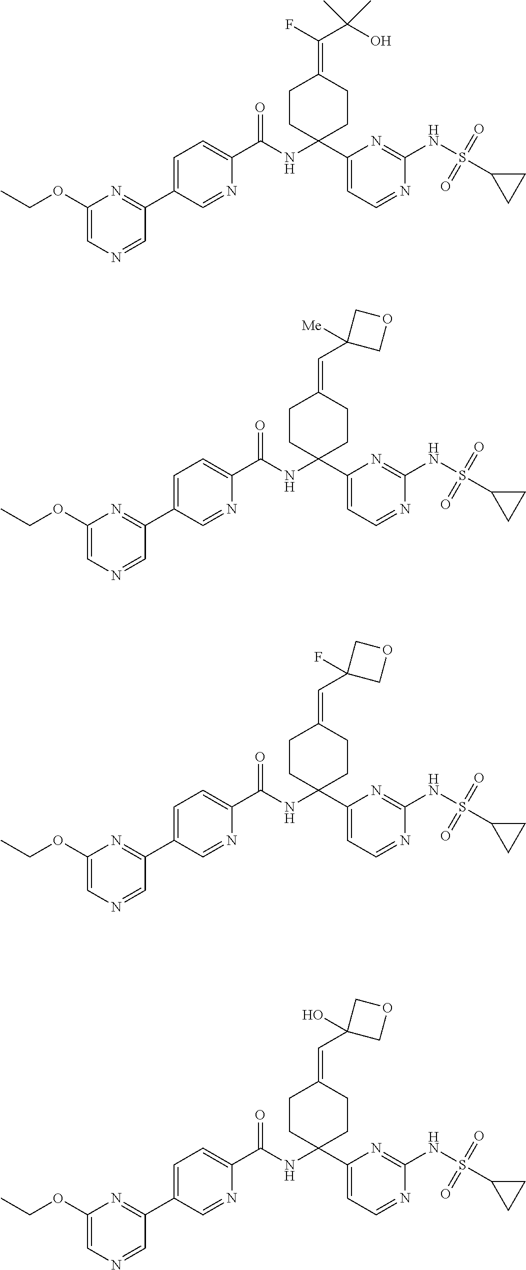 [SMILES: CCOc1cncc(-c2ccc(C(=O)NC3(c4ccnc(NS(=O)(=O)C5CC5)n4)CCC(=C(F)C(C)(C)O)CC3)nc2)n1 ; CCOc1cncc(-c2ccc(C(=O)NC3(c4ccnc(NS(=O)(=O)C5CC5)n4)CCC(=CC4(C)COC4)CC3)nc2)n1 ; CCOc1cncc(-c2ccc(C(=O)NC3(c4ccnc(NS(=O)(=O)C5CC5)n4)CCC(=CC4(F)COC4)CC3)nc2)n1 ; CCOc1cncc(-c2ccc(C(=O)NC3(c4ccnc(NS(=O)(=O)C5CC5)n4)CCC(=CC4(O)COC4)CC3)nc2)n1]

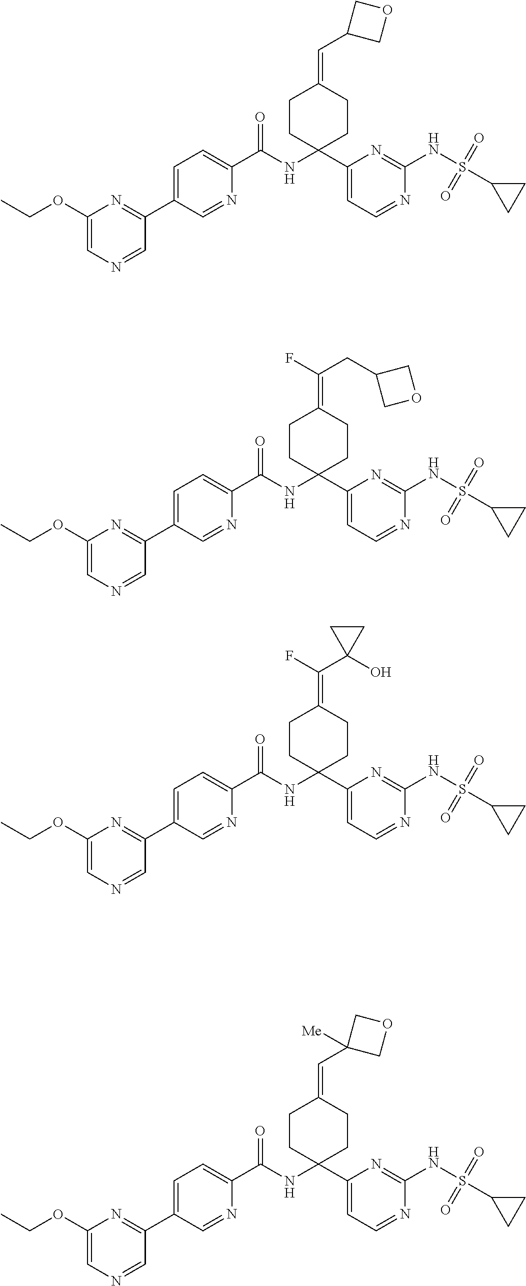 [SMILES: CCOc1cncc(-c2ccc(C(=O)NC3(c4ccnc(NS(=O)(=O)C5CC5)n4)CCC(=CC4COC4)CC3)nc2)n1 ; CCOc1cncc(-c2ccc(C(=O)NC3(c4ccnc(NS(=O)(=O)C5CC5)n4)CCC(=C(F)CC4COC4)CC3)nc2)n1 ; CCOc1cncc(-c2ccc(C(=O)NC3(c4ccnc(NS(=O)(=O)C5CC5)n4)CCC(=C(F)C4(O)CC4)CC3)nc2)n1 ; CCOc1cncc(-c2ccc(C(=O)NC3(c4ccnc(NS(=O)(=O)C5CC5)n4)CCC(=CC4(C)COC4)CC3)nc2)n1]

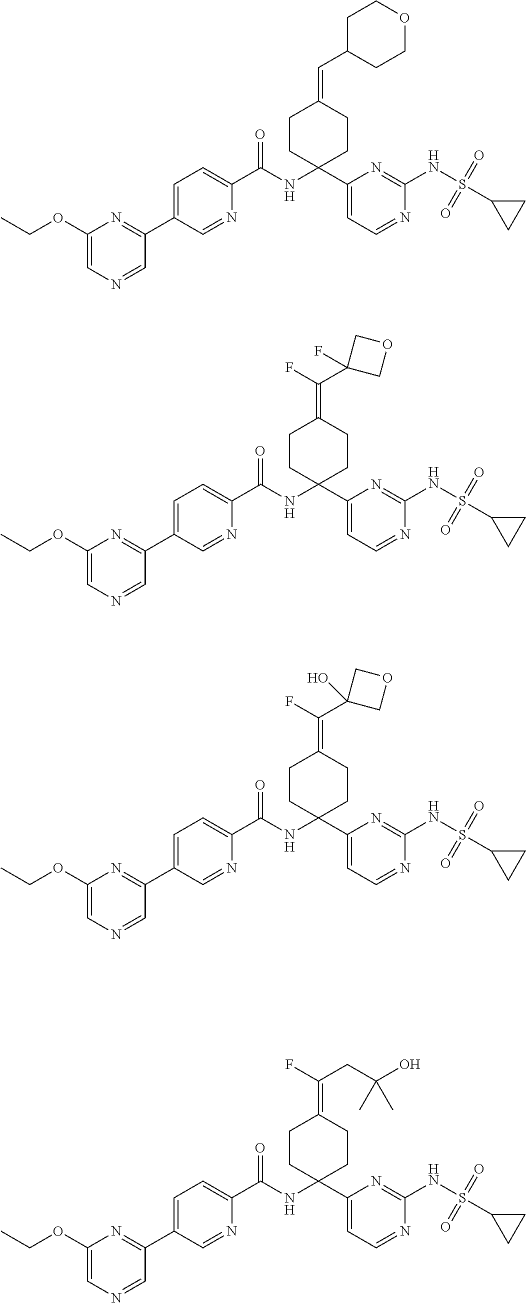 [SMILES: CCOc1cncc(-c2ccc(C(=O)NC3(c4ccnc(NS(=O)(=O)C5CC5)n4)CCC(=CC4CCOCC4)CC3)nc2)n1 ; CCOc1cncc(-c2ccc(C(=O)NC3(c4ccnc(NS(=O)(=O)C5CC5)n4)CCC(=C(F)C4(F)COC4)CC3)nc2)n1 ; CCOc1cncc(-c2ccc(C(=O)NC3(c4ccnc(NS(=O)(=O)C5CC5)n4)CCC(=C(F)C4(O)COC4)CC3)nc2)n1 ; CCOc1cncc(-c2ccc(C(=O)NC3(c4ccnc(NS(=O)(=O)C5CC5)n4)CCC(=C(F)CC(C)(C)O)CC3)nc2)n1]

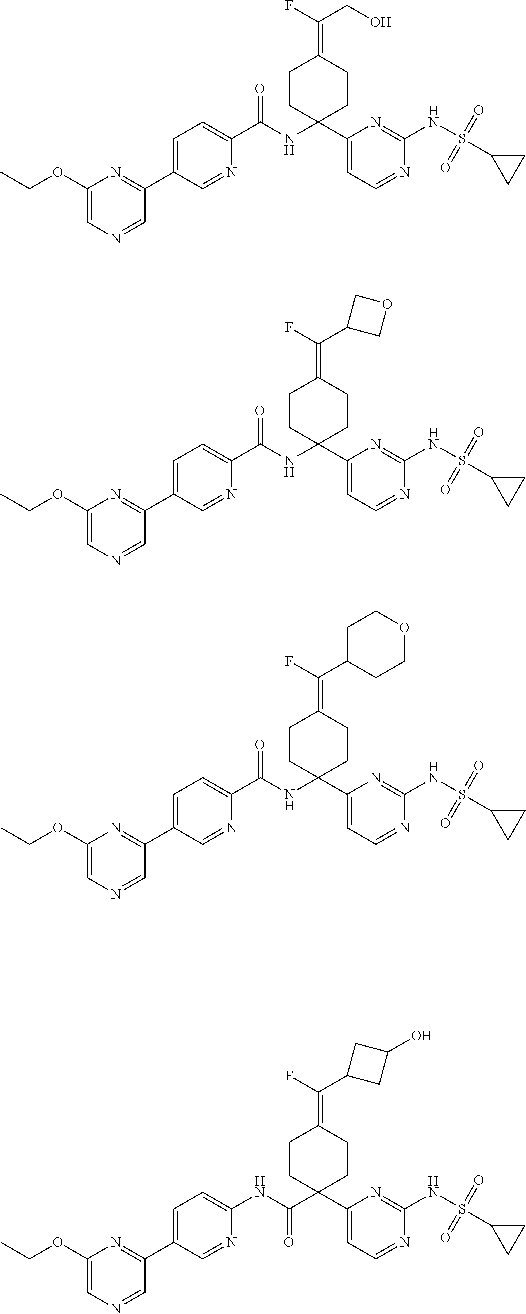 [SMILES: CCOc1cncc(-c2ccc(C(=O)NC3(c4ccnc(NS(=O)(=O)C5CC5)n4)CCC(=C(F)CO)CC3)nc2)n1 ; CCOc1cncc(-c2ccc(C(=O)NC3(c4ccnc(NS(=O)(=O)C5CC5)n4)CCC(=C(F)C4COC4)CC3)nc2)n1 ; CCOc1cncc(-c2ccc(C(=O)NC3(c4ccnc(NS(=O)(=O)C5CC5)n4)CCC(=C(F)C4CCOCC4)CC3)nc2)n1 ; CCOc1cncc(-c2ccc(NC(=O)C3(c4ccnc(NS(=O)(=O)C5CC5)n4)CCC(=C(F)C4CC(O)C4)CC3)nc2)n1]

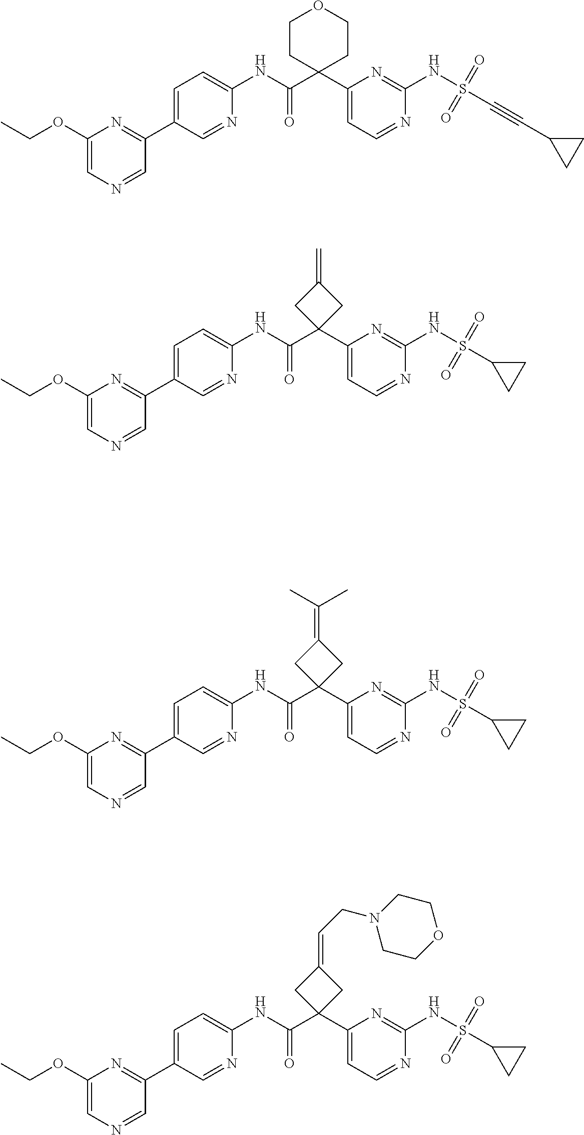 [SMILES: CCOc1cncc(-c2ccc(NC(=O)C3(c4ccnc(NS(=O)(=O)C#CC5CC5)n4)CCOCC3)nc2)n1 ; C=C1CC(C(=O)Nc2ccc(-c3cncc(OCC)n3)cn2)(c2ccnc(NS(=O)(=O)C3CC3)n2)C1 ; CCOc1cncc(-c2ccc(NC(=O)C3(c4ccnc(NS(=O)(=O)C5CC5)n4)CC(=C(C)C)C3)nc2)n1 ; CCOc1cncc(-c2ccc(NC(=O)C3(c4ccnc(NS(=O)(=O)C5CC5)n4)CC(=CCN4CCOCC4)C3)nc2)n1]

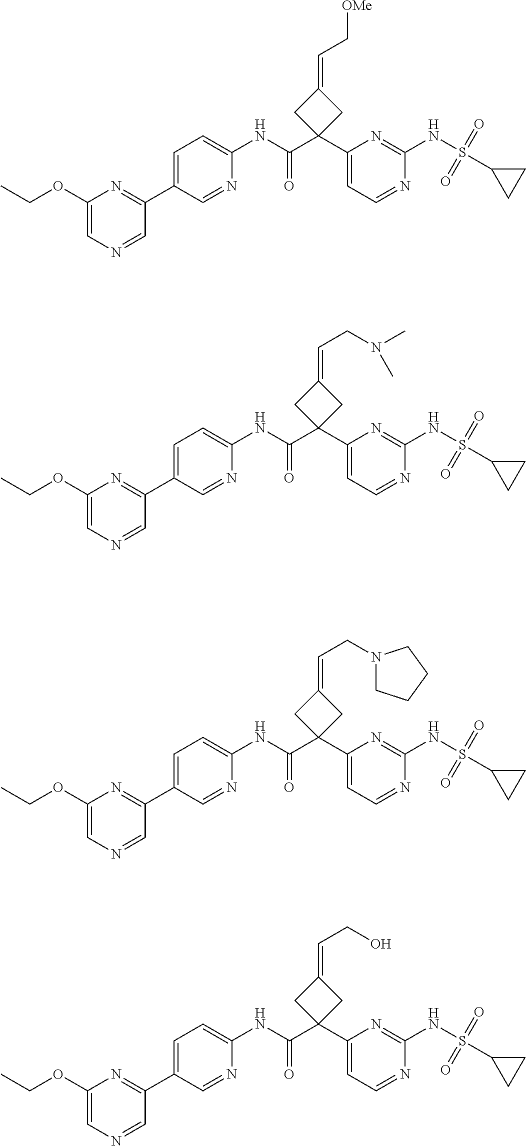 [SMILES: CCOc1cncc(-c2ccc(NC(=O)C3(c4ccnc(NS(=O)(=O)C5CC5)n4)CC(=CCOC)C3)nc2)n1 ; CCOc1cncc(-c2ccc(NC(=O)C3(c4ccnc(NS(=O)(=O)C5CC5)n4)CC(=CCN(C)C)C3)nc2)n1 ; CCOc1cncc(-c2ccc(NC(=O)C3(c4ccnc(NS(=O)(=O)C5CC5)n4)CC(=CCN4CCCC4)C3)nc2)n1 ; CCOc1cncc(-c2ccc(NC(=O)C3(c4ccnc(NS(=O)(=O)C5CC5)n4)CC(=CCO)C3)nc2)n1]

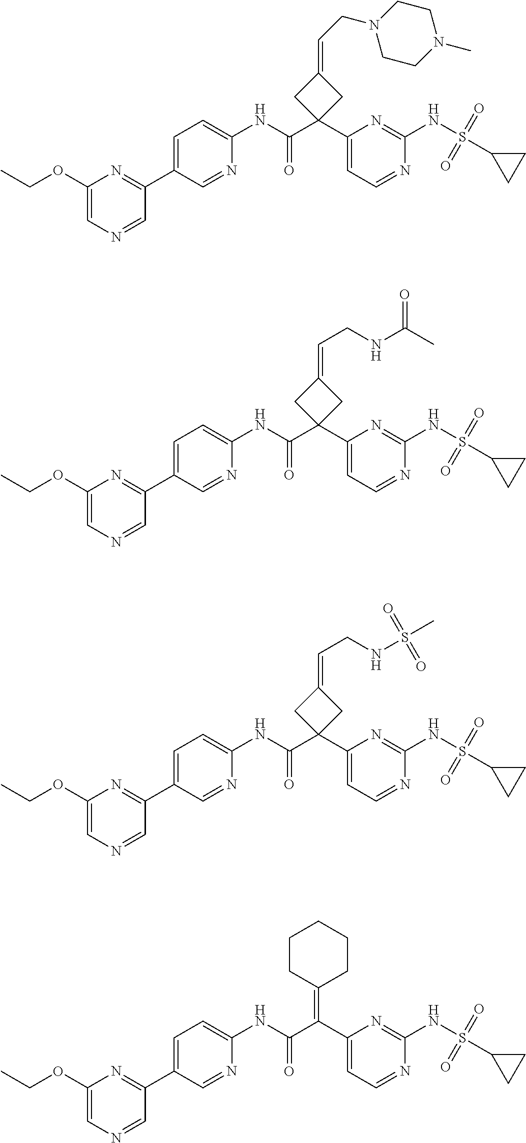 [SMILES: CCOc1cncc(-c2ccc(NC(=O)C3(c4ccnc(NS(=O)(=O)C5CC5)n4)CC(=CCN4CCN(C)CC4)C3)nc2)n1 ; CCOc1cncc(-c2ccc(NC(=O)C3(c4ccnc(NS(=O)(=O)C5CC5)n4)CC(=CCNC(C)=O)C3)nc2)n1 ; CCOc1cncc(-c2ccc(NC(=O)C3(c4ccnc(NS(=O)(=O)C5CC5)n4)CC(=CCNS(C)(=O)=O)C3)nc2)n1 ; CCOc1cncc(-c2ccc(NC(=O)C(=C3CCCCC3)c3ccnc(NS(=O)(=O)C4CC4)n3)nc2)n1]

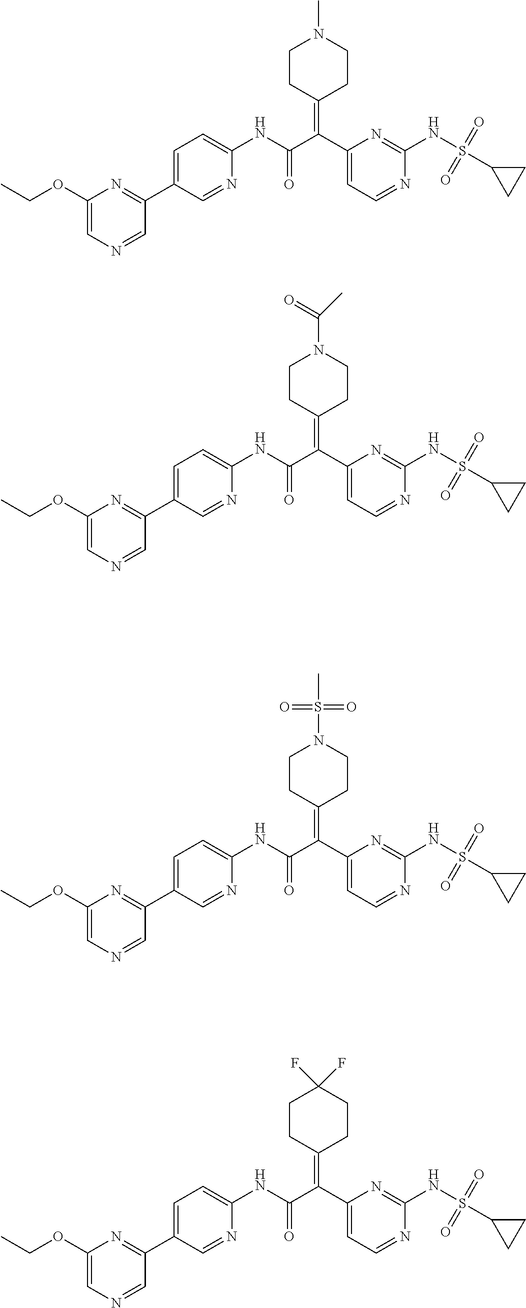 [SMILES: CCOc1cncc(-c2ccc(NC(=O)C(=C3CCN(C)CC3)c3ccnc(NS(=O)(=O)C4CC4)n3)nc2)n1 ; CCOc1cncc(-c2ccc(NC(=O)C(=C3CCN(C(C)=O)CC3)c3ccnc(NS(=O)(=O)C4CC4)n3)nc2)n1 ; CCOc1cncc(-c2ccc(NC(=O)C(=C3CCN(S(C)(=O)=O)CC3)c3ccnc(NS(=O)(=O)C4CC4)n3)nc2)n1 ; CCOc1cncc(-c2ccc(NC(=O)C(=C3CCC(F)(F)CC3)c3ccnc(NS(=O)(=O)C4CC4)n3)nc2)n1]

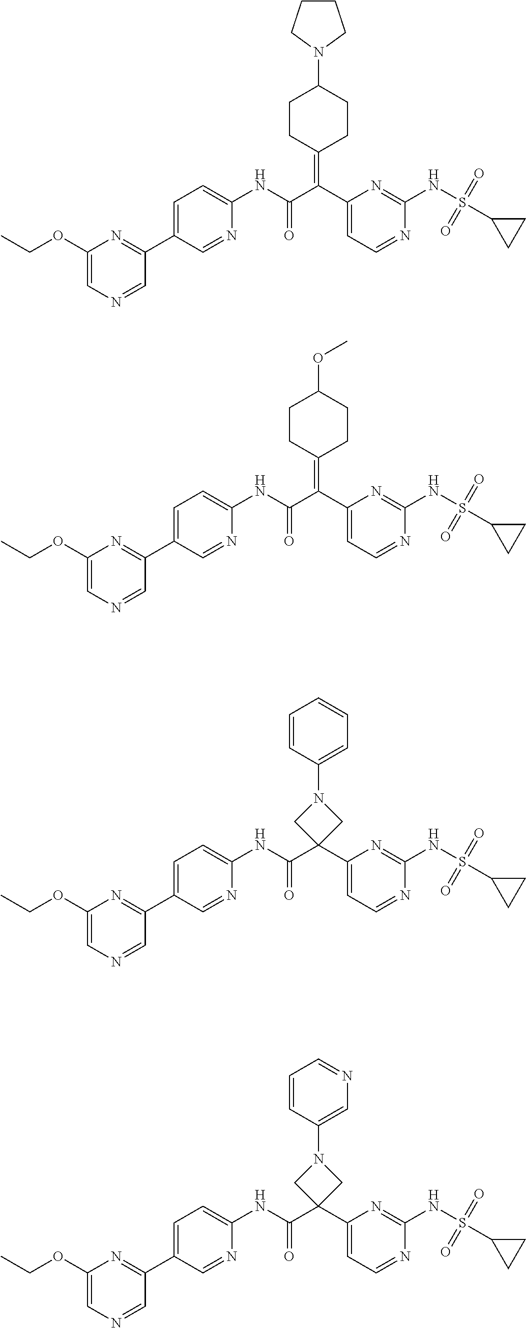 [SMILES: CCOc1cncc(-c2ccc(NC(=O)C(=C3CCC(N4CCCC4)CC3)c3ccnc(NS(=O)(=O)C4CC4)n3)nc2)n1 ; CCOc1cncc(-c2ccc(NC(=O)C(=C3CCC(OC)CC3)c3ccnc(NS(=O)(=O)C4CC4)n3)nc2)n1 ; CCOc1cncc(-c2ccc(NC(=O)C3(c4ccnc(NS(=O)(=O)C5CC5)n4)CN(c4ccccc4)C3)nc2)n1 ; CCOc1cncc(-c2ccc(NC(=O)C3(c4ccnc(NS(=O)(=O)C5CC5)n4)CN(c4cccnc4)C3)nc2)n1]

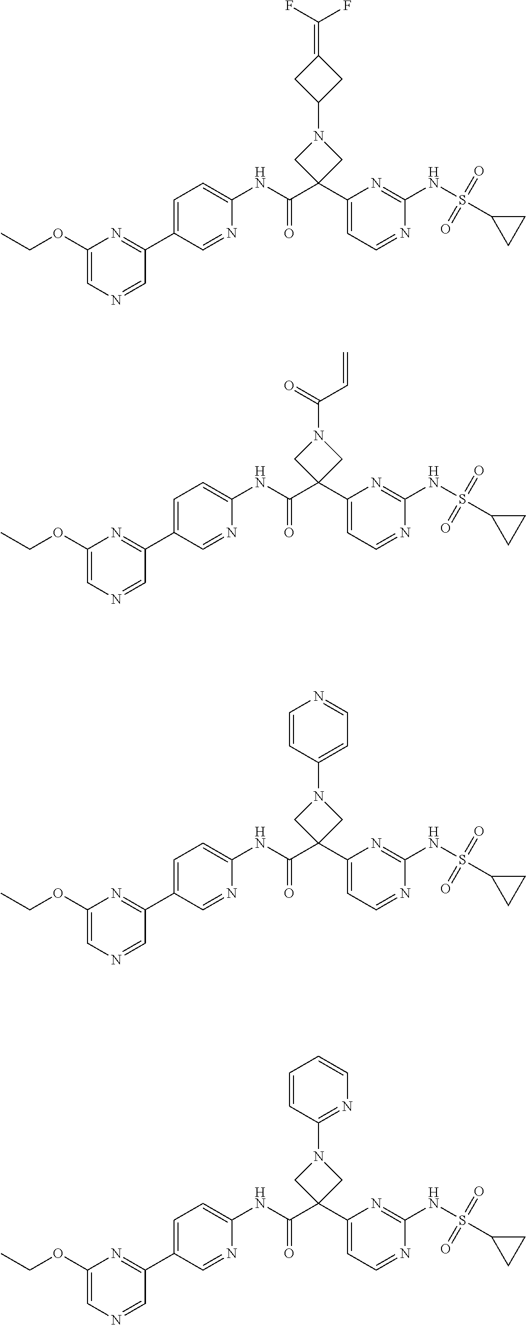 [SMILES: CCOc1cncc(-c2ccc(NC(=O)C3(c4ccnc(NS(=O)(=O)C5CC5)n4)CN(C4CC(=C(F)F)C4)C3)nc2)n1 ; C=CC(=O)N1CC(C(=O)Nc2ccc(-c3cncc(OCC)n3)cn2)(c2ccnc(NS(=O)(=O)C3CC3)n2)C1 ; CCOc1cncc(-c2ccc(NC(=O)C3(c4ccnc(NS(=O)(=O)C5CC5)n4)CN(c4ccncc4)C3)nc2)n1 ; CCOc1cncc(-c2ccc(NC(=O)C3(c4ccnc(NS(=O)(=O)C5CC5)n4)CN(c4ccccn4)C3)nc2)n1]

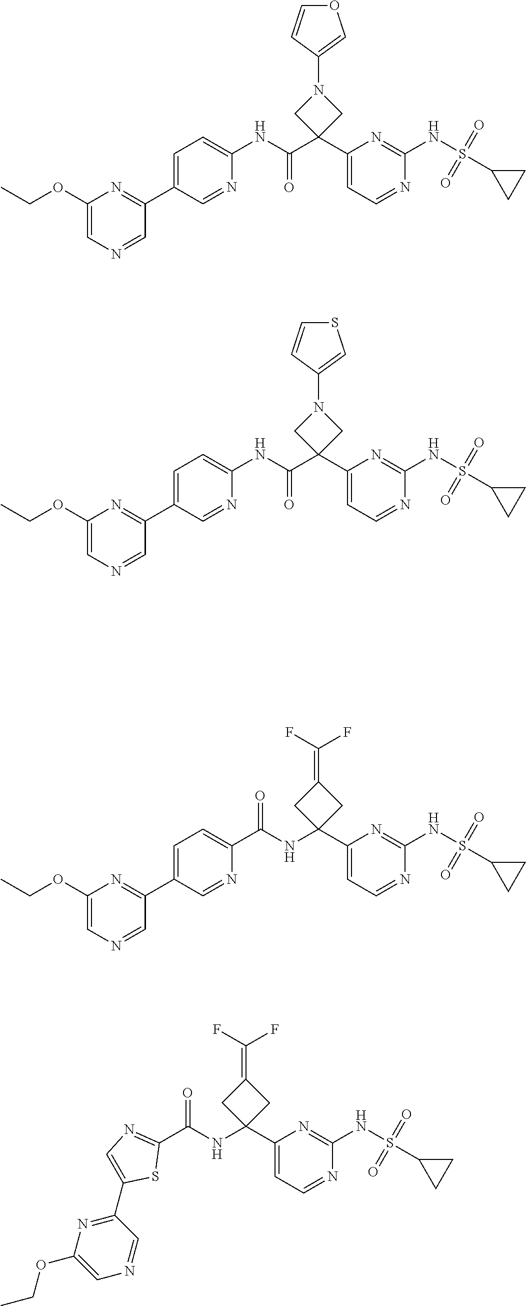 [SMILES: CCOc1cncc(-c2ccc(NC(=O)C3(c4ccnc(NS(=O)(=O)C5CC5)n4)CN(c4ccoc4)C3)nc2)n1 ; CCOc1cncc(-c2ccc(NC(=O)C3(c4ccnc(NS(=O)(=O)C5CC5)n4)CN(c4ccsc4)C3)nc2)n1 ; CCOc1cncc(-c2ccc(C(=O)NC3(c4ccnc(NS(=O)(=O)C5CC5)n4)CC(=C(F)F)C3)nc2)n1 ; CCOc1cncc(-c2cnc(C(=O)NC3(c4ccnc(NS(=O)(=O)C5CC5)n4)CC(=C(F)F)C3)s2)n1]

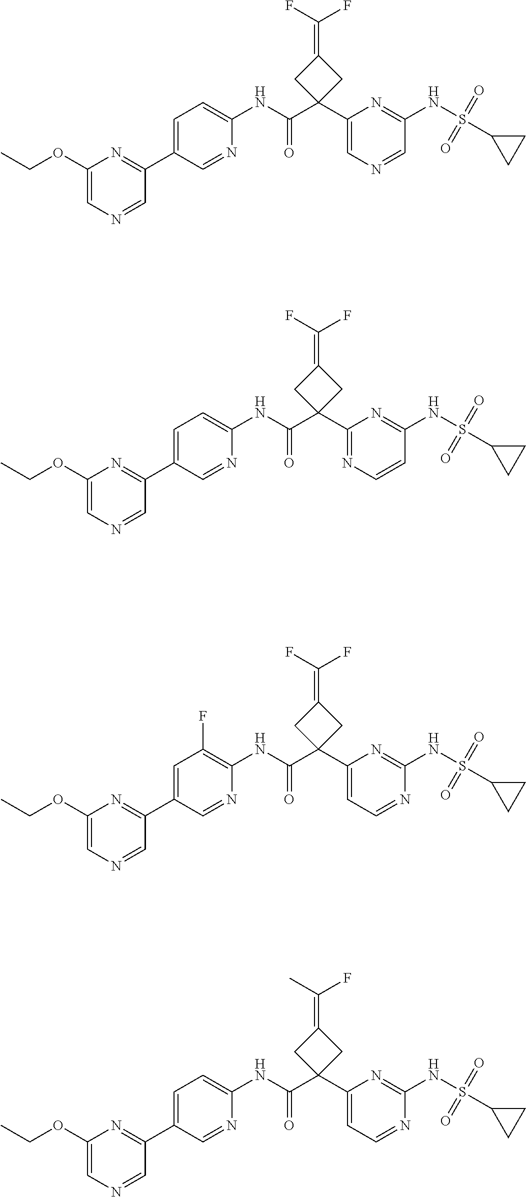 [SMILES: CCOc1cncc(-c2ccc(NC(=O)C3(c4cncc(NS(=O)(=O)C5CC5)n4)CC(=C(F)F)C3)nc2)n1 ; CCOc1cncc(-c2ccc(NC(=O)C3(c4nccc(NS(=O)(=O)C5CC5)n4)CC(=C(F)F)C3)nc2)n1 ; CCOc1cncc(-c2cnc(NC(=O)C3(c4ccnc(NS(=O)(=O)C5CC5)n4)CC(=C(F)F)C3)c(F)c2)n1 ; CCOc1cncc(-c2ccc(NC(=O)C3(c4ccnc(NS(=O)(=O)C5CC5)n4)CC(=C(C)F)C3)nc2)n1]

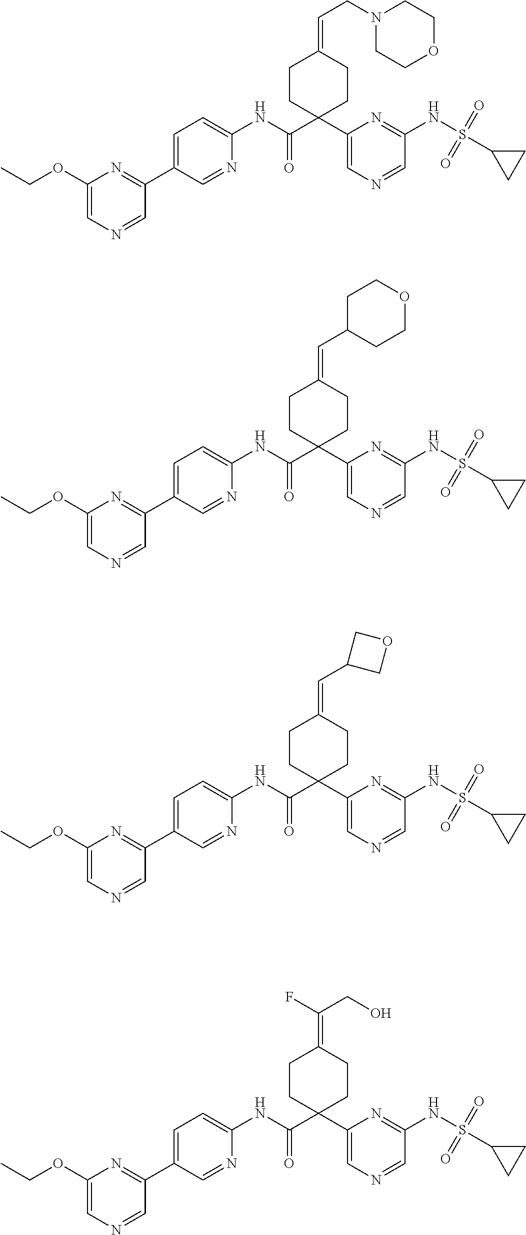 [SMILES: CCOc1cncc(-c2ccc(NC(=O)C3(c4cncc(NS(=O)(=O)C5CC5)n4)CCC(=CCN4CCOCC4)CC3)nc2)n1 ; CCOc1cncc(-c2ccc(NC(=O)C3(c4cncc(NS(=O)(=O)C5CC5)n4)CCC(=CC4CCOCC4)CC3)nc2)n1 ; CCOc1cncc(-c2ccc(NC(=O)C3(c4cncc(NS(=O)(=O)C5CC5)n4)CCC(=CC4COC4)CC3)nc2)n1 ; CCOc1cncc(-c2ccc(NC(=O)C3(c4cncc(NS(=O)(=O)C5CC5)n4)CCC(=C(F)CO)CC3)nc2)n1]

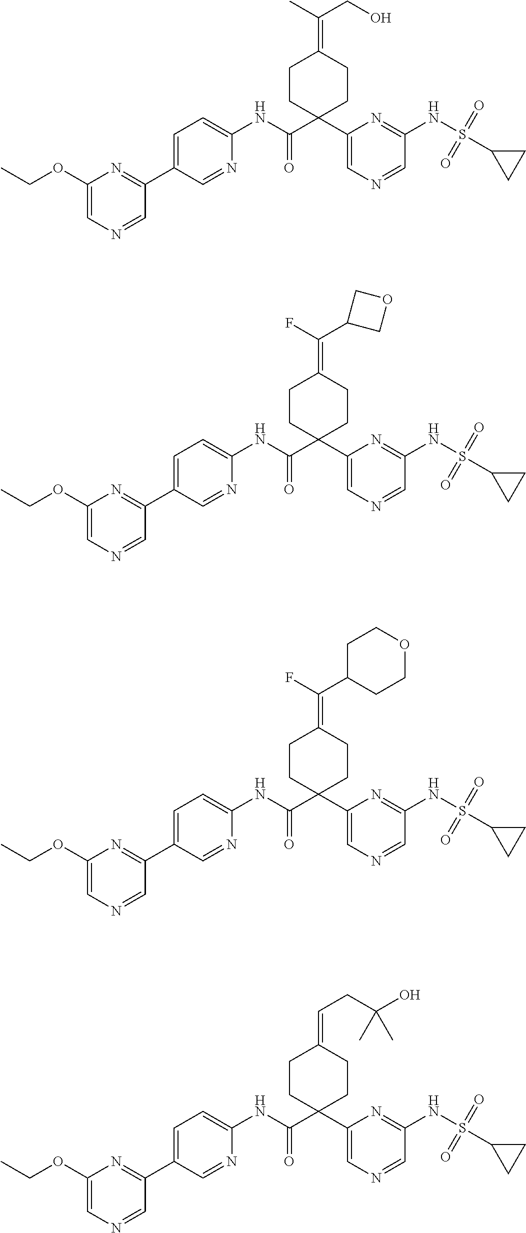 [SMILES: CCOc1cncc(-c2ccc(NC(=O)C3(c4cncc(NS(=O)(=O)C5CC5)n4)CCC(=C(C)CO)CC3)nc2)n1 ; CCOc1cncc(-c2ccc(NC(=O)C3(c4cncc(NS(=O)(=O)C5CC5)n4)CCC(=C(F)C4COC4)CC3)nc2)n1 ; CCOc1cncc(-c2ccc(NC(=O)C3(c4cncc(NS(=O)(=O)C5CC5)n4)CCC(=C(F)C4CCOCC4)CC3)nc2)n1 ; CCOc1cncc(-c2ccc(NC(=O)C3(c4cncc(NS(=O)(=O)C5CC5)n4)CCC(=CCC(C)(C)O)CC3)nc2)n1]

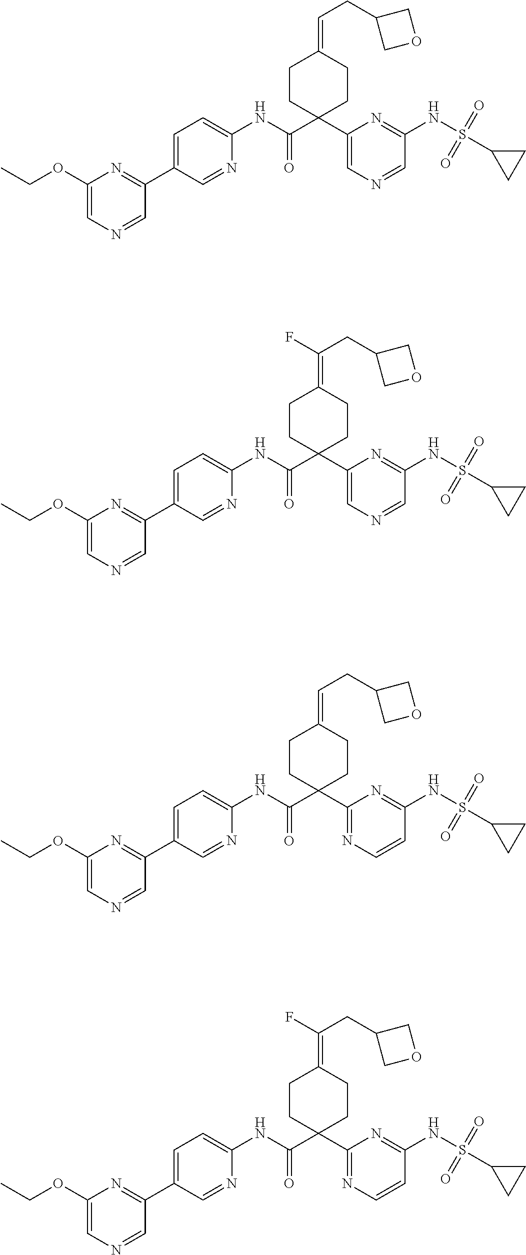 [SMILES: CCOc1cncc(-c2ccc(NC(=O)C3(c4cncc(NS(=O)(=O)C5CC5)n4)CCC(=CCC4COC4)CC3)nc2)n1 ; CCOc1cncc(-c2ccc(NC(=O)C3(c4cncc(NS(=O)(=O)C5CC5)n4)CCC(=C(F)CC4COC4)CC3)nc2)n1 ; CCOc1cncc(-c2ccc(NC(=O)C3(c4nccc(NS(=O)(=O)C5CC5)n4)CCC(=CCC4COC4)CC3)nc2)n1 ; CCOc1cncc(-c2ccc(NC(=O)C3(c4nccc(NS(=O)(=O)C5CC5)n4)CCC(=C(F)CC4COC4)CC3)nc2)n1]

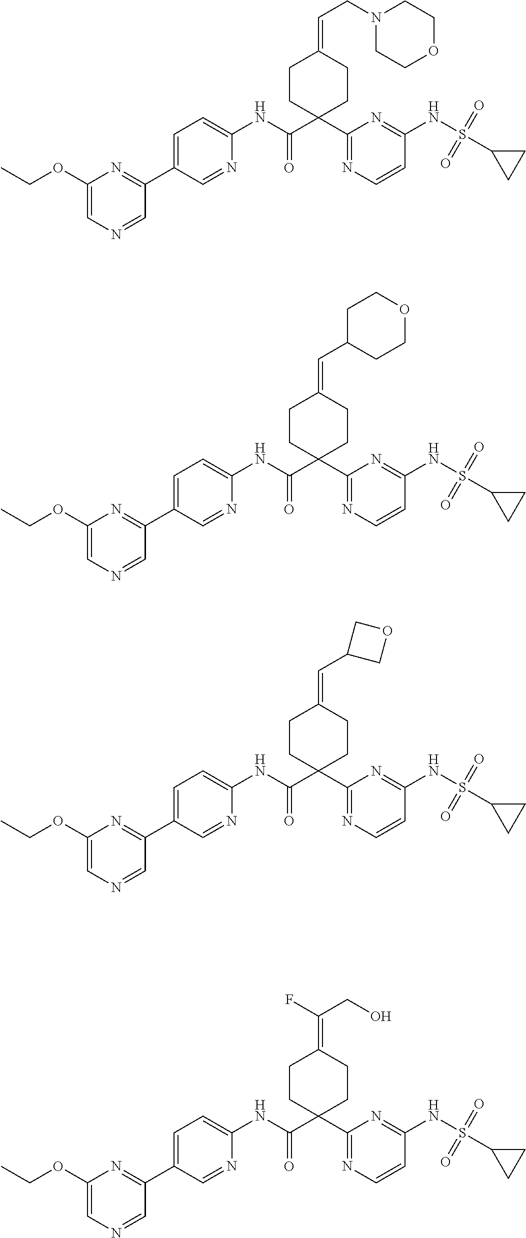 [SMILES: CCOc1cncc(-c2ccc(NC(=O)C3(c4nccc(NS(=O)(=O)C5CC5)n4)CCC(=CCN4CCOCC4)CC3)nc2)n1 ; CCOc1cncc(-c2ccc(NC(=O)C3(c4nccc(NS(=O)(=O)C5CC5)n4)CCC(=CC4CCOCC4)CC3)nc2)n1 ; CCOc1cncc(-c2ccc(NC(=O)C3(c4nccc(NS(=O)(=O)C5CC5)n4)CCC(=CC4COC4)CC3)nc2)n1 ; CCOc1cncc(-c2ccc(NC(=O)C3(c4nccc(NS(=O)(=O)C5CC5)n4)CCC(=C(F)CO)CC3)nc2)n1]

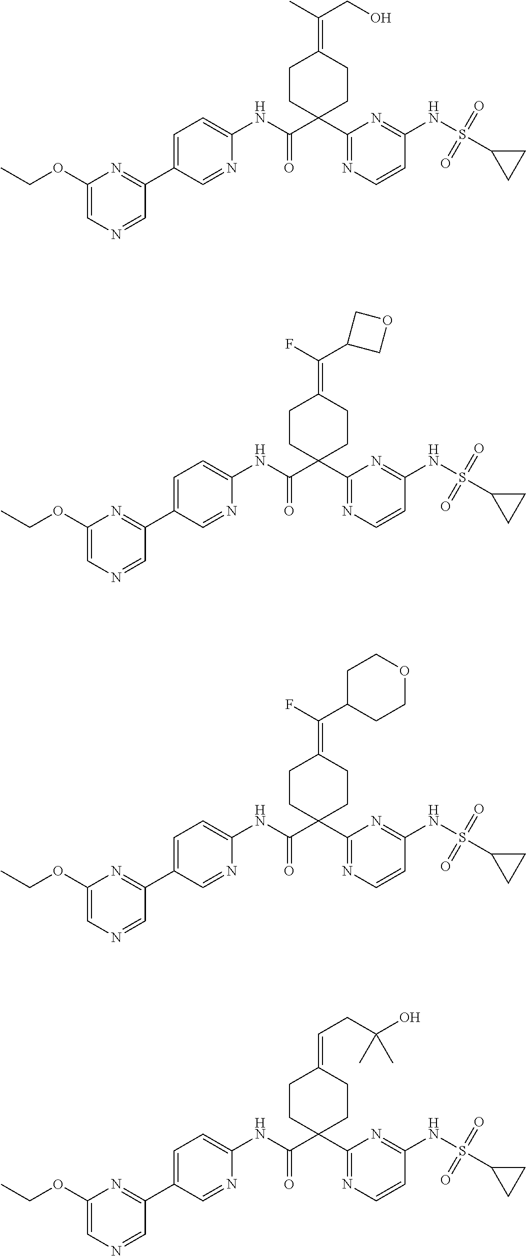 [SMILES: CCOc1cncc(-c2ccc(NC(=O)C3(c4nccc(NS(=O)(=O)C5CC5)n4)CCC(=C(C)CO)CC3)nc2)n1 ; CCOc1cncc(-c2ccc(NC(=O)C3(c4nccc(NS(=O)(=O)C5CC5)n4)CCC(=C(F)C4COC4)CC3)nc2)n1 ; CCOc1cncc(-c2ccc(NC(=O)C3(c4nccc(NS(=O)(=O)C5CC5)n4)CCC(=C(F)C4CCOCC4)CC3)nc2)n1 ; CCOc1cncc(-c2ccc(NC(=O)C3(c4nccc(NS(=O)(=O)C5CC5)n4)CCC(=CCC(C)(C)O)CC3)nc2)n1]

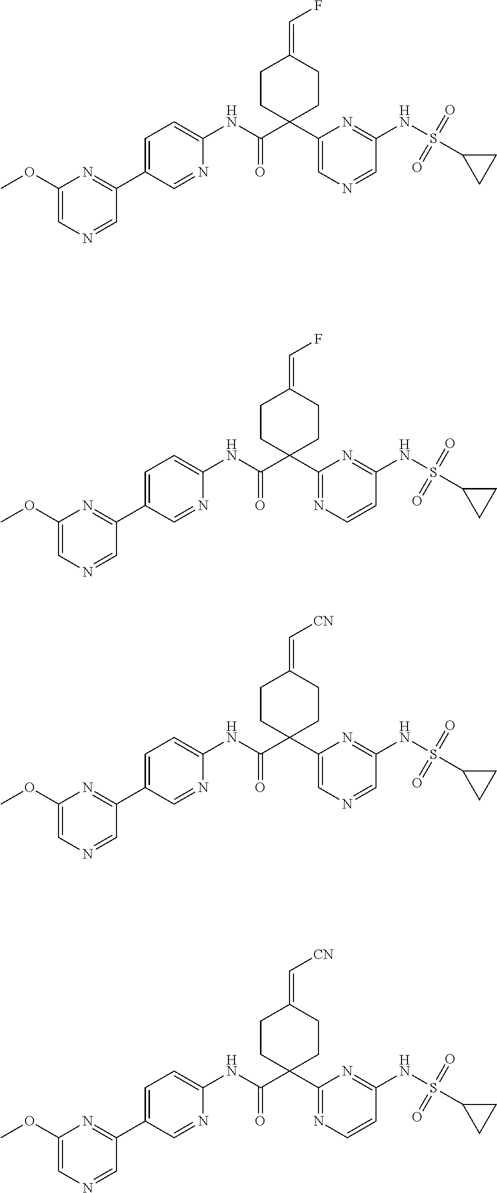 [SMILES: COc1cncc(-c2ccc(NC(=O)C3(c4cncc(NS(=O)(=O)C5CC5)n4)CCC(=CF)CC3)nc2)n1 ; COc1cncc(-c2ccc(NC(=O)C3(c4nccc(NS(=O)(=O)C5CC5)n4)CCC(=CF)CC3)nc2)n1 ; COc1cncc(-c2ccc(NC(=O)C3(c4cncc(NS(=O)(=O)C5CC5)n4)CCC(=CC#N)CC3)nc2)n1 ; COc1cncc(-c2ccc(NC(=O)C3(c4nccc(NS(=O)(=O)C5CC5)n4)CCC(=CC#N)CC3)nc2)n1]

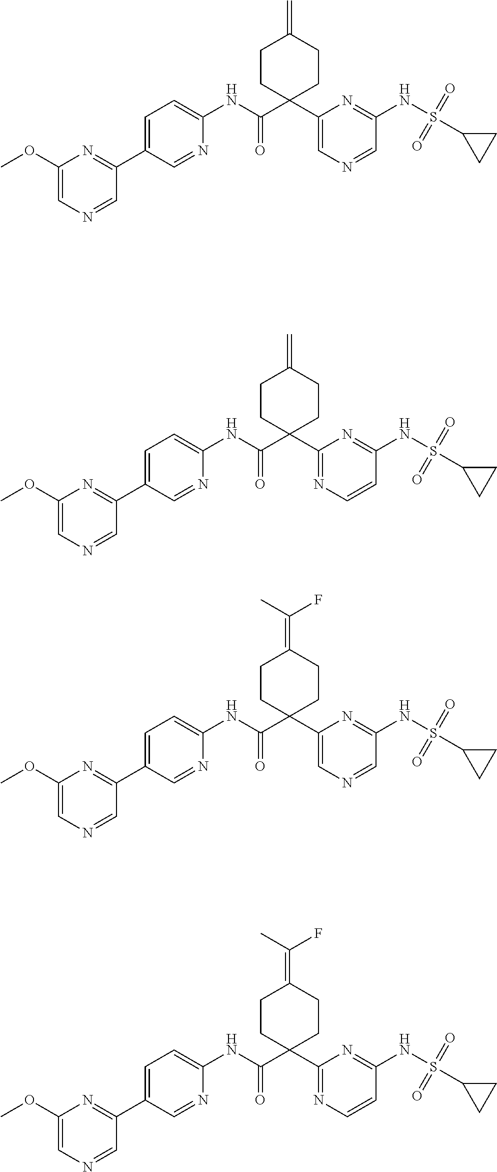 [SMILES: C=C1CCC(C(=O)Nc2ccc(-c3cncc(OC)n3)cn2)(c2cncc(NS(=O)(=O)C3CC3)n2)CC1 ; C=C1CCC(C(=O)Nc2ccc(-c3cncc(OC)n3)cn2)(c2nccc(NS(=O)(=O)C3CC3)n2)CC1 ; COc1cncc(-c2ccc(NC(=O)C3(c4cncc(NS(=O)(=O)C5CC5)n4)CCC(=C(C)F)CC3)nc2)n1 ; COc1cncc(-c2ccc(NC(=O)C3(c4nccc(NS(=O)(=O)C5CC5)n4)CCC(=C(C)F)CC3)nc2)n1]

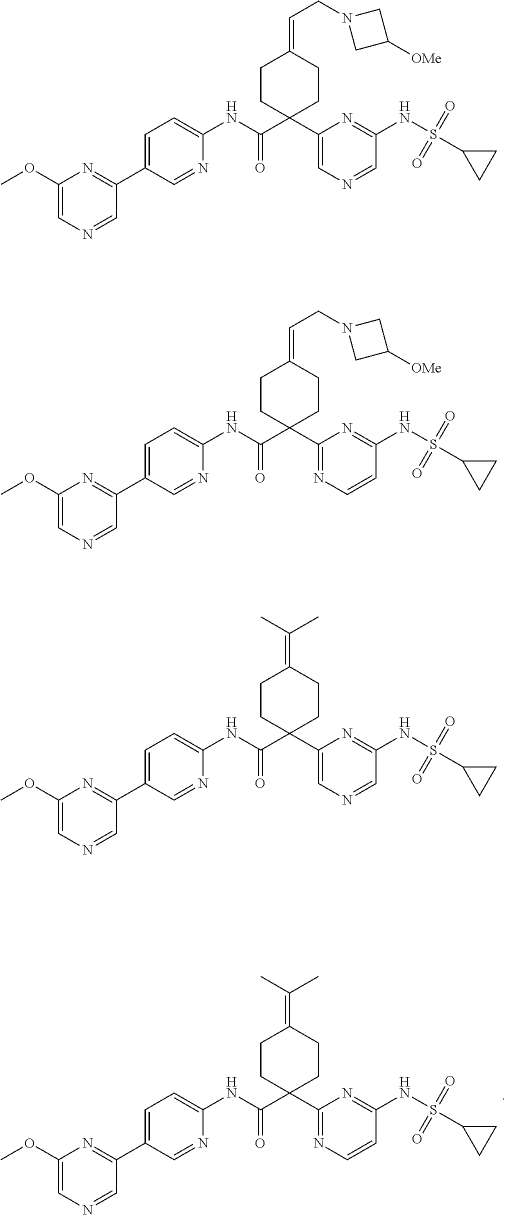 [SMILES: COc1cncc(-c2ccc(NC(=O)C3(c4cncc(NS(=O)(=O)C5CC5)n4)CCC(=CCN4CC(OC)C4)CC3)nc2)n1 ; COc1cncc(-c2ccc(NC(=O)C3(c4nccc(NS(=O)(=O)C5CC5)n4)CCC(=CCN4CC(OC)C4)CC3)nc2)n1 ; COc1cncc(-c2ccc(NC(=O)C3(c4cncc(NS(=O)(=O)C5CC5)n4)CCC(=C(C)C)CC3)nc2)n1 ; COc1cncc(-c2ccc(NC(=O)C3(c4nccc(NS(=O)(=O)C5CC5)n4)CCC(=C(C)C)CC3)nc2)n1]

The second aspect of the present invention provides a pharmaceutical composition comprising a compound according to the first aspect of the present invention, or an optical isomer, pharmaceutically acceptable salt, prodrug, deuterated derivative, hydrate, or solvate thereof, and a pharmaceutically acceptable carrier.

The third aspect of the present invention provides a use of a compound according to the first aspect of the present invention, or an optical isomer, pharmaceutically acceptable salt, prodrug, deuterated derivative, hydrate, or solvate thereof, for use in the preparation of a pharmaceutical composition for treating a disease, disorder, or condition associated with CTPS1 activity or expression level.

In another preferred embodiment, the disease, disorder, or condition is selected from the group consisting of: psoriasis, rheumatoid arthritis, systemic lupus erythematosus, lupus nephritis or cutaneous lupus, myasthenia gravis, multiple sclerosis, scleroderma, alopecia areata, inflammatory bowel disease and other various autoimmune diseases; graft-versus-host disease; promotion of vascular smooth muscle cell proliferation and repair after vascular injury or surgery; T-cell lymphoma, B-cell lymphoma, acute myeloid leukemia, acute lymphoblastic leukemia, chronic lymphocytic leukemia, angioimmunoblastic T-cell lymphoma, B-cell acute lymphoblastic leukemia, Hodgkin lymphoma, T-cell non-Hodgkin lymphoma (including natural killer/T-cell lymphoma, enteropathy-associated T-cell lymphoma, adult T-cell leukemia/lymphoma, hepatosplenic T-cell lymphoma, and cutaneous T-cell lymphoma), T-cell acute lymphoblastic leukemia, B-cell non-Hodgkin lymphoma (including Burkitt lymphoma, diffuse large B-cell lymphoma, follicular lymphoma, mantle cell lymphoma, and marginal zone lymphoma), hairy cell leukemia, lymphoblastic lymphoma, lymphoplasmacytic lymphoma, mucosa-associated lymphoid tissue (MALT) lymphoma, multiple myeloma, myelodysplastic syndrome, plasma cell myeloma, primary mediastinal large B-cell lymphoma, primary myelofibrosis, essential thrombocythemia, and polycythemia vera and other various hematological cancers.

### DETAILED DESCRIPTION

After long-term and intensive research, the inventors of the present invention have unexpectedly discovered a class of novel structural CTPS1 inhibitors, as well as their preparation methods and applications. The compounds of the present invention can be applied to treat various diseases associated with the activity of said CTPS1. Based on this discovery, the inventors have completed the present invention.

### Definitions

As used herein, the word “or” has the meaning of both “or” and “and” and is equivalent to “and/or”—unless otherwise specifically limited to just “or”.

As used herein, unless otherwise stated, a chiral carbon atom (or chiral center) of the compound(s) in the invention is optionally R-type, S-type, or a combination thereof.

As used herein, unless otherwise stated, the term “alkyl” by itself or as part of another substituent (which may include the short form of “alk,” e.g., alkoxy), refers to a straight (i.e. unbranched), branched chain, or cyclic hydrocarbon radical, or combination thereof, which may be fully saturated, mono- or polyunsaturated and can include di- and multivalent radicals. When an alkyl is preceded by a carbon-number modifier, e.g., C₁₋₁₀, its means the alkyl group contains 1 to 10 carbon atoms. For instance, examples of C₁₋₈ alkyl may include a linear or branched alkyl having 1-8 carbon atoms, such as methyl, ethyl, propyl, isopropyl, butyl, isobutyl, sec-butyl, and t-butyl.

As used herein, the term “alkenyl,” by itself or as part of another substituent, refers to a straight chain, or branched hydrocarbon chains having at least one carbon-carbon double bond. An alkenyl group with one double bond can be denoted as —CₙH₂ₙ₋₁ or —CₙH₂ₙ₋₃ with two double bonds. When an alkenyl is preceded by a carbon-number modifier, e.g., C₂₋₈, it means the alkenyl group contains 2 to 8 carbon atoms. For instance, examples of C₂₋₈ alkenyl may include vinyl, allyl, 1,2-butenyl, 2,3-butenyl, and butadienyl etc.

As used herein, the term “alkynyl,” by itself or as part of another substituent, refers to an aliphatic hydrocarbon group with at least one carbon-carbon triple bond. An alkynyl group may be linear or branched or combinations thereof. In some embodiments, it can contain 2 to 12 (e.g., 2 to 8, 2 to 6, or 2 to 4) carbon atoms. When an alkynyl is preceded by a carbon-number modifier, e.g., C₂-8, it means the alkynyl group contains 2 to 8 carbon atoms. Examples of an alkynyl group (e.g., C₂₋₈ alkynyl) may include acetenyl, propynyl, isopropynyl, 1-butynyl, isobutynyl, and sec-butynyl etc.

As used herein, the term “cycloalkyl” by itself or as part of another substituent, refers to a saturated or partially saturated carbocyclic mono-, bi-, or tri-cyclic (fused or bridged or spiral) ring system. It can contain 3 to 12 (e.g., 3 to 10, or 5 to 10) carbon atoms. When a cycloalkyl group is preceded by a carbon-number modifier, e.g., C₃₋₁₀, it means the cycloalkyl group contains 3 to 10 carbon atoms. In some embodiments, the term “C₃₋₁₀ cycloalkyl” may refer to a saturated or partially saturated mono- or bicyclic alkyl ring system containing 3 to 10 carbon atoms, such as cyclopropyl, cyclobutyl, cyclopentyl, cyclohexyl, and cycloheptyl. Below are some examples of cycloalkyl group.

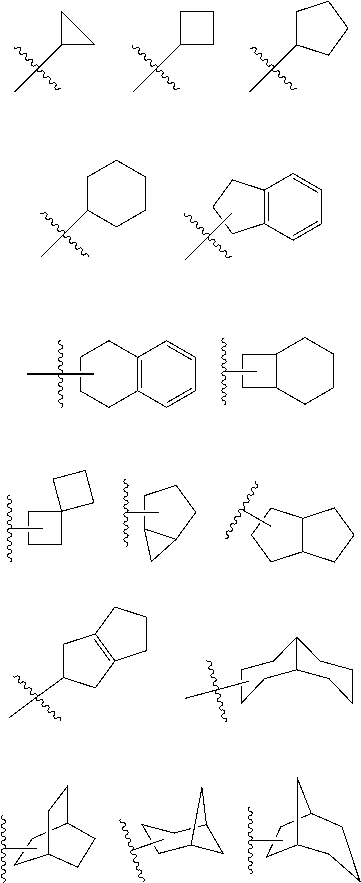

Unless otherwise stated, the following terms used in the specification and claims have the following meanings. “Aryl” means an all-carbon monocyclic or fused polycyclic (ie, a ring that shares a pair of adjacent carbon atoms) groups having a conjugated π-electron system, such as phenyl and naphthyl. The aryl ring may be fused to other cyclic groups (including saturated and unsaturated rings), but may not contain heteroatoms such as nitrogen, oxygen, or sulfur, while the point of attachment to the parent must be on the carbon atoms of the ring in a conjugated π-electron system. The aryl group can be substituted or unsubstituted. The following are some examples of aryl groups, and the present invention is not limited to the aryl groups described below.

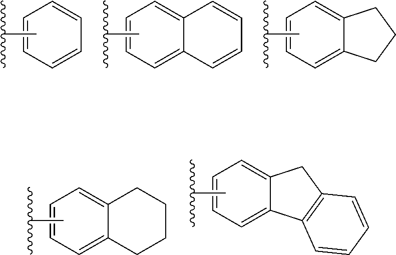

“Heteroaryl” refers to an aromatic monocyclic or polycyclic group containing one to more heteroatoms (optionally from nitrogen, oxygen, and sulfur), or a polycyclic group formed by condensing a heterocyclic group (containing one to more heteroatoms, optionally selected from nitrogen, oxygen, and sulfur) with an aryl group, and the attachment site is located on the aryl group. The heteroaryl group can be optionally substituted or unsubstituted. The following are some examples of heteroaryl groups, and the present invention is not limited to the following heteroaryl groups.

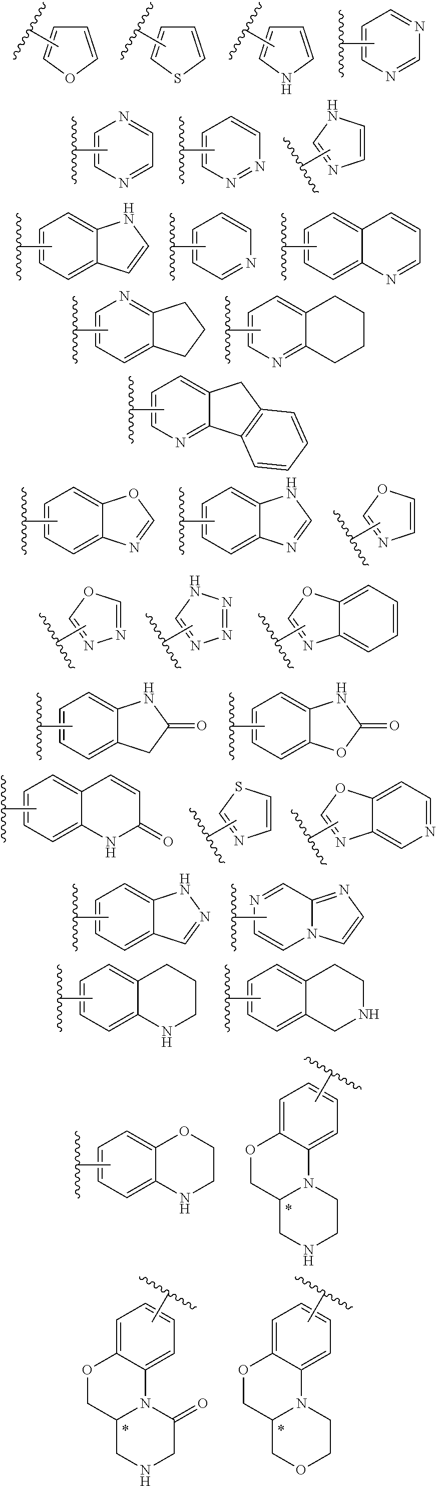

“Heterocyclyl” means a saturated or partially unsaturated monocyclic or polycyclic cyclic hydrocarbon substituent wherein one or more of the ring atoms are selected from nitrogen, oxygen or sulfur and the remaining ring atoms are carbon. Non-limiting examples of monocyclic heterocyclic groups include pyrrolidinyl, piperidinyl, piperazinyl, morpholinyl, thiomorpholinyl, homopiperazinyl. Polycyclic heterocyclic group refers to a heterocyclic group including a spiro ring, a fused ring, and a bridged ring. “Spirocyclic heterocyclyl” refers to a polycyclic heterocyclic group in which each ring of the system shares an atom (referred to as a spiro atom) with other rings in the system, wherein one or more of the ring atoms is selected from the group consisting of nitrogen and oxygen. Or sulfur, the remaining ring atoms are carbon. “Fused ring heterocyclyl” refers to a polycyclic heterocyclic group in which each ring of the system shares an adjacent pair of atoms with other rings in the system, and one or more rings may contain one or more double bonds, but none One ring has a fully conjugated pi-electron system, and wherein one or more ring atoms are selected from nitrogen, oxygen or sulfur, and the remaining ring atoms are carbon. “Bridged heterocyclyl” refers to a polycyclic heterocyclic group in which any two rings share two atoms which are not directly bonded, these may contain one or more double bonds, but none of the rings have a fully conjugated pi-electron system, and wherein one or more of the ring atoms are selected from nitrogen, oxygen or sulfur, and the remaining ring atoms are carbon. If a heterocyclic group has both a saturated ring and an aromatic ring (for example, the saturated ring and the aromatic ring are fused together), the point attached to the parent must be on the saturated ring. Note: When the point attached to the parent is on the aromatic ring, it is called a heteroaryl group and is not called a heterocyclic group. Some examples of the heterocyclic group are as follows, and the present invention is not limited to the following heterocyclic group.

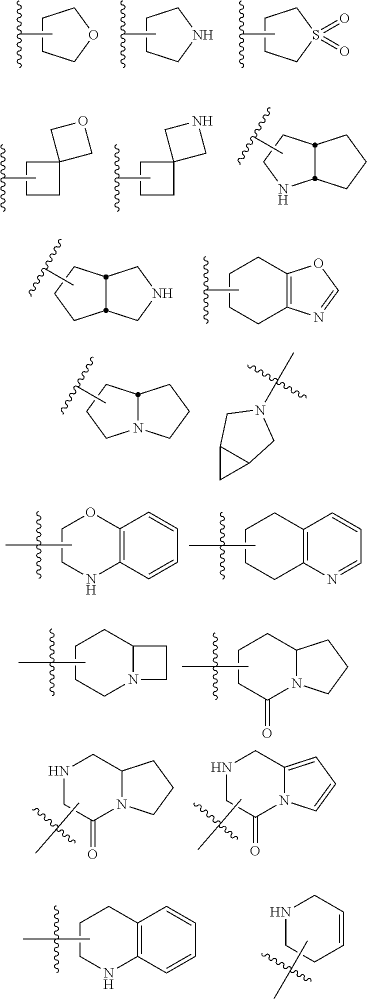

As used herein, the term “halogen”, when used alone or as part of another substituent, refers to F, Cl, Br, and I.

As used herein, the term “substituted” (when with or without “optionally”) means that one or more hydrogen atoms on a particular group are replaced by a particular substituent. Particular substituents are the substituents described above in the corresponding paragraphs, or the substituents which appear in the examples. Unless otherwise stated, an optionally substituted group may have a substituent selected from a particular group at any substitutable position of the group, and the substituents may be the same or different at each position. A cyclic substituent, such as a heterocyclic group, may be attached to another ring, such as a cycloalkyl group, to form a spirobicyclic ring system, i.e., the two rings have a common carbon atom. Those skilled in the art will appreciate that the combinations of substituents contemplated by the present invention are those that are stable or chemically achievable. The substituents are, for example but not limited to, C₁₋₈ alkyl, C₂₋₈ alkenyl, C₂₋₈ alkynyl, C₃₋₈ cycloalkyl, 3- to 12-membered heterocyclyl, aryl, heteroaryl, halogen, hydroxy, carboxy (—COOH), C₁₋₈ aldehyde, C₂₋₁₀ acyl, C₂₋₁₀ ester, amino group.

For convenience and in accordance with conventional understanding, the term “optionally substituted” or “optionally substituted” applies only to sites which are capable of being substituted by a substituent, and does not include those which are not chemically achievable.

As used herein, unless otherwise specified, the term “pharmaceutically acceptable salt” refers to a salt that is suitable for contact with the tissue of a subject (eg, a human) without causing unpleasant side effects. In some embodiments, a pharmaceutically acceptable salt of a compound of the invention includes a salt (eg, a potassium salt, a sodium salt, a magnesium salt, a calcium salt) of a compound of the invention having an acidic group or is a salt of a compound of a compound of the invention having a basic group the invention (e.g., a sulfate, a hydrochloride, a phosphate, a nitrate, a carbonate).

### Application

The present invention provides a class of compounds of formula (I), or their deuterated derivatives, their pharmaceutically acceptable salts, optical isomers (enantiomers or diastereomers, if any case), hydrates, solvates, or pharmaceutical combinations comprising the compound represented by formula (I), its optical isomers, pharmaceutically acceptable salts, prodrugs, deuterated forms, hydrates, and solvates for inhibiting CTPS1 kinase activity.

The compound of the present invention can be used as CTPS1 kinase inhibitor.

The present invention provides a monospecific inhibitor of CTPS1, which achieves the purpose of preventing, alleviating, or curing diseases by modulating the activity of CTPS1. The diseases referred to include, but are not limited to: psoriasis, rheumatoid arthritis, systemic lupus erythematosus, lupus nephritis or cutaneous lupus, myasthenia gravis, multiple sclerosis, scleroderma, alopecia areata, inflammatory bowel disease and other various autoimmune diseases; graft-versus-host disease; promotion of vascular smooth muscle cell proliferation and repair after vascular injury or surgery; T-cell lymphoma, B-cell lymphoma, acute myeloid leukemia, acute lymphoblastic leukemia, chronic lymphocytic leukemia, angioimmunoblastic T-cell lymphoma, B-cell acute lymphoblastic leukemia, Hodgkin lymphoma, T-cell non-Hodgkin lymphoma (including natural killer/T-cell lymphoma, enteropathy-associated T-cell lymphoma, adult T-cell leukemia/lymphoma, hepatosplenic T-cell lymphoma, and cutaneous T-cell lymphoma), T-cell acute lymphoblastic leukemia, B-cell non-Hodgkin lymphoma (including Burkitt lymphoma, diffuse large B-cell lymphoma, follicular lymphoma, mantle cell lymphoma, and marginal zone lymphoma), hairy cell leukemia, lymphoblastic lymphoma, lymphoplasmacytic lymphoma, mucosa-associated lymphoid tissue (MALT) lymphoma, multiple myeloma, myelodysplastic syndrome, plasma cell myeloma, primary mediastinal large B-cell lymphoma, primary myelofibrosis, essential thrombocythemia, and polycythemia vera and other various hematological cancers.

The compounds of the present invention and their deuterated forms, as well as pharmaceutically acceptable salts or isomers (if present) or hydrates and/or compositions thereof can be combined with pharmaceutically acceptable excipients or carriers formulated together, the resulting composition can be administered to humans or animals for the treatment of disorders, symptoms and diseases. The composition can be: tablets, pills, suspensions, solutions, emulsions, capsules, aerosols, sterile injections, sterile powders and the like. In a preferred embodiment, the pharmaceutical composition is a dosage form suitable for oral administration, including but not limited to tablets, solutions, suspensions, capsules, granules, and powders. The amount of the compound or pharmaceutical composition administered to the patient is not fixed, and is usually administered in a pharmaceutically effective amount. At the same time, the amount of the compound actually administered can be determined by the physician according to the actual situation, including the disease to be treated, the route of administration selected, the actual compound administered, the individual condition of the patient, and so on. The dosage of the compound of the present invention depends on the specific use of the treatment, the mode of administration, the state of the patient, and the judgment of the physician. The ratio or concentration of the compound of the present invention in the pharmaceutical composition depends on a variety of factors, including dosage, physical and chemical properties, route of administration, and the like.

It is to be understood that within the scope of the present invention, the various technical features of the present invention and the various technical features specifically described hereinafter (as in the embodiments) may be combined with each other to form a new or preferred technical solution.

### Pharmaceutical Composition and Method of Administration

Since the compound of the present invention has excellent inhibitory activity against CTPS1, the compound of the present invention and its various crystal forms, optical isomers, pharmaceutically acceptable inorganic or organic salts, prodrugs, deuterated forms, hydrates or solvates, and pharmaceutical compositions containing the compounds of the present invention as the main active ingredients can be used to treat, prevent and alleviate diseases associate with the activity or expression of CTPS1.

The pharmaceutical compositions of the present invention comprise a safe or effective amount of a compound of the present invention, or a pharmaceutically acceptable salt thereof, and a pharmaceutically acceptable excipient or carrier. By “safe and effective amount” it is meant that the amount of the compound is sufficient to significantly improve the condition without causing serious side effects. In general, the pharmaceutical compositions contain from 1 to 2000 mg of the compound of the invention per agent, more preferably from 5 to 200 mg of the compound of the invention per agent. Preferably, the “one dose” is a capsule or tablet.

“Pharmaceutically acceptable carrier” means: one or more compatible solid or liquid fillers or gel materials which are suitable for human use and which must be of sufficient purity and of sufficiently low toxicity. By “compatibility” it is meant herein that the components of the composition are capable of intermingling with the compounds of the invention and with each other without significantly reducing the efficacy of the compound. Examples of pharmaceutically acceptable carriers include, but are not limited to, filler (or diluent), disintegrant, lubricant, binder, matrix, emulsifiers, run wet agents, colorants, flavoring agents, stabilizers, antioxidants, preservatives, pyrogen-free water, and the like.

The mode of administration of the compound or pharmaceutical composition of the present invention is not particularly limited, and representative modes of administration include, but are not limited to, oral, intratumoral, rectal, parenteral (intravenous, intramuscular or subcutaneous), and topical administration.

Solid dosage forms for oral administration include capsules, tablets, pills, powders, and granules. In these solid dosage forms, the active compound is mixed with at least one conventional inert excipient (or carrier). In capsules, tablets and pills, the dosage form may also contain a buffer.

Solid dosage forms such as tablets, dragees, capsules, pills, and granules can be prepared with coatings and shells such as enteric coatings and other materials known in the art. They may contain opacifying agents and the release of the active compound or compound in such compositions may be released in a portion of the digestive tract in a delayed manner. Examples of embedding components that can be employed are polymeric and waxy materials. If necessary, the active compound may also be in microencapsulated form with one or more of the above-mentioned excipients.

In addition to these inert diluents, the compositions may contain adjuvants such as wetting agents, emulsifying and suspending agents, sweetening agents, flavoring agents and perfumes.

In addition to the active compound, the suspension may contain suspending agents.

Compositions for parenteral injection may comprise a physiologically acceptable sterile aqueous or nonaqueous solution, dispersion, suspension or emulsion, and a sterile powder for reconstitution into a sterile injectable solution or dispersion. Suitable aqueous and nonaqueous vehicles, diluents, solvents or vehicles include water, ethanol, polyols, and suitable mixtures thereof.

Dosage forms for the compounds of the invention for topical administration include ointments, powders, patches, propellants and inhalants. The active ingredient is admixed under sterile conditions with a physiologically acceptable carrier and any preservatives, buffers, or, if necessary, propellants.

The compounds of the invention may be administered alone or in combination with other pharmaceutically acceptable compounds.

When a pharmaceutical composition is used, a safe and effective amount of a compound of the invention is administered to a mammal (e.g., a human) in need of treatment wherein the dosage is a pharmaceutically effective dosage, for a 60 kg body weight, The dose to be administered is usually from 1 to 2000 mg, preferably from 5 to 500 mg. Of course, specific doses should also consider factors such as the route of administration, the health of the patient, etc., which are within the skill of the skilled physician.

The main advantages of the present invention include:
1. Provided a compound as shown in Formula I.
2. Provided a structurally novel CTPS1 inhibitor, and the preparation and application thereof, said inhibitor being able to inhibit the activity of CTPS1 at a very low concentration.
3. Provided a CTPS1 inhibitor of good oral absorption.
4. Provided a class of pharmaceutical compositions for the treatment of diseases associated with CTPS1 activity.

The invention is further illustrated below in conjunction with specific embodiments. It is to be understood that the examples are not intended to limit the scope of the invention. The experimental methods in the following examples which do not specify the specific conditions are usually in accordance with conventional conditions or according to the conditions recommended by the manufacturer. Percentages and parts are by weight unless otherwise stated.

Some of the representative compounds of the present invention can be prepared by the following synthetic methods, and in each of the following reaction formulas, the reagents and conditions for each step can be selected from those conventional in the field for carrying out such preparation methods, and the above selections can be made by a person skilled in the art according to the knowledge in the field after the structures of the compounds of the present invention are disclosed.

### EXAMPLE

Boc=tert-butoxycarbonyl
t-BuOH=tert-butanol
CN=cyano
m-CPBA=3-chloroperoxybenzoic acid
DAST=diethylaminosulfur trifluoride
DCM=dichloromethane
DIPEA or DIEA=N,N-diisopropylethylamine
DIBAL-H=diisobutylaluminum hydride
DMF=N,N-dimethylformamide
DMSO=dimethyl sulfoxide
EtOAc or EA=ethyl acetate
Et=ethyl
HATU=N,N,N′,N′-tetramethyl-O-(7-azabenzotriazol-1-yl)uranium hexafluorophosphate
H₂O₂=hydrogen peroxide
LiHMDS=lithium bis(trimethylsilyl)amide
LDA=lithium diisopropylamide
MOMBr=bromomethyl methyl ether
NFSI=N-fluorobenzenesulfonimide
NBS=N-bromosuccinimide
Me=methyl
NMP=N-methylpyrrolidone
Ph=phenyl
PMB=p-methoxybenzyl
Pb(OAc)₂=Leadacetate
TBAF=tetrabutylammonium fluoride
TEA=triethylamine
TFA=trifluoroacetic acid
THF=tetrahydrofuran
TsCl=tosyl chloride

### Example 1: Preparation of Compound 1

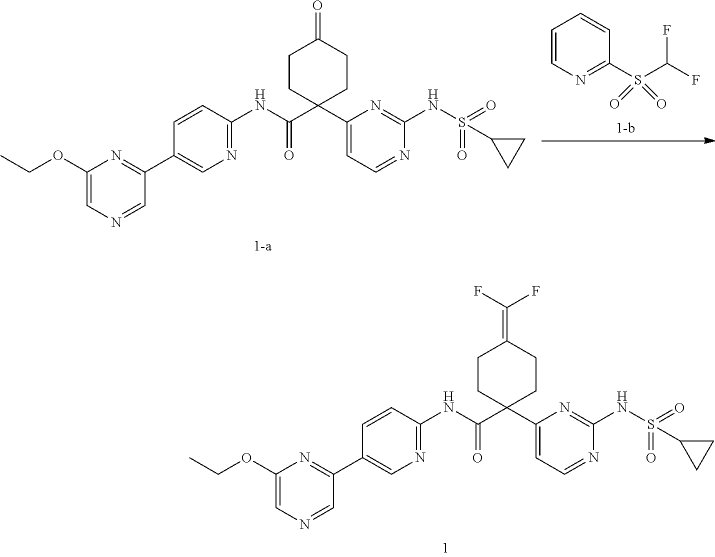 [SMILES: CCOc1cncc(-c2ccc(NC(=O)C3(c4ccnc(NS(=O)(=O)C5CC5)n4)CCC(=O)CC3)nc2)n1 ; O=S(=O)(c1ccccn1)C(F)F ; CCOc1cncc(-c2ccc(NC(=O)C3(c4ccnc(NS(=O)(=O)C5CC5)n4)CCC(=C(F)F)CC3)nc2)n1]

Compound 1-a (90 mg, 0.167 mmol) (the synthesis route of compound 1-a refers to the method described in patent WO 2020/245665 A1) and compound 1-b (49 mg, 0.251 mmol) were dissolved in N,N-dimethylformamide (2.5 mL). The mixture was cooled to −60° C., and a solution of potassium tert-butoxide (38 mg, 0.335 mmol) in N,N-dimethylformamide (0.5 mL) was slowly added dropwise. The reaction mixture was stirred at room temperature for 1 hour, then concentrated under reduced pressure. The crude product was purified by preparative thin-layer chromatography (dichloromethane:methanol=25:1) to afford compound 1 as a white solid (7.0 mg, yield 7%). ¹H NMR (500 MHz, DMSO-d₆) δ 11.28 (br., 1H), 10.15 (s, 1H), 9.02 (d, J=2.0 Hz, 1H), 8.84 (s, 1H), 8.60 (d, J=5.2 Hz, 1H), 8.49 (dd, J=8.8, 2.4 Hz, 1H), 8.25 (s, 1H), 8.19 (d, J=8.8 Hz, 1H), 7.23 (d, J=4.9 Hz, 1H), 4.47 (q, J=7.0 Hz, 2H), 3.27-3.20 (m, 1H), 2.57-2.52 (m, 2H), 2.35-2.26 (m, 2H), 2.25-2.14 (m, 2H), 2.13-1.99 (m, 2H), 1.39 (t, J=7.0 Hz, 3H), 1.06-1.01 (m, 2H), 0.88-0.82 (m, 2H) ppm. MS m/z 572.1 [M+H]⁺.

### Example 2: Preparation of Compound 2

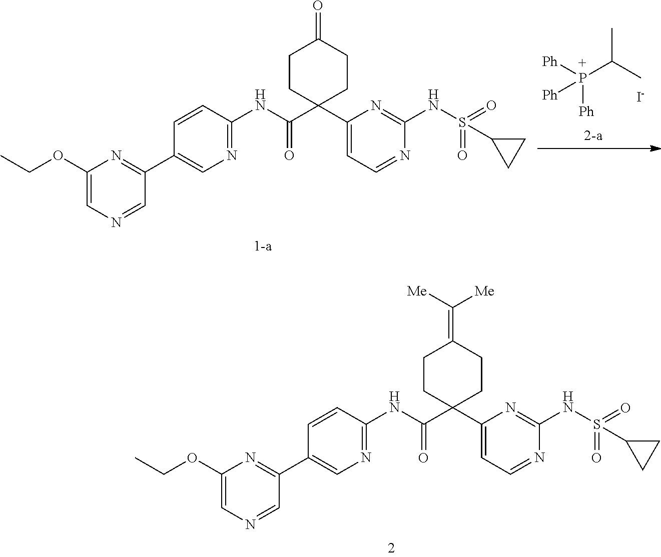

Compound 2-a (282 mg, 0.651 mmol) was dissolved in tetrahydrofuran (4 mL), and the mixture was cooled to 0° C. Lithium bis(trimethylsilyl)amide (1.0 M, 0.65 mL, 0.65 mmol) was added. The reaction mixture was warmed to room temperature and stirred for 30 minutes, then compound 1-a (70 mg, 0.130 mmol) was added. After completion, the reaction mixture was stirred at room temperature for 2 hours. The reaction was quenched with saturated ammonium chloride solution and extracted with ethyl acetate (3×15 mL). The combined organic phases were washed with saturated brine, dried over anhydrous sodium sulfate, filtered, and concentrated under reduced pressure. The crude product was purified by preparative thin-layer chromatography (dichloromethane:methanol=20:1) to afford compound 2 as a white solid (4.9 mg, yield 7%). ¹H NMR (500 MHz, DMSO-d₆) δ 11.24 (br., 1H), 10.01 (s, 1H), 9.01 (d, J=1.9 Hz, 1H), 8.83 (s, 1H), 8.59 (d, J=5.3 Hz, 1H), 8.49 (dd, J=8.8, 2.4 Hz, 1H), 8.25 (s, 1H), 8.19 (d, J=8.8 Hz, 1H), 7.22 (d, J=5.3 Hz, 1H), 4.47 (q, J=7.0 Hz, 2H), 3.29-3.22 (m, 1H), 2.48-2.35 (m, 4H), 2.22-2.10 (m, 2H), 2.06-1.94 (m, 2H), 1.64 (s, 6H), 1.39 (t, J=7.0 Hz, 3H), 1.08-1.02 (m, 2H), 0.90-0.84 (m, 2H) ppm. MS m/z 564.2 [M+H]⁺.

### Example 3: Preparation of Compound 3

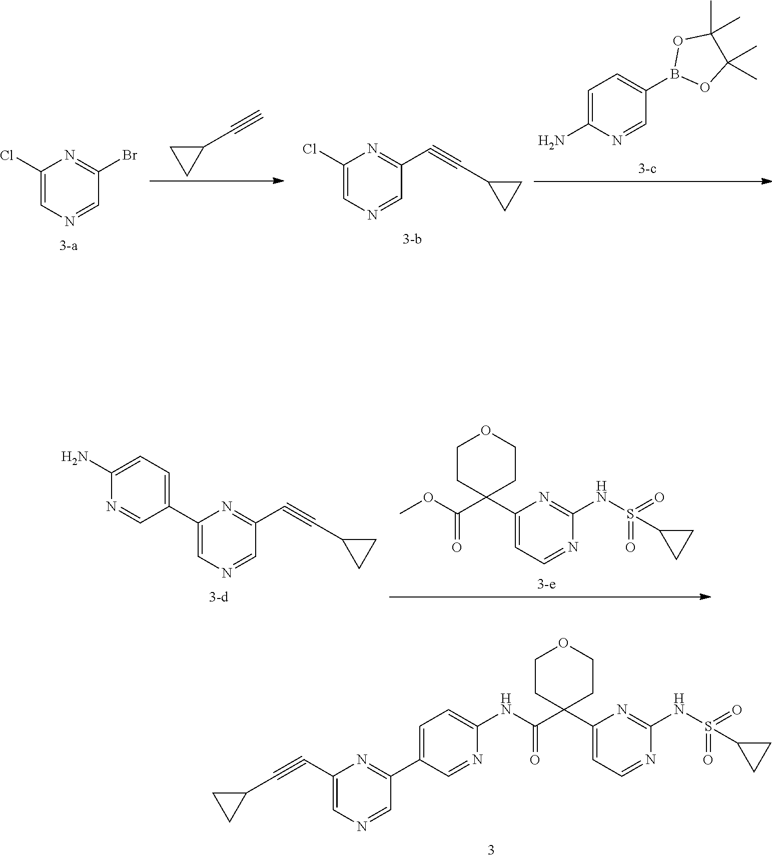

Compound 3-a (160 mg, 0.827 mmol), cyclopropylethyne (60 mg, 0.910 mmol), bis(triphenylphosphine)palladium(II) dichloride (29 mg, 0.041 mmol), copper(I) iodide (32 mg, 0.165 mmol), and triethylamine (167 mg, 1.650 mmol) were dissolved in N,N-dimethylformamide (3 mL). Under nitrogen protection, the reaction mixture was stirred at room temperature for 1 hour. After completion, water was added to dilute the mixture, which was then extracted with ethyl acetate (3×15 mL). The combined organic phases were washed with saturated brine, dried over anhydrous sodium sulfate, filtered, and concentrated under reduced pressure. The crude product was purified by silica gel column chromatography (petroleum ether:ethyl acetate=20:1) to afford compound 3-b as a yellow oil (60 mg, yield 41%). MS m/z 179.1 [M+H]⁺.

Compound 3-b (60 mg, 0.336 mmol), compound 3-c (81 mg, 0.370 mmol), [1,1′-bis(diphenylphosphino)ferrocene]palladium(II) dichloride (24 mg, 0.034 mmol), and potassium carbonate (139 mg, 1.010 mmol) were dissolved in a mixed solution of 1,4-dioxane and water (3/0.5 mL). Under nitrogen protection, the reaction mixture was stirred at 90° C. for 30 minutes. After completion, water was added to dilute the mixture, which was then extracted with ethyl acetate (3×15 mL). The combined organic phases were washed with saturated brine, dried over anhydrous sodium sulfate, filtered, and concentrated under reduced pressure. The crude product was purified by silica gel column chromatography (dichloromethane:methanol=30:1) to afford compound 3-d as a yellow solid (49 mg, yield 62%). MS m/z 237.2 [M+H]⁺.

Compound 3-d (25 mg, 0.106 mmol) and compound 3-e (40 mg, 0.116 mmol) (the synthesis route of intermediate 3-e refers to the method described in patent WO 2020/245665 A1) were dissolved in tetrahydrofuran (3 mL). Lithium bis(trimethylsilyl)amide (1.0 M, 0.21 mmol) was slowly added dropwise at 0° C. After completion, the reaction mixture was stirred at room temperature for 1 hour, then quenched with water. The mixture was adjusted to pH=4 with 2 M hydrochloric acid and concentrated under reduced pressure. The crude product was purified by preparative thin-layer chromatography (dichloromethane:methanol=25:1) to afford compound 3 as a white solid (31 mg, yield 54%). ¹H NMR (500 MHz, DMSO-d₆) δ 11.30 (br., 1H), 10.20 (s, 1H), 9.19 (s, 1H), 9.01 (d, J=2.5 Hz, 1H), 8.64-8.61 (m, 2H), 8.50 (dd, J=8.8, 2.5 Hz, 1H), 8.21 (d, J=9.2 Hz, 1H), 7.26 (d, J=5.3 Hz, 1H), 3.79-3.70 (m, 2H), 3.66-3.57 (m, 2H), 3.29-3.21 (m, 1H), 2.49-2.43 (m, 2H), 2.24-2.15 (m, 2H), 1.72-1.64 (m, 1H), 1.07-1.03 (m, 2H), 1.02-0.96 (m, 2H), 0.90-0.85 (m, 4H) ppm. MS m/z 546.1 [M+H]⁺.

### Example 4: Preparation of Compound 4

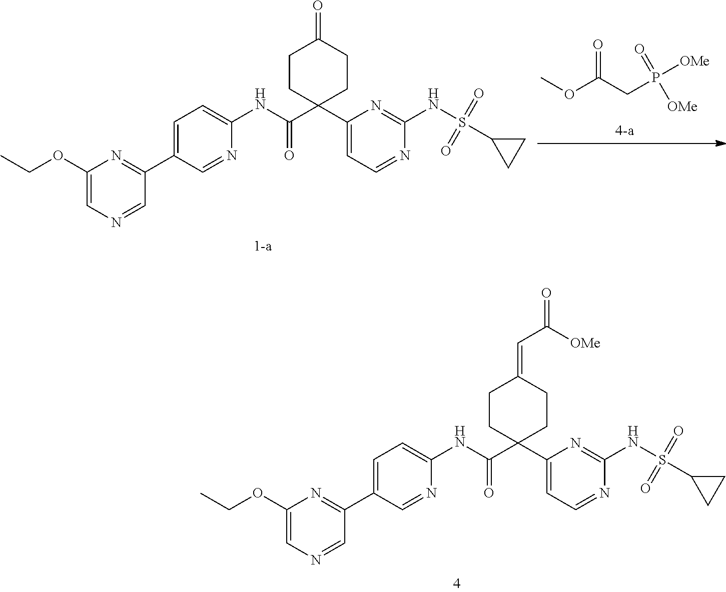 [SMILES: CCOc1cncc(-c2ccc(NC(=O)C3(c4ccnc(NS(=O)(=O)C5CC5)n4)CCC(=O)CC3)nc2)n1 ; COC(=O)CP(=O)(OC)OC ; CCOc1cncc(-c2ccc(NC(=O)C3(c4ccnc(NS(=O)(=O)C5CC5)n4)CCC(=CC(=O)OC)CC3)nc2)n1]

Compound 1-a (45 mg, 0.08 mmol) was dissolved in tetrahydrofuran (2.0 mL), and sodium hydride (6 mg, 0.16 mmol) was slowly added at 0° C. After the addition was complete, the reaction was stirred at 0° C. for 1 hour. Then, triethyl phosphonoacetate was added at −20° C., and the mixture was slowly warmed to room temperature and stirred for an additional 2 hours. The reaction was quenched with water, followed by the addition of 1 mL of glacial acetic acid. The mixture was extracted with ethyl acetate (3×20 mL), and the combined organic phases were washed with saturated brine, dried over anhydrous sodium sulfate, filtered, and concentrated under reduced pressure. The crude product was purified by preparative thin-layer chromatography (dichloromethane:methanol=25:1) to afford the white solid compound 4 (30.0 mg, yield 60%). ¹H NMR (500 MHz, DMSO-d₆) δ 11.27 (s, 1H), 10.15 (s, 1H), 9.02 (d, J=2.0 Hz, 1H), 8.84 (s, 1H), 8.60 (d, J=5.2 Hz, 1H), 8.49 (dd, J=8.8, 2.4 Hz, 1H), 8.25 (s, 1H), 8.19 (d, J=8.9 Hz, 1H), 7.26 (d, J=5.1 Hz, 1H), 5.73 (s, 1H), 4.47 (q, J=7.0 Hz, 2H), 3.61 (s, 3H), 3.27-3.23 (m, 1H), 2.65-2.57 (m, 2H), 2.42-2.35 (m, 2H), 2.28-2.04 (m, 4H), 1.39 (t, J=7.0 Hz, 3H), 1.08-0.99 (m, 2H), 0.91-0.82 (m, 2H) ppm. MS m/z 594.3 [M+H]⁺.

### Example 5: Preparation of Compound 5

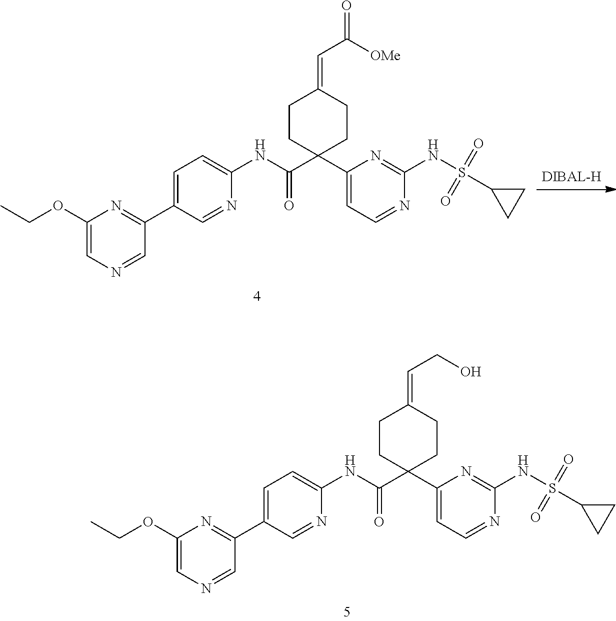 [SMILES: CCOc1cncc(-c2ccc(NC(=O)C3(c4ccnc(NS(=O)(=O)C5CC5)n4)CCC(=CC(=O)OC)CC3)nc2)n1 ; CCOc1cncc(-c2ccc(NC(=O)C3(c4ccnc(NS(=O)(=O)C5CC5)n4)CCC(=CCO)CC3)nc2)n1]

Compound 4 (15 mg, 0.02 mmol) was dissolved in tetrahydrofuran (1.0 mL), and a solution of diisobutylaluminum hydride in n-hexane (1 M, 0.1 mL, 0.1 mmol) was added at −20° C. The reaction was then stirred at −20° C. for an additional 1 hour. After completion, the reaction was quenched with water, followed by the addition of 1 mL glacial acetic acid. The mixture was extracted with ethyl acetate (3×20 mL), and the combined organic layers were washed with saturated brine, dried over anhydrous sodium sulfate, filtered, and concentrated under reduced pressure. The crude product was purified by preparative thin-layer chromatography (dichloromethane:methanol=10:1) to afford the white solid compound 5 (7.0 mg, 49% yield). ¹H NMR (500 MHz, DMSO-d₆) δ 11.31 (s, 1H), 9.99 (s, 1H), 9.02 (d, J=2.2 Hz, 1H), 8.83 (s, 1H), 8.57-8.41 (m, 2H), 8.24 (s, 1H), 8.17 (d, J=8.8 Hz, 1H), 7.14-7.01 (m, 1H), 5.30 (t, J=6.5 Hz, 1H), 4.52-4.38 (m, 3H), 4.00-3.88 (m, 2H), 3.25-3.15 (m, 1H), 2.34-1.94 (m, 8H), 1.39 (t, J=7.0 Hz, 3H), 1.04-0.93 (m, 2H), 0.86-0.75 (m, 2H) ppm. MS m/z 566.2 [M+H]⁺.

### Example 6: Preparation of Compound 6

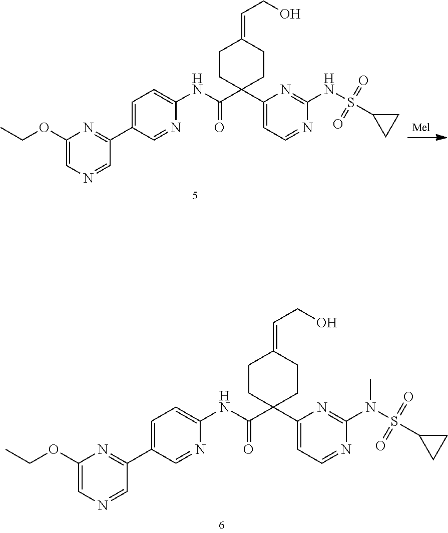 [SMILES: CCOc1cncc(-c2ccc(NC(=O)C3(c4ccnc(NS(=O)(=O)C5CC5)n4)CCC(=CCO)CC3)nc2)n1 ; CCOc1cncc(-c2ccc(NC(=O)C3(c4ccnc(N(C)S(=O)(=O)C5CC5)n4)CCC(=CCO)CC3)nc2)n1]

Compound 5 (20 mg, 0.035 mmol) and silver oxide (16 mg, 0.071 mmol) were dissolved in dichloromethane (3 mL), followed by the addition of iodomethane (10 mg, 0.071 mmol). The reaction was stirred at room temperature for 2.5 hours. The reaction mixture was filtered and washed with dichloromethane. The filtrate was concentrated under reduced pressure and purified by preparative thin-layer chromatography (dichloromethane:methanol=15:1) to afford compound 6 as a white solid (3.7 mg, 18% yield). ¹H NMR (500 MHz, DMSO-d₆) δ 11.08 (br., 1H), 8.84 (s, 1H), 8.72 (s, 1H), 8.47 (s, 1H), 8.35 (d, J=8.8 Hz, 1H), 8.21 (s, 1H), 8.01 (d, J=9.1 Hz, 1H), 7.21 (d, J=2.5 Hz, 1H), 5.31-5.21 (m, 1H), 4.54-4.40 (m, 2H), 3.94 (d, J=6.3 Hz, 2H), 3.66 (s, 2H), 3.33-3.21 (m, 1H), 2.48-2.38 (m, 3H), 2.31-1.91 (m, 7H), 1.42-1.35 (m, 3H), 1.14-1.04 (m, 2H), 1.03-0.94 (m, 2H) ppm. MS m/z 580.3 [M+H]⁺.

### Example 7: Preparation of Compound 7

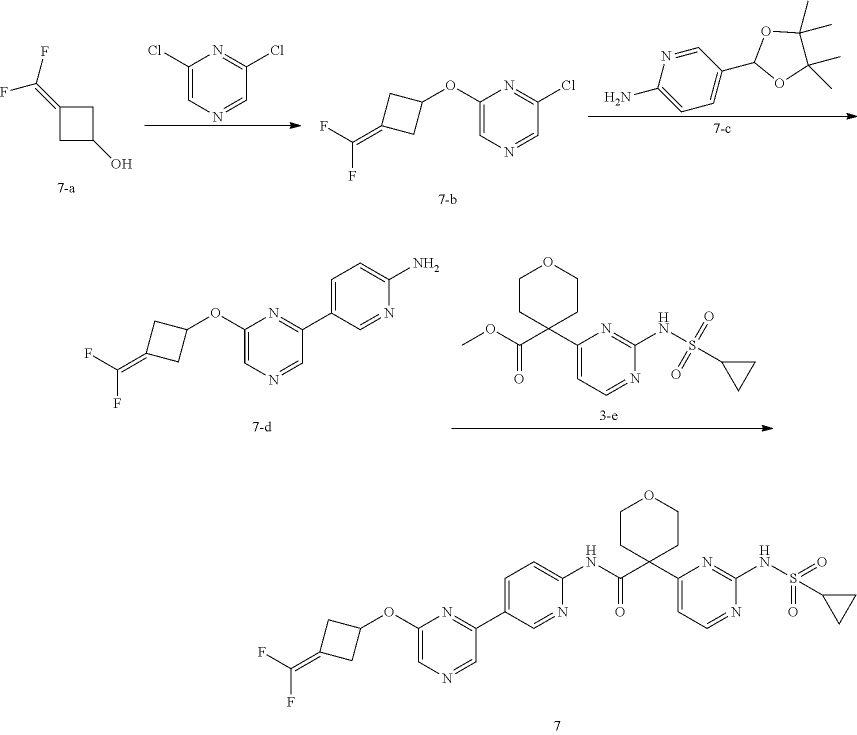

Compound 7-a (40 mg, 0.33 mmol) (the synthetic route of compound 7-a refers to the method described in patent WO 2022/122044) was dissolved in tetrahydrofuran (5.0 mL), and sodium hydride (40 mg, 1.0 mmol) was slowly added at 0° C. The mixture was then warmed to room temperature and stirred for 1 hour. Subsequently, 2,6-dichloropyrazine (98 mg, 0.66 mmol) was added at room temperature, and the reaction was stirred for an additional 2 hours. The reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (petroleum ether:ethyl acetate=5:1) to afford compound 7-b as a yellow oil (50.0 mg, 65% yield). MS m/z 233.1 [M+H]⁺.

Compound 7-b (50 mg, 0.21 mmol), 1,1′-bis(diphenylphosphino)ferrocene palladium(II) dichloride (7 mg, 0.01 mmol), potassium carbonate (87 mg, 0.63 mmol), and 2-amino-5-pyridineboronic acid pinacol ester (92 mg, 0.42 mmol) were dissolved in a mixed solvent of dioxane (2.5 mL) and water (0.5 mL). Under nitrogen protection, the reaction was heated at 90° C. for 1 hour. After completion, the reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by silica gel column chromatography (petroleum ether:ethyl acetate=1:2) to afford compound 7-d as a pale yellow solid (50.0 mg, 80% yield). MS m/z 290.1 [M+H]⁺.

Compound 7-d (20 mg, 0.07 mmol) and compound 3-e (27 mg, 0.08 mmol) were dissolved in tetrahydrofuran (2.0 mL), and lithium bis(trimethylsilyl)amide in tetrahydrofuran (1.0 M, 0.2 mL, 0.2 mmol) was added at 0° C. The mixture was then warmed to room temperature and stirred for 1 hour. The reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (100% ethyl acetate) to afford compound 7 as a white solid (22 mg, 54% yield). ¹H NMR (500 MHz, DMSO-d₆) δ 11.31 (s, 1H), 10.11 (s, 1H), 9.06-8.97 (m, 1H), 8.91 (s, 1H), 8.62 (d, J=4.6 Hz, 1H), 8.51 (dd, J=8.8, 2.3 Hz, 1H), 8.30 (s, 1H), 8.20 (d, J=8.8 Hz, 1H), 7.24 (s, 1H), 5.44-5.33 (m, 1H), 3.80-3.68 (m, 2H), 3.65-3.53 (m, 2H), 3.29-3.19 (m, 3H), 2.97-2.83 (m, 2H), 2.46-2.41 (m, 2H), 2.26-2.13 (m, 2H), 1.12-0.97 (m, 2H), 0.94-0.80 (m, 2H) ppm. MS m/z 600.1 [M+H]⁺.

### Example 8: Preparation of Compound 8

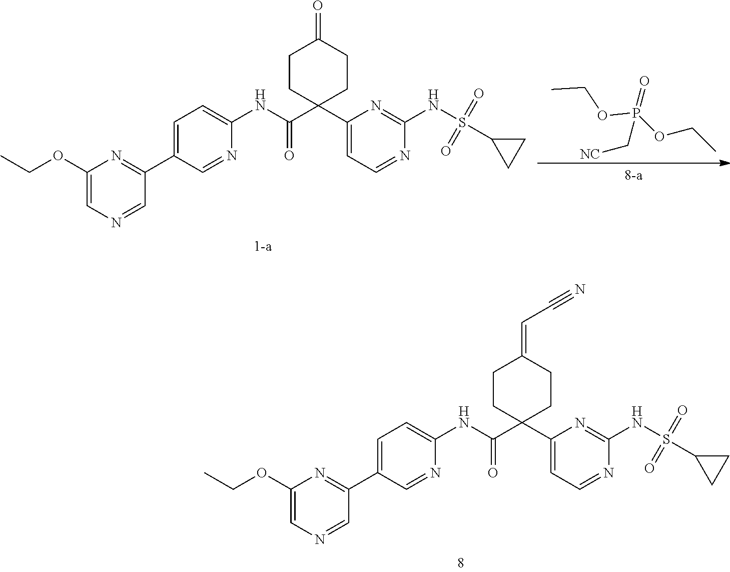 [SMILES: CCOc1cncc(-c2ccc(NC(=O)C3(c4ccnc(NS(=O)(=O)C5CC5)n4)CCC(=O)CC3)nc2)n1 ; CCOP(=O)(CC#N)OCC ; CCOc1cncc(-c2ccc(NC(=O)C3(c4ccnc(NS(=O)(=O)C5CC5)n4)CCC(=CC#N)CC3)nc2)n1]

Compound 1-a (16 mg, 0.089 mmol) was dissolved in tetrahydrofuran (4 mL), and sodium hydride (60%, 5 mg) was slowly added at 0° C. The reaction mixture was stirred at room temperature for 30 minutes, followed by the addition of a solution of compound 8-a (40 mg, 0.074 mmol) in tetrahydrofuran (1 mL). The reaction mixture was stirred at room temperature for an additional 1 hour. After completion, the reaction was quenched with saturated ammonium chloride solution and extracted with ethyl acetate (3×15 mL). The combined organic phases were washed with saturated brine, dried over anhydrous sodium sulfate, filtered, and concentrated under reduced pressure. The crude product was purified by silica gel column chromatography (dichloromethane:methanol=50:1) to afford compound 8 as a white solid (41 mg, 98% yield). ¹H NMR (500 MHz, DMSO-d₆) δ 11.30 (br, 1H), 10.24 (s, 1H), 9.03 (d, J=1.8 Hz, 1H), 8.84 (s, 1H), 8.62 (d, J=5.2 Hz, 1H), 8.50 (dd, J=8.8, 2.3 Hz, 1H), 8.25 (s, 1H), 8.20 (d, J=8.9 Hz, 1H), 7.26 (d, J=5.3 Hz, 1H), 5.52 (s, 1H), 4.47 (q, J=7.0 Hz, 2H), 3.28-3.20 (m, 1H), 2.71-2.53 (m, 4H), 2.48-2.42 (m, 2H), 2.24-2.12 (m, 2H), 1.40 (t, J=7.0 Hz, 3H), 1.09-1.01 (m, 2H), 0.90-0.82 (m, 2H) ppm. MS m/z 561.1 [M+H]⁺.

### Example 9: Preparation of Compound 9

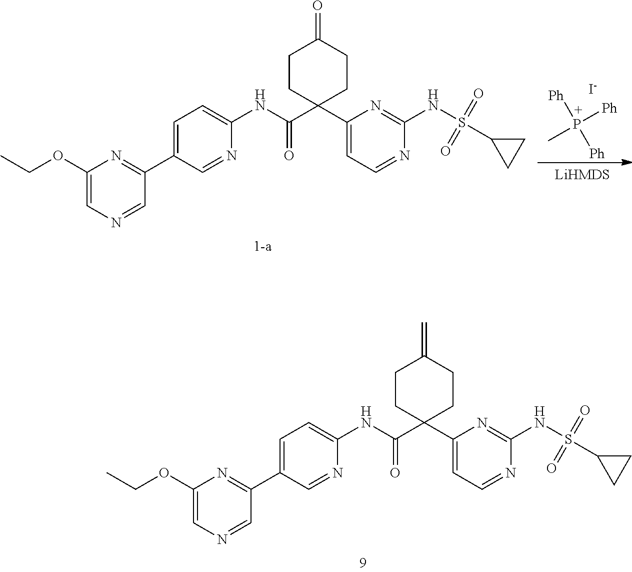

Methyltriphenylphosphonium iodide (54 mg, 0.13 mmol) was dissolved in tetrahydrofuran (2 mL) under nitrogen protection, and lithium bis(trimethylsilyl)amide in tetrahydrofuran (1 M, 0.13 mL) was added dropwise in an ice bath. The reaction was stirred at this temperature for 0.5 hours, followed by the dropwise addition of a solution of compound 1-a (24 mg, 0.04 mmol) in tetrahydrofuran (2 mL). After addition, the reaction mixture was heated at 65° C. for 2 hours. The reaction was quenched with water and extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=25:1) to afford compound 9 as a white solid (9 mg, 38% yield). ¹H NMR (500 MHz, DMSO-d₆) δ 11.28 (s, 1H), 10.08 (s, 1H), 9.02 (d, J=2.0 Hz, 1H), 8.84 (s, 1H), 8.60 (d, J=5.3 Hz, 1H), 8.49 (dd, J=8.8, 2.3 Hz, 1H), 8.25 (s, 1H), 8.19 (d, J=8.8 Hz, 1H), 7.23 (d, J=5.3 Hz, 1H), 4.69 (s, 2H), 4.47 (q, J=7.0 Hz, 2H), 3.28-3.22 (m, 1H), 2.58-2.53 (m, 2H), 2.33-2.21 (m, 4H), 2.11-2.03 (m, 2H), 1.40 (t, J=7.0 Hz, 3H), 1.06-1.02 (m, 2H), 0.91-0.83 (m, 2H) ppm. MS m/z 536.0 [M+H]⁺.

### Example 10: Preparation of Compound 10

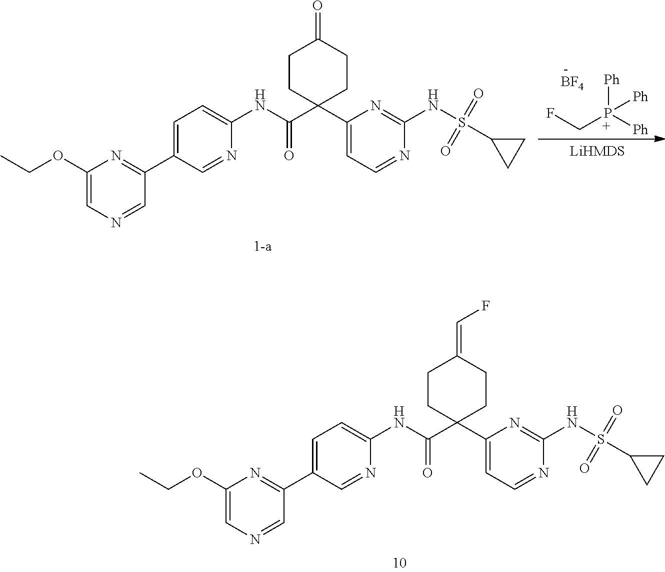

(Fluoromethyl)triphenylphosphonium tetrafluoroborate (58 mg, 0.15 mmol) was dissolved in tetrahydrofuran (2 mL) under nitrogen protection, and lithium bis(trimethylsilyl)amide in tetrahydrofuran (1 M, 0.15 mL) was added dropwise in an ice bath. The reaction was stirred at this temperature for 0.5 hours, followed by the dropwise addition of a solution of compound 1-a (27 mg, 0.05 mmol) in tetrahydrofuran (2 mL). The reaction mixture was stirred at room temperature for 1 hour. The reaction was quenched with water and extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=25:1) to afford compound 10 as a white solid (7 mg, 25% yield). ¹H NMR (500 MHz, DMSO-d) δ 11.29 (s, 1H), 10.15 (s, 1H), 9.08-8.97 (m, 1H), 8.84 (s, 1H), 8.60 (d, J=5.3 Hz, 1H), 8.50 (dd, J=8.8, 2.4 Hz, 1H), 8.25 (s, 1H), 8.20 (d, J=8.9 Hz, 1H), 7.23 (d, J=5.1 Hz, 1H), 6.70 (d, J=87.5 Hz, 1H), 4.47 (q, J=7.0 Hz, 2H), 3.29-3.21 (m, 1H), 2.58-2.51 (m, 2H), 2.23-1.95 (m, 6H), 1.40 (t, J=7.0 Hz, 3H), 1.08-1.00 (m, 2H), 0.89-0.81 (m, 2H) ppm. MS m/z 554.2 [M+H]⁺.

### Example 11: Preparation of Compound 11

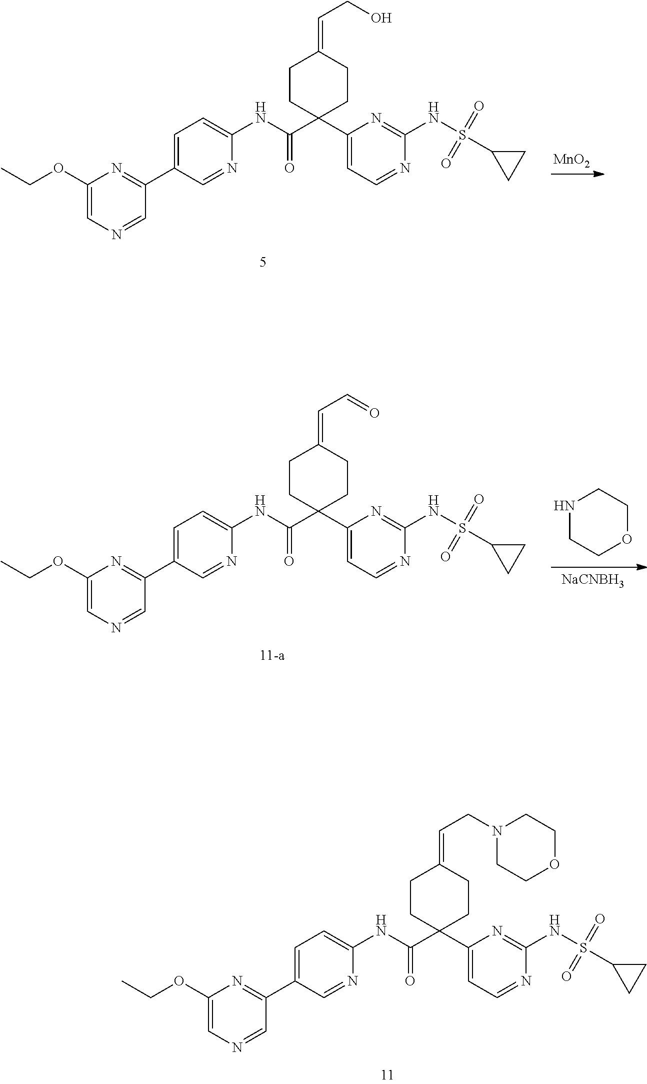

Crude Compound 5 (110 mg, 0.194 mmol) was dissolved in chloroform (6 mL), and then activated manganese dioxide (169 mg, 1.940 mmol) was added. The reaction solution was heated to reflux and stirred overnight. After the reaction was completed, it was cooled to room temperature, and the mixture was filtered and washed. The filtrate was concentrated under reduced pressure to obtain crude Compound 11-a (Note: Compound 11-a is easy to deteriorate during purification), which was directly used in the next reaction. MS m/z 564.2 [M+H]⁺.

Crude Compound 11-a (90 mg, 0.160 mmol) and morpholine (42 mg, 0.479 mmol) were dissolved in 1,2-dichloroethane (4 mL), and 1 drop of acetic acid was added. The reaction was stirred at 50° C. for 1.5 hours, and then sodium cyanoborohydride (30 mg, 0.479 mmol) was added. The mixture was stirred at 50° C. for 30 minutes. After the reaction was completed, it was cooled to room temperature, and the reaction solution was concentrated under reduced pressure. The obtained crude product was separated and purified by preparative thin-layer chromatography (dichloromethane:methanol=20:1) to obtain Compound 11 as a white solid (5.15 mg, yield 5%). ¹H NMR (500 MHz, DMSO-d₆) δ 11.24 (br., 1H), 9.99 (s, 1H), 9.01 (d, J=2.0 Hz, 1H), 8.83 (s, 1H), 8.60 (d, J=5.3 Hz, 1H), 8.48 (dd, J=8.8, 2.3 Hz, 1H), 8.25 (s, 1H), 8.16 (d, J=8.8 Hz, 1H), 7.19 (d, J=5.3 Hz, 1H), 5.59-5.51 (m, 1H), 4.47 (q, J=7.0 Hz, 2H), 3.70-3.45 (m, 4H), 3.25-3.15 (m, 1H), 2.82-2.66 (m, 2H), 2.47-1.77 (m, 12H), 1.39 (t, J=7.0 Hz, 3H), 1.09-0.98 (m, 2H), 0.84-0.73 (m, 2H) ppm. MS m/z 635.2 [M+H]⁺.

### Example 12: Preparation of Compound 12

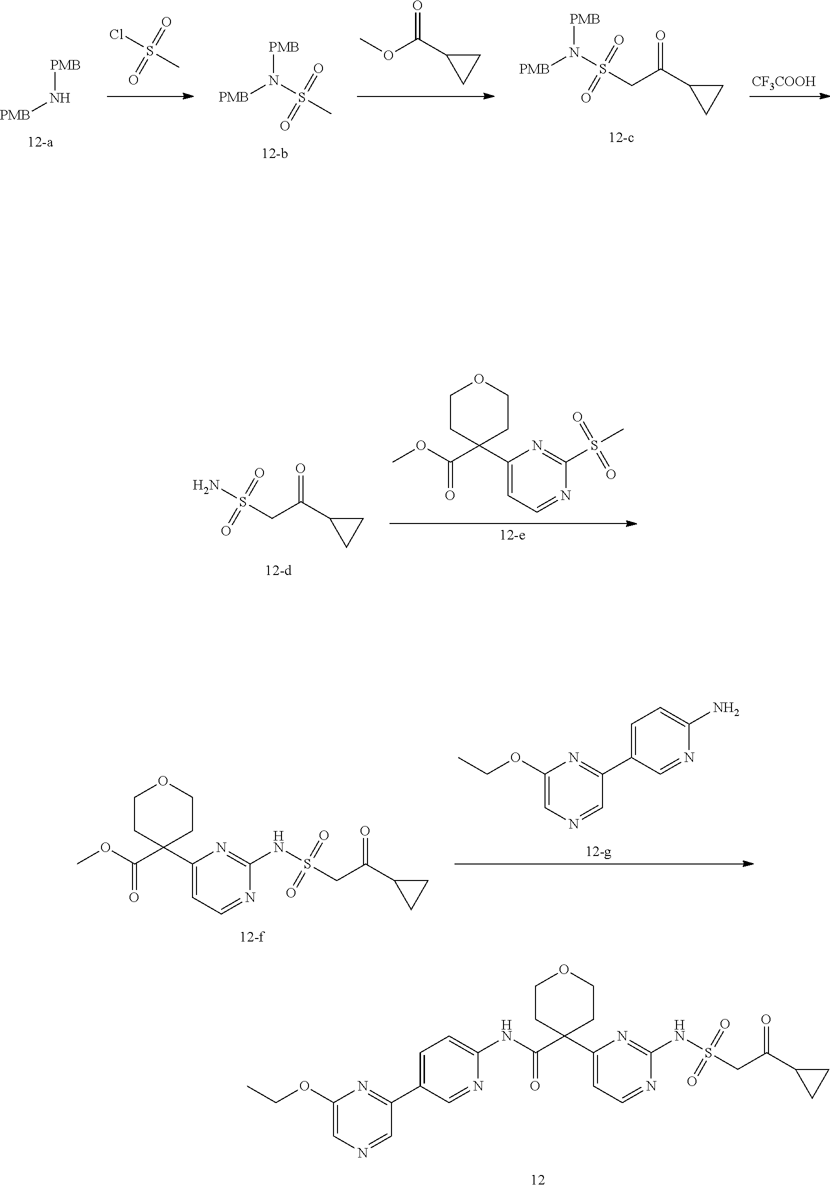

Compound 12-a (1.0 g, 3.89 mmol) and triethylamine (511 mg, 5.05 mmol) were dissolved in dichloromethane (10 mL). Methanesulfonyl chloride (534 mg, 4.66 mmol) was slowly added at 0° C. The reaction solution was stirred at room temperature for 1 hour. After the reaction was completed, the mixture was concentrated under reduced pressure. The obtained crude product was separated and purified by silica gel column chromatography (petroleum ether:ethyl acetate=3:1) to give Compound 12-b as a white solid (1.37 g, yield 100%). MS m/z 336.2 [M+H]⁺.

Compound 12-b (1.37 g, 4.08 mmol) was dissolved in tetrahydrofuran (14 mL). A solution of n-butyllithium in tetrahydrofuran (2.2 M, 2.78 mL) was slowly added at −78° C. After the addition was completed, the reaction was warmed to 0° C. and stirred for 30 minutes, then cooled to −78° C., and a solution of methyl cyclopropanecarboxylate (613 mg, 6.13 mmol) in tetrahydrofuran (3 mL) was added. After the addition was completed, the reaction mixture was stirred at room temperature for 1 hour. After the reaction was completed, the reaction solution was quenched with 2 N hydrochloric acid and extracted with ethyl acetate (3×35 mL). The combined organic phases were washed with saturated brine, dried over anhydrous sodium sulfate, filtered, and concentrated under reduced pressure. The obtained crude product was separated and purified by silica gel column chromatography (petroleum ether:ethyl acetate=4:1) to give Compound 12-c as a yellow oil (1.45 g, yield 88%). MS m/z 404.2 [M+H]⁺.

Compound 12-c (500 mg, 1.24 mmol) was dissolved in trifluoroacetic acid (6 mL), and the reaction mixture was stirred at room temperature for 1 hour. The reaction solution was concentrated under reduced pressure to give a crude product containing Compound 12-d, which was directly used in the next step. MS m/z 164.1 [M+H]⁺.

Compound 12-e (160 mg, 0.533 mmol) (the synthesis of Intermediate 12-e refers to Patent WO 2020/245665 A1) and Compound 12-d (theoretical yield: 202 mg, 1.23 mmol) were dissolved in N-methylpyrrolidone (6 mL). Cesium carbonate (521 mg, 1.60 mmol) was added, and the reaction system was stirred at 60° C. for 1 hour. After the reaction was completed, the reaction solution was diluted with water and washed with methyl tert-butyl ether. The organic phase was separated, acidified, and concentrated under reduced pressure. The obtained crude product was separated and purified by silica gel column chromatography (dichloromethane:methanol=35:1) to give crude Compound 12-f as a yellow oil (400 mg, containing N-methylpyrrolidone). MS m/z 384.2 [M+H]⁺.

Compound 12-f (theoretical yield: 204 mg, 0.532 mmol) and Compound 12-g (207 mg, 0.958 mmol) were dissolved in tetrahydrofuran (6 mL). Lithium bis(trimethylsilyl)amide (1.0 M, 1.6 mL) was slowly added dropwise at 0° C. The reaction solution was stirred at room temperature for 1 hour and quenched with water. The mixture was adjusted to pH=4 with 2 N hydrochloric acid and concentrated under reduced pressure. The obtained crude product was separated and purified by silica gel column chromatography (dichloromethane:methanol=25:1) to give Compound 12 as a white solid (84 mg, yield 28%). ¹H NMR (500 MHz, DMSO-d₆) δ 11.72 (br., 1H), 10.21 (s, 1H), 9.02 (d, J=2.0 Hz, 1H), 8.84 (s, 1H), 8.64 (d, J=4.7 Hz, 1H), 8.49 (dd, J=8.8, 2.3 Hz, 1H), 8.25 (s, 1H), 8.20 (d, J=8.8 Hz, 1H), 7.28 (d, J=4.7 Hz, 1H), 4.88 (s, 2H), 4.47 (q, J=7.0 Hz, 2H), 3.78-3.70 (m, 2H), 3.65-3.55 (m, 2H), 2.48-2.43 (m, 2H), 2.23-2.17 (m, 2H), 2.16-2.11 (m, 1H), 1.39 (t, J=7.0 Hz, 3H), 0.89-0.81 (m, 4H) ppm. MS m/z 568.1 [M+H]⁺.

### Example 13: Preparation of Compound 13

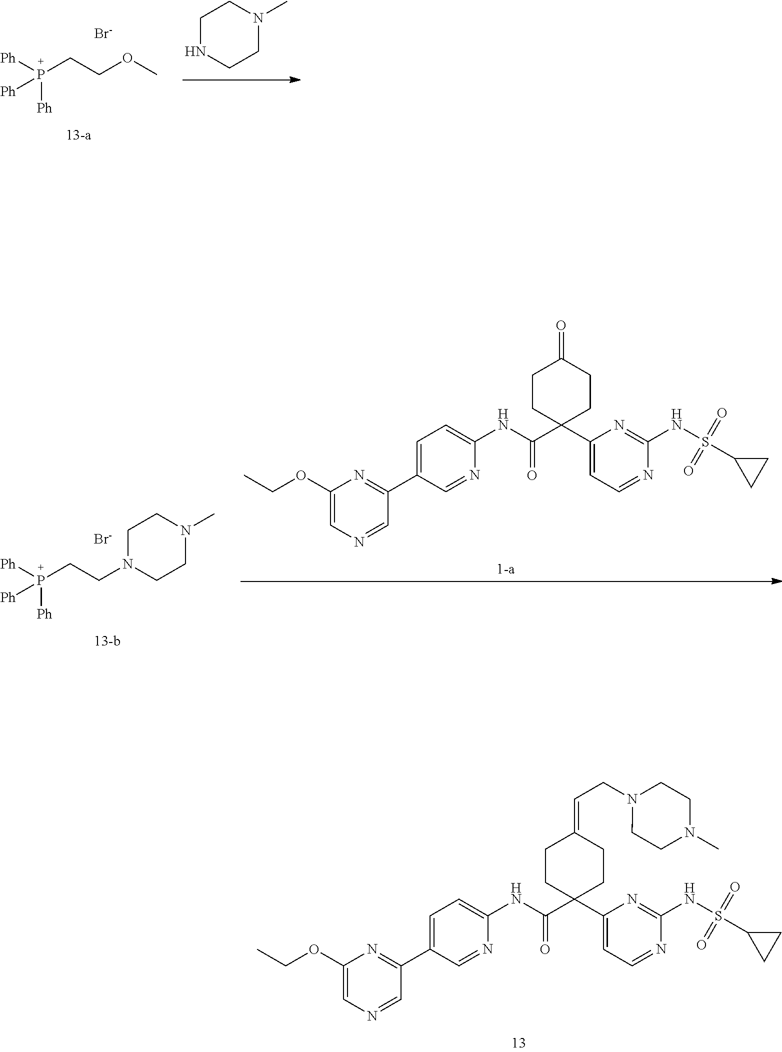

Compound 13-a (60 mg, 0.149 mmol) and N-methylpiperazine (45 mg, 0.448 mmol) were dissolved in water (2 mL), and the reaction solution was stirred at 60° C. for 4 hours (the synthesis method refers to Patent WO2020245665). After the system was cooled to room temperature, the reaction solution was extracted with dichloromethane (3×10 mL). The combined organic phases were washed with saturated brine, dried over anhydrous sodium sulfate, filtered, and concentrated under reduced pressure to give crude Compound 13-b as a white solid (55 mg), which was directly used in the next step. MS m/z 389.2 [M+H]⁺.

Compound 1-a (30 mg, 0.056 mmol) and crude compound 13-b (52 mg, 0.112 mmol) were dissolved in tetrahydrofuran (3 mL). Lithium bis(trimethylsilyl)amide (1.0 M, 0.12 mL) was added at 0° C. The reaction solution was stirred at room temperature for another 2 hours. The reaction solution was quenched with 2 N hydrochloric acid and concentrated under reduced pressure. The obtained crude product was separated and purified by preparative thin-layer chromatography (dichloromethane:methanol=10:1, 2% ammonia water) to give Compound 13 as a white solid (3.87 mg, yield 11%). ¹H NMR (500 MHz, DMSO-d₆) δ 10.09 (s, 1H), 9.02 (d, J=1.8 Hz, 1H), 8.84 (s, 1H), 8.58 (d, J=5.1 Hz, 1H), 8.50 (dd, J=8.8, 2.1 Hz, 1H), 8.25 (s, 1H), 8.19 (d, J=8.8 Hz, 1H), 7.20 (d, J=4.8 Hz, 1H), 5.21 (t, J=6.9 Hz, 1H), 4.47 (q, J=7.0 Hz, 2H), 3.27-3.20 (m, 1H), 3.07-3.00 (m, 1H), 3.00-2.91 (m, 2H), 2.67-2.59 (m, 1H), 2.45-2.25 (m, 8H), 2.25-2.16 (m, 5H), 2.14-1.94 (m, 4H), 1.39 (t, J=7.0 Hz, 3H), 1.06-1.01 (m, 2H), 0.87-0.84 (m, 2H) ppm. MS m/z 648.4 [M+H]⁺.

### Example 14: Preparation of Compound 14

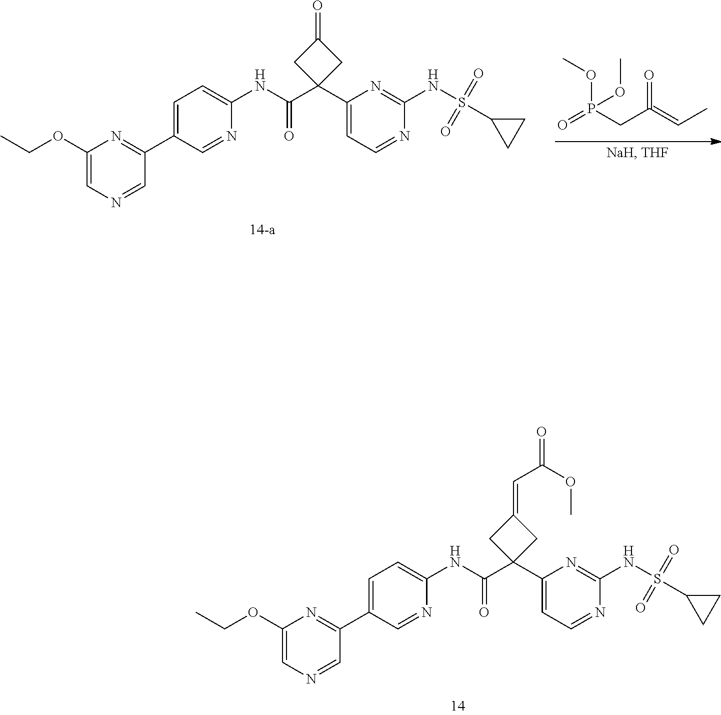

Trimethyl phosphonoacetate (54 mg, 0.30 mmol) was dissolved in tetrahydrofuran (1 mL). 60% sodium hydride (16 mg, 0.40 mmol) was added at 0° C., and the mixture was stirred at 0° C. for 30 minutes, then Compound 14-a (100 mg, 0.20 mmol) was added to the reaction system at 0° C. The reaction solution was stirred at room temperature for 1 hour. After the reaction was completed, it was quenched with water and extracted with ethyl acetate (20 mL×3). The combined organic layers were washed with saturated brine, dried over anhydrous sodium sulfate, filtered, and concentrated under reduced pressure. The obtained crude product was separated and purified by preparative thin-layer plate (dichloromethane:methanol=20:1) to give Compound 14 as a white solid (70 mg, yield 63%). ¹H NMR (500 MHz, DMSO-d₆) δ 11.35 (s, 1H), 10.81 (s, 1H), 9.04 (d, J=2.1 Hz, 1H), 8.85 (s, 1H), 8.60 (d, J=5.1 Hz, 1H), 8.53 (dd, J=8.8, 2.4 Hz, 1H), 8.32-8.18 (m, 2H), 7.23 (d, J=4.9 Hz, 1H), 5.85-5.78 (m, 1H), 4.48 (q, J=7.0 Hz, 2H), 3.89-3.79 (m, 1H), 3.71-3.64 (m, 2H), 3.62 (s, 3H), 3.49-3.39 (m, 1H), 3.26-3.18 (m, 1H), 1.40 (t, J 20=7.0 Hz, 3H), 1.12-1.00 (m, 2H), 0.94-0.82 (m, 2H). MS m/z 566.2 [M+H]⁺.

### Example 15: Preparation of Compound 15

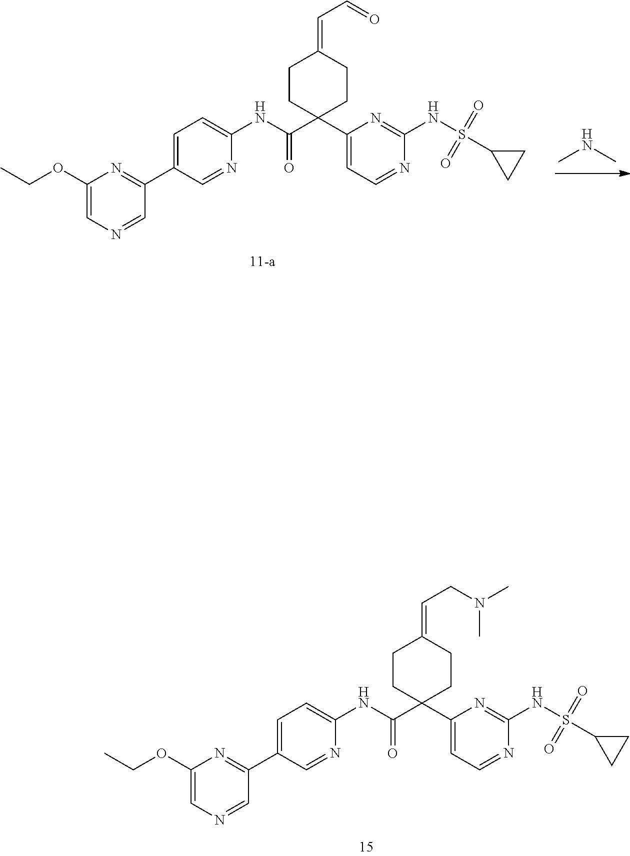

Compound 11-a (25 mg, 0.044 mmol) and a solution of dimethylamine in tetrahydrofuran (2 M, 0.11 mL) were dissolved in 1,2-dichloroethane (2 mL). One drop of acetic acid was added, and the mixture was stirred at 50° C. for 2 hours. Then sodium cyanoborohydride (8.5 mg, 0.133 mmol) was slowly added, and the system was stirred at 50° C. for 15 minutes. After the reaction was completed, the reaction solution was concentrated under reduced pressure. The obtained crude product was separated and purified by preparative thin-layer chromatography (dichloromethane:methanol=2:1) to give Compound 15 as a white solid (2.39 mg, yield 9%). ¹H NMR (500 MHz, DMSO-d₆) δ 10.04 (s, 1H), 9.01 (d, J=2.2 Hz, 1H), 8.84 (s, 1H), 8.58 (d, J=5.3 Hz, 1H), 8.48 (dd, J=8.8, 2.4 Hz, 1H), 8.25 (s, 1H), 8.16 (d, J=8.8 Hz, 1H), 7.17 (d, J=5.3 Hz, 1H), 5.60-5.52 (m, 1H), 4.47 (q, J=7.0 Hz, 2H), 3.24-3.16 (m, 1H), 2.81-2.60 (m, 4H), 2.41 (s, 6H), 2.32-2.24 (m, 2H), 2.21-2.12 (m, 2H), 2.06-1.95 (m, 2H), 1.39 (t, J=7.1 Hz, 3H), 1.06-0.95 (m, 2H), 0.82-0.73 (m, 2H) ppm. MS m/z 593.3 [M+H]⁺.

### Example 16: Preparation of Compound 16

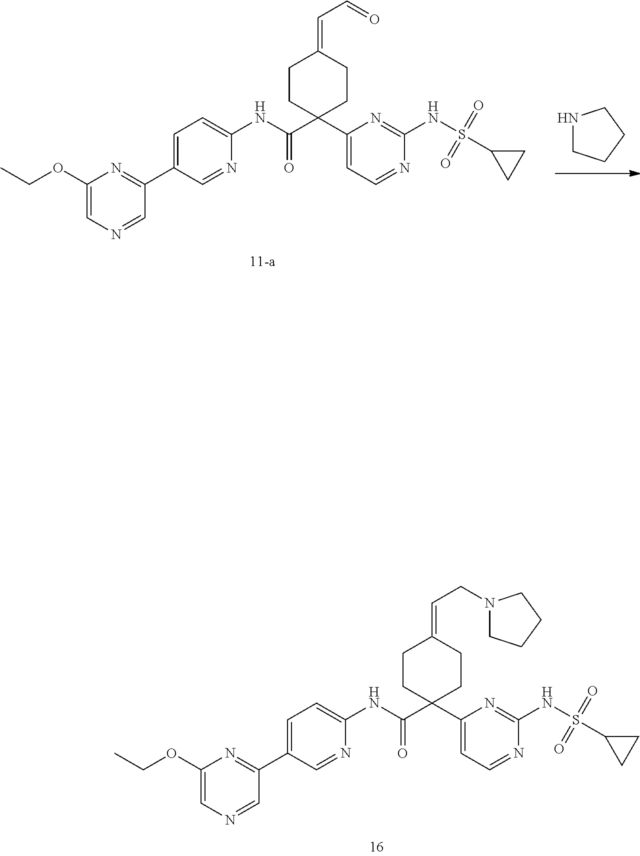 [SMILES: CCOc1cncc(-c2ccc(NC(=O)C3(c4ccnc(NS(=O)(=O)C5CC5)n4)CCC(=CC=O)CC3)nc2)n1 ; C1CCNC1 ; CCOc1cncc(-c2ccc(NC(=O)C3(c4ccnc(NS(=O)(=O)C5CC5)n4)CCC(=CCN4CCCC4)CC3)nc2)n1]

Compound 11-a (25 mg, 0.044 mmol) and tetrahydropyrrole (16 mg, 0.222 mmol) were dissolved in 1,2-dichloroethane (2 mL), and 1 drop of acetic acid was added. The reaction was stirred at 50° C. for 2 hours, then sodium cyanoborohydride (8.5 mg, 0.133 mmol) was added. The reaction mixture was stirred at 50° C. for another 15 minutes. After the reaction was cooled to room temperature, it was concentrated under reduced pressure. The obtained crude product was separated and purified by preparative thin-layer chromatography (dichloromethane:methanol=5:1) to give Compound 16 as a white solid (2.47 mg, yield 9%). ¹H NMR (500 MHz, DMSO-d₆) δ 10.07 (s, 1H), 9.01 (d, J=2.2 Hz, 1H), 8.84 (s, 1H), 8.59 (d, J=5.3 Hz, 1H), 8.49 (dd, J=8.8, 2.4 Hz, 1H), 8.26 (s, 1H), 8.16 (d, J=8.8 Hz, 1H), 7.17 (d, J=5.3 Hz, 1H), 5.63-5.56 (m, 1H), 4.47 (q, J=7.0 Hz, 2H), 3.24-3.16 (m, 1H), 3.08-2.89 (m, 4H), 2.83-2.67 (m, 2H), 2.47-2.43 (m, 2H), 2.34-2.23 (m, 3H), 2.11-1.97 (m, 2H), 1.90-1.79 (m, 5H), 1.39 (t, J=7.0 Hz, 3H), 1.07-0.95 (m, 2H), 0.82-0.73 (m, 2H) ppm. MS m/z 619.2 [M+H]⁺.

### Example 17&18: Preparation of Compound 17&18

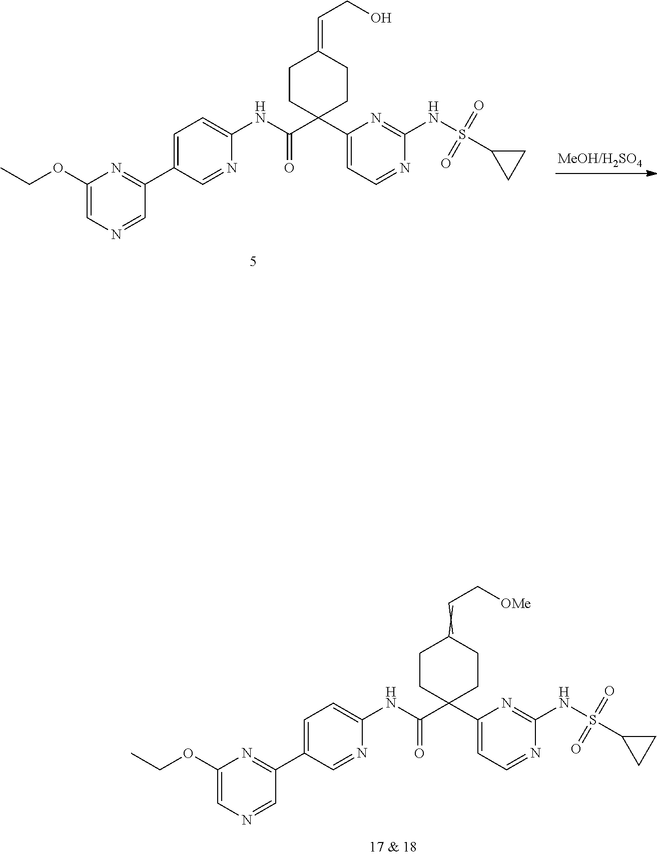 [SMILES: CCOc1cncc(-c2ccc(NC(=O)C3(c4ccnc(NS(=O)(=O)C5CC5)n4)CCC(=CCO)CC3)nc2)n1 ; CCOc1cncc(-c2ccc(NC(=O)C3(c4ccnc(NS(=O)(=O)C5CC5)n4)CCC(=CCOC)CC3)nc2)n1]

Compound 5 (17 mg, 0.030 mmol) was dissolved in methanol, and 2 drops of concentrated sulfuric acid were added under an ice-water bath. The reaction solution was stirred at 50° C. for 24 hours under a nitrogen atmosphere. After the reaction was completed, the reaction solution was concentrated under reduced pressure. The crude product was quenched with saturated sodium bicarbonate solution at 0° C., and then extracted with dichloromethane (3×10 mL). The combined organic phases were washed with saturated brine, dried over anhydrous sodium sulfate, filtered, and concentrated under reduced pressure. The obtained crude product was separated and purified by preparative thin-layer chromatography (dichloromethane:ethyl acetate=1:1) to give two white solid isomer products P1: 17 (1.42 mg); P2: 18 (1.95 mg). P1: MS m/z 580.3 [M+H]⁺; P2: MS m/z 580.3 [M+H]⁺.

### Example 19: Preparation of Compound 19

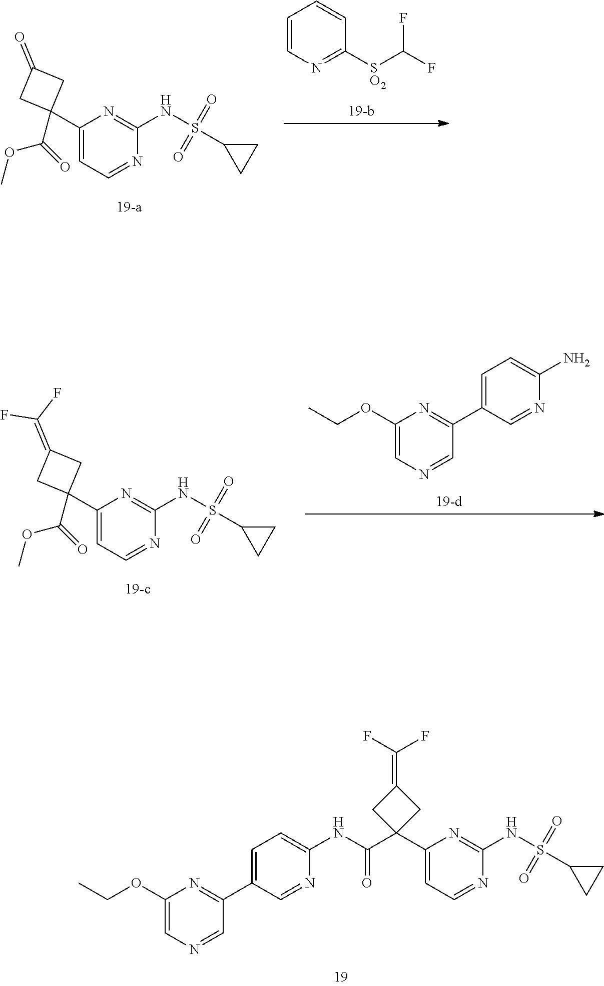 [SMILES: COC(=O)C1(c2ccnc(NS(=O)(=O)C3CC3)n2)CC(=O)C1 ; O=S(=O)(c1ccccn1)C(F)F ; COC(=O)C1(c2ccnc(NS(=O)(=O)C3CC3)n2)CC(=C(F)F)C1 ; CCOc1cncc(-c2ccc(N)nc2)n1 ; CCOc1cncc(-c2ccc(NC(=O)C3(c4ccnc(NS(=O)(=O)C5CC5)n4)CC(=C(F)F)C3)nc2)n1]

Compound 19-a (125 mg, 0.38 mmol) and Compound 19-b (82 mg, 0.42 mmol) were dissolved in DMF (3 mL). Under a nitrogen atmosphere, the mixture was cooled to −60° C., and a solution of potassium tert-butoxide (141 mg, 1.26 mmol) in DMF (2 mL) was added dropwise. After the dropwise addition was completed, the reaction solution was stirred at −60° C. for 0.5 hours. Dilute hydrochloric acid (3 mL, 1M) was added dropwise, and the reaction solution was heated to 70° C. and stirred overnight. After the reaction was completed, it was diluted with water and extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 19-c as a white solid (6 mg, yield 4%). MS m/z 359.1 [M+H]⁺.

Compound 19-c (6 mg, 0.02 mmol) and Compound 19-d (4 mg, 0.02 mmol) were dissolved in tetrahydrofuran (1 mL). Under a nitrogen atmosphere, a solution of lithium bis(trimethylsilyl)amide in tetrahydrofuran (0.1 mL, 1M) was slowly added dropwise. After the dropwise addition was completed, the reaction solution was stirred at room temperature for 0.5 hours. The reaction solution was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 19 as a white solid (2 mg, yield 22%). ¹H NMR (500 MHz, DMSO-d) δ 11.36 (br., 1H), 10.78 (s, 1H), 9.04 (d, J=2.0 Hz, 1H), 8.85 (s, 1H), 8.61 (d, J=4.4 Hz, 1H), 8.53 (dd, J=8.8, 2.4 Hz, 1H), 8.27-8.23 (m, 2H), 7.27-7.23 (m, 1H), 4.48 (q, J=7.0 Hz, 2H), 3.54-3.47 (m, 2H), 3.29-3.26 (m, 2H), 3.24-3.21 (m, 1H), 1.40 (t, J=7.0 Hz, 3H), 1.08-1.04 (m, 2H), 0.93-0.89 (m, 2H). MS m/z 544.0 [M+H]⁺.

### Example 20: Preparation of Compound 20

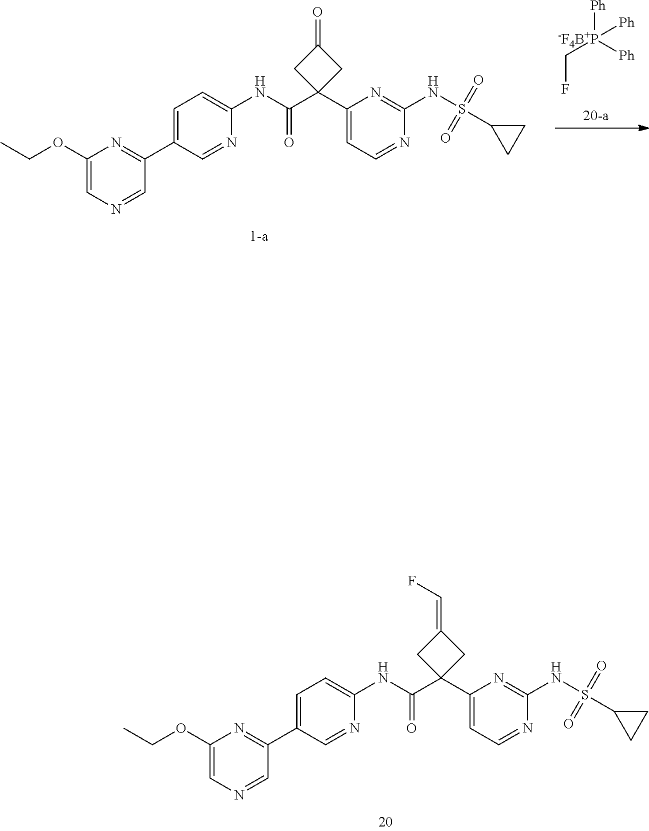

(Fluoromethyl)triphenylphosphonium tetrafluoroborate (46 mg, 0.12 mmol) was dissolved in tetrahydrofuran (1 mL). Under an ice bath and nitrogen atmosphere, a solution of lithium bis(trimethylsilyl)amide in tetrahydrofuran (0.12 mL, 1 M) was added dropwise. The reaction solution was stirred for another 0.5 hours, then a solution of Compound 1-a (20 mg, 0.04 mmol) in tetrahydrofuran (1 mL) was added dropwise. The reaction solution was stirred at room temperature for 1 hour. After the reaction was completed, it was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 20 as a white solid (0.56 mg, yield 3%). MS m/z 526.2 [M+H]⁺.

### Example 21: Preparation of Compound 21

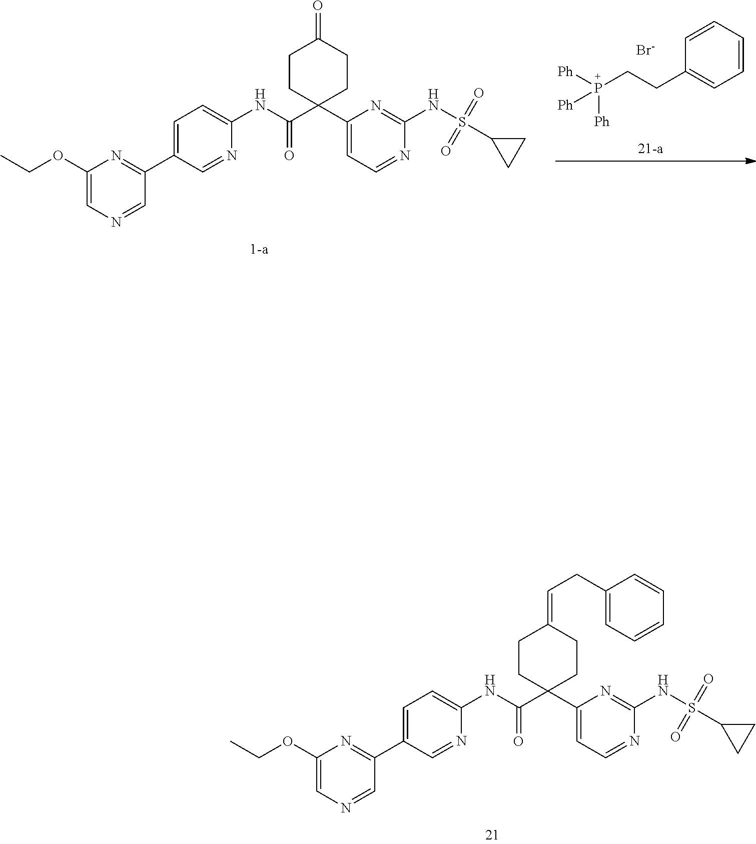

Compound 21-a (25 mg, 0.056 mmol) was dissolved in tetrahydrofuran (1.5 mL). Lithium bis(trimethylsilyl)amide (1.0 M, 0.10 mL) was added at 0° C. The reaction solution was stirred at room temperature for 1 hour, then Compound 1-a (20 mg, 0.130 mmol) was added. The reaction mixture was stirred at room temperature for 30 minutes. After the reaction was completed, the mixture was quenched with 2 N hydrochloric acid and concentrated under reduced pressure. The obtained crude product was separated and purified by preparative thin-layer chromatography (dichloromethane:methanol=20:1) to give Compound 21 as a white solid (3.86 mg, yield 17%). ¹H NMR (500 MHz, DMSO-d₆) δ 11.31 (br., 1H), 10.10 (s, 1H), 9.03 (d, J=2.2 Hz, 1H), 8.85 (s, 1H), 8.58 (d, J=3.7 Hz, 1H), 8.50 (dd, J=8.8, 2.3 Hz, 1H), 8.26 (s, 1H), 8.20 (d, J=8.8 Hz, 1H), 7.32-7.22 (m, 3H), 7.22-7.13 (m, 3H), 5.37-5.27 (m, 1H), 4.47 (q, J=7.0 Hz, 2H), 3.28-3.19 (m, 1H), 2.76-2.60 (m, 2H), 2.56-2.53 (m, 1H), 2.38-2.15 (m, 4H), 2.14-2.03 (m, 2H), 2.03-1.95 (m, 1H), 1.40 (t, J=7.0 Hz, 3H), 1.07-0.99 (m, 2H), 0.90-0.78 (m, 2H) ppm. MS m/z 626.3 [M+H]⁺.

### Example 22: Preparation of Compound 22

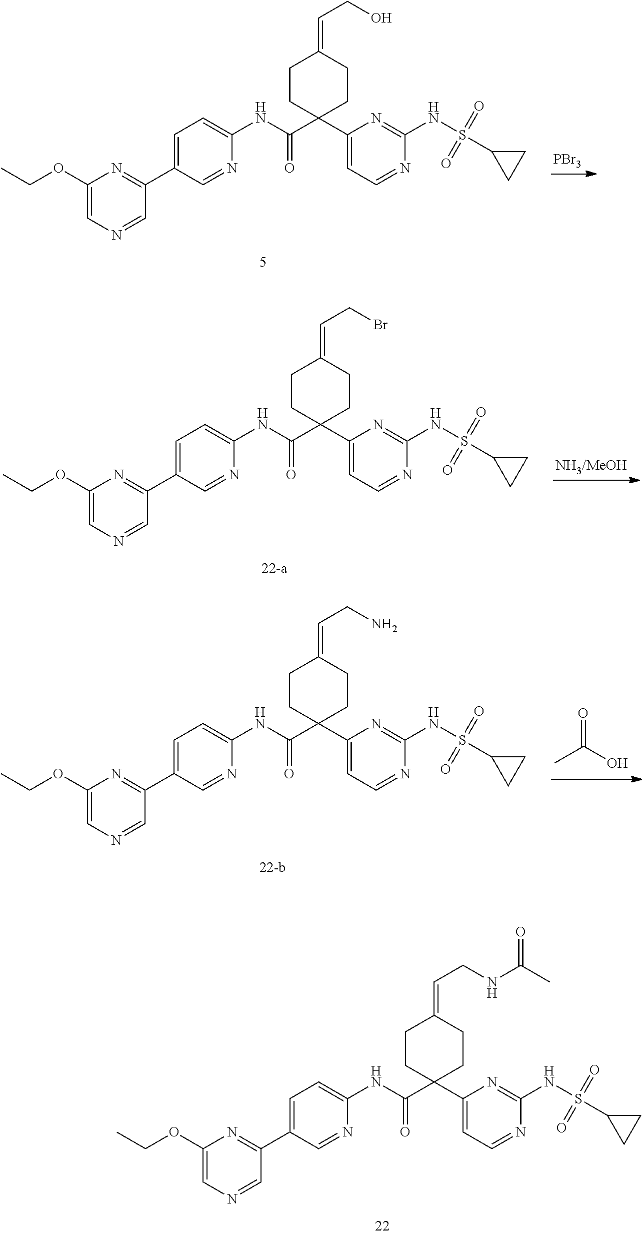

Compound 5 (20 mg, 0.035 mmol) was dissolved in dichloromethane (2 mL). Phosphorus tribromide (29 mg, 0.106 mmol) was added dropwise under an ice-water bath. The reaction solution was stirred at 0° C. for 10 minutes under a nitrogen atmosphere. After the reaction was completed, the reaction solution was directly concentrated under reduced pressure to give crude Compound 22-a as a yellow solid (30 mg). MS m/z 628.2, 630.2 [M+H]⁺.

Crude Compound 22-a (30 mg) was dissolved in ammonia-methanol solution (4 M, 1.5 mL), and the reaction solution was stirred at room temperature for 1 hour. After the reaction was completed, the reaction solution was concentrated under reduced pressure.

The obtained crude product was separated and purified by preparative thin-layer chromatography (dichloromethane:methanol=18:1) to give crude Compound 22-b as a yellow solid (10 mg). MS m/z 565.2 [M+H]⁺.

Compound 22-b (10 mg, 0.018 mmol) was dissolved in N,N-dimethylformamide (1.5 mL). Under a nitrogen atmosphere, N,N,N′,N′-tetramethyl-O-(7-azabenzotriazol-1-yl) uronium hexafluorophosphate (14 mg, 0.036 mmol), acetic acid (3 mg, 0.053 mmol) and triethylamine (5 mg, 0.053 mmol) were added sequentially. The reaction mixture was stirred at room temperature for 30 minutes. After the reaction was completed, the reaction solution was concentrated under reduced pressure. The obtained crude product was separated and purified by preparative thin-layer chromatography (dichloromethane:methanol=20:1) to give Compound 22 as a white solid (1.99 mg, yield 19%). ¹H NMR (500 MHz, DMSO-d₆) δ 11.30 (br., 1H), 10.10 (s, 1H), 9.02 (d, J=2.2 Hz, 1H), 8.84 (s, 1H), 8.59 (d, J=5.2 Hz, 1H), 8.50 (dd, J=8.8, 2.4 Hz, 1H), 8.26 (s, 1H), 8.19 (d, J=8.8 Hz, 1H), 7.87 (t, J=5.2 Hz, 1H), 7.22 (d, J=3.4 Hz, 1H), 5.16 (t, J=6.8 Hz, 1H), 4.47 (q, J=7.1 Hz, 2H), 3.71-3.59 (m, 2H), 3.29-3.21 (m, 1H), 2.47-2.34 (m, 2H), 2.33-2.22 (m, 1H), 2.22-2.14 (m, 2H), 2.12-1.92 (m, 3H), 1.78 (s, 3H), 1.39 (t, J=7.0 Hz, 3H), 1.07-1.00 (m, 2H), 0.88-0.84 (m, 2H) ppm. MS m/z 607.2 [M+H]⁺.

### Example 23: Preparation of Compound 23

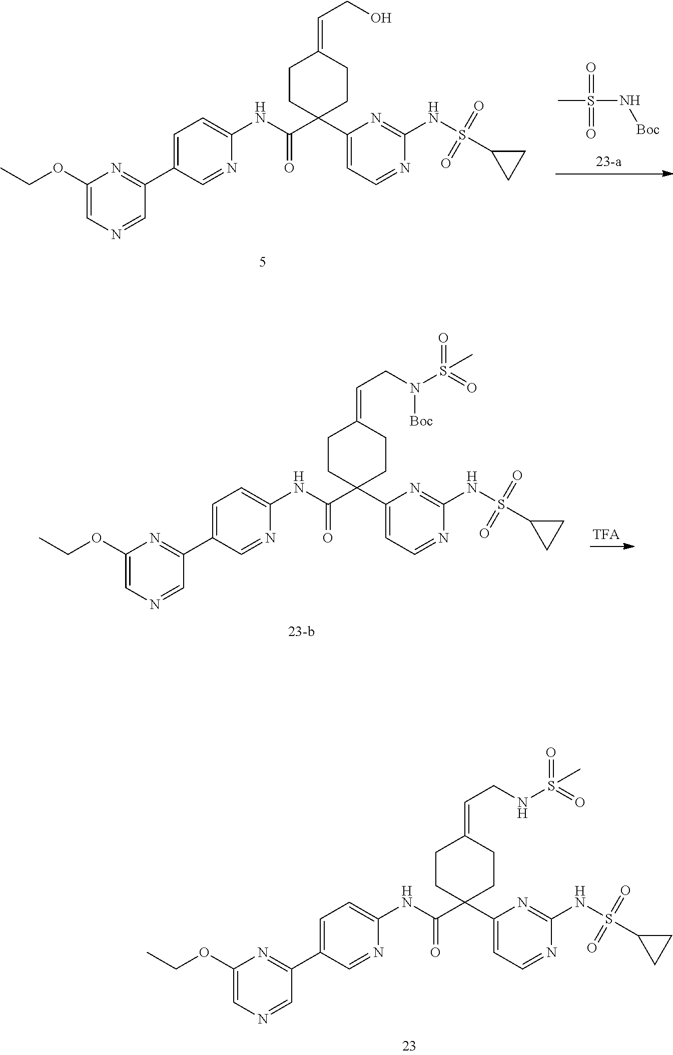 [SMILES: CCOc1cncc(-c2ccc(NC(=O)C3(c4ccnc(NS(=O)(=O)C5CC5)n4)CCC(=CCO)CC3)nc2)n1 ; CC(C)(C)OC(=O)NS(C)(=O)=O ; CCOc1cncc(-c2ccc(NC(=O)C3(c4ccnc(NS(=O)(=O)C5CC5)n4)CCC(=CCN(C(=O)OC(C)(C)C)S(C)(=O)=O)CC3)nc2)n1 ; CCOc1cncc(-c2ccc(NC(=O)C3(c4ccnc(NS(=O)(=O)C5CC5)n4)CCC(=CCNS(C)(=O)=O)CC3)nc2)n1]

Under an ice-water bath, Compound 5 (10 mg, 0.018 mmol), 23-a (4 mg, 0.021 mmol) and triphenylphosphine (9 mg, 0.035 mmol) were sequentially dissolved in dichloromethane (1.5 mL). After diethyl azodicarboxylate (8 mg, 0.035 mmol) was added, the reaction solution was stirred at room temperature for 1 hour. After the reaction was completed, the reaction solution was concentrated under reduced pressure. The obtained crude product was separated and purified by silica gel column chromatography (dichloromethane:methanol=20:1) to give Compound 23-b as a white solid (4 mg, yield 30%). MS m/z 743.3 [M+H]⁺.

Compound 23-b (4 mg, 0.005 mmol) was dissolved in dichloromethane (1 mL). Trifluoroacetic acid (0.2 mL) was added, and the reaction solution was stirred at room temperature for 30 minutes. The reaction solution was concentrated under reduced pressure, and the obtained crude product was separated and purified by preparative thin-layer chromatography (dichloromethane:methanol=18:1) to give Compound 23 as a white solid (2.4 mg, yield 69%). ¹H NMR (500 MHz, DMSO-d₆) δ 11.30 (br., 1H), 10.09 (s, 1H), 9.02 (d, J=2.1 Hz, 1H), 8.84 (s, 1H), 8.58 (d, J=2.4 Hz, 1H), 8.50 (dd, J=8.8, 2.4 Hz, 1H), 8.25 (s, 1H), 8.19 (d, J=8.8 Hz, 1H), 7.18 (br., 1H), 7.02 (t, J=5.8 Hz, 1H), 5.22 (t, J=6.9 Hz, 1H), 4.47 (q, J=7.0 Hz, 2H), 3.64-3.54 (m, 2H), 3.28-3.18 (m, 1H), 2.89 (s, 3H), 2.45-2.34 (m, 2H), 2.32-2.17 (m, 3H), 2.13-1.95 (m, 3H), 1.39 (t, J=7.0 Hz, 3H), 1.05-1.00 (m, 2H), 0.89-0.75 (m, 2H) ppm. MS m/z 643.2 [M+H]⁺.

### Example 24&30: Preparation of Compound 24&30

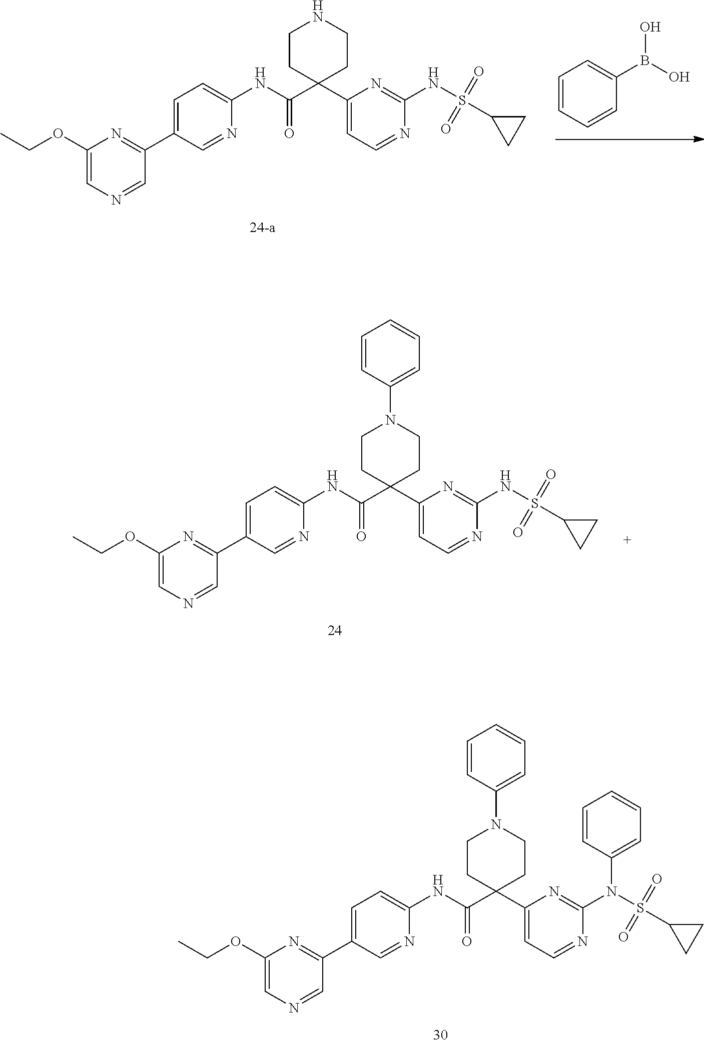

Compound 24-a (20 mg, 0.038 mmol) (the synthesis route of 24-a refers to Patent WO2020245665), phenylboronic acid (7 mg, 0.057 mmol) and copper acetate (14 mg, 0.076 mmol) were dissolved in dichloromethane (2.5 mL). Triethylamine (8 mg, 0.076 mmol) was added, and the reaction solution was stirred at room temperature overnight. After the reaction was completed, the reaction solution was concentrated under reduced pressure. The obtained crude product was separated and purified by preparative thin-layer chromatography (dichloromethane:methanol=30:1) to give Compound 24 as a white solid (1.62 mg, yield 7%) with MS m/z 601.1 [M+H]⁺ and Compound 30 as a white solid (0.60 mg, yield 2.6%) with MS m/z 677.1 [M+H]⁺.

### Example 25: Preparation of Compound 25

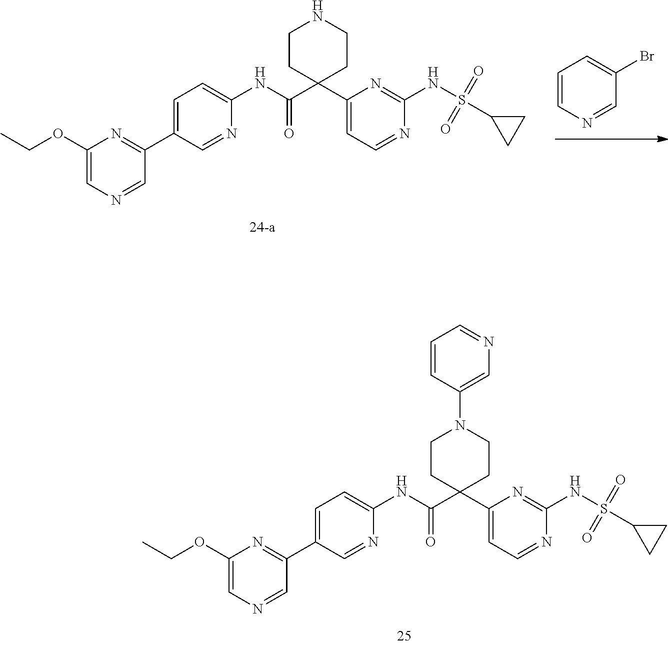

Compound 24-a (55 mg, 0.105 mmol), 3-bromopyridine (33 mg, 0.210 mmol), tris(dibenzylideneacetone)dipalladium (10 mg, 0.010 mmol), 4,5-bis(diphenyl phosphino)-9,9-dimethylxanthene (12 mg, 0.021 mmol) and cesium carbonate (103 mg, 0.315 mmol) were dissolved in 1,4-dioxane (2.5 mL), and nitrogen was bubbled for 2 minutes. The reaction solution was stirred at 95° C. overnight. After the reaction was cooled to room temperature, the reaction solution was concentrated under reduced pressure. The obtained crude product was separated and purified by preparative thin-layer chromatography (dichloromethane:methanol=25:1) to give Compound 25 as a white solid (0.50 mg, yield 1%). MS m/z 602.2 [M+H]⁺.

### Example 26: Preparation of Compound 26

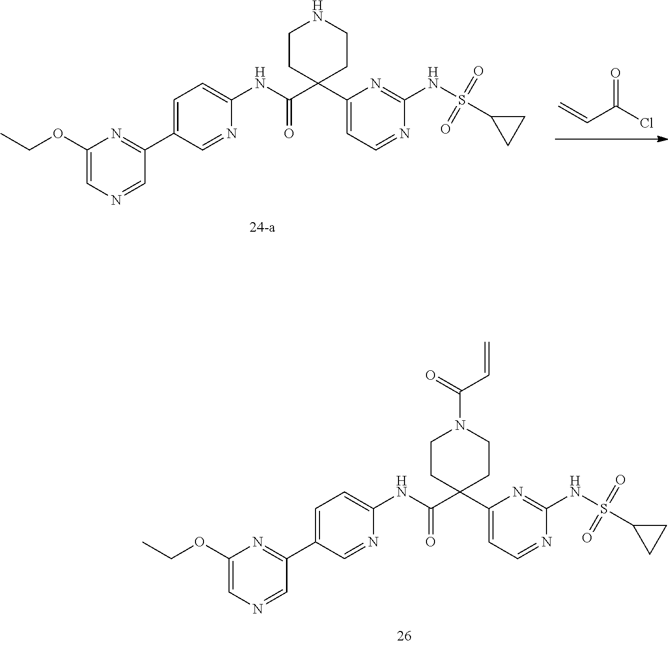

Compound 24-a (20 mg, 0.038 mmol) was dissolved in dichloromethane (2.5 mL). A diluted solution of acryloyl chloride (4 mg, 0.046 mmol) in dichloromethane (1 mL) was slowly added dropwise under an ice-water bath. The reaction solution was stirred at 0° C. for 30 minutes. After the reaction was completed, methanol was added for quenching, and the mixture was concentrated under reduced pressure. The obtained crude product was separated and purified by preparative thin-layer chromatography (dichloromethane:methanol=20:1) to give Compound 26 as a white solid (4.2 mg, yield 19%). ¹H NMR (500 MHz, DMSO-d₆) δ 11.32 (br., 1H), 10.26 (s, 1H), 9.03 (s, 1H), 8.84 (s, 1H), 8.60 (d, J=4.4 Hz, 1H), 8.50 (dd, J=8.7, 1.8 Hz, 1H), 8.25 (s, 1H), 8.19 (d, J=8.8 Hz, 1H), 7.25-7.19 (m, 1H), 6.84 (dd, J=16.6, 10.5 Hz, 1H), 6.11 (d, J=16.7 Hz, 1H), 5.68 (d, J=10.7 Hz, 1H), 4.47 (q, J=7.0 Hz, 2H), 4.05-3.96 (m, 1H), 3.91-3.82 (m, 1H), 3.55-3.44 (m, 1H), 3.30-3.17 (m, 3H), 2.18-2.08 (m, 2H), 2.04-1.93 (m, 1H), 1.39 (t, J=7.0 Hz, 3H), 1.09-0.98 (m, 2H), 0.91-0.82 (m, 2H) ppm. MS m/z 579.2 [M+H]⁺.

### Example 27: Preparation of Compound 27

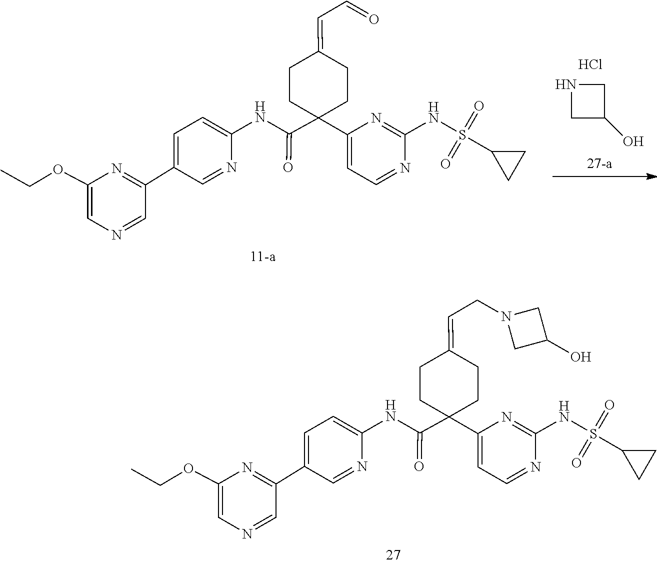 [SMILES: CCOc1cncc(-c2ccc(NC(=O)C3(c4ccnc(NS(=O)(=O)C5CC5)n4)CCC(=CC=O)CC3)nc2)n1 ; OC1CNC1 ; Cl ; CCOc1cncc(-c2ccc(NC(=O)C3(c4ccnc(NS(=O)(=O)C5CC5)n4)CCC(=CCN4CC(O)C4)CC3)nc2)n1]

Compound 11-a (25 mg, 0.044 mmol), 27-a (10 mg, 0.089 mmol), zinc chloride (18 mg, 0.133 mmol) and sodium cyanoborohydride (8 mg, 0.133 mmol) were sequentially dissolved in methanol (2 mL), and the reaction solution was stirred at 50° C. for 15 minutes. After the reaction was completed, the mixture was concentrated under reduced pressure. The obtained crude product was separated and purified by silica gel column chromatography (dichloromethane:methanol=12:1) to give Compound 27 as a white solid (9.7 mg, yield 35%). ¹H NMR (500 MHz, DMSO-d₆) δ 10.10 (s, 1H), 9.02 (d, J=2.4 Hz, 1H), 8.84 (s, 1H), 8.58 (d, J=5.3 Hz, 1H), 8.50 (dd, J=8.8, 2.4 Hz, 1H), 8.25 (s, 1H), 8.19 (d, J=8.9 Hz, 1H), 7.18 (d, J=5.3 Hz, 1H), 5.66 (br., 1H), 5.13 (t, J=7.0 Hz, 1H), 4.47 (q, J=7.0 Hz, 2H), 4.31-4.21 (m, 1H), 3.84-3.74 (m, 2H), 3.28-3.20 (m, 2H), 3.22-3.07 (m, 2H), 2.75-2.64 (m, 2H), 2.48-2.42 (m, 2H), 2.32-2.16 (m, 3H), 2.14-1.98 (m, 2H), 1.39 (t, J=7.0 Hz, 3H), 1.06-0.98 (m, 2H), 0.87-0.80 (m, 2H) ppm. MS m/z 621.2 [M+H]⁺.

### Example 28: Preparation of Compound 28

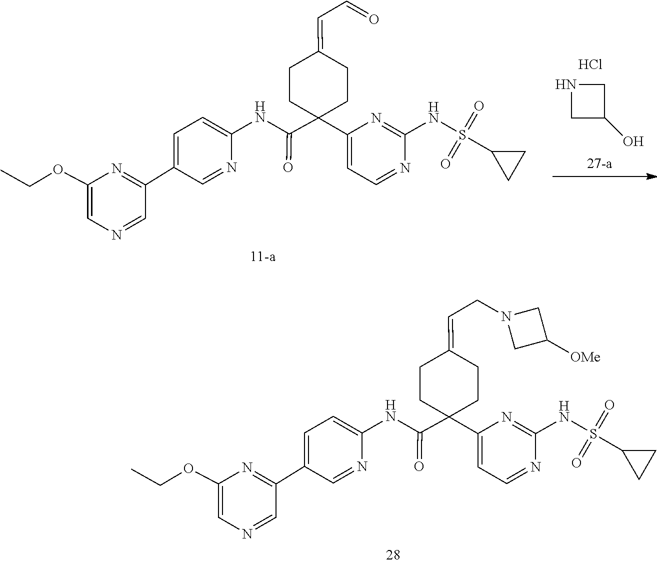 [SMILES: CCOc1cncc(-c2ccc(NC(=O)C3(c4ccnc(NS(=O)(=O)C5CC5)n4)CCC(=CC=O)CC3)nc2)n1 ; OC1CNC1 ; Cl ; CCOc1cncc(-c2ccc(NC(=O)C3(c4ccnc(NS(=O)(=O)C5CC5)n4)CCC(=CCN4CC(OC)C4)CC3)nc2)n1]

The synthesis route of Compound 28 refers to that of Compound 27, giving Compound 28 as a white solid (13 mg, yield 46%). ¹H NMR (500 MHz, DMSO-d₆) δ 10.09 (s, 1H), 9.02 (d, J=2.1 Hz, 1H), 8.84 (s, 1H), 8.57 (d, J=5.3 Hz, 1H), 8.49 (dd, J=8.8, 2.4 Hz, 1H), 8.25 (s, 1H), 8.19 (d, J=8.9 Hz, 1H), 7.18 (d, J=5.3 Hz, 1H), 5.10 (t, J=6.9 Hz, 1H), 4.47 (q, J=7.0 Hz, 2H), 3.99-3.89 (m, 1H), 3.60-3.51 (m, 2H), 3.27-3.20 (m, 2H), 3.14 (s, 3H), 3.13-3.11 (m, 2H), 2.94-2.82 (m, 2H), 2.46-2.34 (m, 2H), 2.32-2.15 (m, 3H), 2.11-1.97 (m, 2H), 1.39 (t, J=7.0 Hz, 3H), 1.04-1.00 (m, 2H), 0.87-0.83 (m, 2H) ppm. MS m/z 635.2 [M+H]⁺.

### Example 29: Preparation of Compound 29

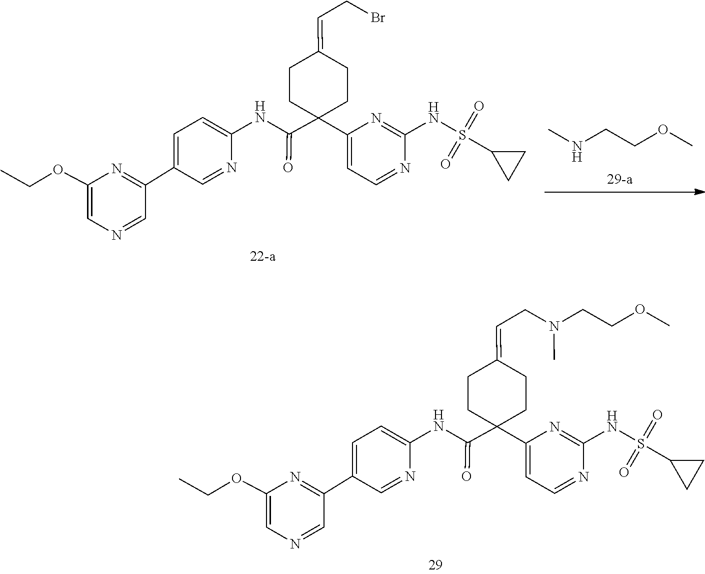

Compound 22-a (8.9 mg, 0.014 mmol) was dissolved in dichloromethane (1.5 mL). 29-a (6.3 mg, 0.071 mmol) was added dropwise under an ice-water bath. The reaction solution was stirred at 0° C. for 10 minutes, and then concentrated under reduced pressure. The obtained crude product was separated and purified by preparative thin-layer chromatography (dichloromethane:methanol=6:1) to give Compound 29 as a white solid (2.52 mg, yield 28%). ¹H NMR (500 MHz, DMSO-d₆) δ 11.12 (br., 1H), 10.08 (s, 1H), 9.02 (d, J=2.0 Hz, 1H), 8.84 (s, 1H), 8.57 (d, J=5.3 Hz, 1H), 8.49 (dd, J=8.8, 2.4 Hz, 1H), 8.25 (s, 1H), 8.19 (d, J=8.8 Hz, 1H), 7.18 (d, J=5.2 Hz, 1H), 5.22 (t, J=6.9 Hz, 1H), 4.47 (q, J=7.0 Hz, 2H), 3.46-3.40 (m, 2H), 3.30-3.27 (m, 1H), 3.23 (s, 3H), 3.09-3.04 (m, 2H), 2.59-2.54 (m, 2H), 2.45-2.39 (m, 2H), 2.33-2.25 (m, 2H), 2.22 (s, 3H), 2.21-2.17 (m, 1H), 2.15-1.95 (m, 3H), 1.39 (t, J=7.0 Hz, 3H), 1.06-1.00 (m, 2H), 0.88-0.81 (m, 2H) ppm. MS m/z 637.2 [M+H]⁺.

### Example 31: Preparation of Compound 31

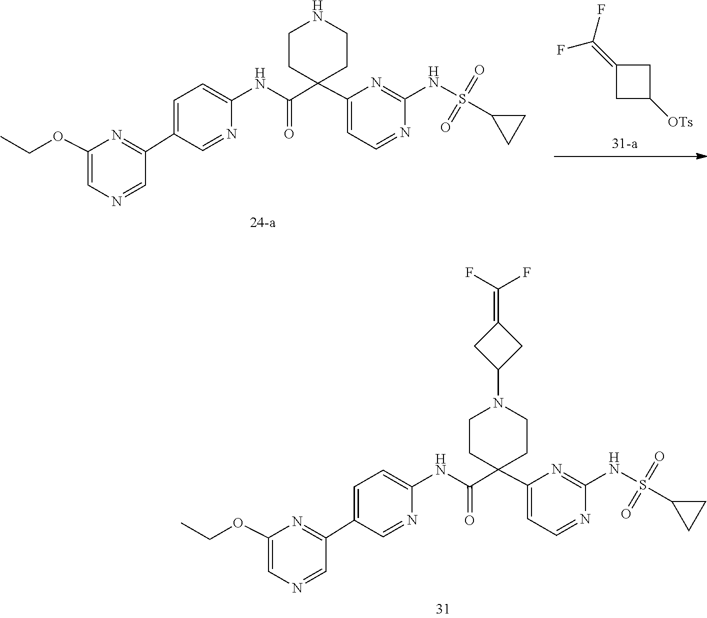 [SMILES: CCOc1cncc(-c2ccc(NC(=O)C3(c4ccnc(NS(=O)(=O)C5CC5)n4)CCNCC3)nc2)n1 ; Cc1ccc(S(=O)(=O)OC2CC(=C(F)F)C2)cc1 ; CCOc1cncc(-c2ccc(NC(=O)C3(c4ccnc(NS(=O)(=O)C5CC5)n4)CCN(C4CC(=C(F)F)C4)CC3)nc2)n1]

Compound 24-a (95 mg, 0.181 mmol), 31-a (89 mg, 0.326 mmol), sodium iodide (68 mg, 0.453 mmol) and cesium carbonate (177 mg, 0.543 mmol) were dissolved in N,N-dimethylformamide (2 mL). After nitrogen was bubbled through the reaction solution for 2 minutes, it was stirred at 80° C. overnight. After cooling to room temperature, the reaction mixture was diluted with water and acidified with 2 N hydrochloric acid, and then extracted with ethyl acetate (3×15 mL). The combined organic phases were washed with saturated brine, dried over anhydrous sodium sulfate, filtered, and concentrated under reduced pressure. The obtained crude product was separated and purified by preparative thin-layer chromatography (dichloromethane:methanol=20:1) to give Compound 31 as a white solid (2 mg, yield 2%). ¹H NMR (500 MHz, DMSO-d₆) δ 11.31 (br., 1H), 10.05 (s, 1H), 9.02 (d, J=2.0 Hz, 1H), 8.84 (s, 1H), 8.56 (d, J=2.9 Hz, 1H), 8.49 (dd, J=8.8, 2.4 Hz, 1H), 8.25 (s, 1H), 8.17 (d, J=8.8 Hz, 1H), 7.27-7.04 (m, 1H), 4.47 (q, J=7.0 Hz, 2H), 3.27-3.18 (m, 2H), 2.85-2.62 (m, 4H), 2.56-2.52 (m, 2H), 2.25-2.10 (m, 4H), 2.03-1.99 (m, 1H), 1.99-1.95 (m, 1H), 1.39 (t, J=7.0 Hz, 3H), 1.06-0.96 (m, 2H), 0.87-0.81 (m, 2H) ppm. MS m/z 627.3 [M+H]⁺.

### Example 32: Preparation of Compound 32

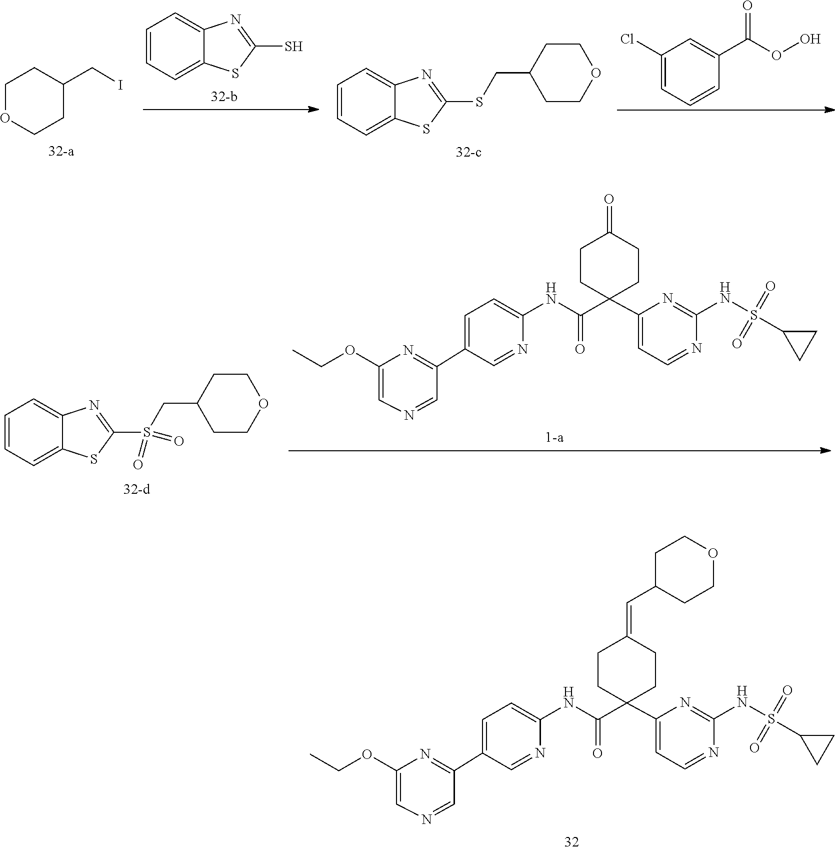

Compound 32-a (100 mg, 0.442 mmol) and 32-b (89 mg, 0.531 mmol) were dissolved in tetrahydrofuran (2.5 mL). Triethylamine (90 mg, 0.885 mmol) was added, and the reaction solution was stirred at 80° C. for 4 hours. After the reaction was completed, it was cooled to room temperature, and the reaction solution was concentrated under reduced pressure. The obtained crude product was separated and purified by silica gel column chromatography (dichloromethane:ethyl acetate=10:1) to give crude Compound 32-c as a colorless oil, which was directly used in the next step. MS m/z 266.1 [M+H]⁺.

Crude Compound 32-c (theoretical yield: 117 mg, 0.442 mmol) was dissolved in dichloromethane (4 mL). m-Chloroperoxybenzoic acid (167 mg, 0.970 mmol) was added, and the reaction solution was stirred at room temperature overnight. After the reaction was completed, it was filtered, and the obtained filtrate was washed sequentially with saturated sodium thiosulfate solution, saturated sodium bicarbonate solution and saturated brine, dried over anhydrous sodium sulfate, filtered, and concentrated under reduced pressure. The obtained crude product was separated and purified by silica gel column chromatography (dichloromethane:ethyl acetate=5:1) to give Compound 32-d as a white solid (79 mg, yield 60%). MS m/z 298.0 [M+H]⁺.

Compound 32-d (17 mg, 0.056 mmol) was dissolved in tetrahydrofuran (2 mL). A solution of lithium bis(trimethylsilyl)amide in tetrahydrofuran (1 M, 0.1 mL) was slowly added at −78° C. After the reaction solution was stirred at −78° C. for 5 minutes, a solution of Compound 1-a (15 mg, 0.028 mmol) in tetrahydrofuran (0.5 mL) was added. The mixture was stirred at −78° C. for 1 hour, then slowly warmed to 0° C. and diluted with water. The obtained mixture was acidified with 2 N hydrochloric acid and extracted with ethyl acetate (3×15 mL). The combined organic phases were washed with saturated brine, dried over anhydrous sodium sulfate, filtered, and concentrated under reduced pressure. The obtained crude product was separated and purified by preparative thin-layer chromatography (dichloromethane:methanol=20:1) to give Compound 32 as a white solid (6.7 mg, yield 39%). ¹H NMR (500 MHz, DMSO-d₆) δ 11.29 (br., 1H), 10.08 (s, 1H), 9.02 (d, J=2.0 Hz, 1H), 8.84 (s, 1H), 8.58 (d, J=5.0 Hz, 1H), 8.49 (dd, J=8.8, 2.2 Hz, 1H), 8.25 (s, 1H), 8.19 (d, J=8.8 Hz, 1H), 7.21 (s, 1H), 5.01 (d, J=8.6 Hz, 1H), 4.47 (q, J=7.0 Hz, 2H), 3.87-3.68 (m, 2H), 3.39-3.35 (m, 1H), 3.32-3.28 (m, 1H), 3.28-3.20 (m, 2H), 2.45-2.34 (m, 2H), 2.28-1.91 (m, 6H), 1.48-1.42 (m, 2H), 1.39 (t, J=7.0 Hz, 3H), 1.33-1.25 (m, 2H), 1.07-0.98 (m, 2H), 0.89-0.80 (m, 2H) ppm. MS m/z 620.4 [M+H]⁺.

### Example 33: Preparation of Compound 33

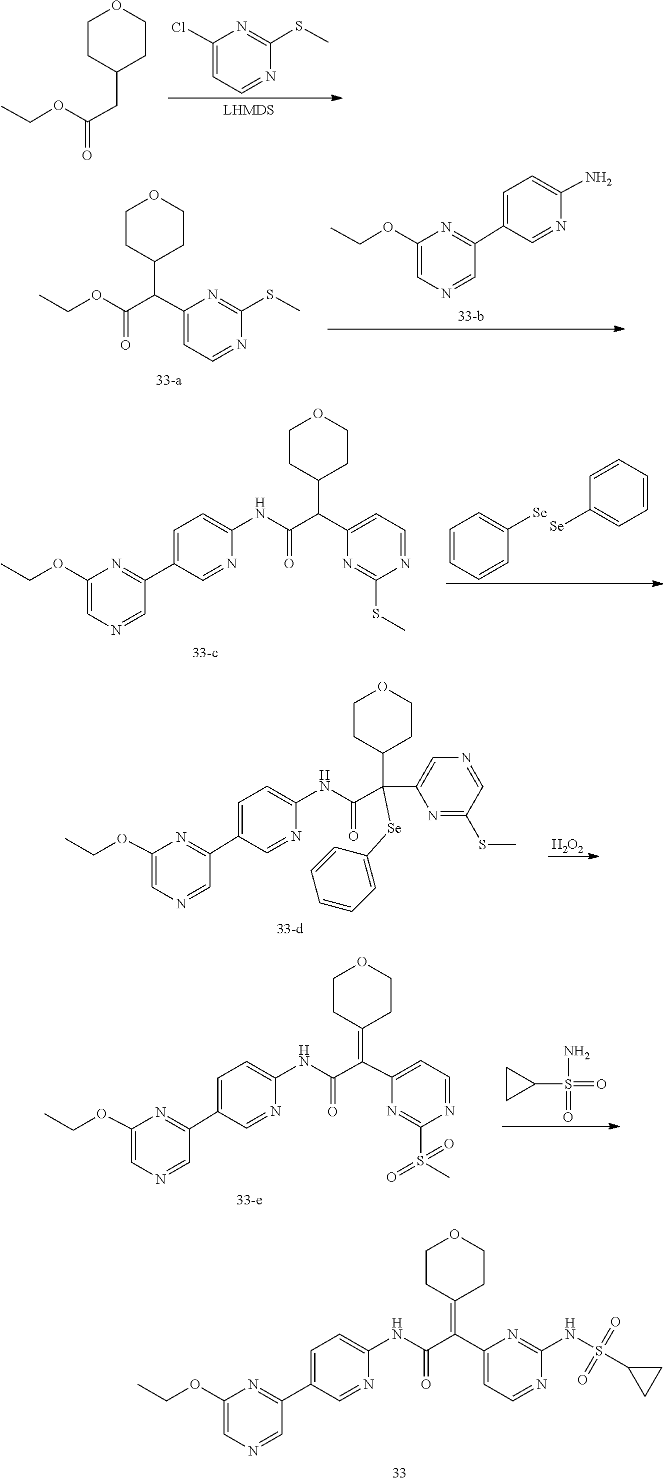

Ethyl tetrahydropyran-4-yl-acetate (500 mg, 2.91 mmol) and 2-methylthio-4-chloropyrimidine (465 mg, 2.91 mmol) were dissolved in tetrahydrofuran (10 mL). A solution of lithium bis(trimethylsilyl)amide in tetrahydrofuran (8.7 mL, 1M) was added dropwise in an ice bath. After the dropwise addition was completed, the reaction solution was stirred at room temperature for 3 hours. After the reaction was completed, it was quenched with water and extracted with ethyl acetate (3×10 mL). The combined organic phases were washed with saturated brine, dried over anhydrous sodium sulfate, filtered, and concentrated under reduced pressure. The obtained crude product was separated and purified by silica gel column chromatography (ethyl acetate:petroleum ether=5:1) to give Compound 33-a as a yellow oil (369 mg, yield 43).

Compound 33-a (100 mg, 0.34 mmol) and Compound 33-b (73 mg, 0.34 mmol) were dissolved in toluene (2 mL). A solution of trimethylaluminum in toluene (2 M, 0.5 mL) was added dropwise in an ice bath. After the dropwise addition was completed, the reaction solution was warmed to 100° C. and stirred for 2 hours. After the reaction was completed, it was quenched with water and extracted with ethyl acetate (3×10 mL). The combined organic phases were washed with saturated brine, dried over anhydrous sodium sulfate, filtered, and concentrated under reduced pressure. The obtained crude product was separated and purified by silica gel column chromatography (ethyl acetate: petroleum ether=2:1) to give Compound 33-c as a yellow oil (44 mg, yield 28%).

Compound 33-c (44 mg, 0.09 mmol) was dissolved in DMSO (2 mL). tert-Butanol (20 mg, 0.18 mmol) and diphenyl diselenide (42 mg, 0.14 mmol) were added. The reaction solution was stirred at room temperature for 6 hours. After the reaction was completed, it was quenched with water and extracted with ethyl acetate (3×5 mL). The combined organic phases were washed with saturated brine, dried over anhydrous sodium sulfate, filtered, and concentrated under reduced pressure. The obtained crude product was separated and purified by preparative thin-layer plate (ethyl acetate:petroleum ether=2:1) to give Compound 33-d as a yellow oil (21 mg, yield 36%).

Compound 33-d (21 mg, 0.03 mmol) was dissolved in tetrahydrofuran (2 mL). An aqueous solution of hydrogen peroxide (30%, 0.5 mL) was added dropwise. The reaction solution was stirred at room temperature for 1 hour. After the reaction was completed, it was quenched with water, and the mixture was extracted with ethyl acetate (3×5 mL). The combined organic phases were washed with saturated brine, dried over anhydrous sodium sulfate, filtered, and concentrated under reduced pressure. The obtained crude product was separated and purified by preparative thin-layer plate (ethyl acetate:petroleum ether=2:1) to give Compound 33-e as a yellow oil (5 mg, yield 29%).

Compound 33-e (5 mg, 0.01 mmol) was dissolved in acetonitrile (1 mL). Cyclopropylsulfonamide (3 mg, 0.02 mmol) and cesium carbonate (10 mg, 0.03 mmol) were added. The reaction solution was heated and stirred at 60° C. for 3 hours. After the reaction was completed, it was concentrated under reduced pressure, and the obtained crude product was separated and purified by reversed-phase preparative chromatography to give Compound 33 as a white solid (0.3 mg, yield 5%). MS m/z 538.2 [M+H]⁺.

### Example 34: Preparation of Compound 34

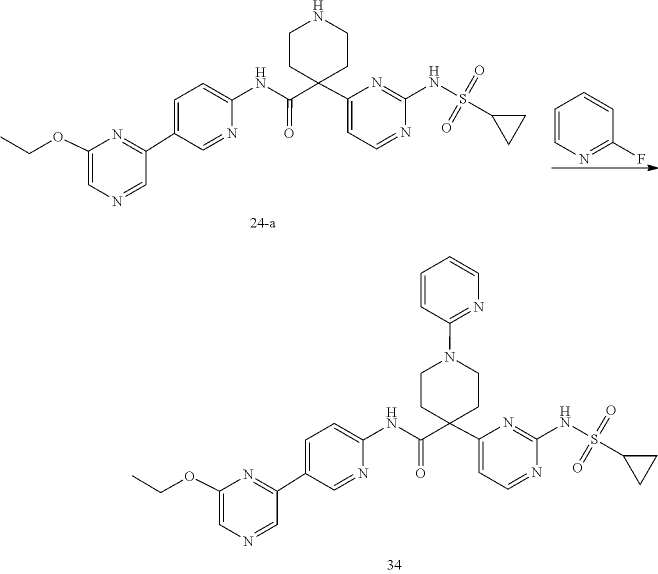

Compound 34-a (40 mg, 0.076 mmol), 2-fluoropyridine (15 mg, 0.153 mmol) and cesium carbonate (75 mg, 0.229 mmol) were dissolved in N,N-dimethylformamide (2 mL). The reaction solution was stirred at 80° C. overnight. After cooling to room temperature, the reaction mixture was diluted with ethyl acetate and acidified with 2 N hydrochloric acid. The mixture was extracted with ethyl acetate (3×15 mL). The combined organic phases were washed with saturated brine, dried over anhydrous sodium sulfate, filtered, and concentrated under reduced pressure. The obtained crude product was separated and purified by preparative thin-layer chromatography (dichloromethane:methanol=18:1) to give Compound 34 as a white solid (7.9 mg, yield 17%). ¹H NMR (500 MHz, DMSO-d₆) δ 11.32 (br., 1H), 10.22 (s, 1H), 9.03 (d, J=2.0 Hz, 1H), 8.84 (s, 1H), 8.60 (d, J=5.2 Hz, 1H), 8.50 (dd, J=8.8, 2.4 Hz, 1H), 8.25 (s, 1H), 8.20 (d, J=8.7 Hz, 1H), 8.11 (dd, J=4.9, 1.4 Hz, 1H), 7.57-7.46 (m, 1H), 7.28-7.18 (m, 1H), 6.88 (d, J=8.7 Hz, 1H), 6.62 (dd, J=6.8, 5.0 Hz, 1H), 4.47 (q, J=7.0 Hz, 2H), 4.02-3.82 (m, 2H), 3.38-3.34 (m, 1H), 3.27-3.18 (m, 1H), 2.59-2.53 (m, 2H), 2.24-2.15 (m, 2H), 2.04-1.95 (m, 1H), 1.39 (t, J=7.0 Hz, 3H), 1.08-1.01 (m, 2H), 0.89-0.84 (m, 2H) ppm. MS m/z 602.2 [M+H]⁺.

### Example 35: Preparation of Compound 35

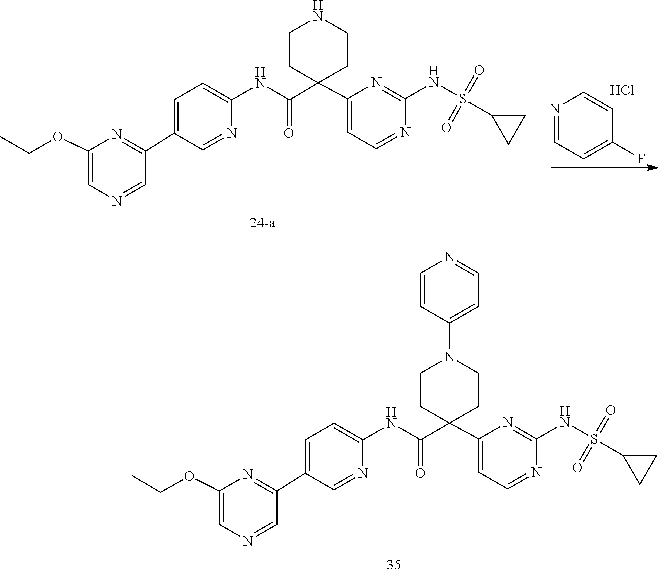

The synthesis of Compound 35 refers to the synthesis method of Compound 34, giving Compound 35 as a white solid (4.2 mg, yield 12%). ¹H NMR (500 MHz, DMSO-d₆) δ 10.23 (s, 1H), 9.03 (d, J=1.9 Hz, 1H), 8.84 (s, 1H), 8.53 (d, J=5.1 Hz, 1H), 8.50 (dd, J=8.8, 2.4 Hz, 1H), 8.25 (s, 1H), 8.18 (d, J=8.9 Hz, 1H), 8.15 (d, J=4.8 Hz, 2H), 7.12-7.08 (m, 1H), 6.87 (d, J=5.5 Hz, 2H), 4.47 (q, J=7.0 Hz, 2H), 3.71-3.62 (m, 2H), 3.31-3.27 (m, 1H), 3.21-3.14 (m, 1H), 2.57-2.52 (m, 2H), 2.30-2.20 (m, 2H), 2.04-1.95 (m, 1H), 1.39 (t, J=7.0 Hz, 3H), 1.03-0.96 (m, 2H), 0.83-0.77 (m, 2H) ppm. MS m/z 602.2 [M+H]⁺.

### Example 36: Preparation of Compound 36

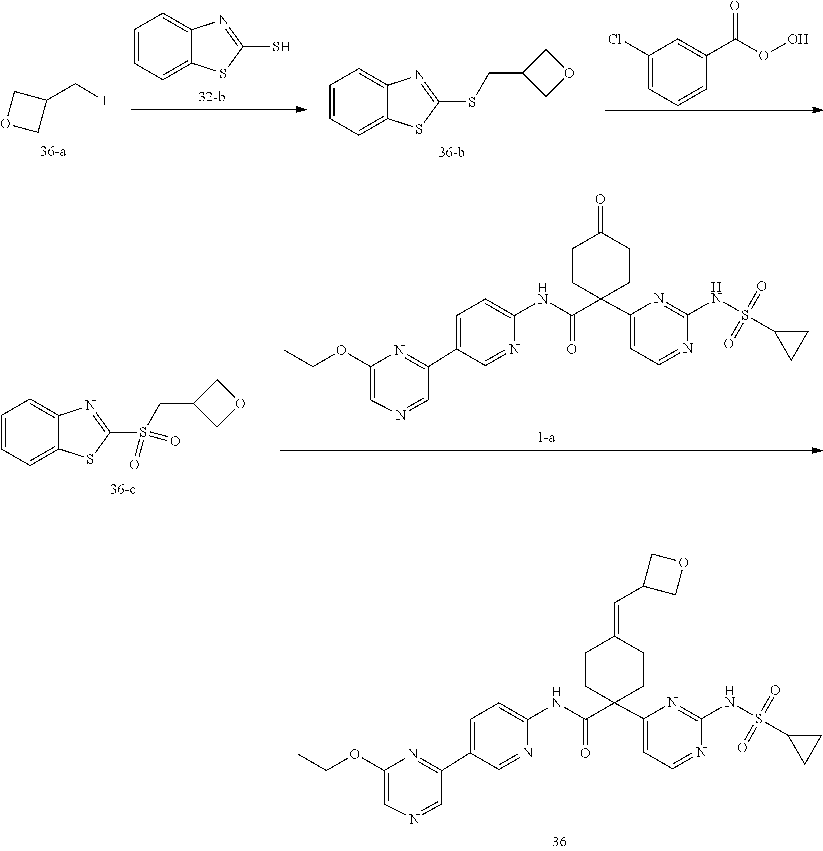

The synthesis of Compound 36 refers to the synthesis route of Compound 32, giving Compound 36 as a white solid (29 mg, yield 36%). ¹H NMR (500 MHz, DMSO-d₆) δ 11.27 (br., 1H), 10.07 (s, 1H), 9.01 (d, J=2.0 Hz, 1H), 8.84 (s, 1H), 8.58 (d, J=5.3 Hz, 1H), 8.49 (dd, J=8.8, 2.3 Hz, 1H), 8.25 (s, 1H), 8.18 (d, J=8.8 Hz, 1H), 7.21 (d, J=5.2 Hz, 1H), 5.55 (d, J=8.5 Hz, 1H), 4.75-4.66 (m, 2H), 4.47 (q, J=7.0 Hz, 2H), 4.39-4.32 (m, 2H), 4.01-3.90 (m, 1H), 3.28-3.20 (m, 1H), 2.32-2.16 (m, 4H), 2.14-1.92 (m, 4H), 1.39 (t, J=7.0 Hz, 3H), 1.06-1.02 (m, 2H), 0.88-0.82 (m, 2H) ppm. MS m/z 592.0 [M+H]⁺.

### Example 37: Preparation of Compound 37

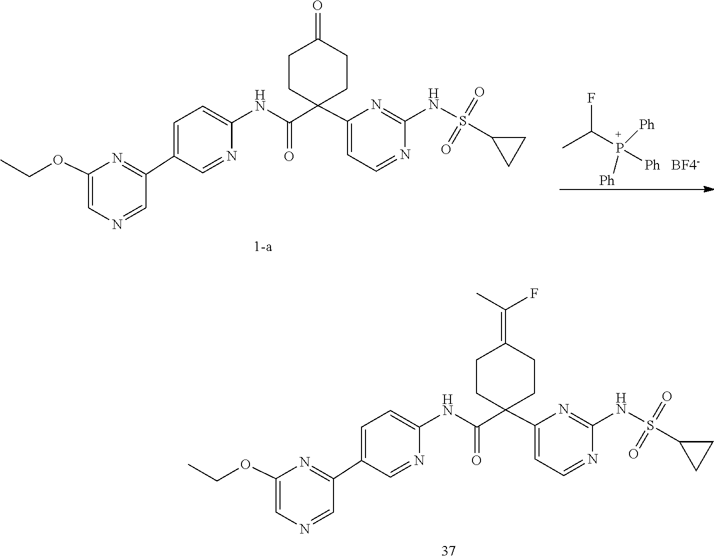

(1-Fluoroethyl)triphenylphosphonium tetrafluoroborate (56 mg, 0.15 mmol) was dissolved in tetrahydrofuran (2 mL). Under an ice bath and nitrogen atmosphere, a solution of lithium bis(trimethylsilyl)amide in tetrahydrofuran (0.15 mL, 1M) was added dropwise. The reaction solution was stirred in the ice bath for another 0.5 hours, then a solution of Compound 1-a (25 mg, 0.05 mmol) in tetrahydrofuran (2 mL) was added dropwise. The reaction solution was stirred at room temperature for 1 hour. After the reaction was completed, it was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 37 as a white solid (3 mg, yield 12%). ¹H NMR (500 MHz, DMSO) δ 11.27 (br., 1H), 10.09 (s, 1H), 9.02 (d, J=1.6 Hz, 1H), 8.84 (s, 1H), 8.59 (d, J=3.9 Hz, 1H), 8.49 (dd, J=8.8, 2.3 Hz, 1H), 8.25 (s, 1H), 8.18 (d, J=8.9 Hz, 1H), 7.24-7.18 (m, 1H), 4.47 (q, J=7.0 Hz, 2H), 3.25-3.24 (m, 1H), 2.24-2.11 (m, 4H), 2.06-1.96 (m, 4H), 1.90 (d, J=18.3 Hz, 3H), 1.39 (t, J=7.0 Hz, 3H), 1.06-1.02 (m, 2H), 0.87-0.83 (m, 2H). MS m/z 568.0 [M+H]⁺.

### Example 38: Preparation of Compound 38

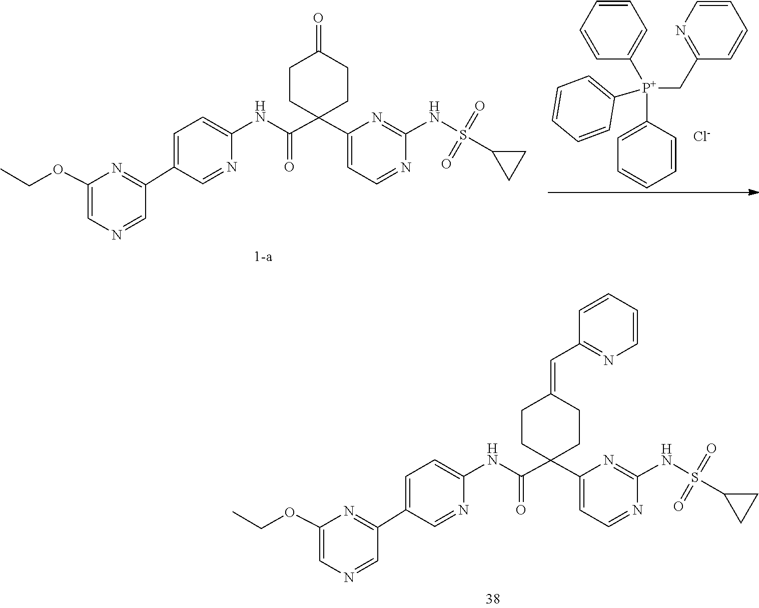 [SMILES: CCOc1cncc(-c2ccc(NC(=O)C3(c4ccnc(NS(=O)(=O)C5CC5)n4)CCC(=O)CC3)nc2)n1 ; c1ccc([P+](Cc2ccccn2)(c2ccccc2)c2ccccc2)cc1 ; [Cl-] ; CCOc1cncc(-c2ccc(NC(=O)C3(c4ccnc(NS(=O)(=O)C5CC5)n4)CCC(=Cc4ccccn4)CC3)nc2)n1]

60% sodium hydride (12 mg, 0.30 mmol) was added to DMSO (1 mL). The mixture was stirred at 80° C. for 60 minutes under a nitrogen atmosphere, then cooled to 15° C. 2-Picolinyltriphenylphosphonium chloride (100 mg, 0.26 mmol) was added at this temperature, and stirring was continued for 10 minutes. Then Compound 1-a (15 mg, 0.028 mmol) was added, and the reaction solution was warmed to 80° C. and stirred for another 3 hours. The system was cooled to room temperature, quenched with water, and extracted with ethyl acetate (10 mL×3). The combined organic layers were washed with saturated brine, dried over anhydrous sodium sulfate, filtered, and concentrated under reduced pressure. The obtained crude product was separated and purified by preparative thin-layer plate (dichloromethane:methanol=10:1) to give Compound 38 as a white solid (4 mg, yield 23%). ¹H NMR (500 MHz, DMSO-d₆) δ 11.33 (s, 1H), 10.11 (s, 1H), 9.03 (s, 1H), 8.84 (s, 1H), 8.64-8.40 (m, 3H), 8.28-8.13 (m, 2H), 7.77-7.68 (m, 1H), 7.29-7.11 (m, 3H), 6.32 (s, 1H), 4.48 (q, J=7.0 Hz, 2H), 3.27-3.23 (m, 1H), 2.76-2.59 (m, 2H), 2.39-2.08 (m, 6H), 1.40 (t, J=7.0 Hz, 3H), 1.08-0.97 (m, 2H), 0.91-0.78 (m, 2H). MS m/z 613.0 [M+H]⁺.

### Example 39&43: Preparation of Compound 39&43

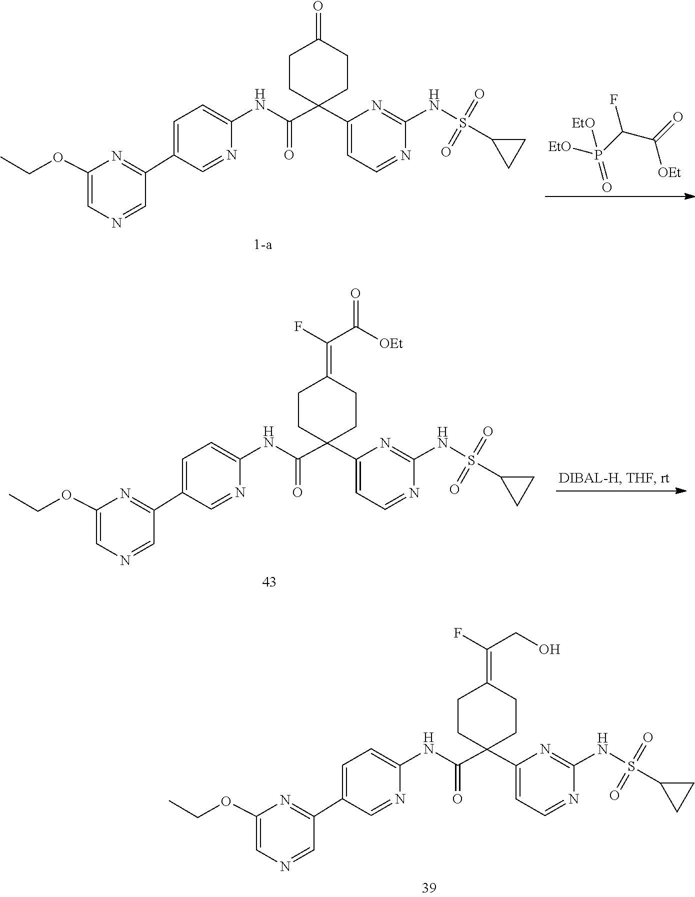

Trimethyl 2-fluoro-2-phosphonoacetate (59 mg, 0.24 mmol) was dissolved in tetrahydrofuran (2 mL). 60% sodium hydride (16 mg, 0.40 mmol) was added under an ice bath and nitrogen atmosphere. The reaction solution was stirred in the ice bath for 0.5 hours, then a solution of Compound 1-a (45 mg, 0.08 mmol) in tetrahydrofuran (2 mL) was added dropwise. The reaction solution was warmed to room temperature and stirred for 1 hour. The reaction solution was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 43 as a white solid (34 mg, yield 65%). MS m/z 626.3 [M+H]⁺.

Compound 43 (18 mg, 0.03 mmol) was dissolved in tetrahydrofuran (2 mL). Under a nitrogen atmosphere, it was cooled to −70° C., and a solution of diisobutylaluminum hydride in n-hexane (1M, 0.1 mL) was added dropwise. The reaction solution was stirred at this temperature for another 0.5 hours. The reaction solution was quenched with sodium sulfate decahydrate, stirred at room temperature for 15 minutes, filtered, and the filtrate was concentrated under reduced pressure. It was separated and purified by preparative thin-layer plate (dichloromethane:methanol=25:1) to give Compound 39 as a white solid (3 mg, yield 18%). ¹H NMR (500 MHz, DMSO-d) δ 11.29 (br., 1H), 10.09 (br., 1H), 9.02 (d, J=1.9 Hz, 1H), 8.84 (s, 1H), 8.57 (br., 1H), 8.49 (dd, J=8.8, 2.4 Hz, 1H), 8.25 (s, 1H), 8.18 (d, J=8.7 Hz, 1H), 7.20 (br., 1H), 5.01 (t, J=5.8 Hz, 1H), 4.47 (q, J=7.0 Hz, 2H), 4.05 (dd, J=24.5, 5.8 Hz, 2H), 3.25-3.22 (m, 1H), 2.27-2.13 (m, 4H), 2.09-1.97 (m, 4H), 1.39 (t, J=7.0 Hz, 3H), 1.05-0.99 (m, 2H), 0.88-0.84 (m, 2H). MS m/z 584.0 [M+H]⁺.

### Example 40: Preparation of Compound 40

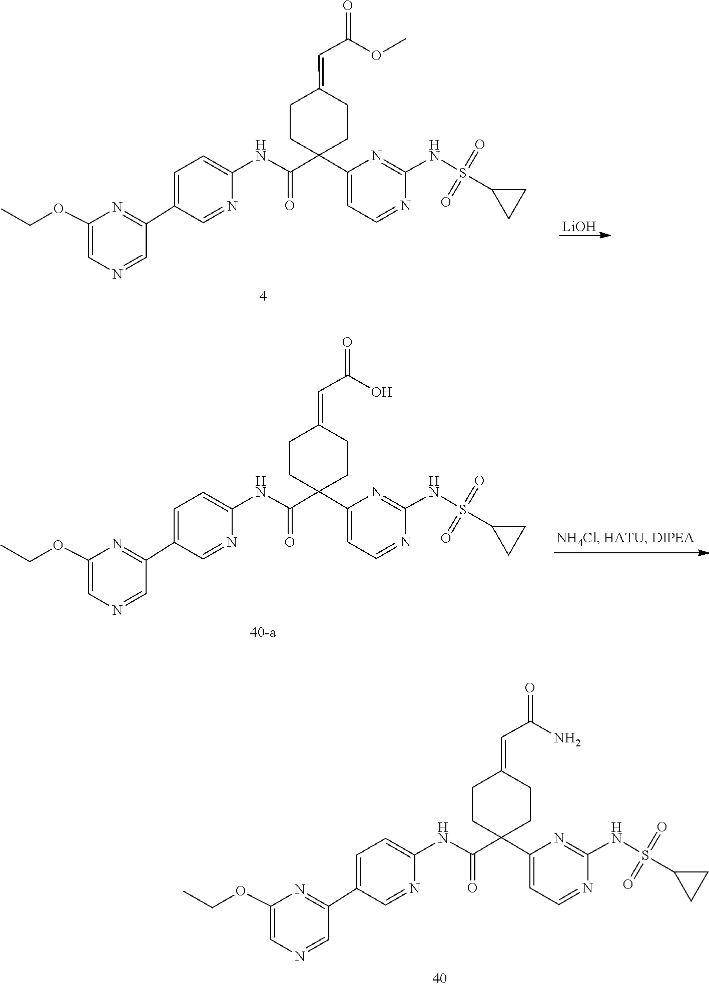

Compound 4 (40 mg, 0.07 mmol) was dissolved in a mixed solution of tetrahydrofuran/methanol (1 mL/1 mL), and then an aqueous solution of lithium hydroxide (1 M, 0.3 mL) was added. The reaction solution was stirred at room temperature overnight. The reaction solution was concentrated under reduced pressure, and the obtained crude Compound 40-a was directly used in the next step.

Crude Compound 40-a was dissolved in DMF (2 mL). N,N-Diisopropylethylamine (27 mg, 0.21 mmol) was added dropwise, then N,N,N′,N′-tetramethyl-O-(7-azabenzotriazol-1-yl)uronium hexafluorophosphate (40 mg, 0.11 mmol) was added. The reaction mixture was stirred at room temperature for 5 minutes, and then ammonium chloride (11 mg, 0.21 mmol) was added. The system was stirred at room temperature for 0.5 hours. After the reaction was completed, it was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 40 as a white solid (3 mg, yield 8%). ¹H NMR (500 MHz, DMSO-d) δ 10.05 (br., 1H), 9.02 (d, J=2.1 Hz, 1H), 8.83 (s, 1H), 8.56-8.46 (m, 2H), 8.25 (s, 1H), 8.18 (d, J=8.7 Hz, 1H), 7.26 (s, 1H), 6.77 (s, 1H), 5.65 (s, 1H), 4.47 (q, J=7.0 Hz, 2H), 3.22-3.19 (m, 1H), 2.38-2.29 (m, 2H), 2.23-2.09 (m, 4H), 2.04-1.95 (m, 2H), 1.39 (t, J=7.0 Hz, 3H), 1.03-0.98 (m, 2H), 0.85-0.80 (m, 2H). MS m/z 579.1 [M+H]⁺.

### Example 41: Preparation of Compound 41

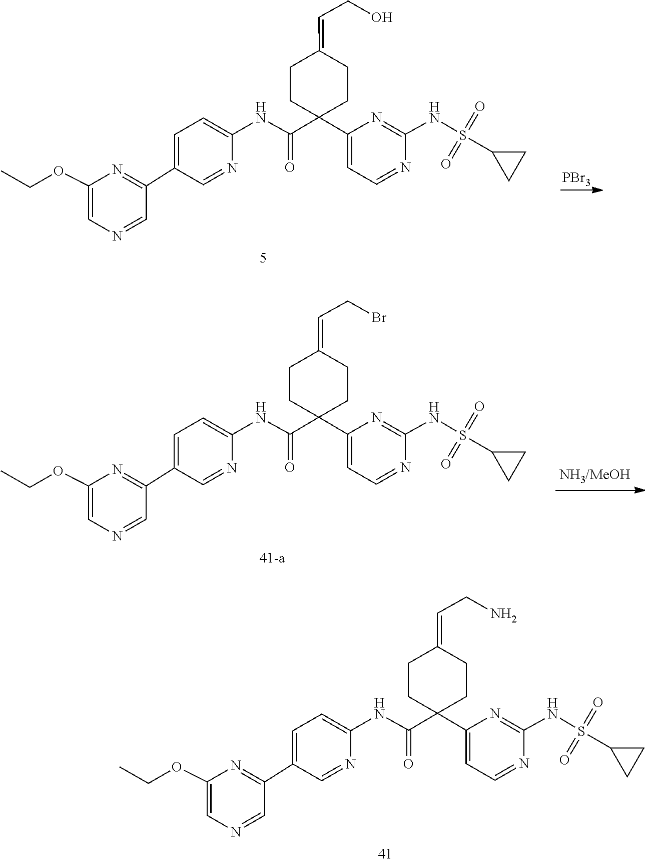

Compound 5 (16 mg, 0.03 mmol) was dissolved in dichloromethane (2 mL). Phosphorus tribromide (16 mg, 0.06 mmol) was added dropwise under an ice bath. The reaction solution was stirred in the ice bath for another 10 minutes. The reaction solution was concentrated under reduced pressure to give crude Compound 41-a as a yellow oil, which was directly used in the next step.

Crude Compound 41-a was dissolved in dichloromethane. A solution of ammonia in methanol (7 M, 0.2 mL) was added dropwise under an ice bath. The reaction solution was stirred at this temperature for 0.5 hours. The reaction solution was concentrated under reduced pressure, and the obtained crude product was separated and purified by preparative thin-layer plate (dichloromethane:methanol=20:1) to give Compound 41 as a white solid (3 mg, yield 19%). ¹H NMR (500 MHz, DMSO-d) δ 10.01 (br., 1H), 9.02 (d, J=1.9 Hz, 1H), 8.83 (s, 1H), 8.51-8.43 (m, 2H), 8.25 (s, 1H), 8.16 (d, J=8.8 Hz, 1H), 6.97 (br., 1H), 5.23 (t, J=6.6 Hz, 1H), 4.47 (q, J=14.1, 7.0 Hz, 2H), 3.45 (d, J=7.1 Hz, 2H), 3.19-3.16 (m, 1H), 2.38-2.10 (m, 9H), 1.39 (t, J=7.0 Hz, 3H), 0.99-0.92 (m, 2H), 0.80-0.74 (m, 2H). MS m/z 565.1 [M+H]⁺.

### Example 42: Preparation of Compound 42

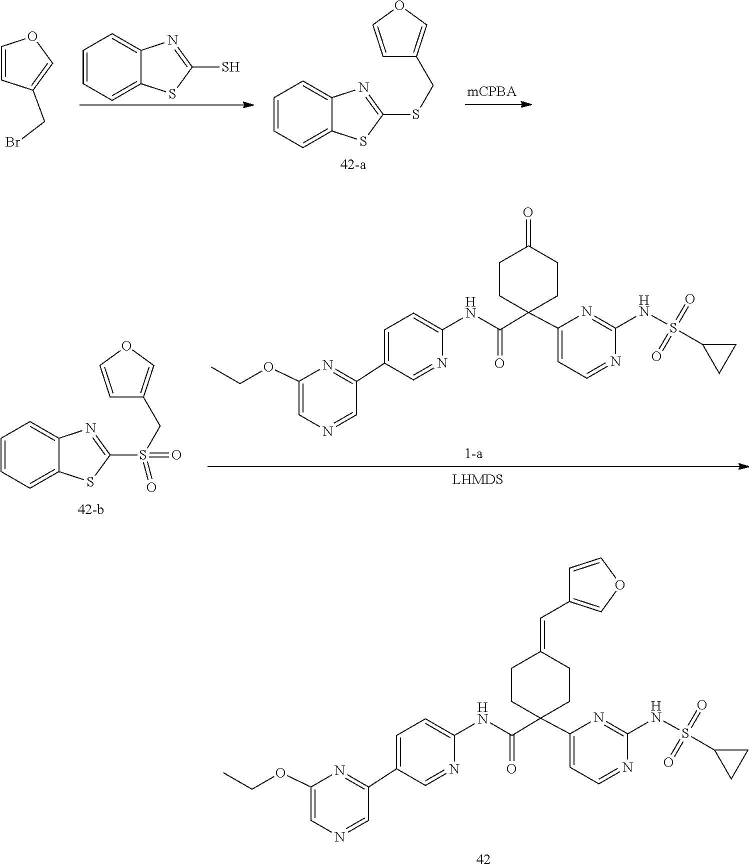

3-Bromomethylfuran (200 mg, 1.25 mmol) and benzo[D]thiazole-2-thiol (209 mg, 1.25 mmol) were dissolved in tetrahydrofuran (5 mL), then triethylamine (379 mg, 3.75 mmol) was added dropwise. The reaction solution was stirred at room temperature for 1 hour. After the reaction was completed, it was quenched with water, and the mixture was extracted with ethyl acetate (3×10 mL). The combined organic phases were washed with saturated brine, dried over anhydrous sodium sulfate, filtered, and concentrated under reduced pressure to give crude Compound 42-a, which was directly used in the next step.

Compound 42-a was dissolved in dichloromethane (5 mL), and m-chloroperoxybenzoic acid (539 mg, 3.13 mmol) was added. The reaction solution was stirred at room temperature overnight. Saturated sodium thiosulfate solution was added to quench the reaction, and the mixture was extracted with dichloromethane (3×10 mL). The combined organic phases were washed with saturated brine, dried over anhydrous sodium sulfate, filtered, and concentrated under reduced pressure. The obtained crude product was separated and purified by silica gel column chromatography (ethyl acetate: petroleum ether=1:1) to give Compound 42-b as a white solid (98 mg, yield 28%).

Compound 42-b (31 mg, 0.11 mmol) and Compound 1-a (30 mg, 0.06 mmol) were dissolved in tetrahydrofuran (2 mL), and cooled to −60° C. under a nitrogen atmosphere. A solution of lithium bis(trimethylsilyl)amide in tetrahydrofuran (0.20 mL, 1M) was added dropwise. The reaction solution was stirred at this temperature for 0.5 hours. After the reaction was completed, it was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 42 as a white solid (6 mg, yield 18%). ¹H NMR (500 MHz, DMSO-d) δ 10.08 (s, 1H), 9.03 (d, J=2.1 Hz, 1H), 8.84 (s, 1H), 8.56-8.47 (m, 2H), 8.25 (s, 1H), 8.20 (d, J=8.8 Hz, 1H), 7.66 (s, 1H), 7.62 (t, J=1.6 Hz, 1H), 7.12 (br., 1H), 6.53 (d, J=1.2 Hz, 1H), 5.99 (s, 1H), 4.48 (q, J=7.0 Hz, 2H), 3.22-3.20 (m, 1H), 2.65-2.58 (m, 2H), 2.46-2.29 (m, 4H), 2.19-2.13 (m, 1H), 2.11-2.04 (m, 1H), 1.40 (t, J=7.0 Hz, 3H), 1.03-0.98 (m, 2H), 0.83-0.78 (m, 2H). MS m/z 602.1 [M+H]⁺.

### Example 44: Preparation of Compound 44

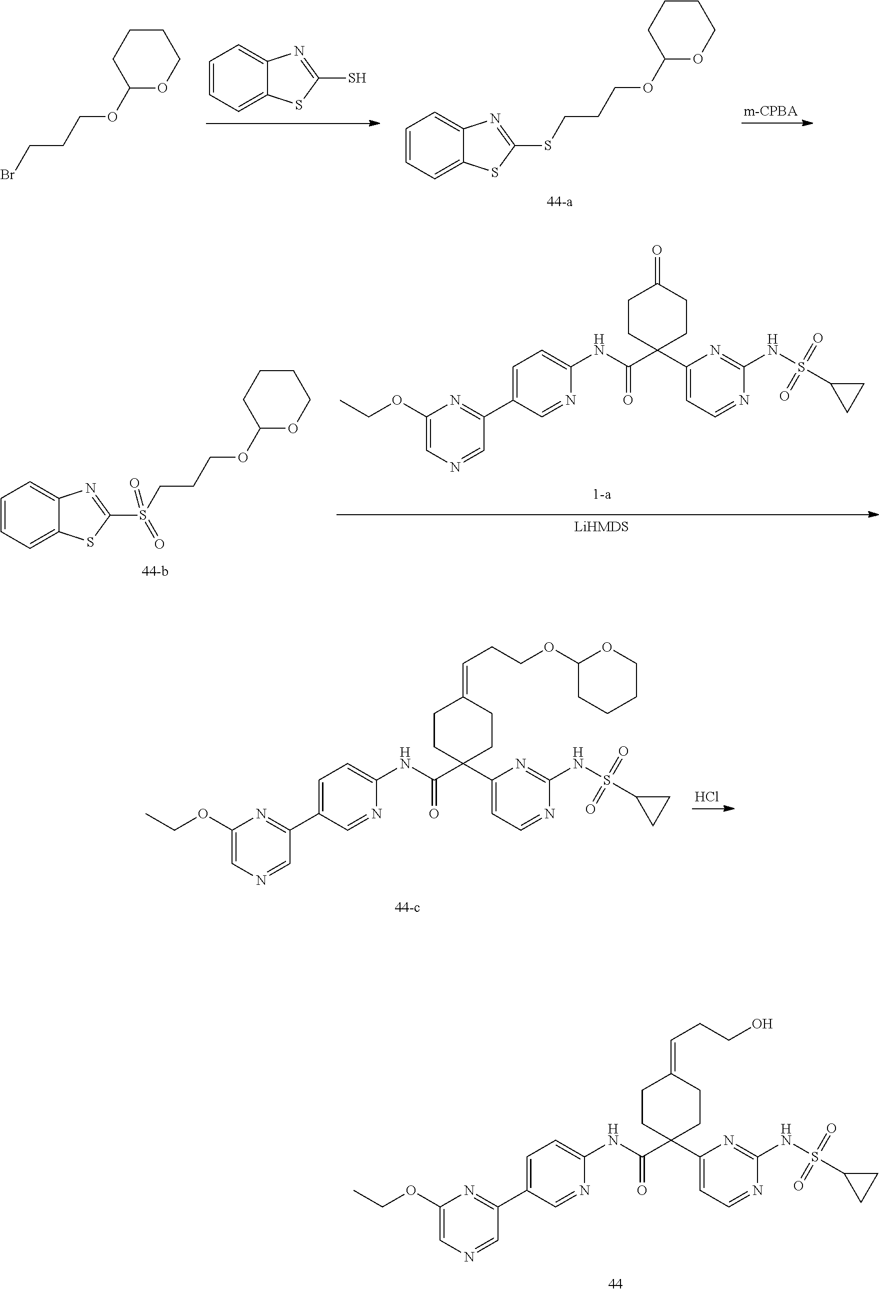

2-(3-Bromopropoxy)tetrahydropyran (300 mg, 1.34 mmol) and benzo[D]thiazole-2-thiol (224 mg, 1.34 mmol) were dissolved in tetrahydrofuran (5 mL), then triethylamine (406 mg, 4.02 mmol) was added dropwise. The reaction solution was heated and stirred at 70° C. for 1 hour. After the reaction was completed, it was quenched with water, and the mixture was extracted with ethyl acetate (3×10 mL). The combined organic phases were washed with saturated brine, dried over anhydrous sodium sulfate, filtered, and concentrated under reduced pressure to give crude Compound 44-a, which was directly used in the next step.

Compound 44-a was dissolved in dichloromethane (5 mL), and m-chloro peroxybenzoic acid (580 mg, 3.35 mmol) was added. The reaction solution was stirred at room temperature overnight. Saturated sodium thiosulfate solution was added to quench the reaction, and the mixture was extracted with dichloromethane (3×10 mL). The combined organic phases were washed with saturated brine, dried over anhydrous sodium sulfate, filtered, and concentrated under reduced pressure. The obtained crude product was separated and purified by silica gel column chromatography (ethyl acetate:petroleum ether=1:1) to give Compound 44-b as a white solid (305 mg, yield 66%).

Compound 44-b (48 mg, 0.14 mmol) and Compound 41-a (40 mg, 0.07 mmol) were dissolved in tetrahydrofuran (2 mL), and cooled to −60° C. under a nitrogen atmosphere. A solution of lithium bis(trimethylsilyl)amide in tetrahydrofuran (0.30 mL, 1M) was added dropwise. The reaction solution was stirred for another 0.5 hours. After the reaction was completed, it was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 44-c (18 mg, yield 37%). MS m/z 664.2 [M+H]⁺.

Compound 44-c (18 mg, 0.03 mmol) was dissolved in dichloromethane (2 mL). A solution of hydrogen chloride in dioxane (4 M, 0.5 mL) was added dropwise. The reaction solution was stirred at room temperature for 0.5 hours. The reaction solution was concentrated under reduced pressure, and the obtained crude product was separated and purified by preparative thin-layer plate (dichloromethane:methanol=25:1) to give Compound 44 as a white solid (4 mg, yield 25%). ¹H NMR (500 MHz, DMSO-d) δ 11.28 (br., 1H), 10.05 (s, 1H), 9.02 (d, J=2.0 Hz, 1H), 8.84 (s, 1H), 8.58 (d, J=5.0 Hz, 1H), 8.49 (dd, J=8.8, 2.4 Hz, 1H), 8.25 (s, 1H), 8.19 (d, J=8.8 Hz, 1H), 7.21 (br., 1H), 5.16 (t, J=7.1 Hz, 1H), 4.47 (q, J=7.1 Hz, 2H), 3.38-3.37 (m, 2H), 3.27-3.22 (m, 1H), 2.40-1.96 (m, 10H), 1.39 (t, J=7.0 Hz, 3H), 1.07-1.02 (m, 2H), 0.89-0.83 (m, 2H). MS m/z 580.0 [M+H]⁺.

### Example 45: Preparation of Compound 45

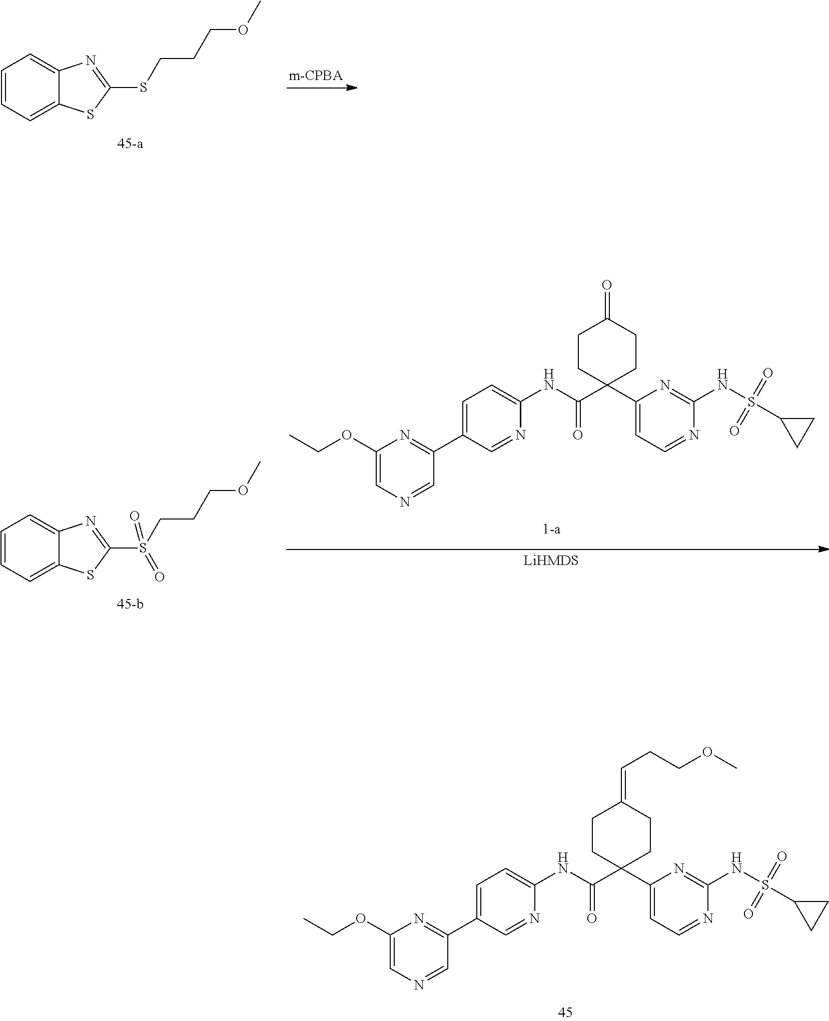

Compound 45-a was dissolved in dichloromethane (5 mL), and m-chloro peroxybenzoic acid (503 mg, 2.48 mmol) was added. The reaction solution was stirred at room temperature overnight. Saturated sodium thiosulfate solution was added to quench the reaction, and the mixture was extracted with dichloromethane (3×10 mL). The combined organic phases were washed with saturated brine, dried over anhydrous sodium sulfate, filtered, and concentrated under reduced pressure. The obtained crude product was separated and purified by silica gel column chromatography (ethyl acetate:petroleum ether=1:1) to give Compound 45-b as a white solid (240 mg, yield 78%).

Compound 45-b (20.2 mg, 0.07 mmol) and Compound 1-a (20 mg, 0.035 mmol) were dissolved in tetrahydrofuran (2 mL), and cooled to −60° C. under a nitrogen atmosphere. A solution of lithium bis(trimethylsilyl)amide in tetrahydrofuran (0.1 mL, 1 M) was added dropwise. The reaction was stirred for another 0.5 hours. After the reaction was completed, it was diluted with water and extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 45 as a white solid (10.12 mg, yield 46%). ¹H NMR (500 MHz, DMSO-d) δ 11.27 (br., 1H), 10.06 (s, 1H), 9.04-9.00 (m, 1H), 8.84 (s, 1H), 8.59 (d, J=5.3 Hz, 1H), 8.49 (dd, J=8.8, 2.4 Hz, 1H), 8.25 (s, 1H), 8.19 (d, J=8.9 Hz, 1H), 7.22 (d, J=5.3 Hz, 1H), 5.15 (t, J=7.2 Hz, 1H), 4.50-4.44 (m, 2H), 3.29 (t, J=6.8 Hz, 2H), 3.25 (dd, J=8.7, 4.0 Hz, 1H), 3.21 (s, 3H), 2.40-1.95 (m, 10H), 1.39 (t, J=7.0 Hz, 3H), 1.08-1.02 (m, 2H), 0.89-0.83 (m, 2H). MS m/z 594.2 [M+H]⁺.

### Example 46&47: Preparation of Compound 46&47

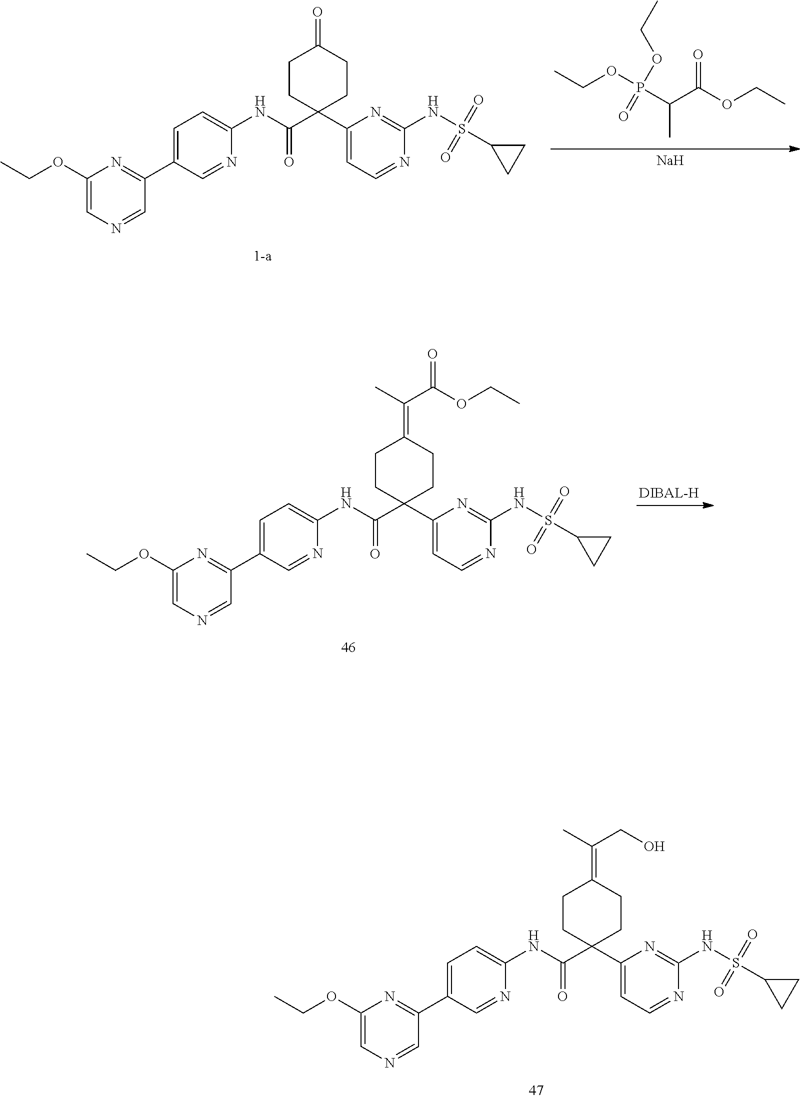

Triethyl 2-phosphonopropionate (443 mg, 1.86 mmol) was dissolved in N,N-dimethylformamide (2 mL). Sodium hydride (74 mg, 1.86 mmol) was added under an ice bath and nitrogen atmosphere. The reaction solution was stirred in the ice bath for 0.5 hours, then a solution of Compound 1-a (100 mg, 0.19 mmol) in N,N-dimethylformamide (2 mL) was added dropwise. The reaction solution was stirred at room temperature for 12 hours. The reaction solution was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 46 as a white solid (20.63 mg, yield 18%) with MS m/z 622.1 [M+H]⁺.

Compound 46 (10 mg, 0.016 mmol) was dissolved in tetrahydrofuran (2 mL). Diisobutylaluminum hydride (1 M, 0.1 mL) was slowly added under a nitrogen atmosphere at −78° C. The mixture was stirred for another hour. After the reaction was completed, the reaction solution was quenched with water and evaporated to dryness to give a crude product, which was separated and purified by preparative thin-layer plate (dichloromethane:methanol=25:1) to give Compound 47 as a white solid (4.93 mg, yield 53%). ¹H NMR (500 MHz, DMSO-d) δ 11.12 (br., 1H), 10.04 (s, 1H), 9.07-8.94 (m, 1H), 8.84 (s, 1H), 8.59 (d, J=5.3 Hz, 1H), 8.49 (dd, J=8.8, 2.4 Hz, 1H), 8.25 (s, 1H), 8.19 (d, J=9.0 Hz, 1H), 7.21 (d, J=5.3 Hz, 1H), 4.51-4.44 (m, 2H), 4.42 (t, J=5.4 Hz, 1H), 3.92 (d, J=5.4 Hz, 2H), 3.28-3.21 (m, 1H), 2.49-2.35 (m, 4H), 2.23-2.12 (m, 2H), 2.03 (t, J=10.2 Hz, 2H), 1.68 (s, 3H), 1.39 (t, J=7.0 Hz, 3H), 1.07-1.01 (m, 2H), 0.89-0.82 (m, 2H). MS m/z 580.0 [M+H]⁺.

### Example 48: Preparation of Compound 48

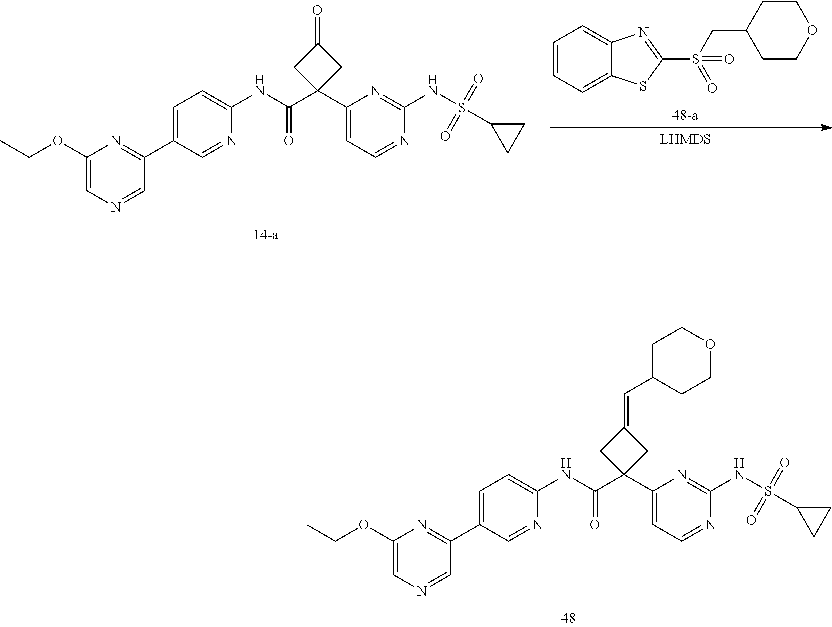 [SMILES: CCOc1cncc(-c2ccc(NC(=O)C3(c4ccnc(NS(=O)(=O)C5CC5)n4)CC(=O)C3)nc2)n1 ; O=S(=O)(CC1CCOCC1)c1nc2ccccc2s1 ; "LHMDS" ; CCOc1cncc(-c2ccc(NC(=O)C3(c4ccnc(NS(=O)(=O)C5CC5)n4)CC(=CC4CCOCC4)C3)nc2)n1]

Compound 14-a (35 mg, 0.07 mmol) and Compound 48-a (42 mg, 0.14 mmol) were dissolved in tetrahydrofuran (2 mL), and cooled to −60° C. under a nitrogen atmosphere. A solution of lithium bis(trimethylsilyl)amide in tetrahydrofuran (0.40 mL, 1 M) was added dropwise. The reaction solution was stirred for another 0.5 hours. After the reaction was completed, it was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 48 as a white solid (3 mg, yield 7%). ¹H NMR (500 MHz, DMSO-d) δ 11.31 (br., 1H), 10.55 (br., 1H), 9.03 (d, J=2.1 Hz, 1H), 8.84 (s, 1H), 8.59-8.54 (m, 1H), 8.51 (dd, J=8.8, 2.4 Hz, 1H), 8.26-8.22 (m, 2H), 7.18 (br., 1H), 5.16-5.12 (m, 1H), 4.48 (q, J=7.0 Hz, 2H), 3.83-3.78 (m, 2H), 3.51-3.45 (m, 2H), 3.29-3.18 (m, 5H), 2.27-2.21 (m, 1H), 1.52-1.47 (m, 2H), 1.40 (t, J=7.0 Hz, 3H), 1.36-1.30 (m, 2H), 1.06-1.01 (m, 2H), 0.88-0.83 (m, 2H). MS m/z 592.0 [M+H]⁺.

### Example 49: Preparation of Compound 49

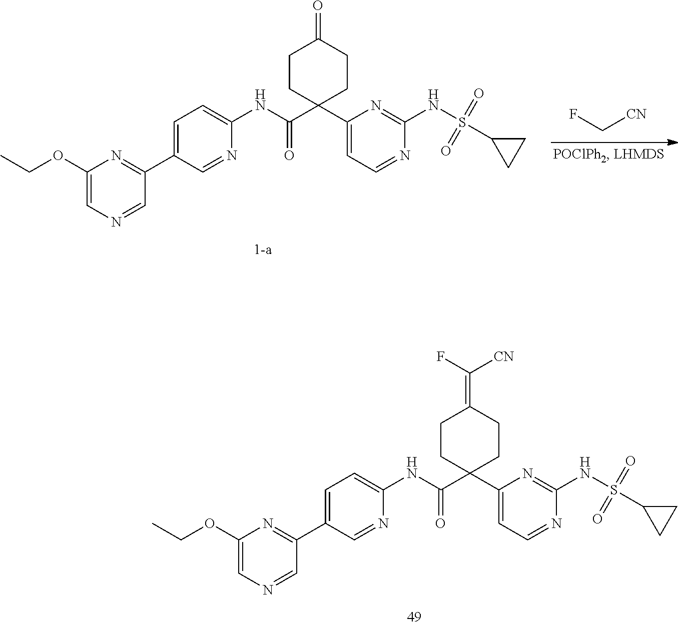

Fluoroacetonitrile (20 mg, 0.35 mmol) and diphenylphosphinyl chloride (83 mg, 0.35 mmol) were dissolved in tetrahydrofuran (2 mL), and cooled to −70° C. under a nitrogen atmosphere. A solution of lithium bis(trimethylsilyl)amide in tetrahydrofuran (0.40 mL, 1M) was added dropwise. The reaction solution was stirred at this temperature for 0.5 hours. Then Compound 1-a (40 mg, 0.07 mmol) was added. The reaction solution was stirred at room temperature for 0.5 hours. After the reaction was completed, it was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 49 as a white solid (7 mg, yield 16%). ¹H NMR (500 MHz, DMSO-d) δ 11.31 (br., 1H), 10.27 (s, 1H), 9.03 (d, J=2.1 Hz, 1H), 8.84 (s, 1H), 8.62 (d, J=5.0 Hz, 1H), 8.50 (dd, J=8.8, 2.4 Hz, 1H), 8.25 (s, 1H), 8.19 (d, J=8.7 Hz, 1H), 7.27-7.23 (m, 1H), 4.47 (q, J=7.1 Hz, 2H), 3.27-3.21 (m, 1H), 2.72-2.57 (m, 4H), 2.46-2.40 (m, 2H), 2.27-2.14 (m, 2H), 1.40 (t, J=7.0 Hz, 3H), 1.08-1.02 (m, 2H), 0.89-0.82 (m, 2H). MS m/z 578.9 [M+H]⁺.

### Example 50: Preparation of Compound 50

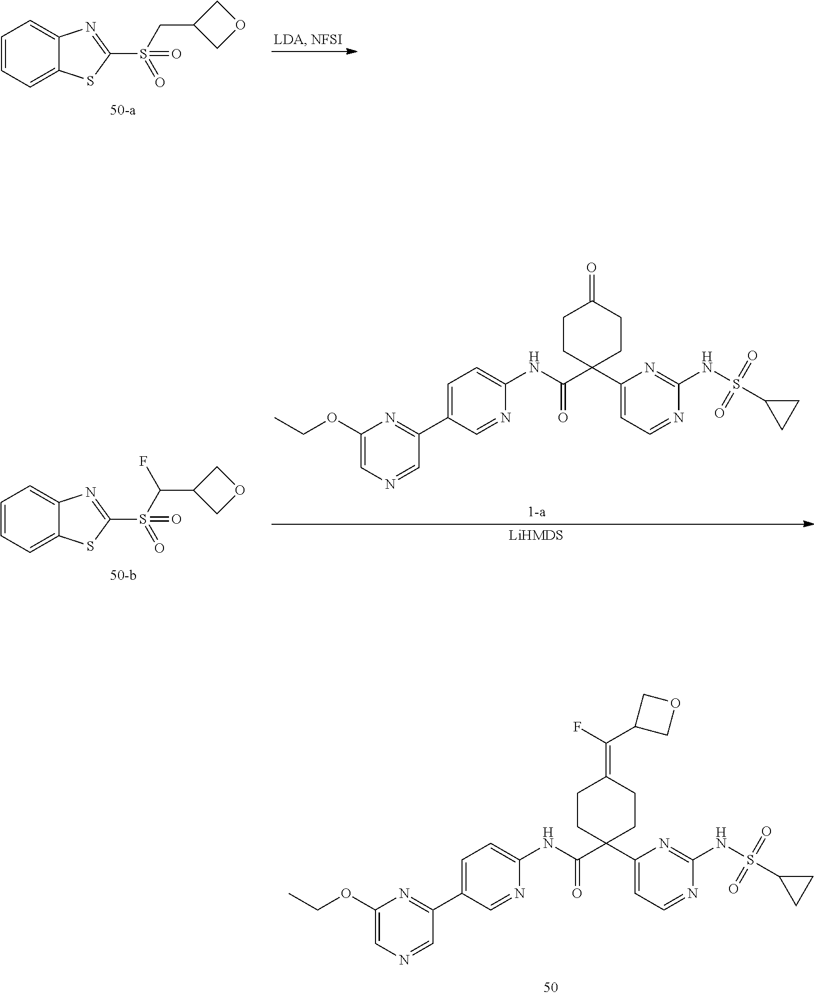 [SMILES: O=S(=O)(CC1COC1)c1nc2ccccc2s1 ; O=S(=O)(c1nc2ccccc2s1)C(F)C1COC1 ; CCOc1cncc(-c2ccc(NC(=O)C3(c4ccnc(NS(=O)(=O)C5CC5)n4)CCC(=O)CC3)nc2)n1 ; CCOc1cncc(-c2ccc(NC(=O)C3(c4ccnc(NS(=O)(=O)C5CC5)n4)CCC(=C(F)C4COC4)CC3)nc2)n1]

Compound 50-a (80 mg, 0.30 mmol) was dissolved in tetrahydrofuran. The reaction solution was cooled to −60° C., and lithium diisopropylamide (2.0 M, 0.2 mL) was slowly added dropwise under a nitrogen atmosphere. The reaction solution was stirred at this temperature for 1 hour, and then N-fluorobenzenesulfonimide (189 mg, 0.60 mmol) was added. The reaction solution was warmed to room temperature and stirred for another 2 hours. The reaction solution was quenched with saturated ammonium chloride solution, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (ethyl acetate:petroleum ether=2:1) to give Compound 50-b as a white solid (33 mg, yield 39%).

Compound 50-b (33 mg, 0.11 mmol) and Compound 1-a (30 mg, 0.06 mmol) were dissolved in tetrahydrofuran (2 mL), and cooled to −60° C. under a nitrogen atmosphere. A solution of lithium bis(trimethylsilyl)amide in tetrahydrofuran (0.20 mL, 1M) was added dropwise. The reaction solution was stirred at this temperature for 0.5 hours. The reaction solution was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 50 as a white solid (4 mg, yield 12%). ¹H NMR (500 MHz, DMSO-d) δ 11.28 (br., 1H), 10.10 (s, 1H), 9.01 (d, J=2.3 Hz, 1H), 8.83 (s, 1H), 8.60-8.54 (m, 1H), 8.49 (dd, J=8.8, 2.4 Hz, 1H), 8.25 (s, 1H), 8.17 (d, J=8.8 Hz, 1H), 7.19 (br., 1H), 4.68-4.59 (m, 4H), 4.47 (q, J=7.0 Hz, 2H), 4.43-4.32 (m, 1H), 3.26-3.20 (m, 1H), 2.28-1.95 (m, 8H), 1.39 (t, J=7.0 Hz, 3H), 1.06-1.00 (m, 2H), 0.87-0.82 (m, 2H). MS m/z 610.0 [M+H]⁺.

### Example 51: Preparation of Compound 51

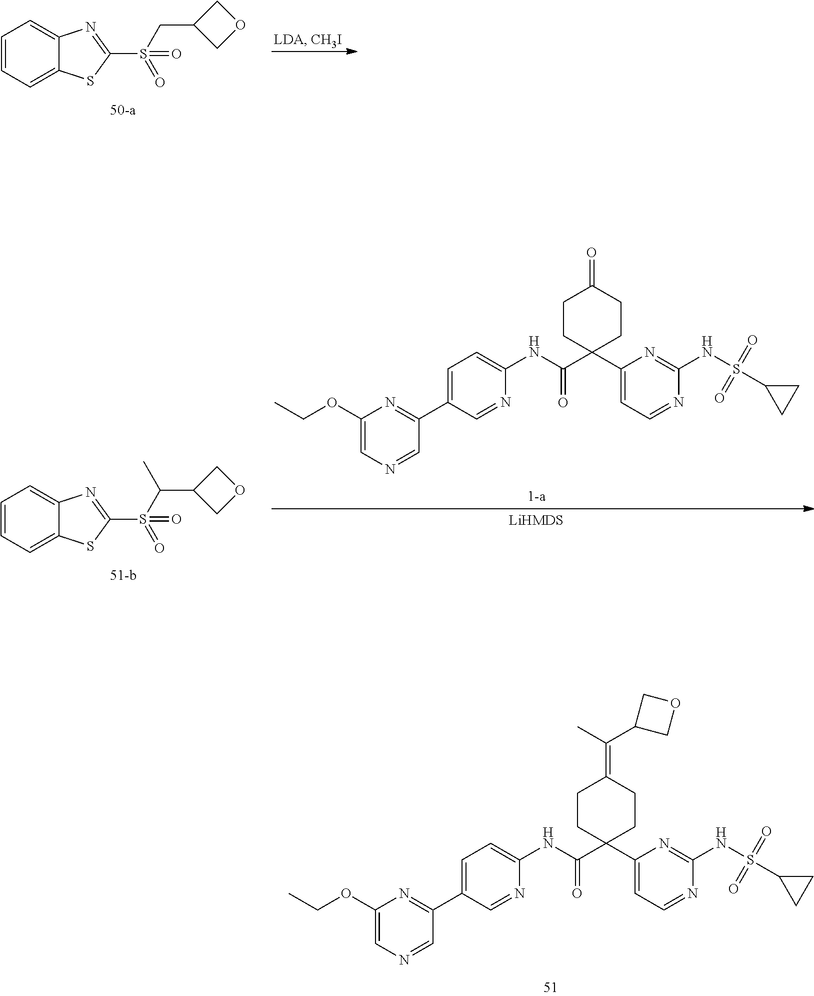 [SMILES: O=S(=O)(CC1COC1)c1nc2ccccc2s1 ; CC(C1COC1)S(=O)(=O)c1nc2ccccc2s1 ; CCOc1cncc(-c2ccc(NC(=O)C3(c4ccnc(NS(=O)(=O)C5CC5)n4)CCC(=O)CC3)nc2)n1 ; CCOc1cncc(-c2ccc(NC(=O)C3(c4ccnc(NS(=O)(=O)C5CC5)n4)CCC(=C(C)C4COC4)CC3)nc2)n1]

Compound 50-a (55 mg, 0.20 mmol) was dissolved in tetrahydrofuran. The reaction solution was cooled to −60° C., and lithium diisopropylamide (2.0 M, 0.13 mL) was slowly added dropwise under a nitrogen atmosphere. The reaction solution was stirred at this temperature for 1 hour, and then methyl iodide (142 mg, 0.40 mmol) was added. The reaction solution was warmed to room temperature and stirred for another 2 hours. The reaction solution was quenched with saturated ammonium chloride solution, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (ethyl acetate:petroleum ether=2:1) to give Compound 51-b as a white solid (41 mg, yield 71%).

Compound 51-b (32 mg, 0.11 mmol) and Compound 1-a (30 mg, 0.06 mmol) were dissolved in tetrahydrofuran (2 mL), and cooled to −60° C. under a nitrogen atmosphere. A solution of lithium bis(trimethylsilyl)amide in tetrahydrofuran (0.20 mL, 1M) was added dropwise. The reaction solution was stirred at this temperature for 0.5 hours. The reaction solution was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 51 as a white solid (4 mg, yield 12%). ¹H NMR (500 MHz, DMSO-d) δ 11.26 (br., 1H), 10.04 (s, 1H), 9.01 (d, J=2.0 Hz, 1H), 8.83 (s, 1H), 8.60-8.55 (m, 1H), 8.49 (dd, J=8.8, 2.4 Hz, 1H), 8.25 (s, 1H), 8.17 (d, J=8.8 Hz, 1H), 7.19 (br., 1H), 4.68-4.63 (m, 2H), 4.53-4.45 (m, 4H), 4.26-4.18 (m, 1H), 3.26-3.23 (m, 1H), 2.46-2.39 (m, 2H), 2.24-2.16 (m, 2H), 2.10-1.95 (m, 4H), 1.82 (s, 3H), 1.39 (t, J=7.0 Hz, 3H), 1.08-1.02 (m, 2H), 0.88-0.83 (m, 2H). MS m/z 606.1 [M+H]⁺.

### Example 52: Preparation of Compound 52

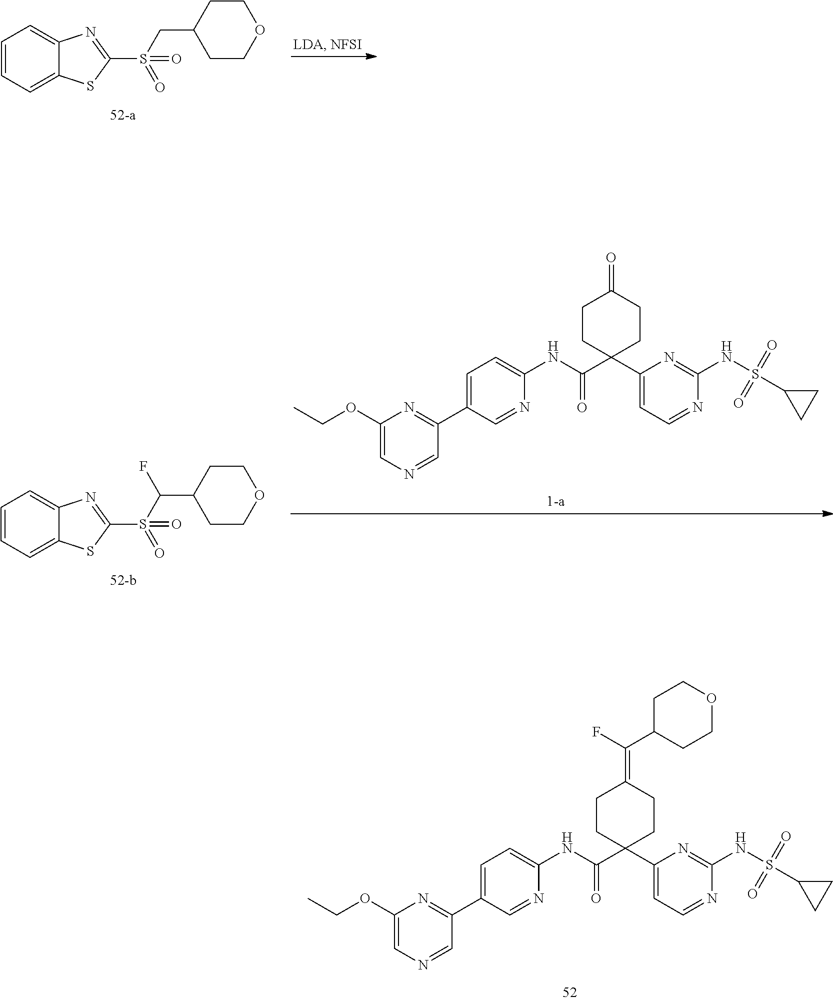 [SMILES: O=S(=O)(CC1CCOCC1)c1nc2ccccc2s1 ; O=S(=O)(c1nc2ccccc2s1)C(F)C1CCOCC1 ; CCOc1cncc(-c2ccc(NC(=O)C3(c4ccnc(NS(=O)(=O)C5CC5)n4)CCC(=O)CC3)nc2)n1 ; CCOc1cncc(-c2ccc(NC(=O)C3(c4ccnc(NS(=O)(=O)C5CC5)n4)CCC(=C(F)C4CCOCC4)CC3)nc2)n1]

Compound 52-a (100 mg, 0.34 mmol) was dissolved in tetrahydrofuran. The reaction solution was cooled to −60° C., and lithium diisopropylamide (2.0 M, 0.2 mL) was slowly added dropwise under a nitrogen atmosphere. The reaction solution was stirred at this temperature for 1 hour, and then N-fluorobenzenesulfonimide (214 mg, 0.68 mmol) was added. The reaction solution was warmed to room temperature and stirred for another 2 hours. The reaction solution was quenched with saturated ammonium chloride solution, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (ethyl acetate:petroleum ether=2:1) to give Compound 52-b as a white solid (38 mg, yield 36%).

Compound 52-b (35 mg, 0.11 mmol) and Compound 1-a (30 mg, 0.06 mmol) were dissolved in tetrahydrofuran (2 mL), and cooled to −60° C. under a nitrogen atmosphere. A solution of lithium bis(trimethylsilyl)amide in tetrahydrofuran (0.20 mL, 1 M) was added dropwise. The reaction solution was stirred at this temperature for 0.5 hours. The reaction solution was quenched with water, and the mixture was extracted with ethyl acetate (3×10 mL). The combined organic phases were washed with saturated brine, dried over anhydrous sodium sulfate, filtered, and concentrated under reduced pressure. The obtained crude product was separated and purified by preparative thin-layer plate (dichloromethane:methanol=25:1) to give Compound 52 as a white solid (3 mg, yield 8%). ¹H NMR (500 MHz, DMSO-d) δ 11.28 (br., 1H), 10.09 (s, 1H), 9.02 (d, J=2.1 Hz, 1H), 8.84 (s, 1H), 8.59 (d, J=4.6 Hz, 1H), 8.49 (dd, J=8.8, 2.4 Hz, 1H), 8.25 (s, 1H), 8.18 (d, J=8.8 Hz, 1H), 7.22 (br., 1H), 4.47 (q, J=7.0 Hz, 2H), 3.88-3.83 (m, 2H), 3.30-3.20 (m, 4H), 2.92-2.78 (m, 2H), 2.38-2.29 (m, 2H), 2.24-2.12 (m, 2H), 2.07-1.97 (m, 2H), 1.67-1.58 (m, 2H), 1.48-1.44 (m, 2H), 1.39 (t, J=7.0 Hz, 3H), 1.07-1.02 (m, 2H), 0.88-0.83 (m, 2H). MS m/z 638.0 [M+H]⁺.

### Example 53: Preparation of Compound 53

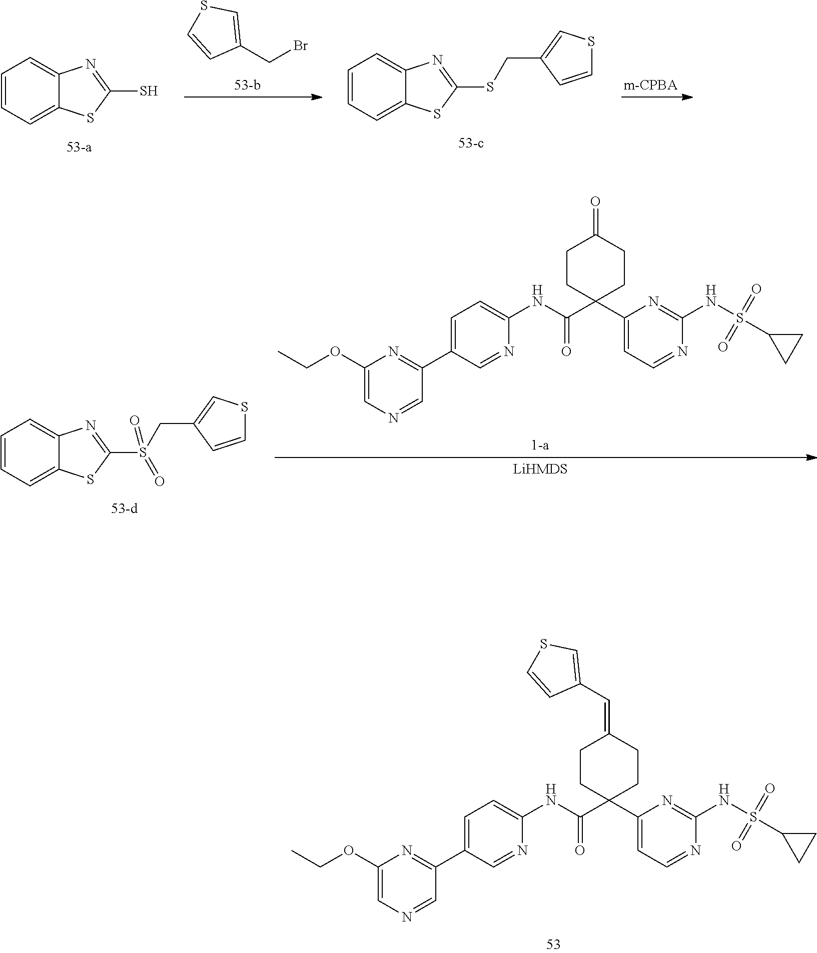 [SMILES: Sc1nc2ccccc2s1 ; BrCc1ccsc1 ; c1ccc2sc(SCc3ccsc3)nc2c1 ; O=S(=O)(Cc1ccsc1)c1nc2ccccc2s1 ; CCOc1cncc(-c2ccc(NC(=O)C3(c4ccnc(NS(=O)(=O)C5CC5)n4)CCC(=O)CC3)nc2)n1 ; CCOc1cncc(-c2ccc(NC(=O)C3(c4ccnc(NS(=O)(=O)C5CC5)n4)CCC(=Cc4ccsc4)CC3)nc2)n1]

Compound 53-a (300 mg, 1.79 mmol) and compound 53-b (318 mg, 1.79 mmol) were dissolved in tetrahydrofuran (10 mL), followed by the addition of triethylamine (0.55 mL, 5.38 mmol). The reaction mixture was stirred at 70° C. for 3 hours. After completion, the reaction was quenched with water and extracted with ethyl acetate (3×10 mL). The combined organic phases were washed with saturated brine, dried over anhydrous sodium sulfate, filtered, and concentrated under reduced pressure. The resulting crude product was directly used in the next step.

Compound 53-c (470 mg, 1.78 mmol) was dissolved in dichloromethane (10 mL), and m-chloroperoxybenzoic acid (905 mg, 4.46 mmol) was slowly added. The reaction mixture was stirred at room temperature overnight. After completion, saturated sodium thiosulfate solution was added for quenching, and the mixture was extracted with dichloromethane (3×20 mL). The combined organic phases were washed with saturated brine, dried over anhydrous sodium sulfate, filtered, and concentrated under reduced pressure. The crude product was purified by silica gel column chromatography (ethyl acetate: petroleum ether=1:1) to give compound 53-d as a white solid (450 mg, yield 85%).

Compound 53-d (22 mg, 0.07 mmol) and compound 1-a (20 mg, 0.035 mmol) were dissolved in tetrahydrofuran (1 mL), and the solution was cooled to −60° C. under a nitrogen atmosphere. A tetrahydrofuran solution of lithium bis(trimethylsilyl)amide (0.1 mL, 1 M) was slowly added dropwise. The reaction mixture was stirred at this temperature for 0.5 hours. The reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=25:1) to give compound 53 as a white solid (2.6 mg, yield 11%). ¹H NMR (500 MHz, DMSO-d) δ 11.29 (br., 1H), 10.09 (br., 1H), 9.03 (d, J=2.1 Hz, 1H), 8.84 (s, 1H), 8.55 (br., 1H), 8.50 (dd, J=8.8, 2.4 Hz, 1H), 8.25 (s, 1H), 8.20 (d, J=8.8 Hz, 1H), 7.52-7.47 (m, 1H), 7.32 (d, J=2.7 Hz, 1H), 7.20 (br., 1H), 7.08 (dd, J=5.0, 1.1 Hz, 1H), 6.23 (s, 1H), 4.51-4.45 (m, 2H), 3.23 (br., 1H), 2.37-1.96 (m, 8H), 1.40 (t, J=7.0 Hz, 3H), 1.01 (br., 2H), 0.82 (br., 2H). MS m/z 618.8 [M+H]⁺.

### Example 54: Preparation of Compound 54

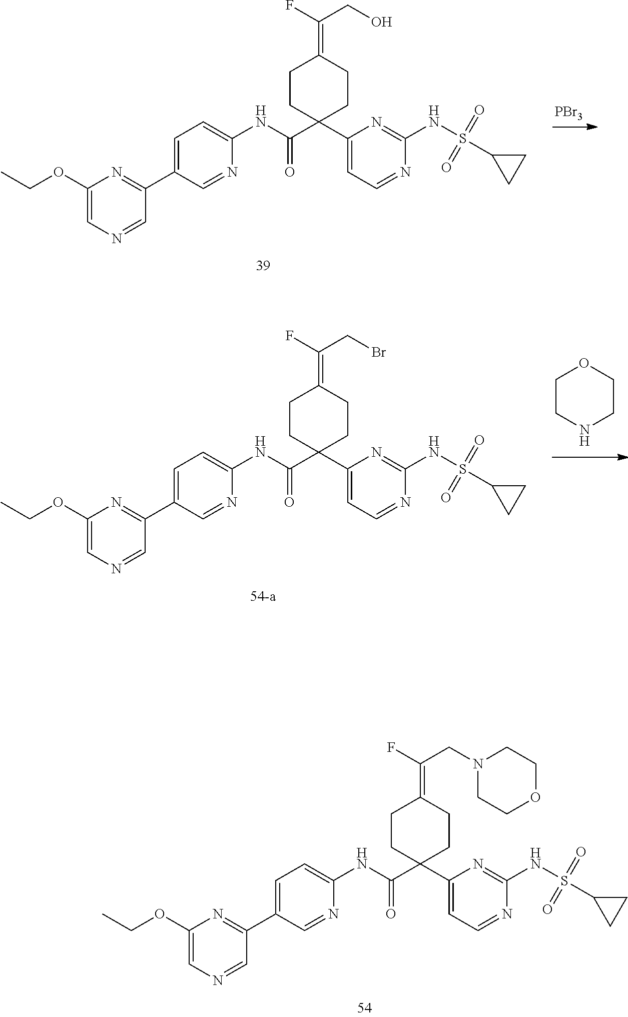 [SMILES: CCOc1cncc(-c2ccc(NC(=O)C3(c4ccnc(NS(=O)(=O)C5CC5)n4)CCC(=C(F)CO)CC3)nc2)n1 ; "PBr3" ; CCOc1cncc(-c2ccc(NC(=O)C3(c4ccnc(NS(=O)(=O)C5CC5)n4)CCC(=C(F)CBr)CC3)nc2)n1 ; C1COCCN1 ; CCOc1cncc(-c2ccc(NC(=O)C3(c4ccnc(NS(=O)(=O)C5CC5)n4)CCC(=C(F)CN4CCOCC4)CC3)nc2)n1]

Compound 39 (18 mg, 0.03 mmol) was dissolved in dichloromethane (2 mL), and phosphorus tribromide (16 mg, 0.06 mmol) was slowly added dropwise under an ice bath. The reaction mixture was stirred at this temperature for 10 minutes. The reaction solution was concentrated under reduced pressure to obtain the crude yellow oily compound 54-a, which was directly used in the next step.

Compound 54-a (19 mg, 0.03 mmol) was dissolved in dichloromethane (2 mL), and N,N-diisopropylethylamine (12 mg, 0.09 mmol) and morpholine (8 mg, 0.09 mmol) were added dropwise under an ice bath. The reaction mixture was stirred continuously under the ice bath for 0.5 hours. The reaction solution was concentrated under reduced pressure, and the crude product was purified by preparative thin-layer chromatography (dichloromethane:methanol=25:1) to give compound 54 as a white solid (3 mg, yield 16%). ¹H NMR (500 MHz, DMSO-d) δ 11.29 (br., 1H), 10.13 (s, 1H), 9.02 (d, J=1.9 Hz, 1H), 8.84 (s, 1H), 8.60 (d, J=5.3 Hz, 1H), 8.50 (dd, J=8.8, 2.4 Hz, 1H), 8.25 (s, 1H), 8.19 (d, J=9.1 Hz, 1H), 7.24 (d, J=5.3 Hz, 1H), 4.47 (q, J=7.0 Hz, 2H), 3.66-3.53 (m, 4H), 3.26-3.09 (m, 6H), 2.47-2.36 (m, 5H), 2.27-2.20 (m, 2H), 2.11-2.02 (m, 2H), 1.39 (t, J=7.0 Hz, 3H), 1.08-1.03 (m, 2H), 0.88-0.85 (m, 2H). MS m/z 653.1 [M+H]⁺.

### Example 55: Preparation of Compound 55

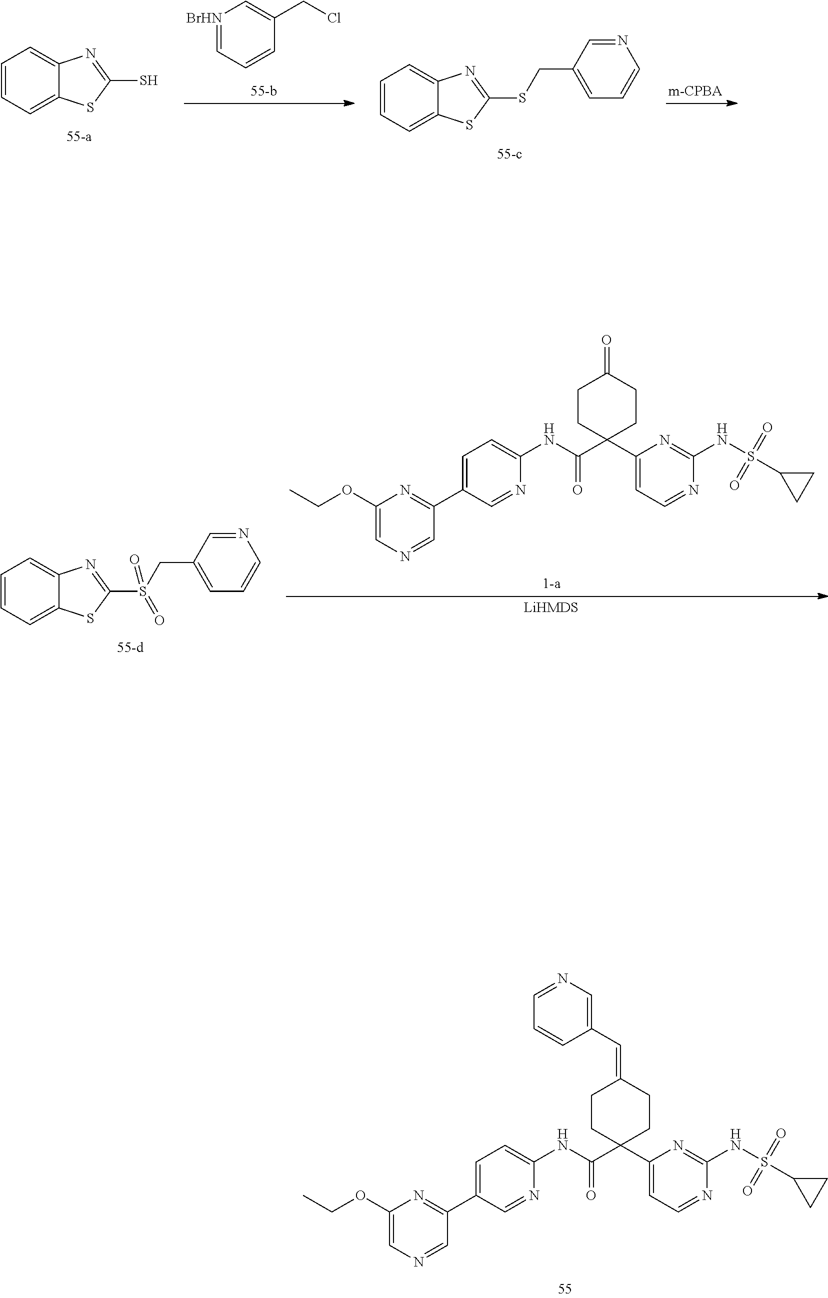

Compound 55-a (200 mg, 1.2 mmol) and compound 55-b (324 mg, 1.55 mmol) were dissolved in tetrahydrofuran (10 mL), followed by the addition of triethylamine (0.5 mL, 3.59 mmol). The reaction mixture was stirred at 70° C. for 3 hours. After completion, the reaction was quenched with water and extracted with ethyl acetate (3×10 mL). The combined organic phases were washed with saturated brine, dried over anhydrous sodium sulfate, filtered, and concentrated under reduced pressure. The resulting crude product was directly used in the next step.

Compound 55-c (308 mg, 1.19 mmol) was dissolved in dichloromethane (10 mL), and m-chloroperoxybenzoic acid (532 mg, 2.62 mmol) was slowly added. The reaction mixture was stirred at room temperature overnight. Saturated sodium thiosulfate solution was added for quenching, and the mixture was extracted with dichloromethane (3×20 mL). The combined organic phases were washed with saturated brine, dried over anhydrous sodium sulfate, filtered, and concentrated under reduced pressure. The crude product was purified by silica gel column chromatography (ethyl acetate: petroleum ether=1:1) to give compound 55-d as a white solid (190 mg, yield 58%).

Compound 55-d (21 mg, 0.07 mmol) and compound 1-a (20 mg, 0.035 mmol) were dissolved in tetrahydrofuran (1 mL), and the solution was cooled to −60° C. under a nitrogen atmosphere. A tetrahydrofuran solution of lithium bis(trimethylsilyl)amide (0.2 mL, 1 M) was added dropwise. The reaction mixture was stirred at this temperature for 0.5 hours. The reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=25:1) to give compound 55 as a white solid (1.21 mg, yield 5.3%). ¹H NMR (500 MHz, DMSO-d) δ 11.30 (br., 1H), 10.15 (s, 1H), 9.03 (d, J=1.9 Hz, 1H), 8.84 (s, 1H), 8.60 (d, J=5.1 Hz, 1H), 8.50 (dd, J=8.8, 2.4 Hz, 1H), 8.45 (s, 1H), 8.41 (d, J=3.5 Hz, 1H), 8.25 (s, 1H), 8.22 (d, J=8.9 Hz, 1H), 7.67-7.62 (m, 1H), 7.38-7.32 (m, 1H), 7.25 (br., 1H), 6.32 (s, 1H), 4.51-4.44 (m, 2H), 3.27-3.22 (m, 1H), 2.46-2.34 (m, 3H), 2.24-1.95 (m, 5H), 1.40 (t, J=7.0 Hz, 3H), 1.07-1.01 (m, 2H), 0.88-0.84 (m, 2H). MS m/z 618.8 [M+H]⁺.

### Example 56: Preparation of Compound 56

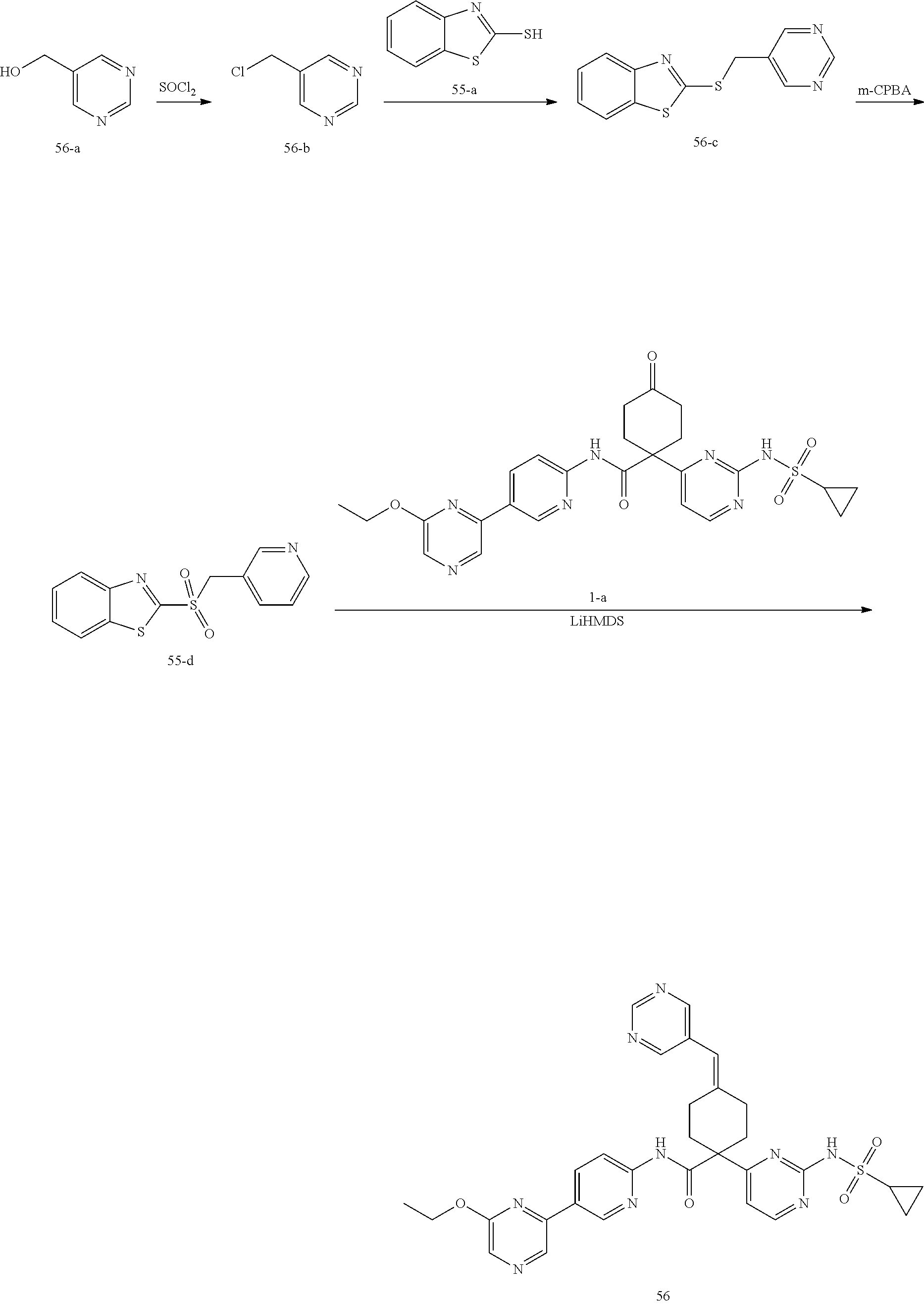

Compound 56-a (200 mg, 1.82 mmol) was dissolved in dichloromethane (5 mL), and thionyl chloride (648 mg, 5.45 mmol) was added dropwise under an ice bath. The reaction mixture was stirred at room temperature for 0.5 hours. After completion, the reaction solution was concentrated under reduced pressure, and the resulting crude product was directly used in the next step.

Compound 56-b (230 mg, 1.79 mmol) and compound 55-a (250 mg, 1.49 mmol) were dissolved in tetrahydrofuran (10 mL), followed by the addition of triethylamine (0.63 mL, 4.48 mmol). The reaction mixture was stirred at 70° C. for 12 hours. After completion, the reaction was quenched with water and extracted with ethyl acetate (3×10 mL). The combined organic phases were washed with saturated brine, dried over anhydrous sodium sulfate, filtered, and concentrated under reduced pressure. The resulting crude product was directly used in the next step.

Compound 56-c (387 mg, 1.49 mmol) was dissolved in dichloromethane (10 mL), and m-chloroperoxybenzoic acid (758 mg, 3.73 mmol) was slowly added. The reaction mixture was stirred at room temperature overnight. Saturated sodium thiosulfate solution was added for quenching, and the mixture was extracted with dichloromethane (3×20 mL). The combined organic phases were washed with saturated brine, dried over anhydrous sodium sulfate, filtered, and concentrated under reduced pressure. The crude product was purified by silica gel column chromatography (ethyl acetate: petroleum ether=1:1) to give compound 56-d as a white solid (190 mg, yield 44%).

Compound 56-d (21 mg, 0.07 mmol) and compound 1-a (20 mg, 0.035 mmol) were dissolved in tetrahydrofuran (1 mL), and the solution was cooled to −60° C. under a nitrogen atmosphere. A tetrahydrofuran solution of lithium bis(trimethylsilyl)amide (0.2 mL, 1 M) was added dropwise. The reaction mixture was stirred at this temperature for 0.5 hours. The reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=25:1) to give compound 56 as a white solid (0.84 mg, yield 3.68%). ¹H NMR (500 MHz, DMSO-d) δ 9.98 (s, 1H), 9.06-8.99 (m, 2H), 8.83 (s, 1H), 8.69 (s, 2H), 8.49 (dd, J=8.8, 2.4 Hz, 1H), 8.38 (s, 1H), 8.24 (s, 1H), 8.16 (d, J=8.7 Hz, 1H), 6.87 (s, 1H), 6.26 (s, 1H), 4.52-4.42 (m, 2H), 3.12 (br., 1H), 2.43-2.14 (m, 6H), 2.03-1.94 (m, 2H), 1.39 (t, J=7.0 Hz, 3H), 0.93-0.87 (m, 2H), 0.76-0.67 (m, 2H). MS m/z 614.0 [M+H]⁺.

### Example 57&60: Preparation of Compound 57&60

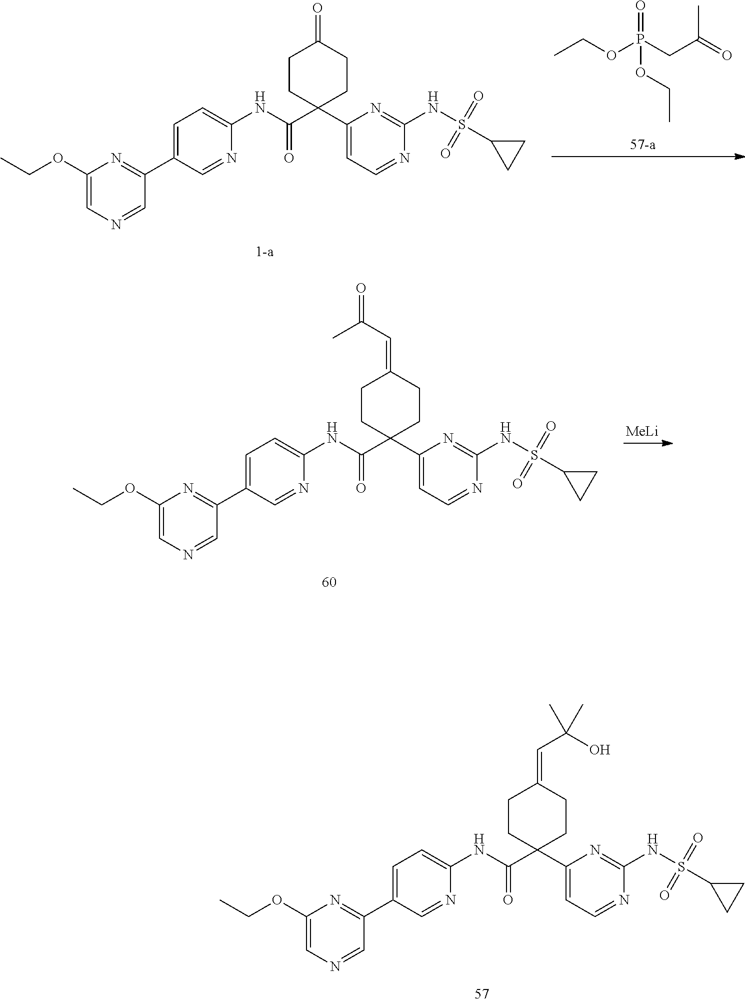

Potassium hydroxide (10 mg, 0.18 mmol) was dissolved in ethanol and water (4:1, 1.5 mL). Under an ice bath, compound 57-a (27 mg, 0.14 mmol) was added, and after stirring for 10 minutes, compound 1-a (50 mg, 0.09 mmol) was added. The reaction mixture was stirred at room temperature for 14 hours. After completion, the reaction solution was adjusted to pH 5-6 with dilute hydrochloric acid, and then extracted with ethyl acetate (3×10 mL). The combined organic phases were washed with saturated brine, dried over anhydrous sodium sulfate, filtered, and concentrated under reduced pressure. The crude product was purified by silica gel column chromatography (dichloromethane:methanol=25:1) to give compound 60 as a white solid (35 mg, yield 65%), MS m/z 578.0 [M+H]⁺.

Compound 60 (25 mg, 0.04 mmol) was dissolved in tetrahydrofuran (1 mL), and the solution was cooled to −60° C. under a nitrogen atmosphere. A methyl lithium solution (0.05 mL, 1.6 M) was added dropwise. The reaction mixture was stirred at 0° C. for 2 hours. After completion, the reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=20:1) to give compound 57 as a white solid (4.10 mg, yield 16%). ¹H NMR (500 MHz, DMSO-d) δ 11.24 (br., 1H), 9.95 (s, 1H), 9.00 (d, J=2.4 Hz, 1H), 8.83 (s, 1H), 8.58 (d, J=4.8 Hz, 1H), 8.48 (dd, J=8.8, 2.4 Hz, 1H), 8.24 (s, 1H), 8.15 (d, J=8.9 Hz, 1H), 7.19 (br., 1H), 5.47 (s, 1H), 4.50-4.44 (m, 2H), 4.07 (s, 1H), 3.23-3.17 (m, 1H), 2.81-2.66 (m, 2H), 2.44-2.14 (m, 6H), 1.39 (t, J=7.0 Hz, 3H), 1.07-1.00 (m, 2H), 0.98 (d, J=11.2 Hz, 6H), 0.88-0.81 (m, 2H). MS m/z 593.9 [M+H]⁺.

### Example 58: Preparation of Compound 58

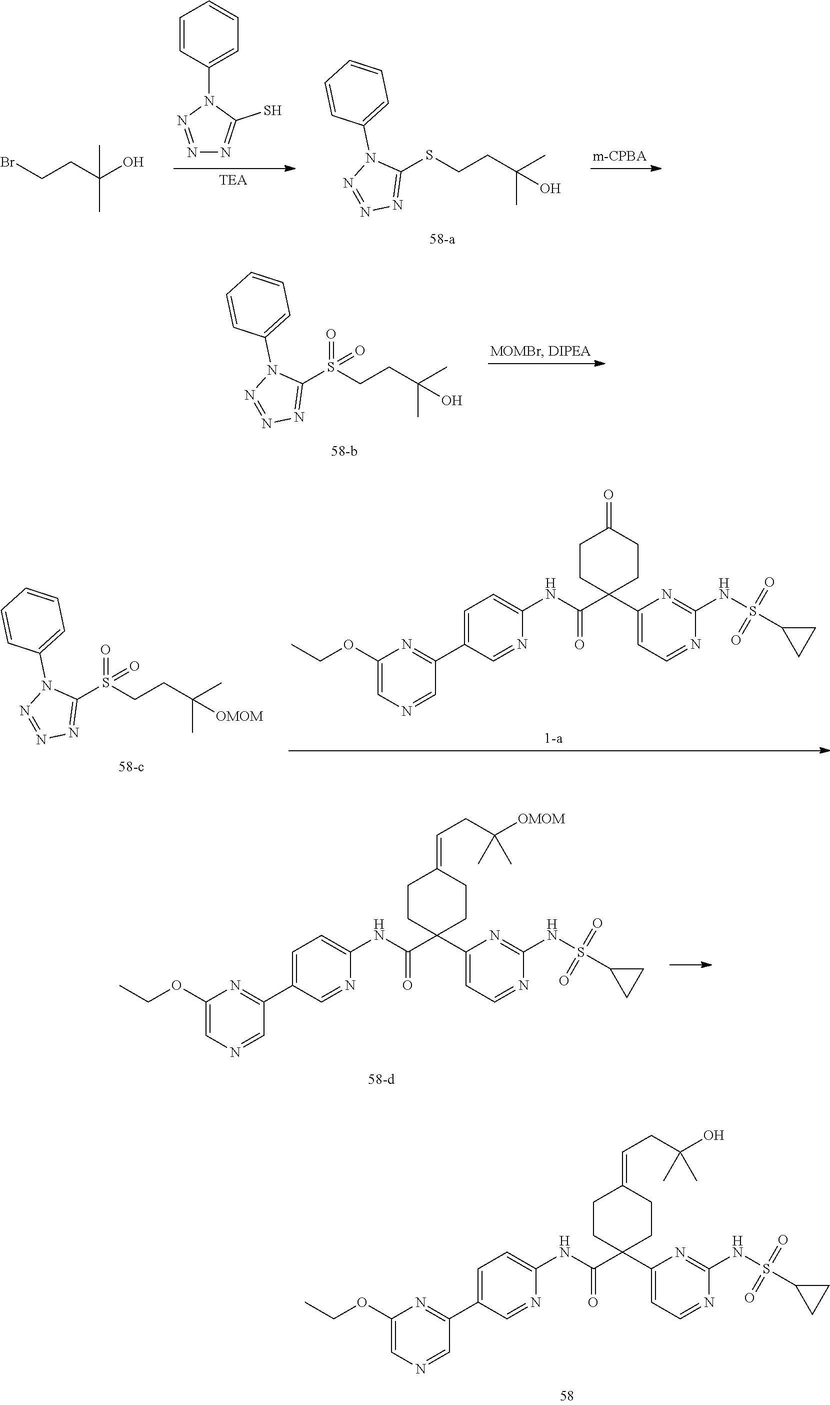

4-Bromo-2-methyl-2-butanol (406 mg, 2.43 mmol) and 1-phenyl-1H-tetrazole-5(2H)-thione (433 mg, 2.43 mmol) were dissolved in tetrahydrofuran (5 mL), and triethylamine (736 mg, 7.29 mmol) was added dropwise. The reaction mixture was heated and stirred at 70° C. for 1 hour. After completion, the reaction solution was diluted with water, and the mixture was extracted with ethyl acetate (3×10 mL). The combined organic phases were washed with saturated brine, dried over anhydrous sodium sulfate, filtered, and concentrated under reduced pressure. The resulting crude compound 58-a was directly used in the next step.

Compound 58-a (211 mg, 0.80 mmol) was dissolved in dichloromethane (3 mL), and m-chloroperoxybenzoic acid (304 mg, 1.76 mmol) was added. The reaction mixture was stirred at room temperature overnight. The reaction system was quenched with saturated sodium thiosulfate solution, and the mixture was extracted with dichloromethane (3×10 mL). The combined organic phases were washed with saturated brine, dried over anhydrous sodium sulfate, filtered, and concentrated under reduced pressure. The crude product was purified by silica gel column chromatography (ethyl acetate: petroleum ether=1:1) to give compound 58-b as a white solid (166 mg, yield 70%).

Compound 58-b (166 mg, 0.56 mmol) and N,N-diisopropylethylamine (217 mg, 1.68 mmol) were dissolved in dichloromethane (3 mL). Bromomethyl methyl ether (140 mg, 1.12 mmol) was added dropwise under an ice bath. The reaction mixture was stirred at room temperature overnight. The reaction solution was concentrated under reduced pressure, and the crude product was purified by silica gel column chromatography (ethyl acetate: petroleum ether=5:1) to obtain compound 58-c (123 mg, yield 64%).

Compound 58-c (38 mg, 0.11 mmol) and compound 1-a (30 mg, 0.06 mmol) were dissolved in tetrahydrofuran (2 mL), and the solution was cooled to −60° C. under a nitrogen atmosphere. A tetrahydrofuran solution of lithium bis(trimethylsilyl)amide (0.30 mL, 1 M) was added dropwise. The reaction mixture was stirred at this temperature for 0.5 hours. The reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=25:1) to give compound 58-d as a white solid (6 mg, yield 17%).

Compound 58-d (6 mg, 0.01 mmol) was dissolved in dichloromethane (2 mL), and trifluoroacetic acid (0.5 mL) was added dropwise. The reaction mixture was stirred at room temperature for 3 hours. The reaction solution was concentrated under reduced pressure, and the crude product was purified by preparative thin-layer chromatography (dichloromethane:methanol=25:1) to give compound 58 as a white solid (2 mg, yield 36%). ¹H NMR (500 MHz, DMSO-d) δ 11.29 (br., 1H), 10.04 (s, 1H), 9.02 (d, J=2.2 Hz, 1H), 8.84 (s, 1H), 8.60-8.55 (m, 1H), 8.49 (dd, J=8.8, 2.4 Hz, 1H), 8.25 (s, 1H), 8.19 (d, J=8.8 Hz, 1H), 7.19 (br., 1H), 5.25 (t, J=7.6 Hz, 1H), 4.47 (q, J=7.0 Hz, 2H), 4.20 (s, 1H), 3.25-3.22 (m, 1H), 2.38-2.34 (m, 2H), 2.31-2.23 (m, 2H), 2.22-2.14 (m, 2H), 2.09-2.06 (m, 2H), 2.01-1.96 (m, 2H), 1.39 (t, J=7.0 Hz, 3H), 1.08-1.01 (m, 8H), 0.87-0.84 (m, 2H). MS m/z 608.0 [M+H]⁺.

### Example 59: Preparation of Compound 59

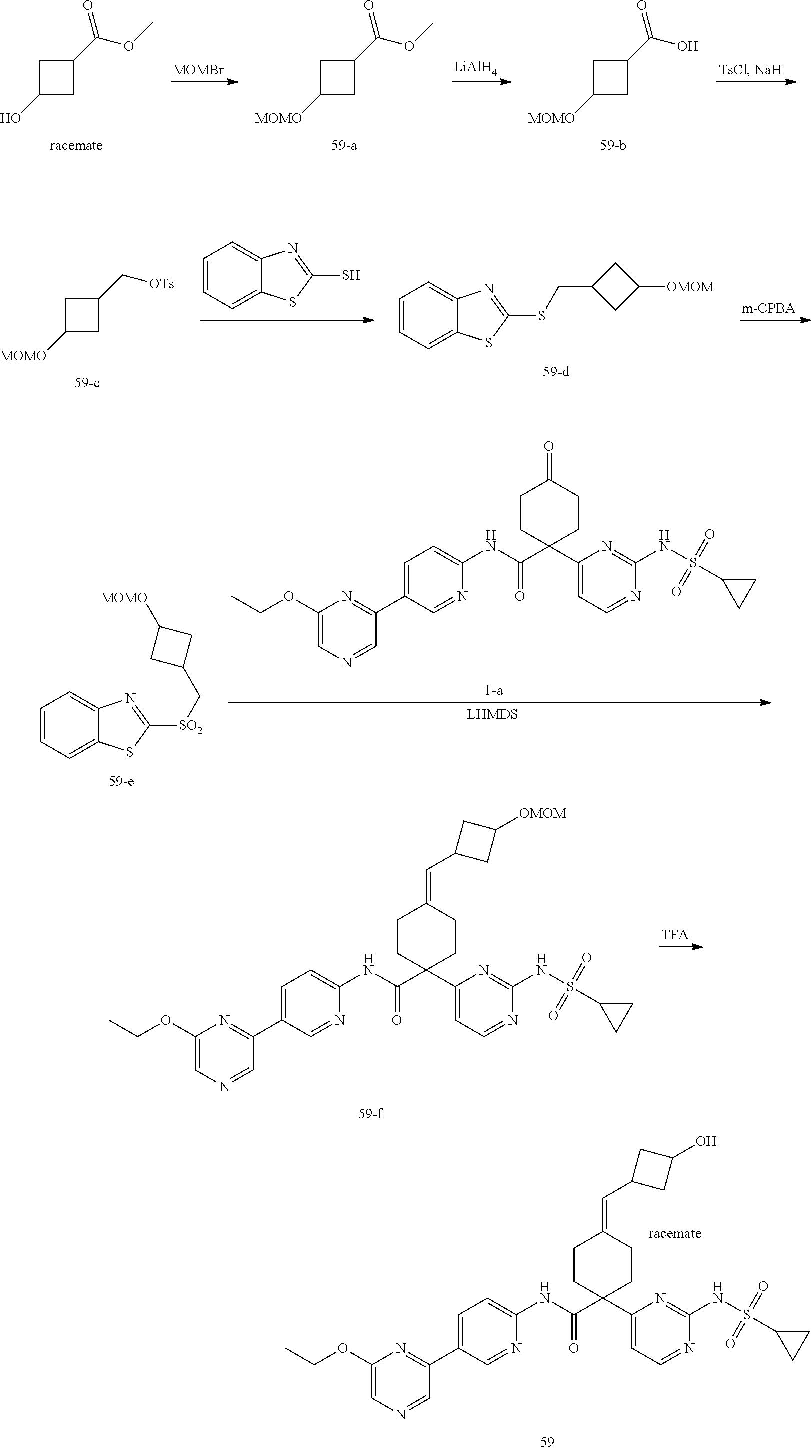

Methyl 3-hydroxycyclobutanecarboxylate (500 mg, 3.84 mmol) and N,N-diisopropylethylamine (1.49 g, 11.52 mmol) were dissolved in dichloromethane (10 mL), and bromomethyl methyl ether (960 mg, 7.68 mmol) was added dropwise under an ice bath. The reaction mixture was stirred at room temperature overnight. The reaction solution was concentrated under reduced pressure, and the crude product was purified by silica gel column chromatography (ethyl acetate: petroleum ether=5:1) to obtain compound 59-a (483 mg, yield 72%).

Compound 59-a (483 mg, 2.77 mmol) was dissolved in tetrahydrofuran (10 mL), and lithium aluminum hydride (211 mg, 5.54 mmol) was slowly added. The reaction mixture was stirred at room temperature for 2 hours. The reaction mixture was quenched with sodium sulfate decahydrate, filtered through Celite, and the filtrate was concentrated under reduced pressure. The product was purified by silica gel column chromatography (ethyl acetate: petroleum ether=2:1) to obtain compound 59-b (365 mg, yield 90%).

Compound 59-b (206 mg, 1.41 mmol) and p-toluenesulfonyl chloride (281 mg, 1.48 mmol) were dissolved in dichloromethane (5 mL). Sodium hydride (60%, 113 mg, 2.82 mmol) was added under an ice bath. The reaction mixture was stirred at room temperature for 0.5 hours. The reaction mixture was quenched with saturated ammonium chloride solution, and the mixture was extracted with dichloromethane (3×10 mL). The combined organic phases were washed with saturated brine, dried over anhydrous sodium sulfate, filtered, and concentrated under reduced pressure. The resulting crude compound 59-c was directly used in the next step.

Compound 59-c (423 mg, 1.41 mmol) and benzo[D]thiazole-2-thiol (235 mg, 1.41 mmol) were dissolved in tetrahydrofuran (10 mL), and triethylamine (427 mg, 4.23 mmol) was added dropwise. The reaction mixture was heated and stirred at 70° C. for 1 hour. After completion, the reaction solution was diluted with water, and the mixture was extracted with ethyl acetate (3×10 mL). The combined organic phases were washed with saturated brine, dried over anhydrous sodium sulfate, filtered, and concentrated under reduced pressure. The product was purified by silica gel column chromatography (ethyl acetate: petroleum ether=2:1) to obtain compound 59-d (115 mg, yield 28%).

Compound 59-d (115 mg, 0.39 mmol) was dissolved in dichloromethane (3 mL), and m-chloroperoxybenzoic acid (148 mg, 0.86 mmol) was added. The reaction mixture was stirred at room temperature overnight. The reaction solution was quenched with saturated sodium thiosulfate solution, and the mixture was extracted with dichloromethane (3×10 mL). The combined organic phases were washed with saturated brine, dried over anhydrous sodium sulfate, filtered, and concentrated under reduced pressure. The crude product was purified by silica gel column chromatography (ethyl acetate: petroleum ether=1:1) to give compound 59-e as a white solid (103 mg, yield 81%).

Compound 59-e (36 mg, 0.11 mmol) and compound 1-a (30 mg, 0.06 mmol) were dissolved in tetrahydrofuran (2 mL), and the solution was cooled to −60° C. under a nitrogen atmosphere. A tetrahydrofuran solution of lithium bis(trimethylsilyl)amide (0.30 mL, 1 M) was added dropwise. The reaction mixture was stirred at this temperature for 0.5 hours. The reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=25:1) to give compound 59-f as a white solid (12 mg, yield 33%).

Compound 59-f (6 mg, 0.01 mmol) was dissolved in dichloromethane (2 mL), and trifluoroacetic acid (0.5 mL) was added dropwise. The reaction mixture was stirred at room temperature for 3 hours. The reaction solution was concentrated under reduced pressure, and the crude product was purified by preparative thin-layer chromatography (dichloromethane:methanol=25:1) to give compound 59 as a white solid (2 mg, yield 36%). ¹H NMR (500 MHz, DMSO-d) δ 11.25 (br., 1H), 10.04 (s, 1H), 9.01 (d, J=1.9 Hz, 1H), 8.84 (s, 1H), 8.59-8.53 (m, 1H), 8.49 (dd, J=8.8, 2.4 Hz, 1H), 8.25 (s, 1H), 8.18 (d, J=8.8 Hz, 1H), 7.17 (br., 1H), 5.12 (d, J=8.4 Hz, 1H), 4.96 (d, J=6.7 Hz, 1H), 4.47 (q, J=7.0 Hz, 2H), 3.91-3.84 (m, 1H), 3.25-3.22 (m, 1H), 2.37-1.92 (m, 12H), 1.57-1.52 (m, 1H), 1.39 (t, J=7.0 Hz, 3H), 1.05-1.01 (m, 2H), 0.87-0.84 (m, 2H). MS m/z 606.0 [M+H]⁺.

### Example 61: Preparation of Compound 61

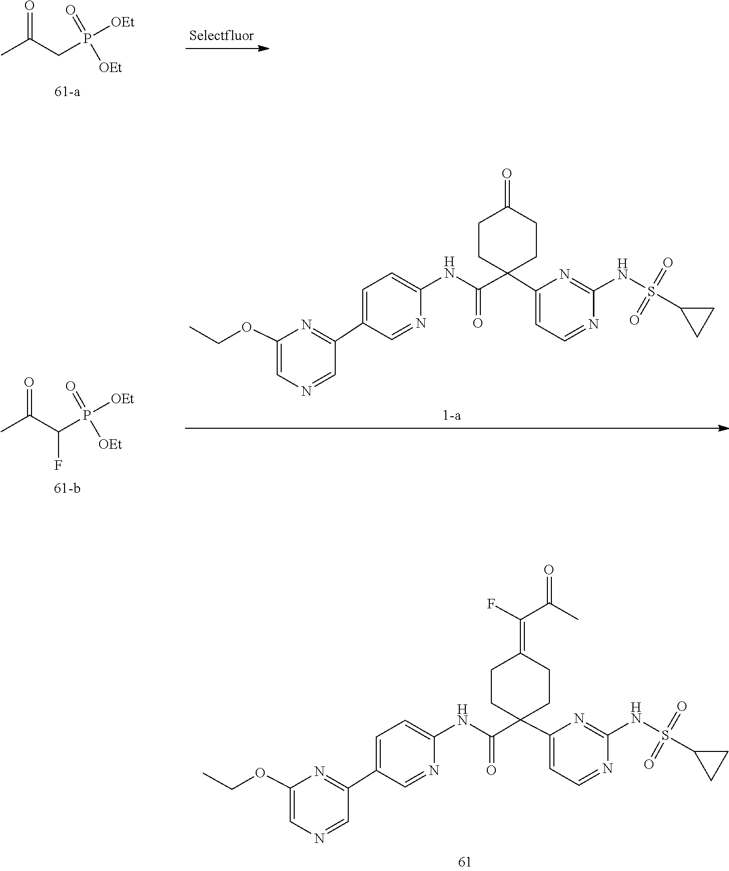 [SMILES: CCOP(=O)(CC(C)=O)OCC ; CCOP(=O)(OCC)C(F)C(C)=O ; CCOc1cncc(-c2ccc(NC(=O)C3(c4ccnc(NS(=O)(=O)C5CC5)n4)CCC(=O)CC3)nc2)n1 ; CCOc1cncc(-c2ccc(NC(=O)C3(c4ccnc(NS(=O)(=O)C5CC5)n4)CCC(=C(F)C(C)=O)CC3)nc2)n1]

Diethyl acetonylphosphonate (593 mg, 3.05 mmol) was dissolved in acetonitrile (5 mL), and Selectfluor (2.16 g, 6.10 mmol) was added. The reaction mixture was stirred at 80° C. for 24 hours. After completion, the reaction was quenched with water and extracted with ethyl acetate (3×15 mL). The combined organic phases were washed with saturated brine, dried over anhydrous sodium sulfate, filtered, and concentrated under reduced pressure. The crude product was purified by silica gel column chromatography (petroleum ether: ethyl acetate=10:1) to give compound 61-b as an oily liquid (110 mg, yield 17%).

Potassium hydroxide (8 mg, 0.15 mmol) was dissolved in ethanol and water (4:1, 1.5 mL). Under an ice bath, compound 61-b (19 mg, 0.09 mmol) was added, and after stirring for 10 minutes, compound 1-a (40 mg, 0.07 mmol) was added. The reaction mixture was stirred at room temperature for 14 hours. After completion, the reaction solution was adjusted to pH 5-6 with dilute hydrochloric acid, and then extracted with ethyl acetate (3×10 mL). The combined organic phases were washed with saturated brine, dried over anhydrous sodium sulfate, filtered, and concentrated under reduced pressure. The crude product was purified by silica gel column chromatography (dichloromethane:methanol=25:1) to give compound 61 as a white solid (20 mg, yield 45%). ¹H NMR (500 MHz, DMSO-d) δ 11.28 (br., 1H), 10.18 (s, 1H), 9.02 (d, J=2.0 Hz, 1H), 8.84 (s, 1H), 8.60 (d, J=5.4 Hz, 1H), 8.49 (dd, J=8.8, 2.4 Hz, 1H), 8.25 (s, 1H), 8.19 (d, J=8.8 Hz, 1H), 7.23 (br., 1H), 4.51-4.43 (m, 2H), 3.26-3.21 (m, 1H), 2.65-2.54 (m, 4H), 2.26 (d, J=5.5 Hz, 3H), 2.21-2.06 (m, 4H), 1.39 (t, J=7.0 Hz, 3H), 1.07-1.00 (m, 2H), 0.89-0.82 (m, 2H). MS m/z 595.9 [M+H]⁺.

### Example 62: Preparation of Compound 62

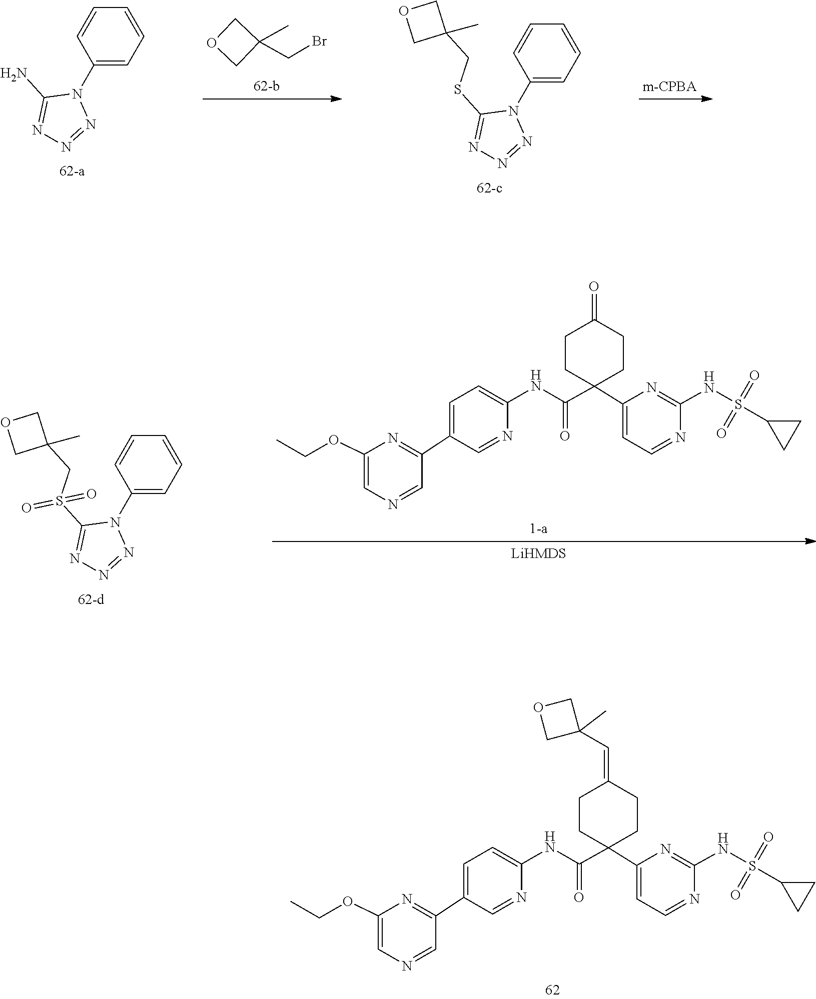

Compound 62-a (260 mg, 1.46 mmol) and compound 62-b (313 mg, 1.90 mmol) were dissolved in tetrahydrofuran (10 mL), followed by the addition of triethylamine (0.6 mL, 4.38 mmol). The reaction mixture was stirred at 70° C. for 3 hours. After completion, the reaction was quenched with water and extracted with ethyl acetate (3×10 mL). The combined organic phases were washed with saturated brine, dried over anhydrous sodium sulfate, filtered, and concentrated under reduced pressure. The resulting crude product was directly used in the next step.

Compound 62-c (382 mg, 1.46 mmol) was dissolved in dichloromethane (10 mL), and m-chloroperoxybenzoic acid (650 mg, 3.20 mmol) was added. The reaction mixture was stirred at room temperature overnight. The reaction solution was quenched with saturated sodium thiosulfate solution, and the mixture was extracted with dichloromethane (3×20 mL). The combined organic phases were washed with saturated brine, dried over anhydrous sodium sulfate, filtered, and concentrated under reduced pressure. The crude product was purified by silica gel column chromatography (ethyl acetate: petroleum ether=1:1) to give compound 62-d as a white solid (130 mg, yield 30%).

Compound 62-d (66 mg, 0.22 mmol) and compound 1-a (30 mg, 0.06 mmol) were dissolved in tetrahydrofuran (1 mL), and the solution was cooled to −60° C. under a nitrogen atmosphere. A tetrahydrofuran solution of lithium bis(trimethylsilyl)amide (0.3 mL, 1 M) was added dropwise. The reaction mixture was stirred at this temperature for 0.5 hours. The reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=25:1) to give compound 62 as a white solid (4.15 mg, yield 12%). ¹H NMR (500 MHz, DMSO-d) δ 11.32 (br., 1H), 10.08 (s, 1H), 9.02 (d, J=1.9 Hz, 1H), 8.84 (s, 1H), 8.57 (d, J=5.3 Hz, 1H), 8.49 (dd, J=8.8, 2.4 Hz, 1H), 8.25 (s, 1H), 8.19 (d, J=8.7 Hz, 1H), 7.19 (d, J=5.1 Hz, 1H), 5.41 (s, 1H), 4.53 (t, J=5.1 Hz, 2H), 4.50-4.43 (m, 2H), 4.37-4.28 (m, 2H), 3.26-3.20 (m, 1H), 2.49-2.43 (m, 1H), 2.27-1.89 (m, 7H), 1.43-1.37 (m, 6H), 1.05-1.00 (m, 2H), 0.88-0.81 (m, 2H). MS m/z 605.9 [M+H]⁺.

### Example 63: Preparation of Compound 63

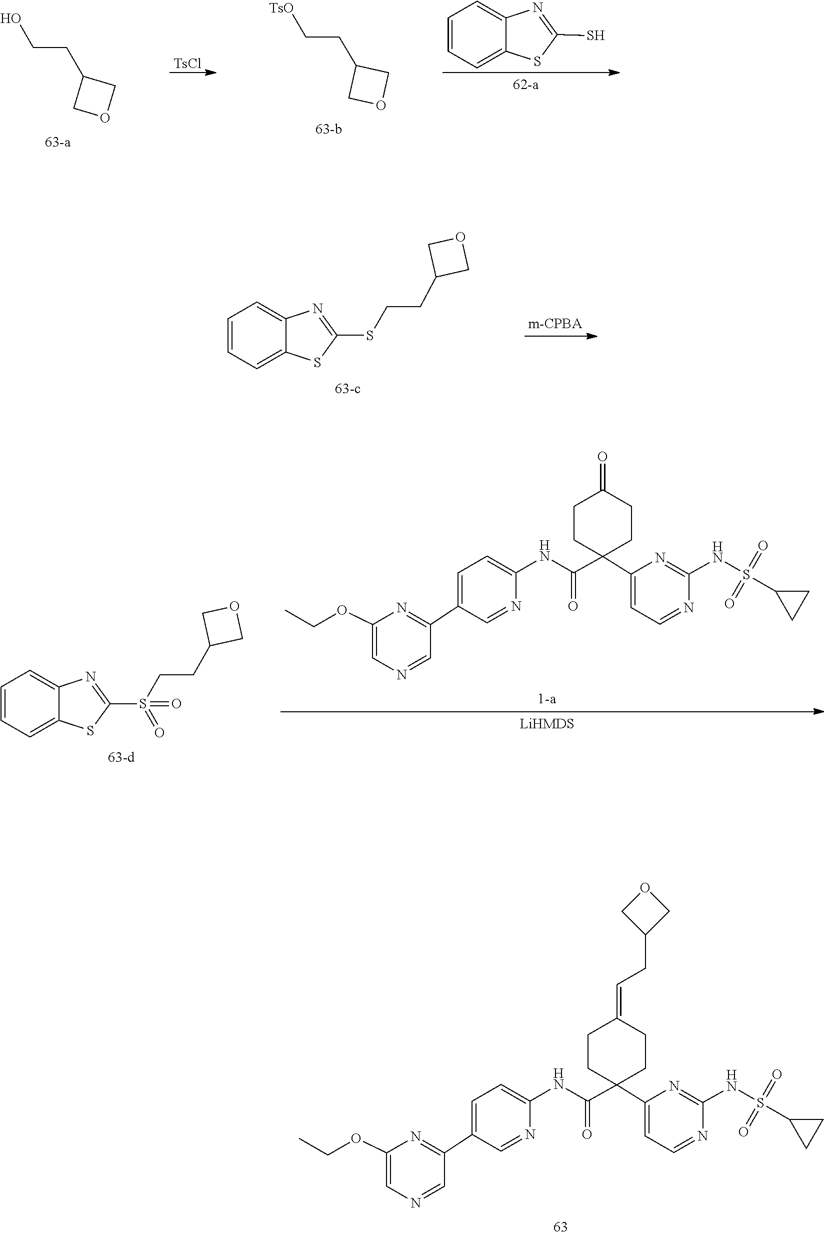

Compound 63-a (142 mg, 1.39 mmol) was dissolved in dichloromethane (5 mL). Under an ice bath, p-toluenesulfonyl chloride (291 mg, 1.53 mmol) was added, followed by sodium hydride (167 mg, 4.17 mmol). The reaction mixture was stirred at room temperature for 0.5 hours. After completion, the reaction was quenched with water and extracted with dichloromethane (3×10 mL). The combined organic phases were washed with saturated brine, dried over anhydrous sodium sulfate, filtered, and concentrated under reduced pressure. The resulting crude product was directly used in the next step.

Compound 63-b (239 mg, 0.93 mmol) and compound 62-a (130 mg, 0.78 mmol) were dissolved in tetrahydrofuran (10 mL), followed by the addition of triethylamine (0.3 mL, 2.33 mmol). The reaction mixture was stirred at 70° C. for 12 hours. After completion, water was added to the reaction solution, and the mixture was extracted with ethyl acetate (3×10 mL). The combined organic phases were washed with saturated brine, dried over anhydrous sodium sulfate, filtered, and concentrated under reduced pressure. The crude product was purified by silica gel column chromatography (ethyl acetate: petroleum ether=5:1) to give compound 63-c as a white solid (90 mg, yield 46%).

Compound 63-c (90 mg, 0.36 mmol) was dissolved in dichloromethane (10 mL), and m-chloroperoxybenzoic acid (160 mg, 0.79 mmol) was added. The reaction mixture was stirred at room temperature overnight. The reaction solution was quenched with saturated sodium thiosulfate solution, and the mixture was extracted with dichloromethane (3×20 mL). The combined organic phases were washed with saturated brine, dried over anhydrous sodium sulfate, filtered, and concentrated under reduced pressure. The crude product was purified by silica gel column chromatography (ethyl acetate: petroleum ether=2:1) to give compound 63-d as a white solid (60 mg, yield 59%).

Compound 63-d (42 mg, 0.15 mmol) and compound 1-a (30 mg, 0.056 mmol) were dissolved in tetrahydrofuran (1 mL), and the solution was cooled to −60° C. under a nitrogen atmosphere. A tetrahydrofuran solution of lithium bis(trimethylsilyl)amide (0.3 mL, 1 M) was added dropwise. The reaction mixture was stirred at this temperature for 0.5 hours. The reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=25:1) to give compound 63 as a white solid (11.22 mg, yield 33%). ¹H NMR (500 MHz, DMSO-d) δ 11.26 (br., 1H), 10.05 (s, 1H), 9.02 (s, 1H), 8.83 (s, 1H), 8.59 (d, J=4.7 Hz, 1H), 8.49 (d, J=8.6 Hz, 1H), 8.25 (s, 1H), 8.19 (d, J=8.7 Hz, 1H), 7.23 (br., 1H), 5.10 (d, J=8.6 Hz, 1H), 4.47 (dd, J=13.9, 6.9 Hz, 2H), 3.82-3.71 (m, 2H), 3.70-3.63 (m, 2H), 3.25 (s, 1H), 3.19 (dd, J=17.0, 8.6 Hz, 1H), 3.09-2.99 (m, 1H), 2.45-2.35 (m, 2H), 2.30-2.15 (m, 3H), 2.09-1.96 (m, 3H), 1.57-1.49 (m, 1H), 1.39 (t, J=7.0 Hz, 3H), 1.10-1.02 (m, 2H), 0.92-0.81 (m, 2H). MS m/z 605.9 [M+H]⁺.

### Example 64: Preparation of Compound 64

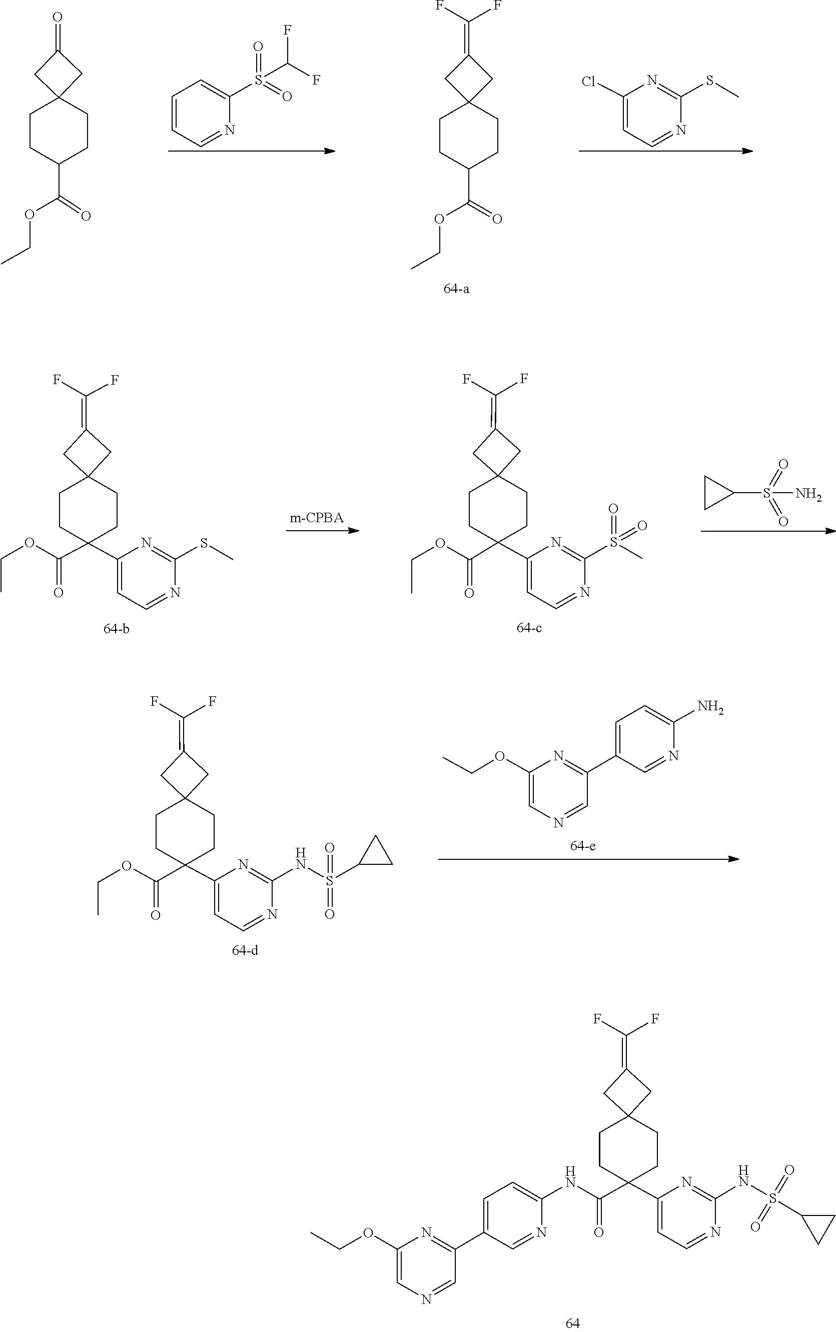

Ethyl 2-oxaspiro[3.5]nonane-7-carboxylate (680 mg, 3.24 mmol) and difluoromethyl (2-pyridyl)sulfone (625 mg, 3.24 mmol) were dissolved in DMF (6 mL). Under a nitrogen atmosphere, the solution was cooled to −60° C., and a solution of potassium tert-butoxide (727 mg, 6.48 mmol) in DMF (6 mL) was added dropwise. After the dropwise addition, the reaction system was stirred at this temperature for 0.5 hours. Dilute hydrochloric acid (10 mL, 1 M) was added dropwise, and the reaction solution was heated and stirred at 60° C. overnight. The reaction solution was diluted with water, and the mixture was extracted with ethyl acetate (3×20 mL). The combined organic phases were washed with saturated brine, dried over anhydrous sodium sulfate, filtered, and concentrated under reduced pressure. The crude product was purified by preparative thin-layer chromatography (ethyl acetate: petroleum ether=10:1) to give compound 64-a as a yellow oil (152 mg, yield 19%).

Compound 64-a (150 mg, 0.61 mmol) and 2-methylthio-4-chloropyrimidine (98 mg, 0.61 mmol) were dissolved in tetrahydrofuran (3 mL). Lithium bis(trimethylsilyl)amide (1 M, 0.8 mL) was added dropwise. The reaction mixture was stirred at room temperature for 1 hour. The reaction solution was concentrated under reduced pressure, and the crude product was purified by preparative thin-layer chromatography (ethyl acetate: petroleum ether=2:1) to give compound 64-b as a yellow solid (86 mg, yield 38%). MS m/z 369.1 [M+H]⁺.

Compound 64-b (86 mg, 0.23 mmol) was dissolved in dichloromethane (3 mL), and m-chloroperoxybenzoic acid (100 mg, 0.58 mmol) was added. The reaction mixture was stirred at room temperature overnight. The reaction solution was quenched with saturated sodium thiosulfate, and the mixture was extracted with dichloromethane (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=25:1) to give compound 64-c as a yellow solid (56 mg, yield 60%). MS m/z 401.4 [M+H]⁺.

Compound 64-c (44 mg, 0.11 mmol), cyclopropylsulfonamide (20 mg, 0.17 mmol), and cesium carbonate (107 mg, 0.33 mmol) were dissolved in acetonitrile (2 mL). The reaction mixture was heated and stirred at 60° C. for 1 hour. The reaction solution was filtered, and the filtrate was concentrated under reduced pressure. The crude product was purified by preparative thin-layer chromatography (dichloromethane:methanol=20:1) to give compound 64-d as a yellow solid (26 mg, yield 54%). MS m/z 442.1 [M+H]⁺.

Compound 64-d (12 mg, 0.03 mmol) and compound 64-e (6 mg, 0.03 mmol) were dissolved in tetrahydrofuran (1 mL). Lithium bis(trimethylsilyl)amide (1 M, 0.1 mL) was added dropwise. The reaction mixture was stirred at room temperature for 1 hour. The reaction solution was concentrated under reduced pressure, and the crude product was purified by preparative thin-layer chromatography (dichloromethane:methanol=20:1) to give compound 64 as a white solid (6 mg, yield 36%). ¹H NMR (500 MHz, DMSO-d₆) δ 11.28 (s, 1H), 9.97 (s, 1H), 9.02 (d, J=2.1 Hz, 1H), 8.83 (s, 1H), 8.59 (d, J=5.1 Hz, 1H), 8.48 (dd, J=8.8, 2.4 Hz, 1H), 8.25 (s, 1H), 8.17 (d, J=8.8 Hz, 1H), 7.23 (s, 1H), 4.47 (q, J=7.0 Hz, 2H), 3.27-3.23 (m, 1H), 2.42-2.35 (m, 6H), 2.08-1.99 (m, 2H), 1.68-1.58 (m, 4H), 1.39 (t, J=7.0 Hz, 3H), 1.07-1.03 (m, 2H), 0.90-0.84 (m, 2H). MS m/z 612.0 [M+H]⁺.

### Example 65: Preparation of Compound 65

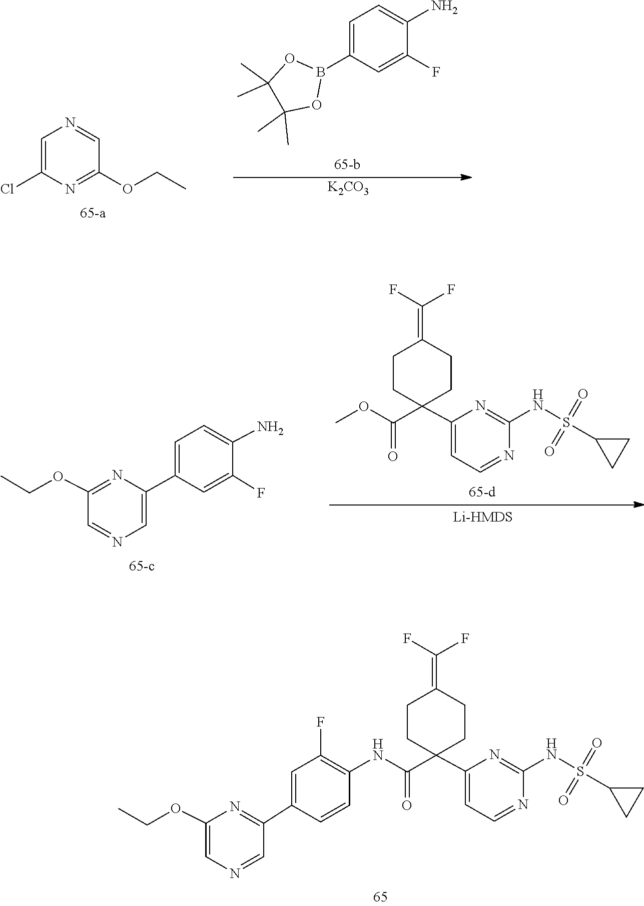

Compound 65-a (100 mg, 0.63 mmol), compound 65-b (324 mg, 1.55 mmol), bis(triphenylphosphine)palladium dichloride (46 mg, 0.063 mmol), and potassium carbonate (261 mg, 1.89 mmol) were dissolved in 1,4-dioxane and water (4:1). The reaction was heated to 90° C. for 2 hours. After completion, the reaction solution was extracted three times with ethyl acetate and water. The combined organic phases were washed with saturated brine, dried over anhydrous sodium sulfate, filtered, and the filtrate was concentrated under reduced pressure to obtain a crude product. The crude product was purified by column chromatography (dichloromethane:methanol=30:1) to obtain compound 65-c (70 mg, yield 47%).

Compound 65-c (10 mg, 0.042 mmol) and compound 65-d (16 mg, 0.042 mmol) were dissolved in tetrahydrofuran. Lithium bis(trimethylsilyl)amide (0.08 mL, 1 M) was added at room temperature, and the reaction solution was stirred for 1 hour. After completion, the reaction solution was extracted three times with ethyl acetate and water. The combined organic phases were washed with saturated brine, dried over anhydrous sodium sulfate, filtered, and the filtrate was concentrated under reduced pressure to obtain a crude product. The crude product was purified by thin-layer chromatography silica gel plate (dichloromethane:methanol=20:1) to obtain compound 65 (3.67 mg, yield 14%). ¹H NMR (500 MHz, DMSO-d₆) δ 11.33 (br., 1H), 9.49 (s, 1H), 8.84 (s, 1H), 8.61 (br., 1H), 8.25 (s, 1H), 8.01-7.94 (m, 2H), 7.61 (t, J=8.1 Hz, 1H), 7.21 (br., 1H), 4.48 (q, J=7.0 Hz, 2H), 3.27 (br., 1H), 2.35-2.03 (m, 8H), 1.40 (t, J=7.0 Hz, 3H), 1.13-1.07 (m, 2H), 1.03-0.97 (m, 2H). MS m/z 588.9 [M+H]⁺.

### Example 66: Preparation of Compound 66

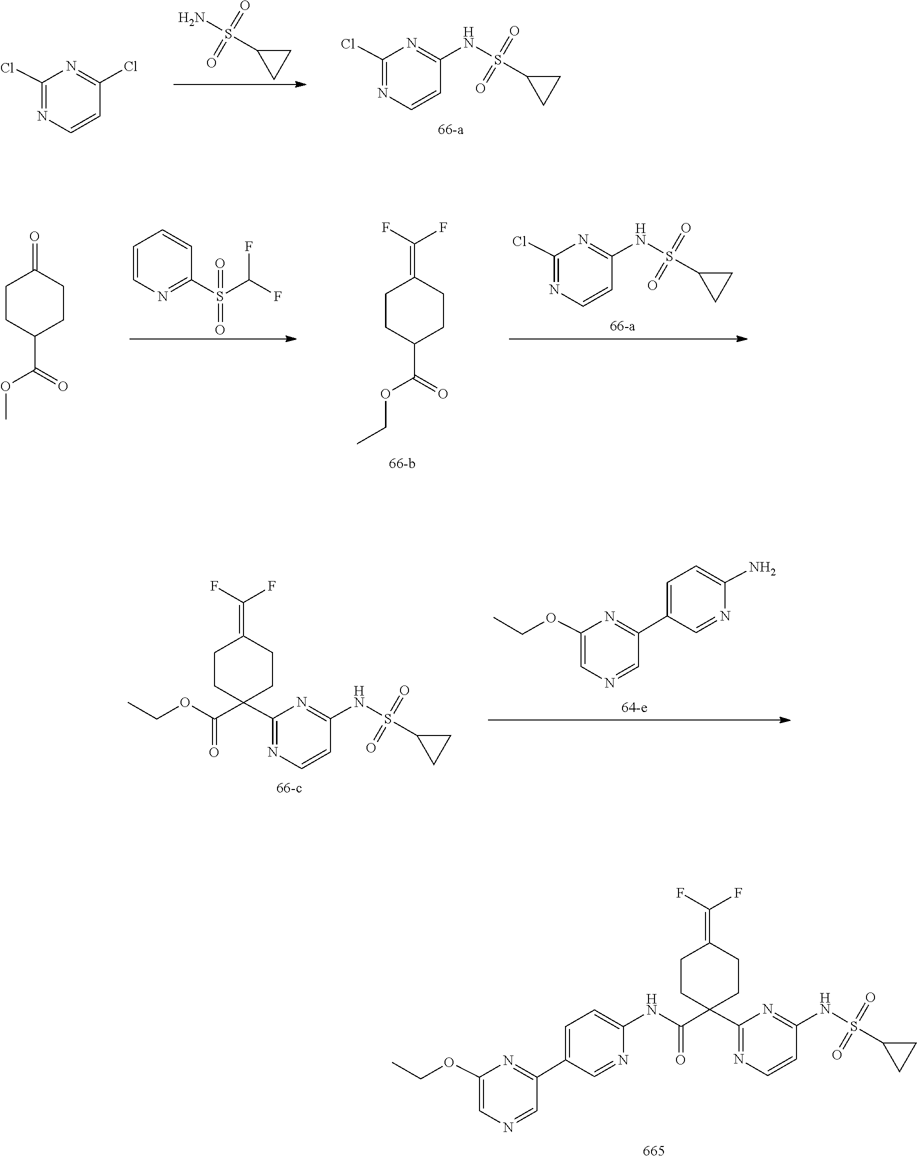

2,4-Dichloropyrimidine (2.0 g, 13.51 mmol), cyclopropylsulfonamide (1.8 g, 14.86 mmol), and potassium carbonate (4.7 g, 33.78 mmol) were dissolved in acetonitrile (30 mL). The reaction mixture was heated and stirred at 60° C. for 1 hour. The reaction mixture was filtered, and the filtrate was concentrated under reduced pressure. The crude product was purified by silica gel column chromatography to obtain oily compound 66-a (1.1 g, yield 35%). MS m/z 234.6 [M+H]⁺.

Ethyl 4-cyclohexanonecarboxylate (1.0 g, 5.88 mmol) and difluoromethyl (2-pyridyl)sulfone (1.1 g, 5.88 mmol) were dissolved in DMF (15 mL). Under a nitrogen atmosphere, the solution was cooled to −60° C., and a solution of potassium tert-butoxide (1.3 g, 11.76 mmol) in DMF (10 mL) was added dropwise. After the dropwise addition, the reaction solution was stirred continuously at this temperature for 0.5 hours. Dilute hydrochloric acid (20 mL, 1 M) was added dropwise, and the reaction solution was stirred at room temperature for 0.5 hours. It was diluted with water, and the mixture was extracted with ethyl acetate (3×30 mL). The combined organic phases were washed with saturated brine, dried over anhydrous sodium sulfate, filtered, and concentrated under reduced pressure. The crude product was purified by preparative thin-layer chromatography (ethyl acetate: petroleum ether=10:1) to give compound 66-b as an oil (588 mg, yield 49%).

Compound 66-b (93 mg, 0.46 mmol) was dissolved in tetrahydrofuran (3 mL). The reaction solution was cooled to −60° C. under a nitrogen atmosphere, and lithium diisopropylamide (2.0 M, 0.25 mL) was added dropwise. The reaction solution was stirred at this temperature for 0.5 hours, and then a tetrahydrofuran (2 mL) solution of compound 66-a (107 mg, 0.46 mmol) was added dropwise. After the dropwise addition, the reaction solution was warmed to room temperature and stirred for 2 hours. The reaction solution was quenched with saturated aqueous ammonium chloride solution, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (ethyl acetate: petroleum ether=1:1) to give compound 66-c as a yellow solid (15 mg, yield 8%). MS m/z 402.1 [M+H]⁺.

Compound 66-c (15 mg, 0.04 mmol) and compound 64-e (8 mg, 0.04 mmol) were dissolved in tetrahydrofuran (1 mL). Lithium bis(trimethylsilyl)amide (1 M, 0.1 mL) was added dropwise. The reaction mixture was stirred at room temperature for 1 hour. The reaction solution was concentrated under reduced pressure, and the crude product was purified by preparative thin-layer chromatography (dichloromethane:methanol=20:1) to give compound 66 as a white solid (4 mg, yield 19%). ¹H NMR (500 MHz, DMSO-d₆) δ 11.29 (br., 1H), 10.15 (s, 1H), 9.02 (d, J=2.0 Hz, 1H), 8.84 (s, 1H), 8.60 (d, J=5.2 Hz, 1H), 8.49 (dd, J=8.8, 2.4 Hz, 1H), 8.25 (s, 1H), 8.19 (d, J=8.8 Hz, 1H), 7.22 (d, J=4.1 Hz, 1H), 4.47 (q, J=7.0 Hz, 2H), 3.26-3.21 (m, 1H), 2.57-2.52 (m, 2H), 2.34-2.27 (m, 2H), 2.23-2.15 (m, 2H), 2.09-2.02 (m, 2H), 1.39 (t, J=7.0 Hz, 3H), 1.06-1.00 (m, 2H), 0.88-0.83 (m, 2H). MS m/z 571.9 [M+H]⁺.

### Example 67: Preparation of Compound 67

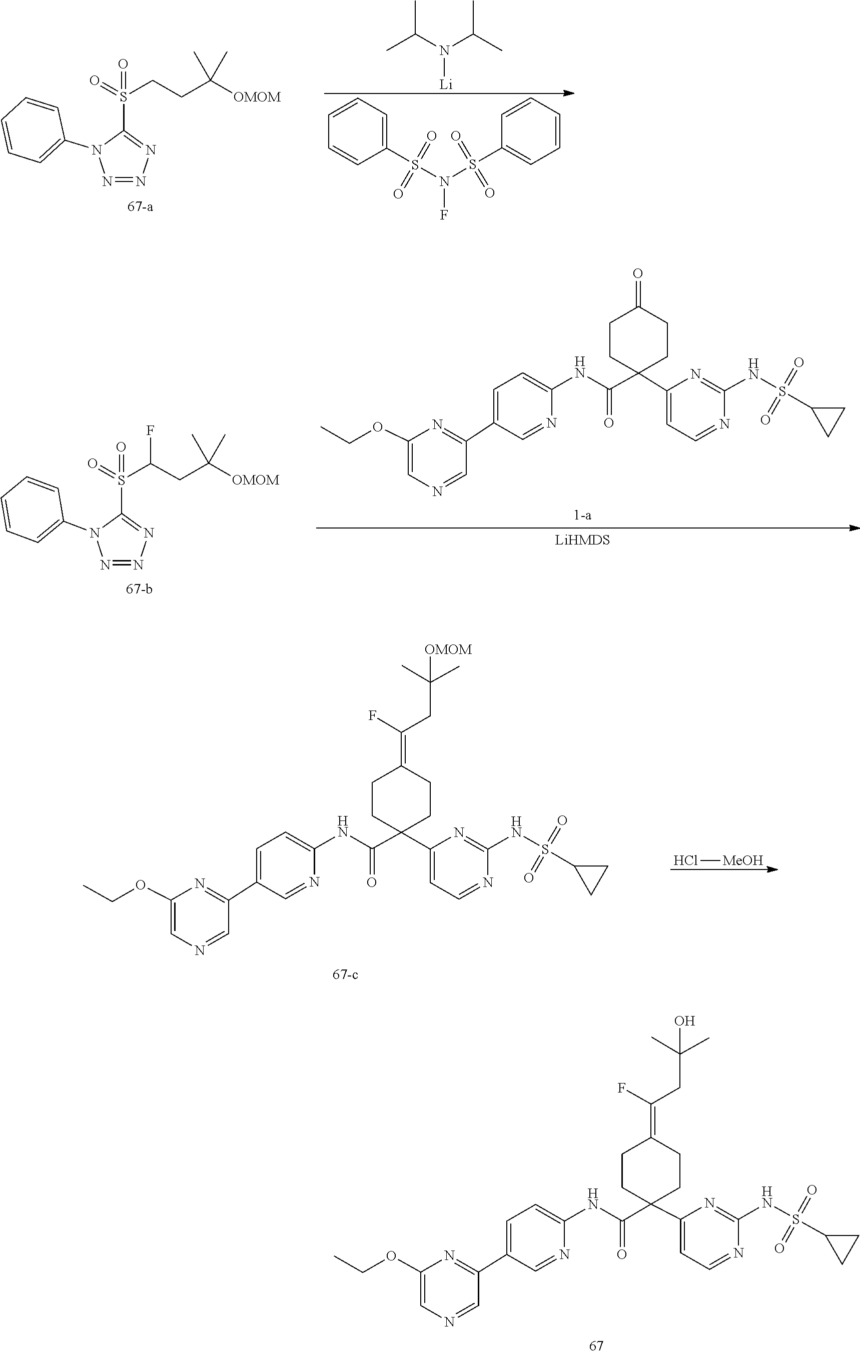

Compound 67-a (120 mg, 0.35 mmol) was dissolved in tetrahydrofuran (3 mL). Under a nitrogen atmosphere at −78° C., lithium diisopropylamide (0.2 mL, 2 M) was added dropwise. After stirring for another half an hour under this atmosphere, a tetrahydrofuran solution of N-fluorobenzenesulfonimide (222 mg, 0.70 mmol) was added dropwise. The reaction solution was slowly warmed to room temperature and stirred for 5 hours. After completion, water was added to the reaction solution, and it was extracted with ethyl acetate (3×15 mL). The combined organic phases were washed with saturated brine, dried over anhydrous sodium sulfate, filtered, and concentrated under reduced pressure. The crude product was purified by silica gel column chromatography (ethyl acetate: petroleum ether=3:1) to give compound 67-b as a white solid (85 mg, yield 67%).

Compound 67-b (100 mg, 0.28 mmol) and compound 1-a (50 mg, 0.09 mmol) were dissolved in tetrahydrofuran (1 mL), and the solution was cooled to −60° C. under a nitrogen atmosphere. A tetrahydrofuran solution of lithium bis(trimethylsilyl)amide (0.3 mL, 1 M) was added dropwise. The reaction mixture was stirred at this temperature for 0.5 hours. The reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=30:1) to give compound 67-c as a white solid (10 mg, yield 16%).

Compound 67-c (10 mg, 0.014 mmol) was dissolved in methanol (1 mL), and a methanolic hydrochloric acid solution (2.05 mg, 4 M) was added dropwise under an ice bath. The reaction mixture was stirred at room temperature for 0.5 hours. The reaction solution was adjusted to pH 7-9 with saturated sodium bicarbonate solution, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=20:1) to give compound 67 as a white solid (3.2 mg, yield 34%). ¹H NMR (500 MHz, DMSO-d₆) δ 11.27 (br., 1H), 10.07 (s, 1H), 9.02 (d, J=2.2 Hz, 1H), 8.84 (s, 1H), 8.60 (d, J=5.2 Hz, 1H), 8.49 (dd, J=8.8, 2.4 Hz, 1H), 8.25 (s, 1H), 8.19 (d, J=8.8 Hz, 1H), 7.22 (d, J=5.1 Hz, 1H), 4.47 (q, J=7.0 Hz, 2H), 4.39 (s, 1H), 3.25-3.23 (m, 1H), 2.40-2.24 (m, 4H), 2.23-1.99 (m, 6H), 1.39 (t, J=7.0 Hz, 3H), 1.11 (s, 6H), 1.06-1.02 (m, 2H), 0.88-0.85 (m, 2H). MS m/z 626.0 [M+H]⁺.

### Example 68: Preparation of Compound 68

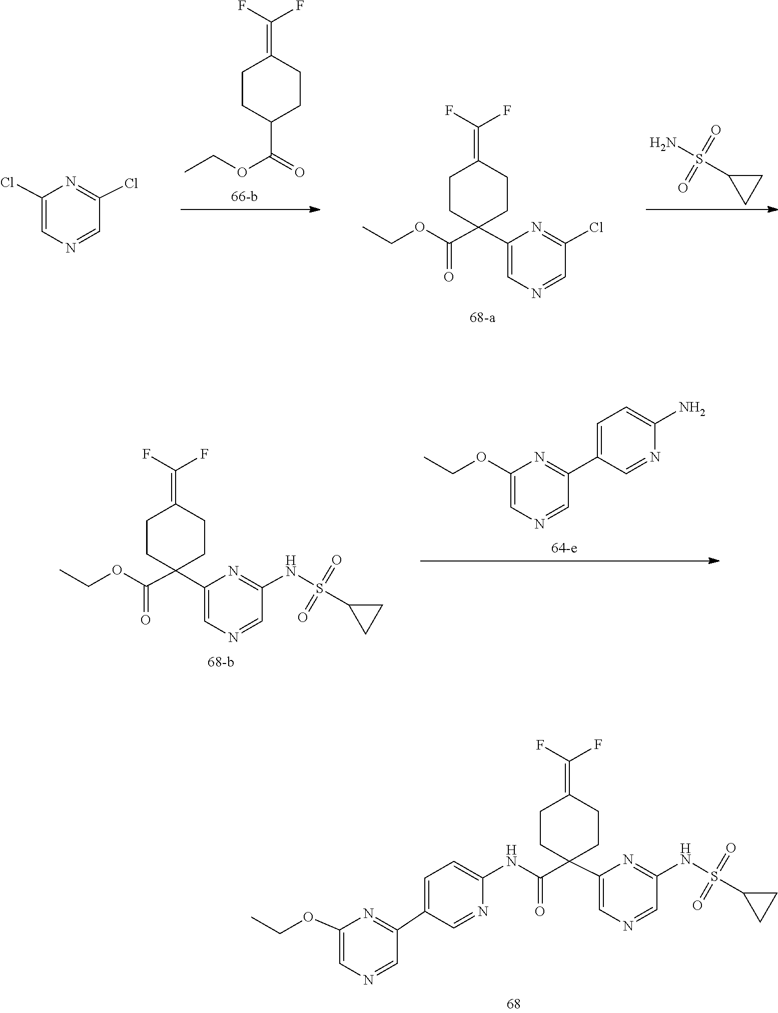

2,6-Dichloropyrazine (700 mg, 4.73 mmol) and compound 66-b (965 mg, 4.73 mmol) were dissolved in tetrahydrofuran (10 mL). Lithium bis(trimethylsilyl)amide (1 M, 6.1 mL) was added dropwise. The reaction mixture was stirred at room temperature for 1 hour. The reaction solution was concentrated under reduced pressure, and the crude product was purified by preparative thin-layer chromatography (ethyl acetate: petroleum ether=2:1) to give compound 68-a as a yellow solid (628 mg, yield 34%). MS m/z 317.7 [M+H]⁺.

Compound 68-a (100 mg, 0.32 mmol), cyclopropylsulfonamide (58 mg, 0.48 mmol), 4,5-bis(diphenylphosphino)-9,9-dimethylxanthene, tris (dibenzyl ideneacetone)dipalladium (27 mg, 0.03 mmol), and cesium carbonate (313 mg, 0.96 mmol) were dissolved in dioxane (3 mL). The reaction mixture was heated and stirred at 100° C. for 1 hour under a nitrogen atmosphere. The reaction solution was filtered through Celite, and the filtrate was concentrated under reduced pressure. The crude product was purified by preparative thin-layer chromatography (ethyl acetate: petroleum ether=2:1) to give compound 68-b as a yellow solid (39 mg, yield 31%). MS m/z 402.0 [M+H]⁺.

Compound 68-b (20 mg, 0.05 mmol) and compound 64-e (11 mg, 0.05 mmol) were dissolved in tetrahydrofuran (2 mL). Lithium bis(trimethylsilyl)amide (1 M, 0.2 mL) was added dropwise. The reaction mixture was stirred at room temperature for 1 hour. The reaction solution was concentrated under reduced pressure, and the crude product was purified by preparative thin-layer chromatography (dichloromethane:methanol=20:1) to give compound 68 as a white solid (8 mg, yield 28%). ¹H NMR (500 MHz, DMSO-d₆) δ 11.05 (s, 1H), 10.16 (s, 1H), 9.00 (d, J=2.1 Hz, 1H), 8.83 (s, 1H), 8.49 (dd, J=8.8, 2.1 Hz, 1H), 8.41 (s, 1H), 8.25 (s, 1H), 8.22-8.12 (m, 2H), 4.47 (q, J=7.0 Hz, 2H), 3.11-3.04 (m, 1H), 2.64-2.58 (m, 2H), 2.38-2.29 (m, 2H), 2.25-2.17 (m, 2H), 2.12-2.04 (m, 2H), 1.39 (t, J=7.0 Hz, 3H), 1.05-0.98 (m, 2H), 0.84-0.75 (m, 2H). MS m/z 571.9 [M+H]⁺.

### Example 69: Preparation of Compound 69

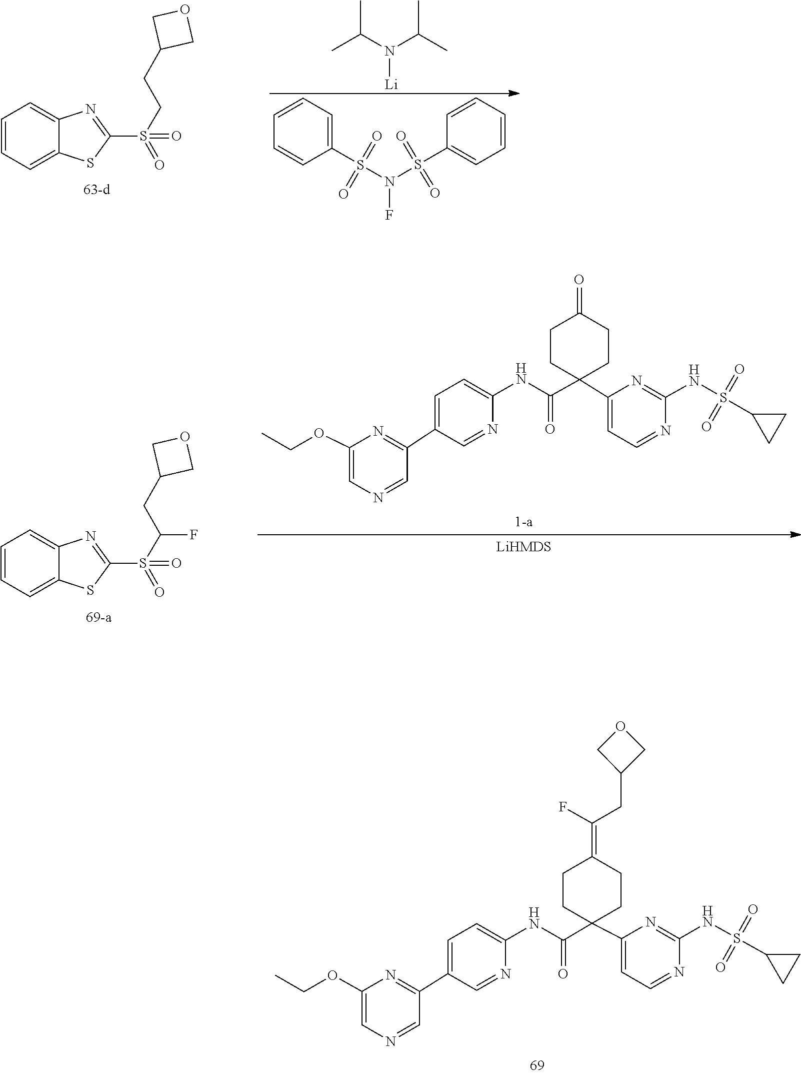

Compound 63-d (85 mg, 0.30 mmol) was dissolved in tetrahydrofuran (2 mL). Under a nitrogen atmosphere at −78° C., lithium diisopropylamide (0.2 mL, 2 M) was added dropwise. After stirring for another half an hour under this atmosphere, a tetrahydrofuran solution of N-fluorobenzenesulfonimide (208 mg, 0.66 mmol) was added dropwise. The reaction solution was slowly warmed to room temperature and stirred for 5 hours. After completion, water was added to the reaction solution, and it was extracted with ethyl acetate (3×15 mL). The combined organic phases were washed with saturated brine, dried over anhydrous sodium sulfate, filtered, and concentrated under reduced pressure. The crude product was purified by silica gel column chromatography (ethyl acetate:petroleum ether=3:1) to give compound 69-a as a white solid (55 mg, yield 61%).

Compound 69-a (22 mg, 0.074 mmol) and compound 1-a (20 mg, 0.037 mmol) were dissolved in tetrahydrofuran (1 mL), and the solution was cooled to −60° C. under a nitrogen atmosphere. A tetrahydrofuran solution of lithium bis(trimethylsilyl)amide (0.1 mL, 1 M) was added dropwise. The reaction mixture was stirred at this temperature for 0.5 hours. The reaction was quenched with water, and the mixture was extracted with ethyl acetate (3×10 mL). The combined organic phases were washed with saturated brine, dried over anhydrous sodium sulfate, filtered, and concentrated under reduced pressure. The crude product was purified by preparative thin-layer chromatography (dichloromethane:methanol=30:1) to give compound 69 as a white solid (4 mg, yield 17%). ¹H NMR (500 MHz, DMSO-d₆) δ 11.29 (br., 1H), 10.10 (s, 1H), 9.02 (d, J=2.1 Hz, 1H), 8.84 (s, 1H), 8.58 (d, J=4.5 Hz, 1H), 8.49 (dd, J=8.8, 2.4 Hz, 1H), 8.25 (s, 1H), 8.18 (d, J=8.8 Hz, 1H), 7.20 (br., 1H), 4.67-4.59 (m, 2H), 4.47 (q, J=7.0 Hz, 2H), 4.32-4.25 (m, 2H), 3.26-3.21 (m, 1H), 3.17-3.08 (m, 1H), 2.65 (dd, J=24.1, 7.5 Hz, 2H), 2.49-2.43 (m, 2H), 2.39-2.26 (m, 2H), 2.25-2.10 (m, 2H), 2.08-1.95 (m, 2H), 1.39 (t, J=7.0 Hz, 3H), 1.07-1.00 (m, 2H), 0.88-0.80 (m, 2H). MS m/z 624.0 [M+H]⁺.

### Example 70: Preparation of Compound 70

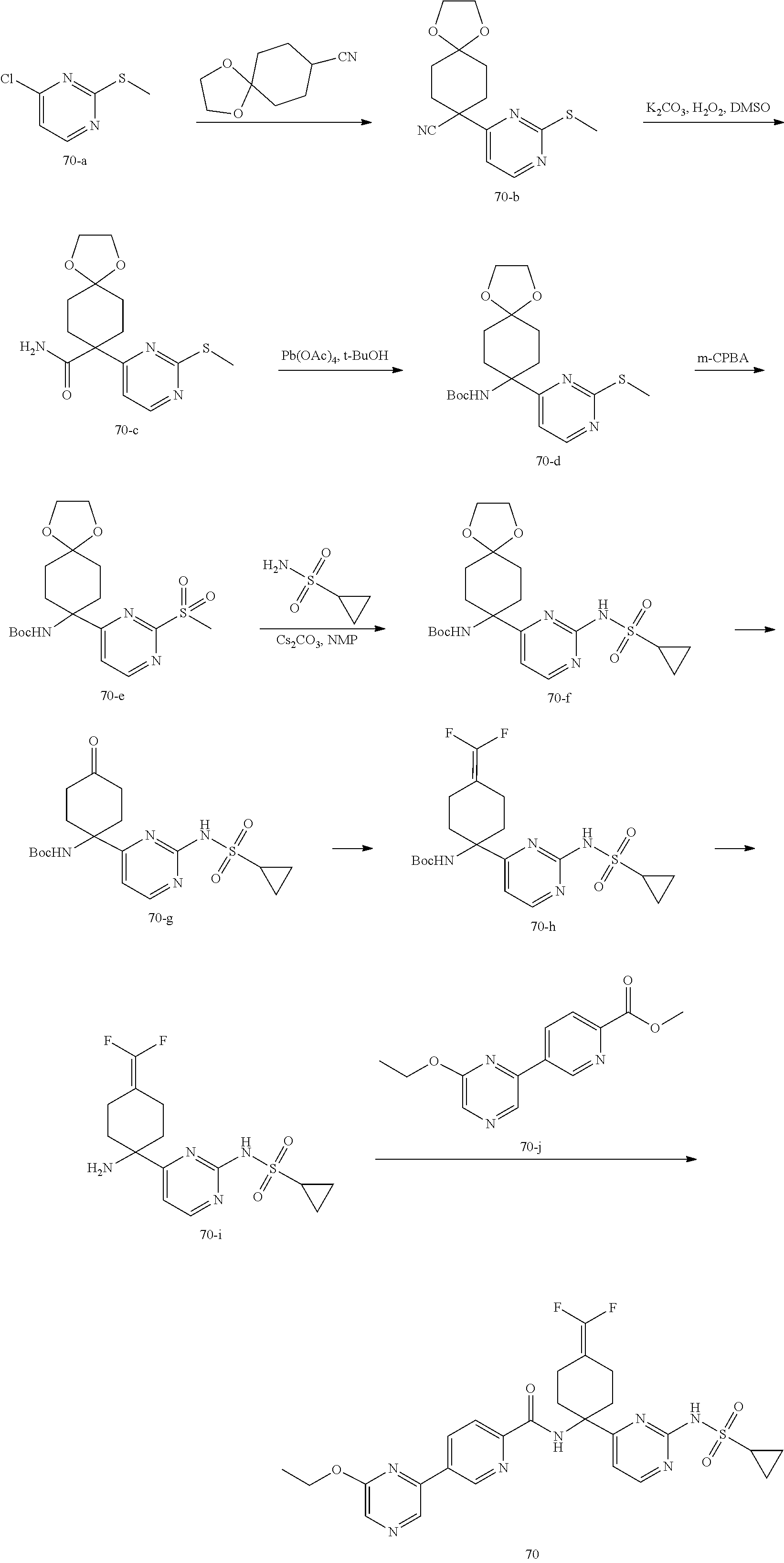

Compound 70-a (3.8 g, 23.66 mmol) and 1,4-dioxaspiro[4.5]nonane-8-carbonitrile (3.96 g, 23.66 mmol) were dissolved in tetrahydrofuran (50 mL), followed by the addition of a tetrahydrofuran solution of lithium bis(trimethylsilyl)amide (26 mL, 1 M). The mixture was stirred at room temperature for 2 hours. After completion, the reaction solution was adjusted to pH 5-6 with dilute hydrochloric acid, and then extracted with ethyl acetate three times. The combined organic phases were washed with saturated brine, dried over anhydrous sodium sulfate, filtered, and concentrated under reduced pressure. The crude product was purified by silica gel column chromatography (ethyl acetate:petroleum ether=5:1) to obtain compound 70-b (2.4 g, yield 35%).

Compound 70-b (2.8 g, 9.61 mmol) was dissolved in dimethyl sulfoxide (30 mL), and potassium carbonate (3.98 g, 28.83 mmol) was added. A hydrogen peroxide solution (5.45 g, 30%) was added under an ice bath, and stirring was continued at room temperature for 12 hours. After completion, water was added to the reaction solution, and it was extracted with ethyl acetate three times. The combined organic phases were washed with saturated brine, dried over anhydrous sodium sulfate, filtered, and the filtrate was concentrated under reduced pressure to obtain a crude product, which was directly used in the next step. The resulting crude product was purified by silica gel column chromatography to obtain compound 70-c (1.88 g, yield 63%).

Compound 70-c (1.88 g, 6.08 mmol) was dissolved in tert-butanol (20 mL), and lead acetate (3.5 g, 7.90 mmol) was added. The reaction was carried out at room temperature overnight. After completion, the reaction solution was filtered, and the filtrate was concentrated under reduced pressure. The crude product was purified by silica gel column chromatography to obtain compound 70-d (1.14 g, yield 49%).

Compound 70-d (1.1 g, 2.88 mmol) was dissolved in dichloromethane (15 mL), and m-chloroperoxybenzoic acid (1.29 g, 6.34 mmol) was added. The reaction was carried out at room temperature overnight. After completion, the reaction solution was washed with 10% sodium thiosulfate solution, extracted with dichloromethane twice. The combined organic phases were washed with 10% sodium bicarbonate solution twice, and the organic phase was concentrated under reduced pressure to obtain a crude product, which was purified by silica gel column chromatography to obtain compound 70-e (1.03 g, yield 86%).

Compound 70-e (1.04 g, 2.52 mmol) and cyclopropanesulfonamide (2.46 g, 7.55 mmol) were dissolved in anhydrous N-methylpyrrolidone (10 mL), and cesium carbonate (2.46 g, 7.55 mmol) was added. The reaction was carried out at 60° C. for 2 hours under nitrogen protection. After completion, the reaction solution was adjusted to pH 5-6 with hydrochloric acid solution, then extracted with ethyl acetate. The combined organic phases were washed with saturated brine, dried over anhydrous sodium sulfate to obtain a crude product, which was purified by silica gel column chromatography to obtain compound 70-f (0.23 g, yield 23%).

Compound 70-f (200 mg, 0.44 mmol) was dissolved in a mixed solution of tetrahydrofuran and water (4:1), and p-toluenesulfonic acid (151 mg, 0.88 mmol) was added. The reaction was carried out at room temperature overnight. After completion, extraction was performed with ethyl acetate three times. The combined organic phases were washed with saturated brine, dried over anhydrous sodium sulfate, filtered, and the filtrate was concentrated under reduced pressure to obtain a crude product, which was purified by silica gel column chromatography to obtain compound 70-g (0.12 g, yield 66%).

Potassium tert-butoxide (71 mg, 0.633 mmol) was dissolved in N,N-dimethylformamide (1 mL). A mixed solution of compound 70-g (65 mg, 0.158 mmol) and difluoromethyl (2-pyridyl)sulfone (36 mg, 0.19 mmol) was added at −78° C. under nitrogen atmosphere, and stirring was continued at this temperature for half an hour. After completion, the reaction solution was adjusted to pH 5-6 with dilute hydrochloric acid (1 M), then extracted with ethyl acetate three times. The combined organic phases were washed with saturated brine, dried over anhydrous sodium sulfate, filtered, and the filtrate was concentrated under reduced pressure to obtain a crude product, which was purified by thin-layer chromatography silica gel plate (dichloromethane:methanol=30:1) to obtain compound 70-h (49 mg, yield 70%).

Compound 70-h (49 mg, 0.11 mmol) was dissolved in dichloromethane (2 mL), and a dioxane solution of hydrochloric acid (4 M) was added at room temperature. The reaction was stirred at room temperature for 1 hour. After completion, the reaction solution was adjusted to pH 7-9 with saturated sodium bicarbonate solution, then extracted with dichloromethane three times. The combined organic phases were washed with saturated brine, dried over anhydrous sodium sulfate, filtered, and the filtrate was concentrated under reduced pressure to obtain a crude product, which was purified by thin-layer chromatography silica gel plate (dichloromethane:methanol=20:1) to obtain compound 70-i (25 mg, yield 66%).

Compound 70-i (10 mg, 0.03 mmol) and compound 70-j (9 mg, 0.03 mmol) were dissolved in tetrahydrofuran (1 mL). A solution of lithium bis(trimethylsilyl)amide (1 M, 0.1 mL) was added dropwise at room temperature, and the reaction was carried out at room temperature for half an hour. After completion, the reaction solution was adjusted to pH 5-6 with dilute hydrochloric acid (2 M), then extracted with ethyl acetate three times. The combined organic phases were washed with saturated brine, dried over anhydrous sodium sulfate, filtered, and the filtrate was concentrated under reduced pressure to obtain a crude product, which was purified by thin-layer chromatography silica gel plate (dichloromethane:methanol=30:1) to obtain compound 70 (6 mg, yield 36%). ¹H NMR (500 MHz, DMSO-d₆) δ 11.19 (br., 1H), 9.42 (d, J=1.6 Hz, 1H), 8.86 (s, 1H), 8.86 (s, 1H), 8.68 (dd, J=8.2, 2.2 Hz, 1H), 8.52 (d, J=5.2 Hz, 1H), 8.38 (s, 1H), 8.08 (d, J=8.2 Hz, 1H), 7.17 (d, J=4.2 Hz, 1H), 4.52 (q, J=7.0 Hz, 2H), 3.20 (br., J=12.7, 8.1, 4.8 Hz, 1H), 2.60-2.53 (m, 2H), 2.44-2.38 (m, 2H), 2.21-2.11 (m, 2H), 2.05-1.97 (m, 2H), 1.42 (t, J=7.0 Hz, 3H), 1.07-1.02 (m, 2H), 0.92-0.86 (m, 2H). MS m/z 572.0 [M+H]⁺.

### Example 71: Preparation of Compound 71

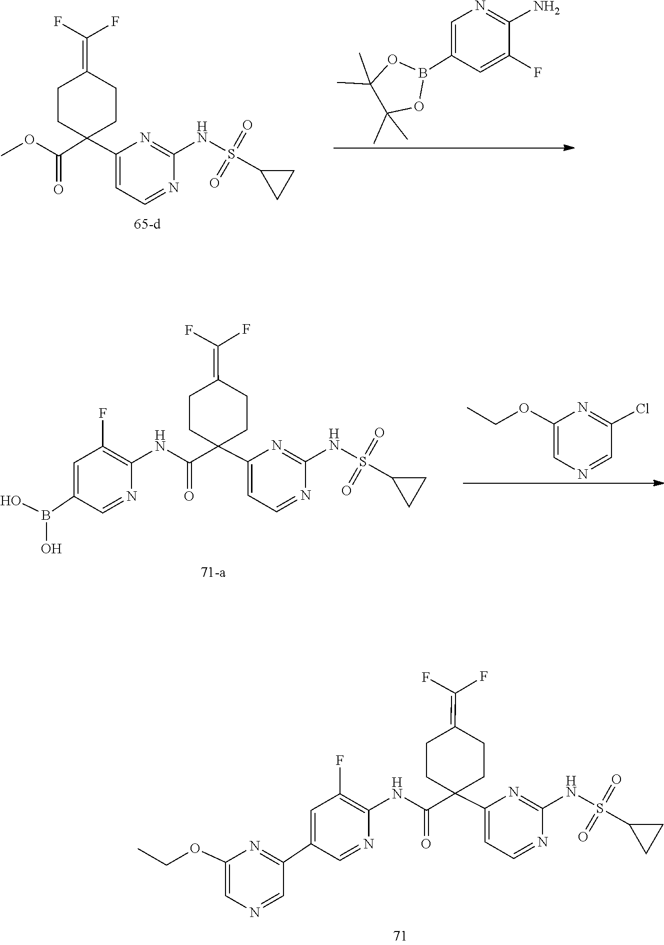

Compound 65-d (161 mg, 0.42 mmol) and 3-fluoro-2-aminopyridine-5-boronic acid pinacol ester (148 mg, 0.62 mmol) were dissolved in tetrahydrofuran (3 mL). Lithium bis(trimethylsilyl)amide (1 M, 1.3 mL) was added dropwise. The reaction mixture was stirred at room temperature for 1 hour. The reaction solution was concentrated under reduced pressure, and the crude product was purified by preparative thin-layer chromatography (dichloromethane:methanol=20:1) to obtain compound 71-a as a yellow solid (44 mg, yield 21%). MS m/z 512.1 [M+H]⁺.

Compound 71-a (30 mg, 0.06 mmol), 2-chloro-6-ethoxypyrazine (12 mg, 0.08 mmol), [1,1′-bis(diphenylphosphino)ferrocene]dichloropalladium (7 mg, 0.01 mmol), and potassium carbonate (25 mg, 0.18 mmol) were dissolved in a mixed solution of dioxane/water (2 mL/0.5 mL). The reaction mixture was heated and stirred at 100° C. for 1 hour under nitrogen atmosphere. The reaction mixture was filtered through Celite, and the filtrate was concentrated under reduced pressure. The crude product was purified by preparative thin-layer chromatography (dichloromethane:methanol=20:1) to obtain compound 71 as a white solid (12 mg, yield 34%). ¹H NMR (500 MHz, DMSO-d₆) δ 11.33 (br., 1H), 10.24 (s, 1H), 8.99 (d, J=1.3 Hz, 1H), 8.92 (s, 1H), 8.63 (d, J=5.2 Hz, 1H), 8.44 (dd, J=10.9, 1.8 Hz, 1H), 8.32 (s, 1H), 7.17 (d, J=4.8 Hz, 1H), 4.49 (q, J=7.0 Hz, 2H), 3.30-3.28 (m, 1H), 2.51 (s, 2H), 2.39-2.30 (m, 2H), 2.26-2.18 (m, 2H), 2.08-2.02 (m, 2H), 1.40 (t, J=7.0 Hz, 3H), 1.11-1.07 (m, 2H), 1.03-0.97 (m, 2H). MS m/z 590.0 [M+H]⁺.

### Example 72: Preparation of Compound 72

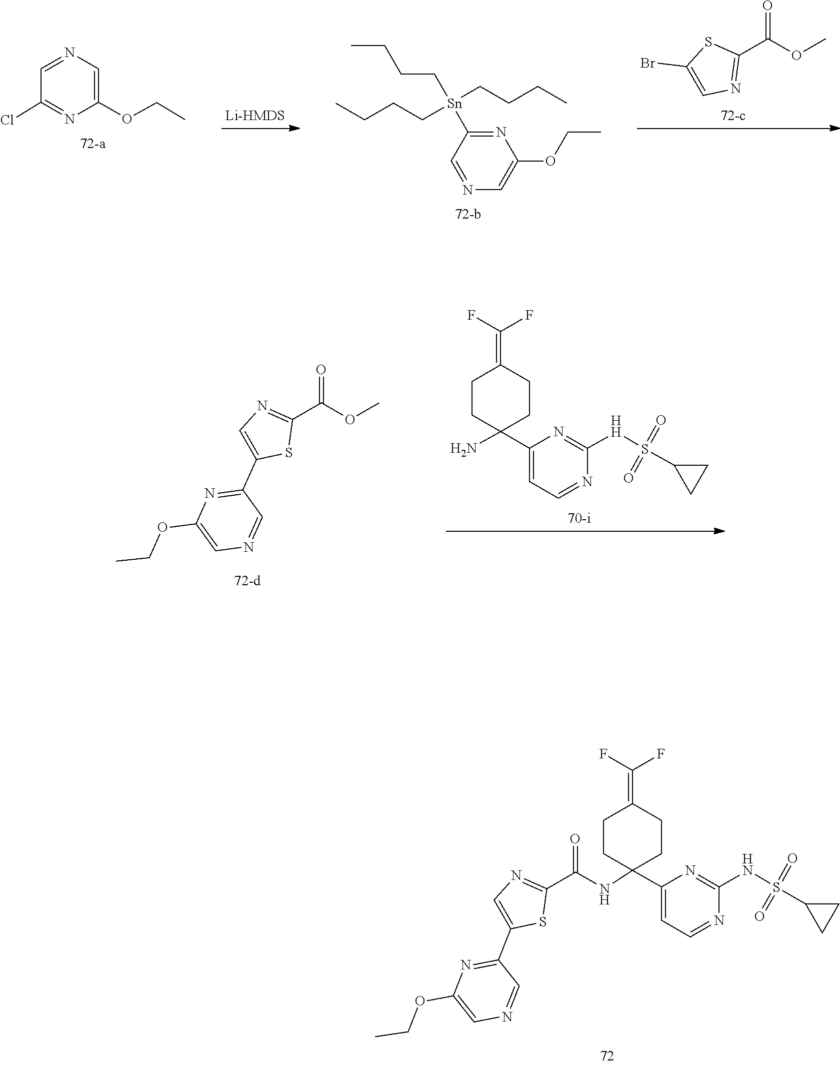

Compound 72-a (100 mg, 0.63 mmol) was dissolved in tetrahydrofuran (15 mL). LiHMDS (1 M, 0.76 mmol) was added at −78° C. under nitrogen protection, and stirring was continued at this temperature for 30 minutes. Then hexabutylditin (548 mg, 0.95 mmol) was added, and the reaction was carried out at room temperature overnight. After completion, water was added to the reaction solution, and extraction was performed with ethyl acetate. The combined organic phases were washed with saturated brine, dried over anhydrous sodium sulfate, filtered, and the filtrate was concentrated under reduced pressure to obtain a crude product, which was purified by silica gel column chromatography (petroleum ether:ethyl acetate=10:1) to obtain compound 72-b (132 mg, yield 51%).

Compound 72-b (60 mg, 0.27 mmol), compound 72-c (134 mg, 0.32 mmol), and tetrakistriphenylphosphine palladium (31 mg, 0.027 mmol) were dissolved in toluene (1 mL) and placed in a sealed tube. The reaction was heated to 100° C. for 16 hours under nitrogen protection. After completion, water was added to the reaction solution, and extraction was performed with ethyl acetate. The combined organic phases were washed with saturated brine, dried over anhydrous sodium sulfate, filtered, and the filtrate was concentrated under reduced pressure to obtain a crude product, which was purified by silica gel column chromatography (petroleum ether:ethyl acetate=3:1) to obtain compound 72-d (50 mg, yield 70%). MS m/z 266.0 [M+H]⁺.

Compound 72-d (8 mg, 0.03 mmol) and compound 70-i (10 mg, 0.03 mmol) were dissolved in tetrahydrofuran (2 mL). Lithium bis(trimethylsilyl)amide (1 M, 0.1 mL) was added dropwise. The reaction mixture was stirred at room temperature for 1 hour. The reaction solution was concentrated under reduced pressure, and the crude product was purified by preparative thin-layer chromatography (dichloromethane:methanol=20:1) to obtain compound 72 as a white solid (5 mg, yield 29%). ¹H NMR (500 MHz, DMSO-d₆) δ 11.20 (s, 1H), 8.97 (s, 1H), 8.94 (s, 1H), 8.81 (s, 1H), 8.53 (d, J=5.2 Hz, 1H), 8.29 (s, 1H), 7.16 (d, J=5.2 Hz, 1H), 4.41 (q, J=7.0 Hz, 2H), 3.27-3.23 (m, 1H), 2.59-2.53 (m, 2H), 2.40-2.35 (m, 2H), 2.22-2.15 (m, 2H), 2.01-1.95 (m, 2H), 1.38 (t, J=7.0 Hz, 3H), 1.09-1.05 (m, 2H), 0.98-0.93 (m, 2H). MS m/z 577.9 [M+H]⁺.

### Example 73: Preparation of Compound 73

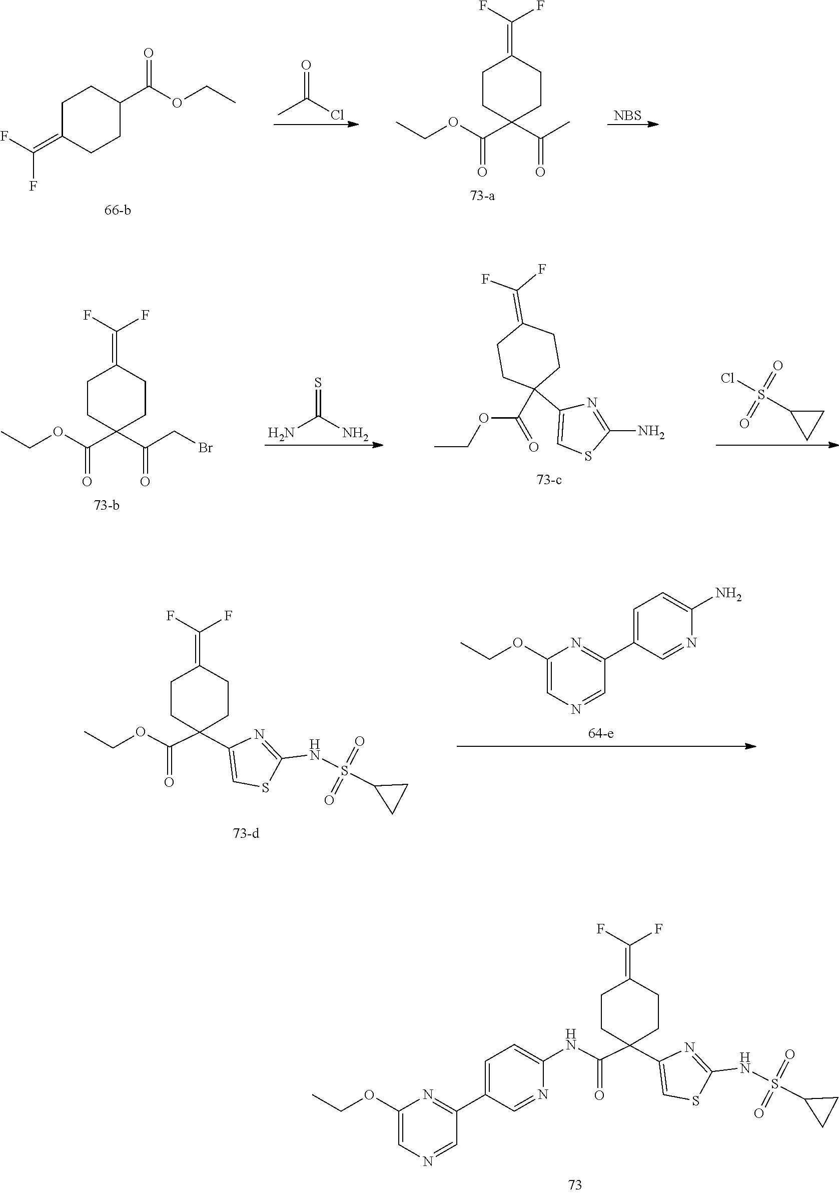

Compound 66-b (1.1 g, 5.39 mmol) was dissolved in tetrahydrofuran (20 mL). The reaction solution was cooled to −60° C. under nitrogen atmosphere, and lithium diisopropylamide (2.0 M, 3.5 mL) was added dropwise. The reaction solution was stirred at this temperature for 0.5 hours, and then acetyl chloride (841 mg, 10.78 mmol) was added dropwise. After the dropwise addition, the reaction solution was stirred at this temperature for 1 hour. The reaction solution was quenched with saturated aqueous ammonium chloride solution, and the mixture was extracted with ethyl acetate (3×30 mL). The combined organic phases were washed with saturated brine, dried over anhydrous sodium sulfate, filtered, and concentrated under reduced pressure. The crude product was purified by preparative thin-layer chromatography (ethyl acetate:petroleum ether=10:1) to obtain compound 73-a as an oil (1.0 g, yield 77%).

Compound 73-a (1.0 g, 4.06 mmol) was dissolved in acetonitrile (10 mL), and N-bromosuccinimide (801 mg, 4.5 mmol) was added. The reaction mixture was heated and stirred at 50° C. for 5 hours. The reaction solution was diluted with water, and the mixture was extracted with ethyl acetate (3×30 mL). The combined organic phases were washed with saturated brine, dried over anhydrous sodium sulfate, filtered, and concentrated under reduced pressure to obtain crude compound 73-b (1.4 g), which was directly used in the next step.

The crude compound 73-b (1.4 g) and thiourea (309 mg, 4.06 mmol) were dissolved in ethanol (20 mL). The reaction mixture was heated and stirred at 40° C. overnight. The reaction solution was concentrated under reduced pressure, then diluted with water, and extracted with ethyl acetate (3×40 mL). The combined organic phases were washed with saturated brine, dried over anhydrous sodium sulfate, filtered, and concentrated under reduced pressure. The crude product was purified by preparative thin-layer chromatography (ethyl acetate:petroleum ether=2:1) to obtain compound 73-c as a yellow solid (53 mg, yield 4%). MS m/z 303.1 [M+H]⁺.

Compound 73-c (50 mg, 0.17 mmol) was dissolved in pyridine (2 mL), and cyclopropylsulfonyl chloride (36 mg, 0.26 mmol) was added dropwise. The reaction mixture was heated and stirred in a sealed tube at 40° C. for 48 hours. The reaction solution was concentrated under reduced pressure, and the crude product was purified by preparative thin-layer chromatography (dichloromethane:methanol=20:1) to obtain compound 73-d as a yellow solid (24 mg, yield 36%). MS m/z 407.1 [M+H]⁺.

Compound 73-d (15 mg, 0.04 mmol) and compound 64-e (9 mg, 0.04 mmol) were dissolved in tetrahydrofuran (1 mL). Lithium bis(trimethylsilyl)amide (1 M, 0.1 mL) was added dropwise. The reaction mixture was stirred at room temperature for 1 hour. The reaction solution was concentrated under reduced pressure, and the crude product was purified by preparative thin-layer chromatography (dichloromethane:methanol=20:1) to obtain compound 73 as a white solid (7 mg, yield 33%). ¹H NMR (500 MHz, DMSO-d₆) δ 12.52 (s, 1H), 10.07 (br., 1H), 9.06 (s, 1H), 8.84 (s, 1H), 8.51 (d, J=7.0 Hz, 1H), 8.25 (s, 1H), 8.17 (d, J=8.7 Hz, 1H), 6.69 (br., 1H), 4.48 (q, J=7.0 Hz, 2H), 3.28-3.25 (m, 1H), 2.61-2.55 (m, 1H), 2.39-2.33 (m, 1H), 2.25-2.09 (m, 6H), 1.40 (t, J=7.0 Hz, 3H), 0.93-0.80 (m, 4H). MS m/z 576.9 [M+H]⁺.

### Example 74: Preparation of Compound 74

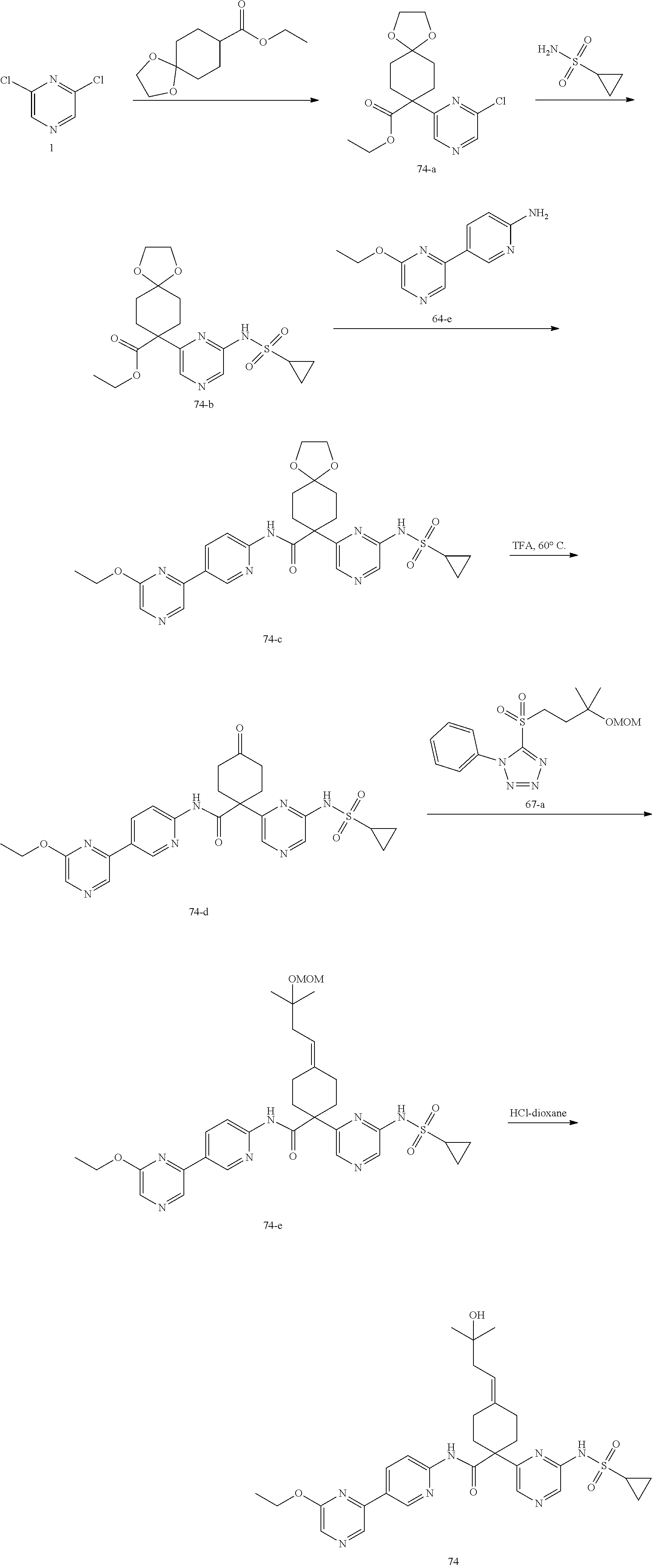

2,6-Dichloropyrazine (2 g, 13.43 mmol) and ethyl 1,4-dioxaspiro[4.5]nonane-8-carboxylate (2.88 g, 13.43 mmol) were dissolved in tetrahydrofuran (25 mL), followed by the addition of a tetrahydrofuran solution of lithium bis(trimethylsilyl)amide (18 mL, 1 M). The mixture was stirred at room temperature for 2 hours. After completion, the reaction solution was adjusted to pH 5-6 with dilute hydrochloric acid, and then extracted with ethyl acetate three times. The combined organic phases were washed with saturated brine, dried over anhydrous sodium sulfate, filtered, and concentrated under reduced pressure. The crude product was purified by silica gel column chromatography (ethyl acetate:petroleum ether=4:1) to obtain compound 74-a (1.4 g, yield 32%).

Compound 74-a (1.3 g, 3.98 mmol), cyclopropylsulfonamide (482 mg, 3.98 mmol), tris(dibenzylideneacetone)dipalladium (182 mg, 0.20 mmol), 4,5-bisdiphenylphosphino-9,9-dimethylxanthene (229 mg, 0.398 mmol), and cesium carbonate (3.89 g, 11.93 mmol) were dissolved in 1,4-dioxane solution (25 mL). The reaction was heated to 110° C. for 2 hours. After completion, the reaction solution was filtered through Celite, and the filtrate was concentrated under reduced pressure. The resulting crude product was purified by silica gel column chromatography to obtain compound 74-b (575 mg, yield 35%).

Compound 74-b (575 mg, 1.39 mmol) and compound 64-e (250 mg, 1.16 mmol) were dissolved in tetrahydrofuran (10 mL). A solution of lithium bis(trimethylsilyl)amide (1 M, 2.3 mL) was added dropwise at room temperature, and the reaction was carried out at room temperature for 1 hour. After completion, the reaction solution was adjusted to pH 5-6 with dilute hydrochloric acid (2 M), and then extracted with ethyl acetate three times. The combined organic phases were washed with saturated brine, dried over anhydrous sodium sulfate, filtered, and the filtrate was concentrated under reduced pressure. The crude product was purified by thin-layer chromatography silica gel plate (dichloromethane:methanol=30:1) to obtain compound 74-c (360 mg, yield 54%).

Compound 74-c (360 mg, 0.618 mmol) was dissolved in trifluoroacetic acid (5 mL), and the reaction was heated to 60° C. for 1 hour. After completion, the reaction solution was concentrated under reduced pressure to obtain a crude product, which was purified by silica gel column chromatography to obtain compound 74-d (260 mg, yield 78%).

Compound 74-d (70 mg, 0.13 mmol) and compound 67-a (88 mg, 0.26 mmol) were dissolved in tetrahydrofuran (1 mL). A solution of lithium bis(trimethylsilyl)amide (1 M, 0.6 mL) was added dropwise at room temperature, and the reaction was heated to 50° C. for 1 hour. After completion, the reaction solution was adjusted to pH 5-6 with dilute hydrochloric acid (2 M), and then extracted with ethyl acetate three times. The combined organic phases were washed with saturated brine, dried over anhydrous sodium sulfate, filtered, and the filtrate was concentrated under reduced pressure. The crude product was purified by thin-layer chromatography silica gel plate (dichloromethane:methanol=30:1) to obtain compound 74-e (46 mg, yield 54%).

Compound 74-e (46 mg, 0.07 mmol) was dissolved in dichloromethane (5 mL). A methanolic hydrochloric acid (4 M, 0.5 mL) solution was added at room temperature, and the reaction was stirred at room temperature for 1 hour. After completion, the reaction solution was adjusted to pH 7-8 with saturated sodium bicarbonate solution, and then extracted with dichloromethane three times. The combined organic phases were washed with saturated brine, dried over anhydrous sodium sulfate, filtered, and the filtrate was concentrated under reduced pressure. The crude product was purified by thin-layer chromatography silica gel plate (dichloromethane:methanol=20:1) to obtain compound 74 (25 mg, yield 58%). MS m/z 608.0 [M+H]⁺. ¹H NMR (500 MHz, DMSO-d₆) δ 11.05 (s, 1H), 10.07 (s, 1H), 9.01 (d, J=1.9 Hz, 1H), 8.84 (s, 1H), 8.49 (dd, J=8.8, 2.4 Hz, 1H), 8.41 (s, 1H), 8.25 (s, 1H), 8.19 (d, J=8.7 Hz, 1H), 8.17 (s, 1H), 5.25 (t, J=7.6 Hz, 1H), 4.47 (q, J=7.0 Hz, 2H), 4.19 (s, 1H), 3.14-3.06 (m, 1H), 2.64-2.53 (m, 2H), 2.44-2.36 (m, 1H), 2.33-2.26 (m, 1H), 2.22-2.10 (m, 3H), 2.10-2.05 (m, 2H), 2.04-1.96 (m, 1H), 1.39 (t, J=7.0 Hz, 3H), 1.06 (s, 6H), 1.05-1.01 (m, 2H), 0.86-0.78 (m, 2H).

### Example 75: Preparation of Compound 75

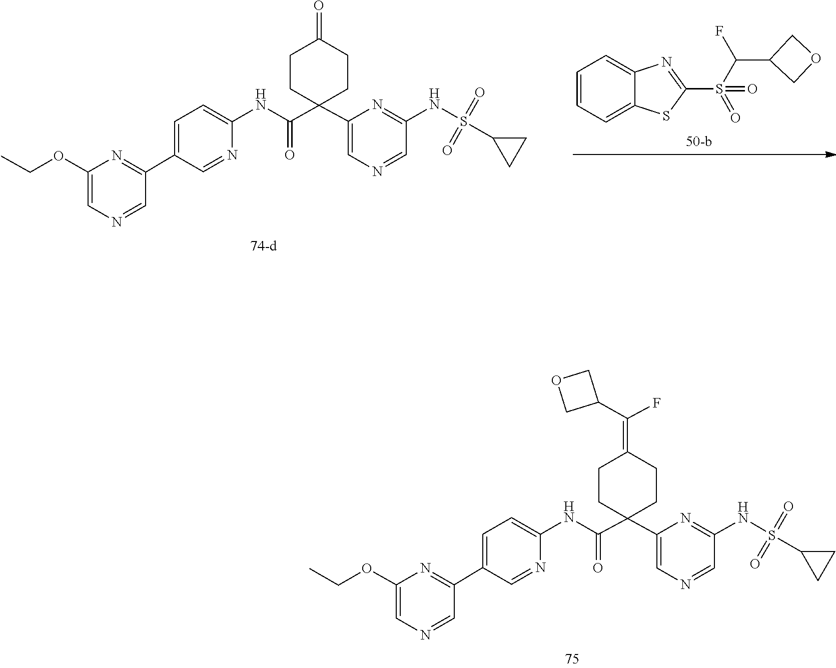 [SMILES: CCOc1cncc(-c2ccc(NC(=O)C3(c4cncc(NS(=O)(=O)C5CC5)n4)CCC(=O)CC3)nc2)n1 ; O=S(=O)(c1nc2ccccc2s1)C(F)C1COC1 ; CCOc1cncc(-c2ccc(NC(=O)C3(c4cncc(NS(=O)(=O)C5CC5)n4)CCC(=C(F)C4COC4)CC3)nc2)n1]

Compound 74-d (20 mg, 0.037 mmol) and compound 50-b (22 mg, 0.074 mmol) were dissolved in tetrahydrofuran (1 mL). A solution of lithium bis(trimethylsilyl)amide (1 M, 0.1 mL) was added dropwise at −60° C., and the reaction was carried out at room temperature for 1 hour. After completion, the reaction solution was adjusted to pH 5-6 with dilute hydrochloric acid (2 M), and then extracted with ethyl acetate three times. The combined organic phases were washed with saturated brine, dried over anhydrous sodium sulfate, filtered, and the filtrate was concentrated under reduced pressure. The crude product was purified by thin-layer chromatography silica gel plate (dichloromethane:methanol=20:1) to obtain compound 75 (5 mg, yield 22%). MS m/z 609.9 [M+H]⁺. ¹H NMR (500 MHz, DMSO-d₆) δ 11.04 (s, 1H), 10.13 (s, 1H), 9.00 (d, J=1.8 Hz, 1H), 8.83 (s, 1H), 8.52-8.46 (m, 1H), 8.38 (s, 1H), 8.25 (s, 1H), 8.20-8.10 (m, 2H), 4.70-4.57 (m, 3H), 4.51-4.43 (m, 2H), 4.43-4.28 (m, 1H), 3.12-3.04 (m, 2H), 2.61-2.53 (m, 2H), 2.30-2.05 (m, 4H), 2.07-1.95 (m, 2H), 1.38 (t, 3H), 1.06-0.98 (m, 2H), 0.84-0.74 (m, 2H).

### Example 76: Inhibition of CTPS1 Enzyme Activity by Compounds

The enzyme activity of CTPS1 was tested using the ADP-Glo method. Compounds were serially diluted in DMSO with 3-fold gradients across ten concentrations. Transfer 0.1 μL of diluted compound to a 384-well plate, centrifuge at 1000 rpm for 1 min. Add 2.5 μL of hCTPS1 enzyme working solution, centrifuge at 1000 rpm for 1 min, and incubate at 25° C. for 10 min. Add 2.5 μL of ATP & UTP solution and incubate at 25° C. for 10 min. Add 5 μL of substrate solution to initiate the reaction and incubate at 25° C. for 90 min. Add 10 μL of ADP-Glo solution to the 384-well plate, centrifuge at 1000 rpm for 1 min, incubate at 25° C. for 40 min, add 20 μL of detection solution, centrifuge at 1000 rpm for 1 min, and incubate at 25° C. for 60 min. Fluorescence values were read using a BMG microplate reader. Inhibition rate (%) was calculated as follows: Inhibition rate (%)=[(DMSO control well signal−Compound well signal)/(DMSO control well signal−Blank control well signal)]×100%. IC₅₀ values for compounds were analyzed by nonlinear regression in XLFit 5.5.0 software using the formula: Y=Bottom+(Top−Bottom)/(1+10\hat{ }((LogIC₅₀−X)*HillSlope)) where Y is the inhibition rate and X is the log value of compound concentration. The activities of some representative compounds are shown in Table 1.

TABLE 1
Inhibitory Activity of Compounds against CTPS1 (IC₅₀, nM)

| Compounds | CTPS1 (IC₅₀, nM) |
|---|---|
| 1 | <25 |
| 2 | <50 |
| 11 | <25 |
| 8 | <50 |
| 10 | <25 |
| 13 | <50 |
| 14 | <50 |
| 15 | <100 |
| 16 | <50 |
| 18 | <50 |
| 22 | <50 |
| 23 | <50 |
| 26 | <50 |
| 27 | <50 |
| 28 | <50 |
| 29 | <100 |
| 31 | <100 |
| 32 | <25 |
| 36 | <25 |
| 37 | <25 |
| 39 | <25 |
| 44 | <25 |
| 45 | <25 |
| 47 | <25 |
| 48 | <100 |
| 50 | <25 |
| 52 | <25 |
| 54 | <50 |
| 55 | <25 |
| 58 | <25 |
| 62 | <25 |
| 63 | <25 |
| 67 | <50 |
| 68 | <25 |
| 69 | <50 |
| 70 | <200 |

### Example 77: Inhibition of Jurkat Cell Proliferation by Compounds

Select Jurkat cells in good growth condition and digest them with trypsin. Add fresh culture medium, mix thoroughly, and centrifuge at 1000 rpm for 5 minutes. Seed the cells into a 96-well plate at a density of 10,000 cells per well, and incubate overnight in a 37° C. incubator. On the following day, take out of the culture plate and treat the cells with compounds prepared in a five-fold serial dilution. Return the plate to the 37° C. incubator and continue incubation for 72 hours.

After incubation, bring out the cell culture plate and equilibrate it to room temperature. Add 100 μL of CellTiter-Glo reagent to each well, shake in the dark for 2 minutes, and incubate for 10 minutes. Measure the luminescence using an Enspire plate reader. Calculate the inhibition rate using the following formula: Inhibition rate (%)=(1−(RLU_compound−RLU_blank)/(RLU_DMSO−RLU_blank))×100%. Plot the dose-response curve and calculate the IC₅₀ value using XLFit. The cell inhibitory activity data for some representative compounds are shown in Table 2.

TABLE 2
Inhibitory Activity of Compounds against Jurkat Cell Proliferation

| Compounds | Jurkat (IC₅₀, nM) |
|---|---|
| 1 | <30 |
| 2 | <50 |
| 4 | <50 |
| 5 | <15 |
| 6 | <100 |
| 7 | <750 |
| 11 | <15 |
| 8 | <15 |
| 9 | <50 |
| 10 | <30 |
| 13 | <50 |
| 14 | <30 |
| 15 | <50 |
| 16 | <50 |
| 17 | <200 |
| 18 | <50 |
| 20 | <200 |
| 21 | <1000 |
| 22 | <30 |
| 23 | <50 |
| 25 | <30 |
| 26 | <30 |
| 27 | <100 |
| 28 | <15 |
| 29 | <30 |
| 31 | <50 |
| 32 | <15 |
| 33 | <1000 |
| 35 | <200 |
| 36 | <5 |
| 37 | <30 |
| 38 | <30 |
| 39 | <15 |
| 40 | <200 |
| 41 | <100 |
| 42 | <50 |
| 43 | <50 |
| 44 | <15 |
| 45 | <30 |
| 47 | <15 |
| 48 | <30 |
| 49 | <50 |
| 50 | <15 |
| 51 | <30 |
| 52 | <15 |
| 53 | <100 |
| 54 | <50 |
| 55 | <50 |
| 56 | <50 |
| 57 | <50 |
| 58 | <15 |
| 59 | <15 |
| 60 | <30 |
| 61 | <50 |
| 62 | <15 |
| 63 | <15 |
| 64 | <50 |
| 65 | <50 |
| 66 | <50 |
| 67 | <50 |
| 68 | <15 |
| 69 | <15 |
| 71 | <200 |
| 72 | <200 |
| 73 | <30 |

### Example 78: Pharmacokinetic Study in Rats

Instrumentation: SCIEX Triple Quad 6500+ triple quadrupole liquid chromatography-mass spectrometry system (LC-MS/MS), operating software is Analyst 1.7.2 (Applied Biosystems, USA); ExionLC liquid chromatography system; Microsoft Excel was used for data calculation and processing. Pharmacokinetic parameters were calculated using the statistical moment method with WinNonlin 8.2 software. Key kinetic parameters included Tmax, T_{1/2}, Cmax, AUC_{0-24 h} etc. Column: Synergi 4 μm Fusion-RP 80 Å Luna C18 2 mm*50 mm, 4 μm; Column temperature: 40° C.; Mobile phase A: Water (0.1% formic acid); Mobile phase B: Acetonitrile; Flow rate: 0.8 mL/min; Gradient elution is adopted, the elution gradient is 0.10 min: 15% B; 1.60 min: 95% B; 1.90 min: 95% B; 1.91 min: 15% B; 2.20 min: 15% B. Injection volume: 1 μL.

Animals: 3 male SD rats with a weight range of 200-220 g were purchased and kept in the laboratory of the Experimental Animal Center for 3 days and then used. They were fasted for 12 hours predose and 4 hours after dosing. Drinking water was free during the test. After the rats were gavage, blood samples were taken according to the established time point.

Solvent: 0.4% ethanol+0.4% Tween80+99.2% (0.5% methylcellulose M450). Preparation of the solution for intragastric administration: Compounds were accurately weighed and added to the solvent. The mixture was then subjected to vortex mixing and sonication until a homogeneous suspension was achieved, thereby preparing medicinal solutions of the desired concentrations.

Compound samples: Typically, multiple structurally analogous compounds (with molecular weights differing by more than 2 units) were accurately weighed and administered together (cassette PK). This approach allows for the simultaneous screening of multiple compounds and comparison of their oral absorption rates. Single compound administration was also employed to study the pharmacokinetics of drug samples in rats.

After intragastric administration, blood was collected from the jugular vein at 0.25, 0.5, 1, 2, 4, 8, 10 and 24 hours. Transfer 20 μL of plasma sample (for blank samples and internal standard blank samples, add 20 μL of blank plasma) to a 1.5 mL centrifuge tube, add 200 μL of internal standard solution (50% methanol in acetonitrile (concentration 100 ng/mL)) (for Double blank samples, add 200 μL of 50% methanol in acetonitrile solution). Vortex the samples for 5 minutes, then centrifuge at 14000 rpm and 4° C. for 5 minutes. Transfer 80 μL and add to 80 μL of water, mix uniformly, and submit for LC-MS/MS analysis.

The compounds were accurately weighed to prepare different concentrations, and quantitative analysis on mass spectrometry was performed to establish a standard curve, and then the concentration of the above-mentioned compound in the plasma was tested to obtain its concentration at different time points. All measurement data were collected and processed by relevant software, and the statistical moment method was used to calculate the pharmacokinetic parameters (mainly including kinetic parameters Tmax, T_{1/2}, Cmax, AUC_{0-24 h} etc). The kinetic parameters of some representative compounds are shown in Table 3.

TABLE 3
Pharmacokinetic Parameters of Compounds in Rats

| | Dosage | T_{1/2} | Tₘₐₓ | Cₘₐₓ | AUC₀₋₂₄ |
|---|---|---|---|---|---|
| Compounds | (oral, mg/Kg) | (h) | (h) | (ng/mL) | (h*ng/mL) |
| 1 | 3 | 4.98 | 0.83 | 11332.97 | 68765.95 |
| 5 | 3 | 1.73 | 0.25 | 359.31 | 416.83 |
| 10 | 3 | 4.95 | 3.33 | 9333 | 87194 |
| 22 | 3 | 1.64 | 0.25 | 177 | 137 |
| 26 | 3 | 1.84 | 0.5 | 117.53 | 288.1 |
| 28 | 3 | 1.8 | 0.25 | 33.1 | 46.7 |
| 32 | 3 | 1.34 | 0.83 | 4017 | 15691 |
| 36 | 3 | 2.8 | 0.25 | 3773 | 6253 |
| 39 | 2 | 2.48 | 1.33 | 837 | 1472 |
| 44 | 2 | 1.77 | 1.17 | 10.5 | 33 |
| 47 | 2 | 2.01 | 0.25 | 1815 | 1412 |
| 50 | 2 | 1.13 | 0.25 | 1015 | 2158 |
| 51 | 2 | 1.56 | 4 | 14.5 | 62.7 |
| 52 | 1.5 | 2.01 | 2 | 1212 | 7640 |
| 58 | 3 | 2.53 | 2.17 | 4540 | 25065 |
| 63 | 3 | 3.03 | 2.33 | 4817 | 37609 |
| 68 | 3 | 1.83 | 2.17 | 6813 | 40850 |
| 69 | 2 | 1.89 | 1.83 | 2333 | 11255 |

All documents cited in the present invention are incorporated herein by reference as if each such document were individually incorporated by reference. Furthermore, it should be understood that after reading the above teachings of the present invention, those skilled in the art may make various changes or modifications to the invention, and such equivalent forms likewise fall within the scope defined by the appended claims of the present application.

1. A compound of formula (I), or an optical isomer, pharmaceutically acceptable salt, prodrug, deuterated derivative, hydrate, or solvate thereof: 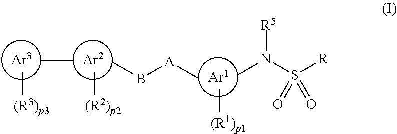
wherein in formula (I):
A is selected from Formula (Ia), Formula (Ib), or Formula (Ic): 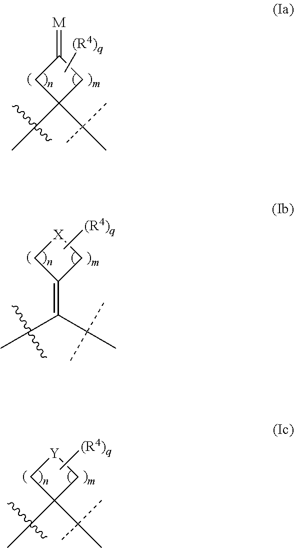
wherein in Formula (Ia), Formula (Ib), and Formula (Ic), “ [symbol] ” represents the point which attach to Ar¹ in the compound of Formula (I); “ [symbol] ” represents the point which attach to B in the compound of Formula (I);
B is —NHC(═O)— or —C(═O)NH—;
Ar¹, Ar² and Ar³ are each independently selected from aryl and heteroaryl;
R is selected from C₁₋₆ alkyl, C₃₋₆ cycloalkyl, 3- to 6-membered heterocyclyl, C₃₋₆ cycloalkyl-C₁₋₄ alkyl, 3- to 6-membered heterocyclyl-C₁₋₄ alkyl, C₃₋₆ cycloalkyl-C₂₋₄alkynyl, 3- to 6-membered heterocyclyl-C₂₋₄alkynyl, and C₃₋₆ cycloalkyl-C(═O)—C₁₋₂ alkyl;
R¹ is selected from hydrogen, halogen, C₁₋₄ alkyl, C₁₋₄ haloalkyl, C₁₋₄ alkoxy and CN;
R² is selected from hydrogen, halogen, C₁₋₄ alkyl, C₁₋₄ haloalkyl, C₁₋₄ alkoxy, C₁₋₄ haloalkoxy, and CN;
R³ is selected from hydrogen, halogen, C₁₋₄ alkyl, C₁₋₄ haloalkyl, C₂₋₄ alkenyl, C₂₋₄ alkynyl, C₃₋₆ cycloalkyl, C₃₋₆ cycloalkyl-C₁₋₄ alkyl, C₃₋₆ cycloalkyl-C₂₋₄alkynyl, 3- to 6-membered heterocyclyl, 3- to 6-membered heterocyclyl-C₂₋₄alkynyl, OR^{a}, SR^{a}, NR^{c}R^{c}, CN, or a group of Formula (Ie) (as shown below); wherein R^{a} is selected from hydrogen, C₁₋₄ alkyl, C₁₋₄ haloalkyl, C₃₋₆ cycloalkyl, or C₃₋₆ cycloalkyl-C₁₋₄ alkyl; each R^{c} is independently selected from hydrogen, C₁₋₄ alkyl, and C₁₋₄ haloalkyl;
R⁴ is selected from hydrogen, halogen, C₁₋₄ alkyl, C₁₋₄ haloalkyl, hydroxy, C₁₋₄ alkoxy, and CN;
R⁵ is selected from hydrogen, C₁₋₄ alkyl, C₃₋₆ cycloalkyl, 3- to 6-membered heterocyclyl, aryl, and heteroaryl;
M is selected from CR^{h}Rⁱ; wherein R^{h} and Rⁱ are each independently selected from hydrogen, halogen, C₁₋₄ alkyl, C₃₋₆ cycloalkyl, 3- to 8-membered heterocyclyl, aryl, heteroaryl, CN, C(O)R^{g}, C(O)OR^{f}, or C(O)NR^{d}R^{d}; wherein said alkyl, cycloalkyl, heterocyclyl, aryl, and heteroaryl are each optionally substituted by one or more substituents independently selected from the group consisting of: halogen, C₁₋₄ alkyl, C₂₋₄ alkenyl, C₂₋₄ alkynyl, C₃₋₆ cycloalkyl, 3- to 6-membered heterocyclyl, aryl, heteroaryl, CN, OR^{f}, SR^{f}, NR^{d}R^{d}, C(O)R^{g}, C(O)O^{f}, OC(O)R^{g}, C(O)NR^{d}R^{d}, NR^{d}C(O)R^{g}, NR^{d}C(O)NR^{d}R^{d}, OC(O)NR^{d}R^{d}, NR^{d}C(O)OR^{f}, OC(O)OR^{f}, S(O)₂NR^{d}R^{d}, NR^{d}S(O)₂R^{g}, and NR^{d}S(O)₂NR^{d}R^{d}; or R^{h} and Rⁱ together with the carbon atom to which they are attached form a 3- to 8-membered cyclic structure, said cyclic structure optionally containing 0, 1, or 2 heteroatoms independently selected from N, O and S, and optionally substituted by one or more substituents independently selected from the group consisting of halogen, C₁₋₄ alkyl, CN, OR^{f}, SR^{f}, NR^{d}R^{d}, and =M, wherein M is as defined above; wherein each R^{d} is independently selected from hydrogen, C₁₋₄ alkyl, C₁₋₄ haloalkyl, C₁₋₄ alkoxyC₂₋₄alkyl, C₃₋₆ cycloalkyl, and 3- to 6-membered heterocyclyl; each Rf is independently selected from hydrogen, C₁₋₄ alkyl, C₁₋₄ haloalkyl, C₃₋₆ cycloalkyl, and 3- to 6-membered heterocyclyl; each R^{g} is independently selected from hydrogen, C₁₋₄ alkyl, C₁₋₄ haloalkyl, C₂₋₄ alkenyl, C₂₋₄ alkynyl, C₃₋₆ cycloalkyl, 3- to 6-membered heterocyclyl, aryl, and heteroaryl;
X and Y are each independently selected from O, CR^{e}R^{e} and NR^{b}; wherein each R^{e} is independently selected from hydrogen, halogen, C₁₋₄ alkyl, C₃₋₆ cycloalkyl, 3- to 6-membered heterocyclyl, aryl, heteroaryl, CN, OR^{f}, SR^{f}, NR^{d}R^{d}, C(O)R^{g}, C(O)OR^{f}, OC(O)R^{g}, C(O)NR^{d}R^{d}, NR^{d}C(O)R^{g}, NR^{d}C(O)NR^{d}R^{d}, OC(O)NR^{d}R^{d}, NR^{d}C(O)OR^{f}, OC(O)OR^{f}, S(O)₂NR^{d}R^{d}, NR^{d}S(O)₂R^{g} and NR^{d}S(O)₂NR^{d}R^{d}; R^{b} is selected from hydrogen, C₁₋₄ alkyl, C₃₋₆ cycloalkyl, 3- to 6-membered heterocyclyl, aryl, heteroaryl, C(O)R^{g}, C(O)OR^{f}, C(O)NR^{d}R^{d}, S(O)₂NR^{d}R^{d} and S(O)₂R^{g}; the alkyl in R^{e} or R^{b} is optionally substituted by one or more substituents selected from the group consisting of halogen, C₃₋₆ cycloalkyl, 3- to 6-membered heterocyclyl, aryl, heteroaryl, CN, OR^{f}, SR^{f}, NR^{d}R^{d}, C(O)R^{g}, C(O)O^{f}, OC(O)R^{g}, C(O)NR^{d}R^{d}, NR^{d}C(O)R^{g}, NR^{d}C(O)NR^{d}R^{d}, OC(O)NR^{d}R^{d}, NR^{d}C(O)OR^{f}, OC(O)OR^{f}, S(O)₂NR^{d}R^{d}, NR^{d}S(O)₂R^{g}, and NR^{d}S(O)₂NR^{d}R^{d}; R^{d}, R^{f}, R^{g} are defined as above;
provided that when A is formula (Ic) and Y is NR^{b}, then NR^{b} is selected from aryl, heteroaryl, C(O)R^{g′}, or Formula (Id), 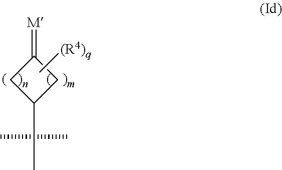
wherein “ [symbol] ” represents the point which attach to N in Formula (Id); R^{g′} is selected from C₂₋₄ alkenyl, C₂₋₄ alkynyl, C₃₋₆ cycloalkyl, or 3- to 6-membered heterocyclyl; M′ is selected from O or CR^{h}Rⁱ; R⁴, R^{h} and Rⁱ are as defined above;
with the proviso that, when A is selected from formula (Ic), Y is selected from O, CR^{e}R^{e}, or NR^{b}, and R^{b} is other than aryl, heteroaryl, C(O)R^{g′}, and Formula (Id), then R is selected from C₃₋₆ cycloalkyl-C₂₋₄ alkynyl, 3- to 6-membered heterocyclyl C₂₋₄ alkynyl, or C₃₋₆ cycloalkyl C(═O)C₁₋₂ alkyl; or R³ is selected from C₃₋₆ cycloalkyl-C₂₋₄ alkynyl, 3- to 6-membered heterocyclyl C₂₋₄ alkynyl, or formula (Ie); 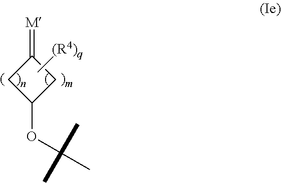
wherein “ [symbol] ” represents the point which attach to Ar³ in Formula (Ie); and R^{e}, R^{b}, R^{g′}, M′, and R⁴ are as defined above;
wherein p1, p2 and p3 are each independently selected from 0, 1, 2 and 3;
each m and each n are independently selected from 0, 1, 2, 3, 4, 5 and 6; provided that m and n are not both 0;
each q is independently selected from 0, 1, 2, 3 and 4;
wherein each of the aforementioned alkyl, alkenyl, alkynyl, cycloalkyl, heterocyclyl, cyclic structure, aryl, and heteroaryl groups is optionally and independently substituted by 1 to 3 substituents independently selected from the group consisting of: halogen, C₁₋₄ alkyl, C₁₋₄ haloalkyl, C₂₋₄ alkenyl, C₂₋₄ alkynyl, C₃₋₈ cycloalkyl, 3- to 8-membered heterocyclyl, aryl, heteroaryl, CN, NO₂, OR^{f}, SR^{f}, NR^{d}R^{d}, C(O)R^{g}, C(O)OR^{f}, C(O)NR^{d}R^{d}, NR^{d}C(O)R^{g}, NR^{d}S(O)₂R^{g} and S(O)₂R^{g}, provided that the resulting chemical structure is stable and chemically meaningful; wherein R^{d}, R^{f}, and R^{g} are as defined above;
unless otherwise specified, the aforementioned aryl is an aromatic group containing 6 to 12 carbon atoms; heteroaryl is a 5- to 15-membered heteroaromatic group; and cyclic structure is a saturated or unsaturated cyclic group which optionally containing heteroatoms.

2. The compound of claim 1, characterized in that Formula (I) is Formula (IIa) or Formula (IIb): 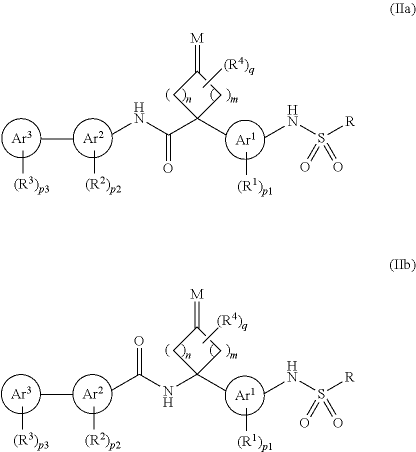
wherein the definitions of the groups in Formula (IIa) and Formula (IIb) are as defined in claim 1.

3. The compound according to any one of claims 1-2, characterized in that Formula (I) is Formula (III): 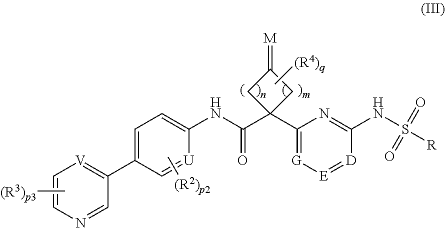
D, E, and G are each independently selected from N or CR¹, provided that no more than two of D, E, and G are N;
U is N or CR²;
V is N or CR³;
R¹, R², R³, and the definitions of the remaining groups in Formula (III) are as defined in claim 1.

4. The compound according to any one of claims 1-3, characterized in that formula (I) is formula (IVa), formula (IVb), or formula (IVc): 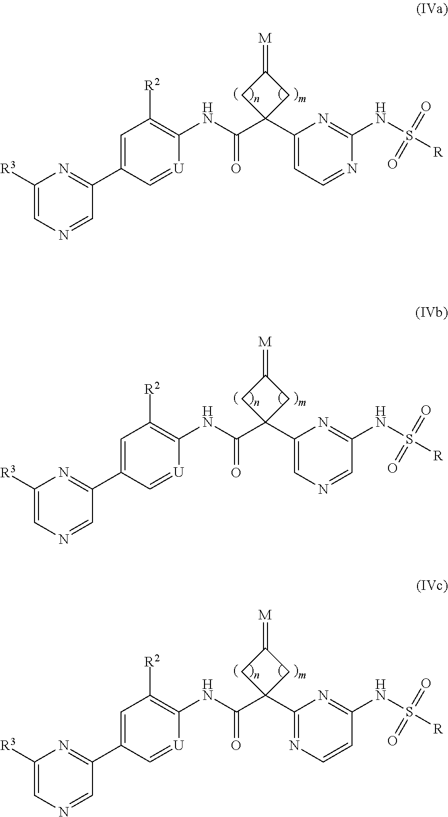
U is N or CH;
the remaining groups in Formula (IVa), Formula (IVb), and Formula (IVc) are as defined in claim 1.

5. The compound according to claim 1, characterized in that formula (I) is formula (V): 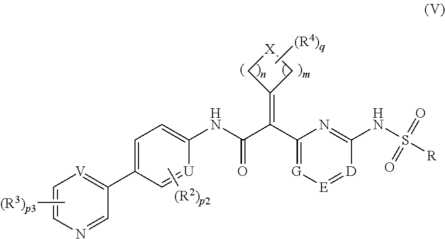
D, E and G are each independently selected from N or CR¹, provided that no more than two of D, E, and G are N;
U is N or CR²;
V is N or CR³;
R¹, R², R³, and the remaining groups in Formula (V) are as defined in claim 1.

6. The compound according to any one of claims 1 and 5, characterized in that Formula (I) is formula (VIa), formula (VIb), or formula (VIc): 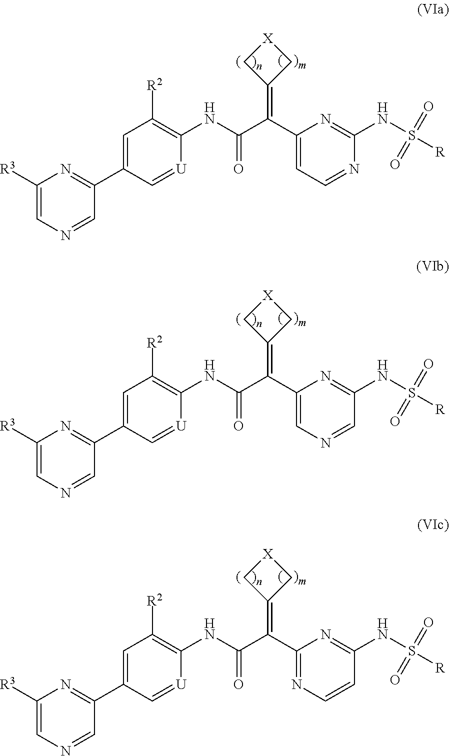
U is N or CH;
the remaining groups in Formula (VIa), Formula (VIb), and Formula (VIc) are as defined in claim 1.

7. The compound according to claim 1, characterized in that formula (I) is formula (VII): 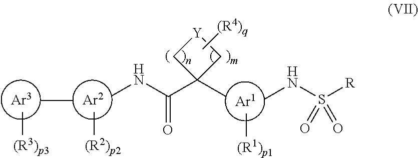
the remaining groups in formula (VII) and provisos are as defined in claim 1.

8. The compound according to claim 1 or 7, characterized in that formula (I) is formula (VIII): 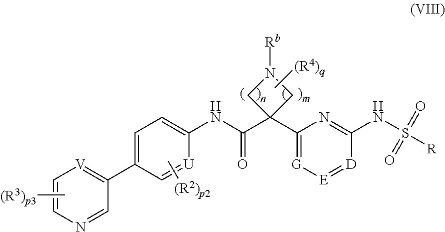
D, E and G are each independently selected from N or CR¹, provided that no more than two of D, E, and G are N;
U is N or CR²;
V is N or CR³;
R¹, R², R³, and the remaining groups in Formula (VIII) are as defined in claim 1.

9. The compound according to any one of claims 1 and 7-8, characterized in that formula (I) is formula (IXa), formula (IXb), formula (IXc): 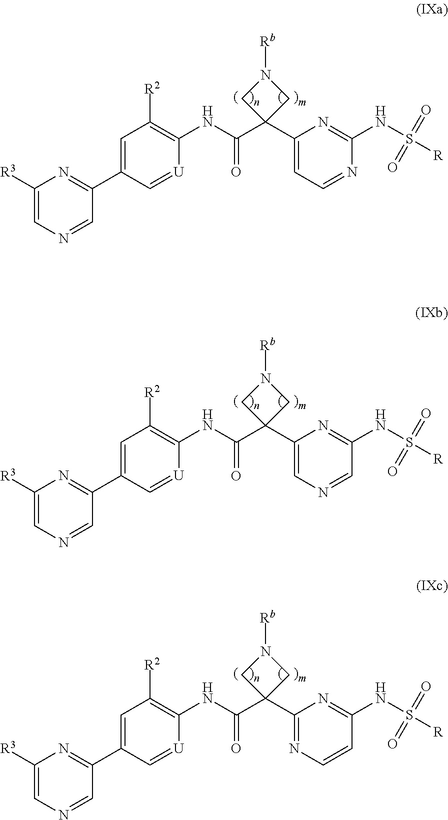
U is N or CH;
the remaining groups in Formula (IXa), Formula (IXb), and Formula (IXc) and the provisos are as defined in claim 1.

10. The compound according to any one of claims 1-2, characterized in that Formula (I) is Formula (Xa) or Formula (Xb): 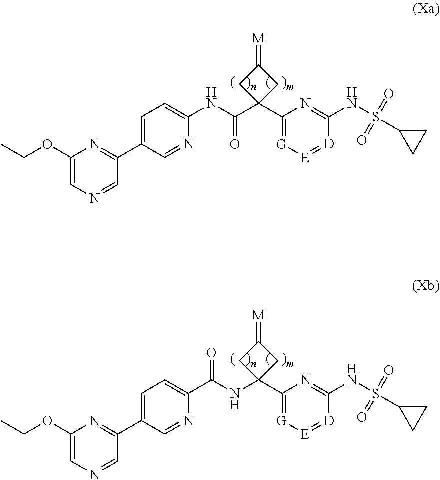
D, E, and G are each independently selected from N or CH, provided that no more than two of D, E, and G are N;
M is selected from CR^{h}Rⁱ; wherein R^{h} and Rⁱ are each independently selected from hydrogen, halogen, C₁₋₄ alkyl, C₃₋₆ cycloalkyl, 3- to 8-membered heterocyclyl, aryl, heteroaryl, CN, C(O)R^{g}, C(O)OR^{f}, or C(O)NR^{d}R^{d}; wherein said alkyl, cycloalkyl, heterocyclyl, aryl, and heteroaryl are each optionally substituted by one or more substituents independently selected from the group consisting of halogen, C₁₋₄ alkyl, C₂₋₄ alkenyl, C₂₋₄ alkynyl, C₃₋₆ cycloalkyl, 3- to 6-membered heterocyclyl, aryl, heteroaryl, CN, OR^{f}, SR, NR^{d}R^{d}, C(O)R^{g}, C(O)O^{f}, OC(O)R^{g}, C(O)NR^{d}R^{d}, NR^{d}C(O)R^{g}, NR^{d}C(O)NR^{d}R^{d}, OC(O)NR^{d}R^{d}, NR^{d}C(O)OR^{f}, OC(O)OR^{f}, S(O)₂NR^{d}R^{d}, NR^{d}S(O)₂R^{g} and NR^{d}S(O)₂NR^{d}R^{d}; or
R^{h} and Rⁱ, together with the carbon atom to which they are attached, form a 3- to 8-membered cyclic structure, said cyclic structure optionally containing 0, 1 or 2 heteroatoms independently selected from N, O and S, and optionally substituted by one or more substituents independently selected from the group consisting of: halogen, C₁₋₄ alkyl, CN, OR^{f}, SR^{f}, NR^{d}R^{d} and =M, wherein M is as defined above;
each R^{d} is independently selected from hydrogen, C₁₋₄ alkyl, C₁₋₄ haloalkyl, C₁₋₄ alkoxy-C₂₋₄ alkyl, C₃₋₆ cycloalkyl, or 3- to 6-membered heterocyclyl; each R^{f} is independently selected from hydrogen, C₁₋₄ alkyl, C₁₋₄ haloalkyl, C₃₋₆ cycloalkyl, or 3- to 6-membered heterocyclyl; each R^{g} is independently selected from hydrogen, C₁₋₄ alkyl, C₁₋₄ haloalkyl, C₂₋₄ alkenyl, C₂₋₄ alkynyl, C₃₋₆ cycloalkyl, 3- to 6-membered heterocyclyl, aryl, or heteroaryl;
m and n are each independently selected from 0, 1, 2, 3, 4, 5, or 6.

11. The compound according to any one of claims 1-2 and 9, characterized in that formula (I) is formula (XIa), formula (XIb), formula (XIc), formula (XId), formula (XIe), or formula (XIf): 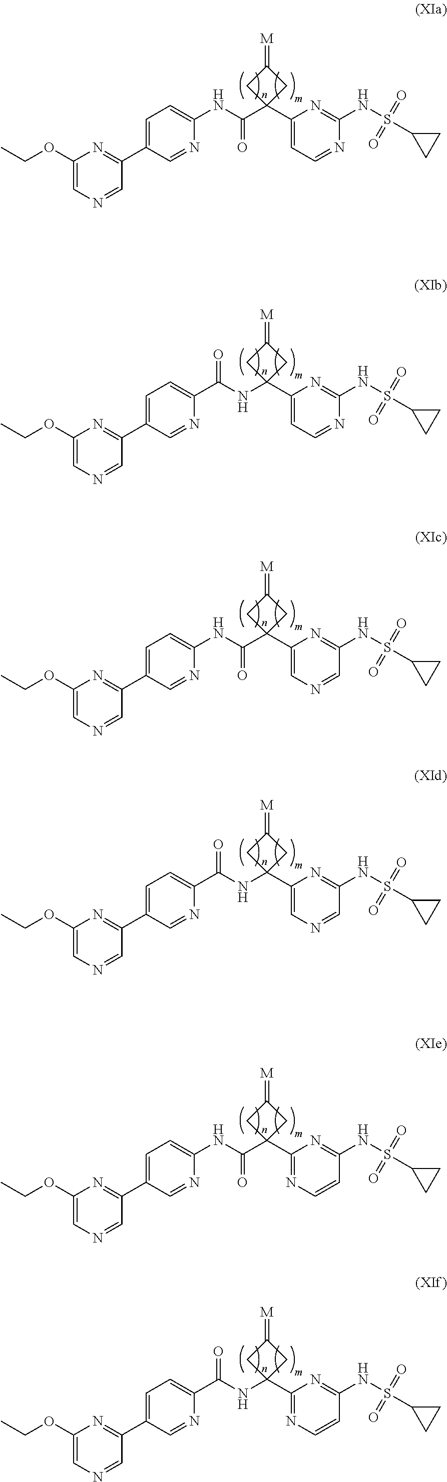
wherein M is selected from CR^{h}Rⁱ; wherein R^{h} and Rⁱ are each independently selected from hydrogen, halogen, C₁₋₄ alkyl, C₃₋₆ cycloalkyl, 3- to 8-membered heterocyclyl, aryl, heteroaryl, CN, C(O)R^{g}, C(O)OR^{f}, or C(O)NR^{d}R^{d};
wherein said alkyl, cycloalkyl, heterocyclyl, aryl, and heteroaryl are each optionally substituted by one or more substituents selected from the group consisting of: halogen, C₁₋₄ alkyl, C₂₋₄ alkenyl, C₂₋₄ alkynyl, C₃₋₆ cycloalkyl, 3- to 6-membered heterocyclyl, aryl, heteroaryl, CN, OR^{f}, SR^{f}, NR^{d}R^{d}, C(O)R^{g}, C(O)OR^{f}, OC(O)R^{g}, C(O)NR^{d}R^{d}, NR^{d}C(O)R^{g}, NR^{d}C(O)NR^{d}R^{d}, OC(O)NR^{d}R^{d}, NR^{d}C(O)OR^{f}, OC(O)OR^{f}, S(O)₂NR^{d}R^{d}, NR^{d}S(O)₂R^{g}, and NR^{d}S(O)₂NR^{d}R^{d};
or R^{h} and Rⁱ together with the carbon atom to which they are attached form a 3- to 8-membered cyclic structure, said cyclic structure optionally containing 0, 1 or 2 heteroatoms selected from N, O, and S, and optionally substituted by one or more substituents selected from the group consisting of halogen, C₁₋₄ alkyl, CN, OR^{f}, SR, NR^{d}R^{d} and =M, wherein M is as defined above; wherein each R^{d} is independently selected from hydrogen, C₁₋₄ alkyl, C₁₋₄ haloalkyl, C₁₋₄ alkoxy-C₂₋₄ alkyl, C₃₋₆ cycloalkyl, or 3- to 6-membered heterocyclyl; each R^{f} is independently selected from hydrogen, C₁₋₄ alkyl, C₁₋₄ haloalkyl, C₃₋₆ cycloalkyl, or 3- to 6-membered heterocyclyl; each R^{g} is independently selected from hydrogen, C₁₋₄ alkyl, C₁₋₄ haloalkyl, C₂₋₄ alkenyl, C₂₋₄ alkynyl, C₃₋₆ cycloalkyl, 3- to 6-membered heterocyclyl, aryl, or heteroaryl;
m and n are each independently selected from 0, 1, 2, 3, 4, 5 and 6.

12. The compound according to claim 11, characterized in that:
M is CR^{h}Rⁱ; wherein R^{h} and Rⁱ are each independently selected from hydrogen, halogen, C₁₋₄ alkyl, C₃₋₆ cycloalkyl, 3- to 6-membered heterocyclyl, aryl, heteroaryl, CN, C(O)R^{g}, C(O)OR^{f}, and C(O)NR^{d}R^{d}; wherein said alkyl, cycloalkyl, heterocyclyl, aryl, and heteroaryl are each optionally substituted by one or more substituents selected from the group consisting of halogen, C₁₋₄ alkyl, C₂₋₄ alkenyl, C₂₋₄ alkynyl, C₃₋₆ cycloalkyl, 3- to 6-membered heterocyclyl, CN, OR^{f}, NR^{d}C(O)R^{g}, and NR^{d}S(O)₂R^{g};
wherein R^{d}, R^{f}, and R^{g} are as defined in claim 11;
and wherein m and n are each independently 1 or 2.

13. The compound according to any one of claims 1-2, characterized in that formula (I) is formula (XIIa) or formula (XIIb): 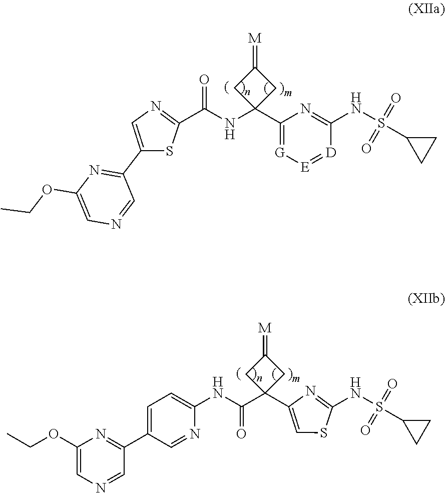
D, E, and G are each independently selected from N or CH, provided that no more than two of D, E, and G are N;
M, in and n are as defined in claim 12.

14. The compound according to claim 1, or an optical isomer, pharmaceutically acceptable salt, prodrug, deuterated derivative, hydrate or solvate thereof, characterized in that selected from the group consisting of:

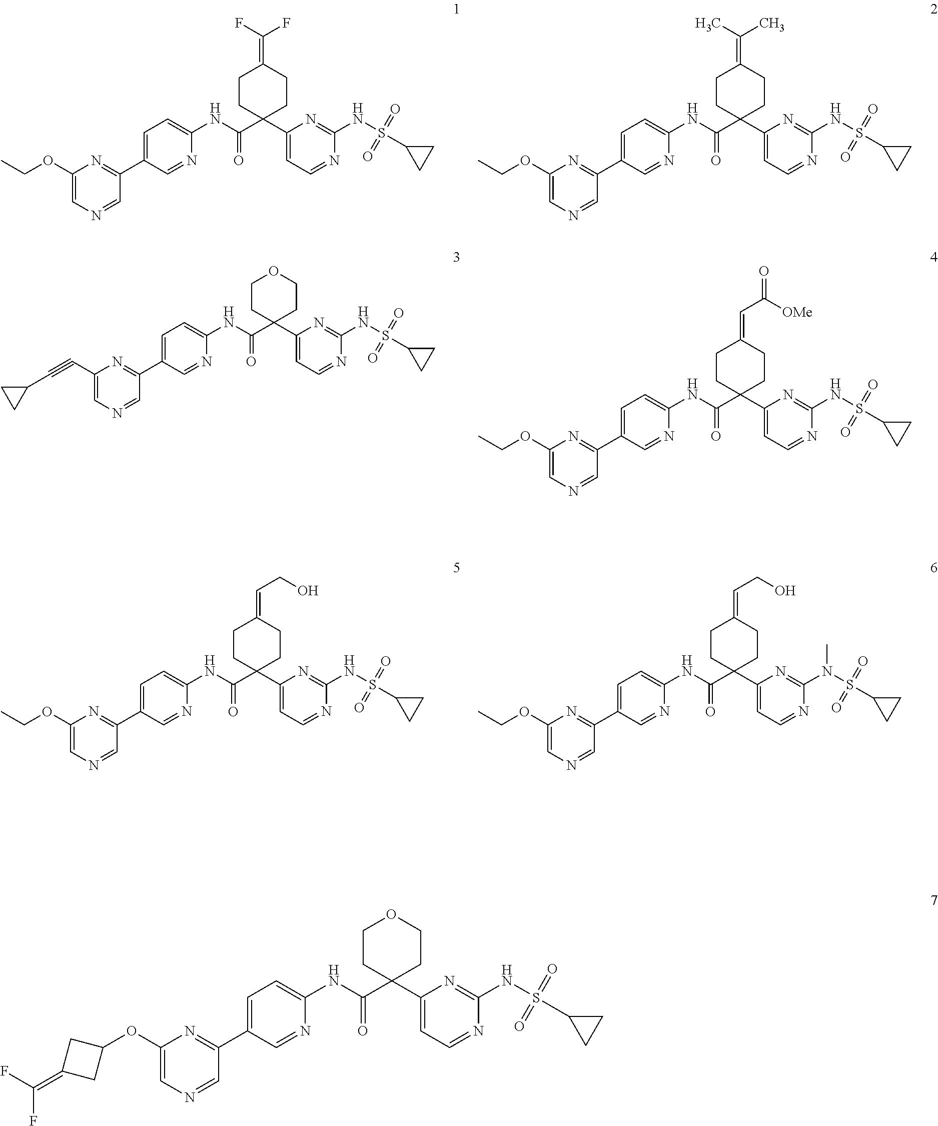

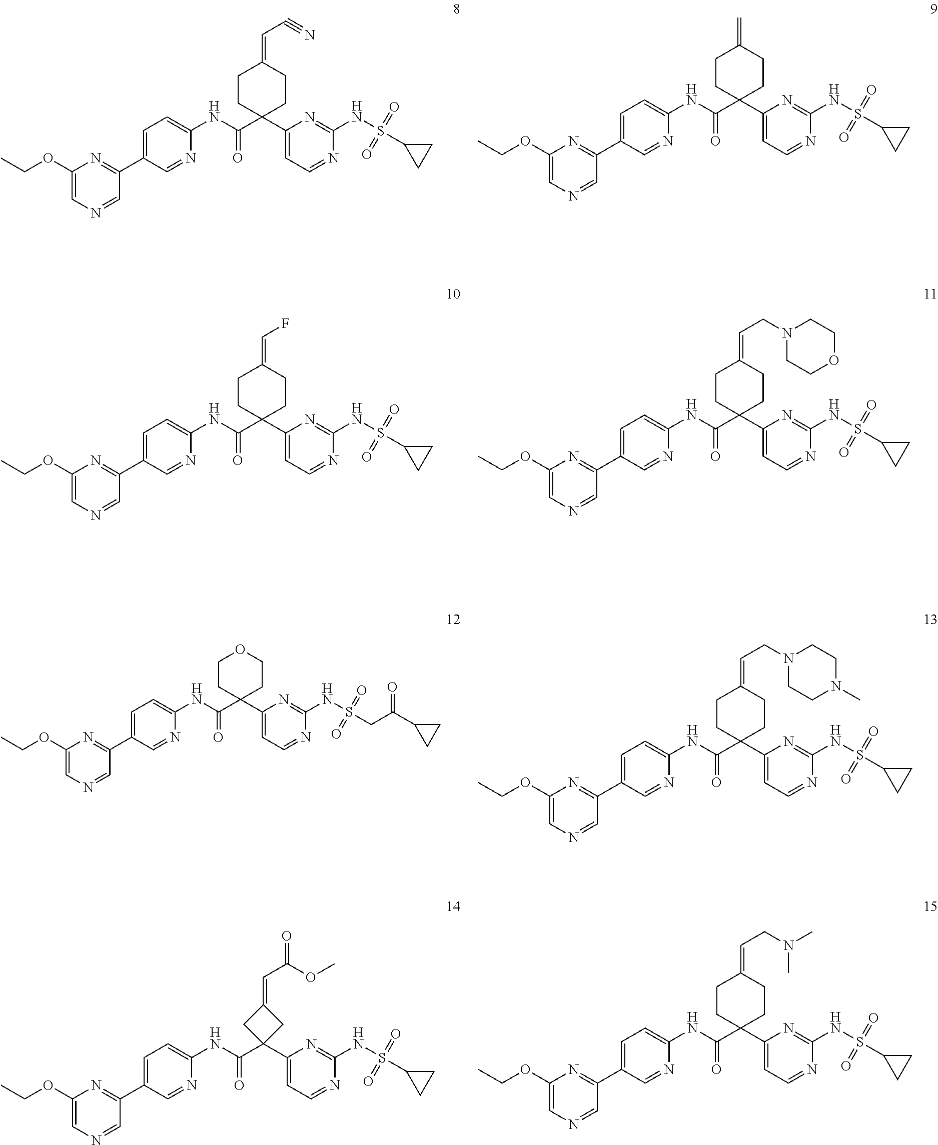

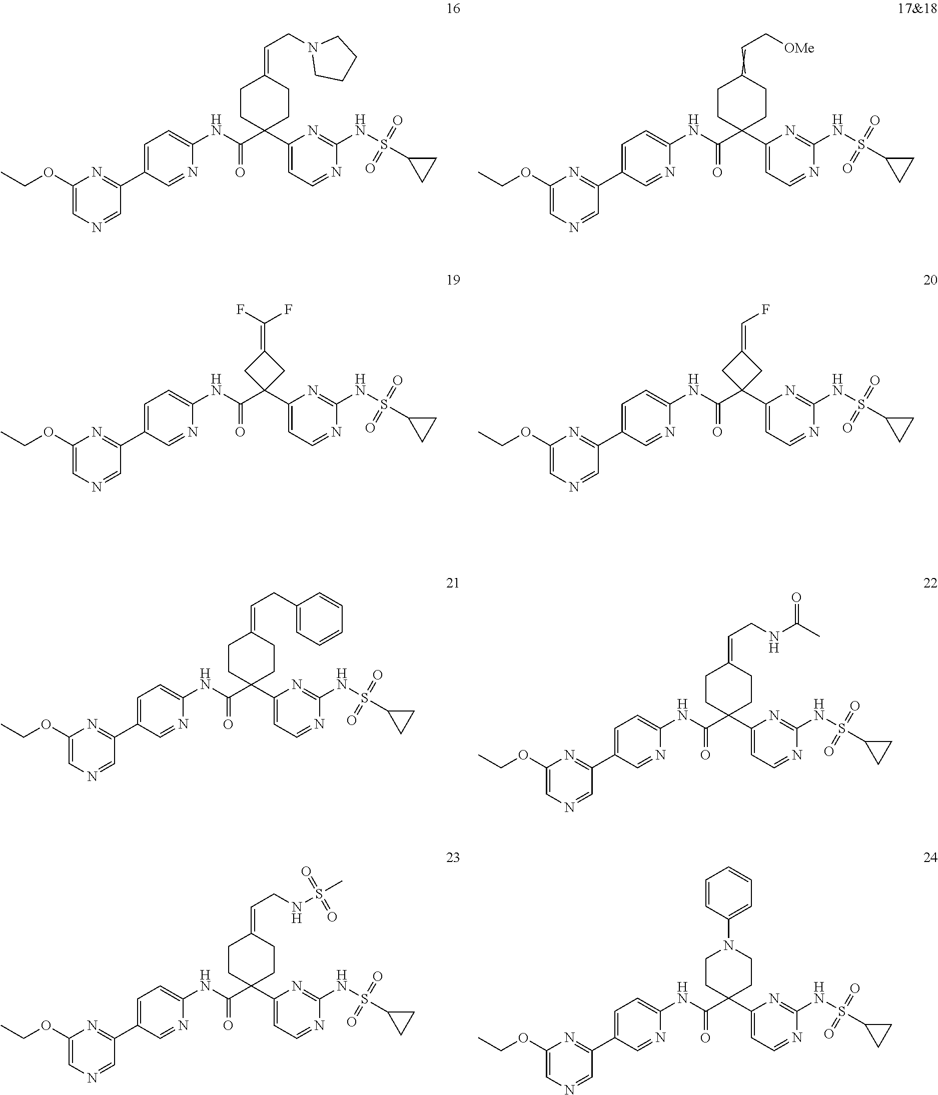

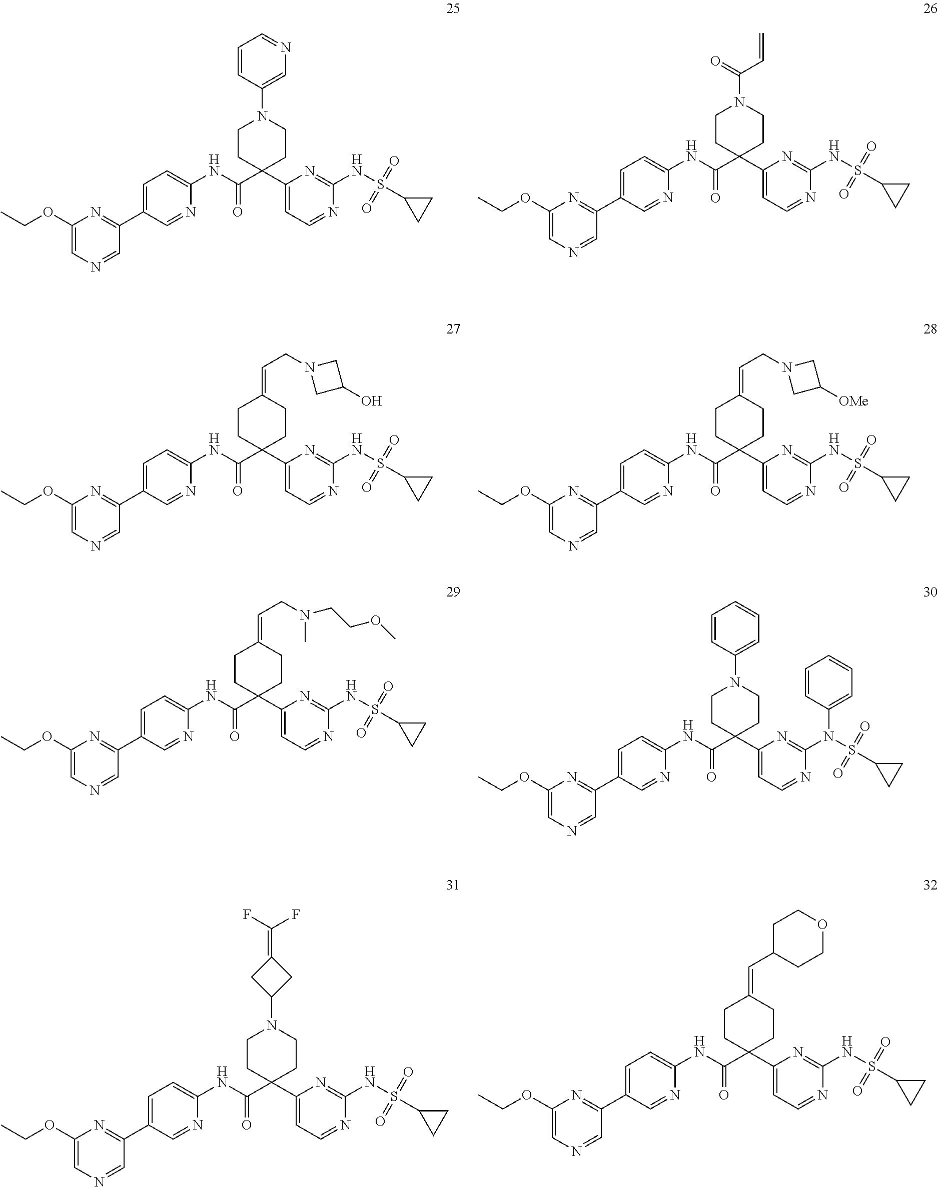

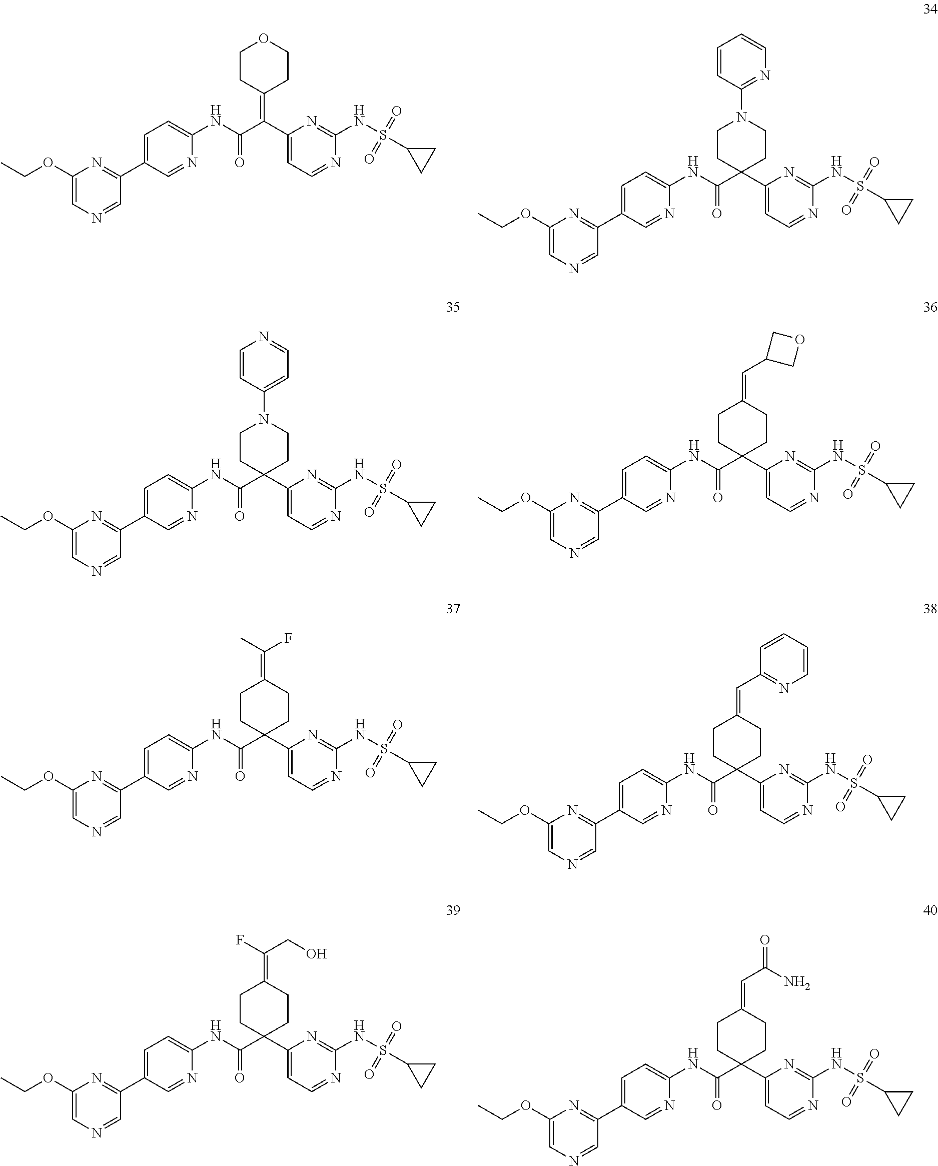

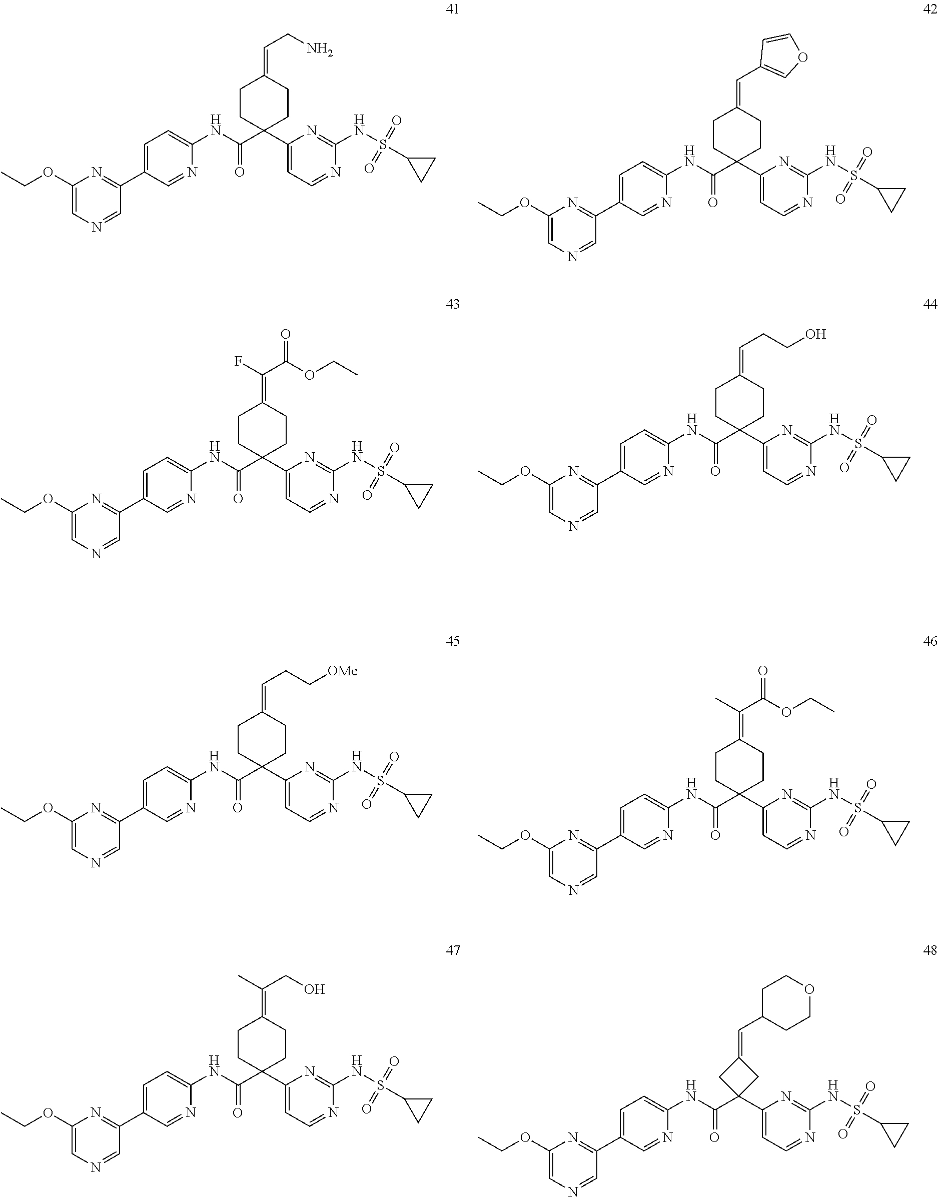

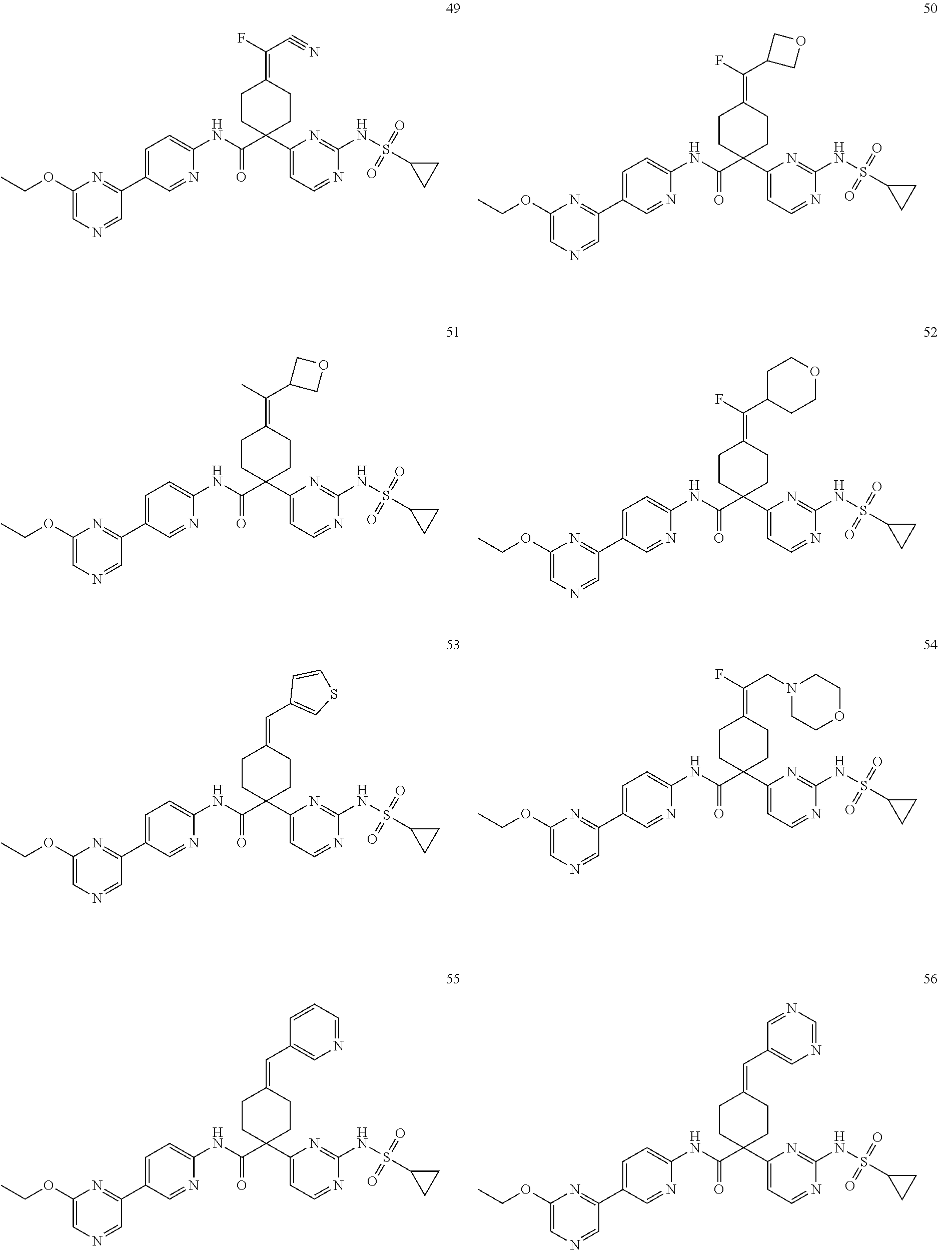

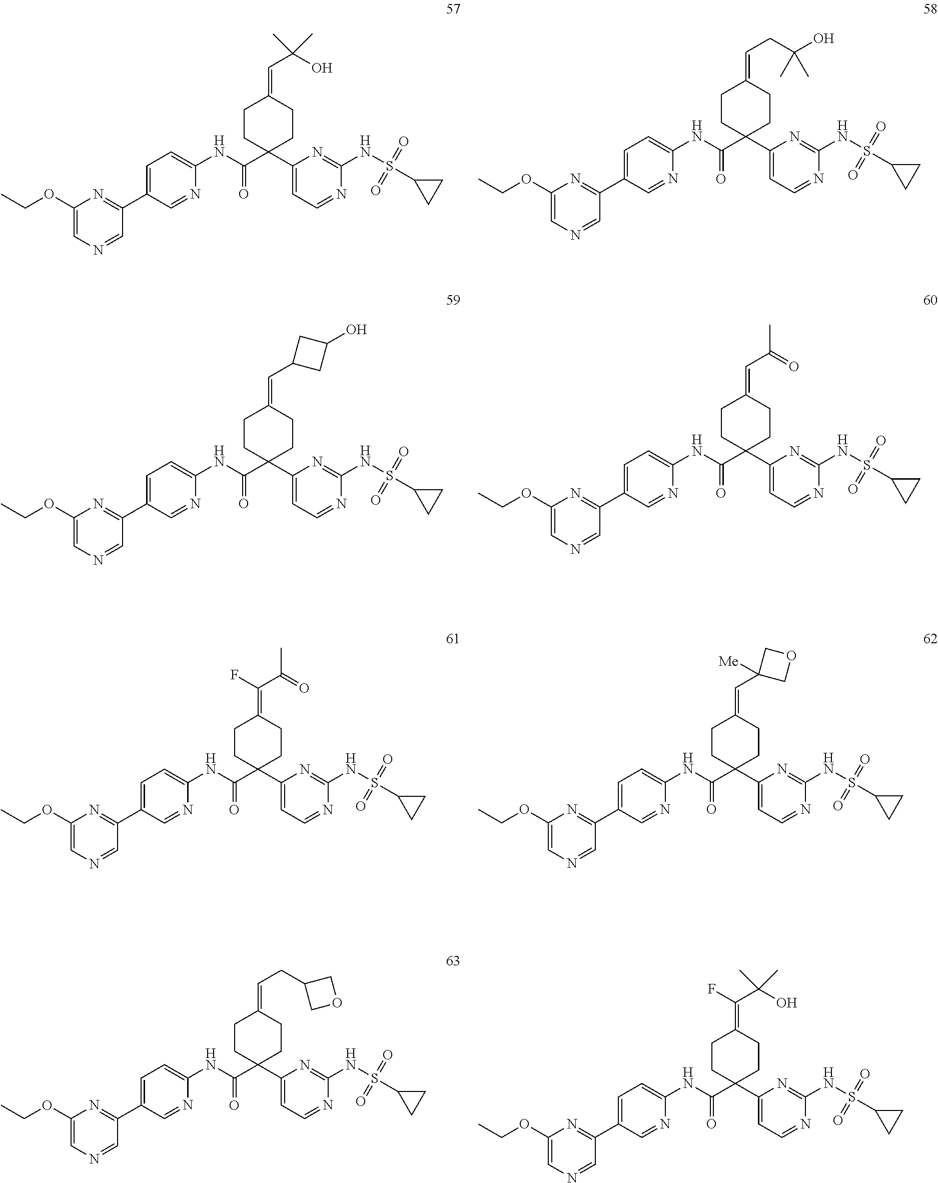

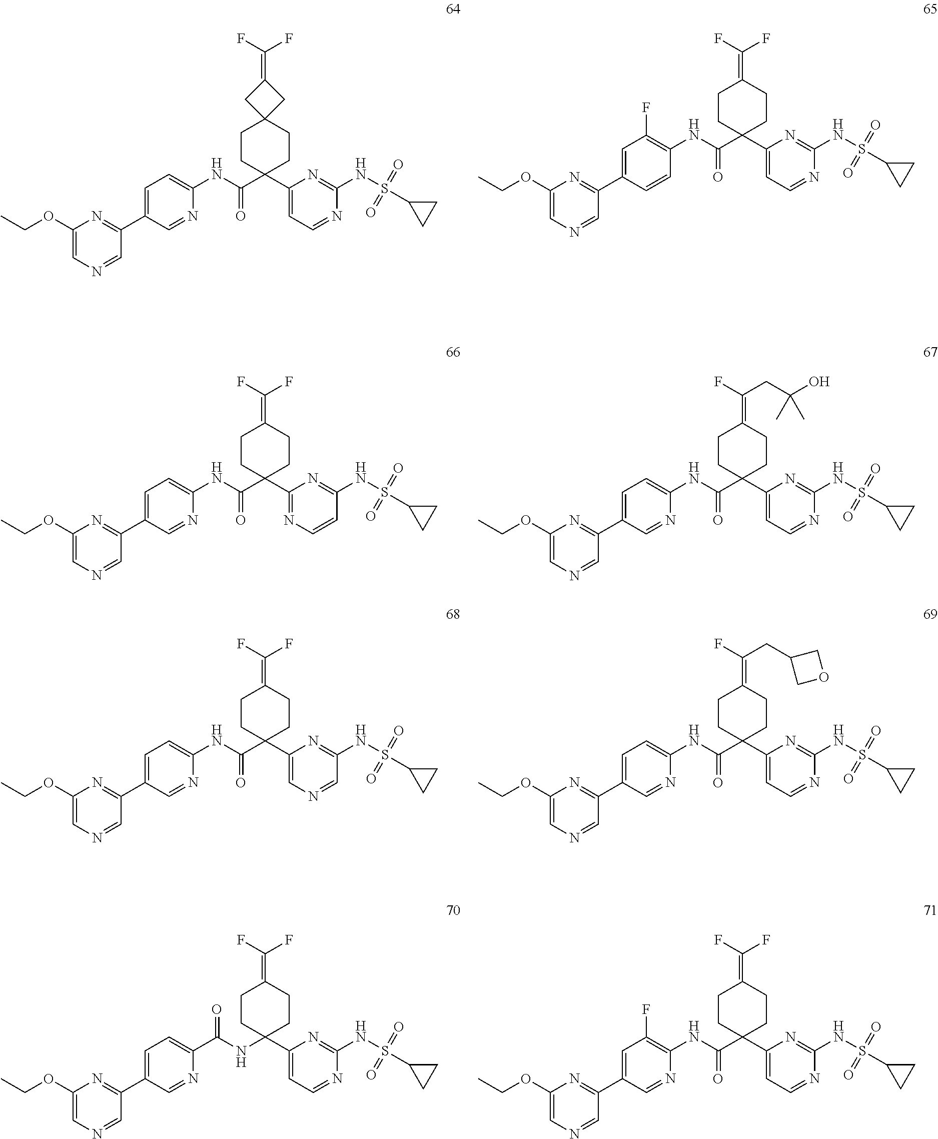

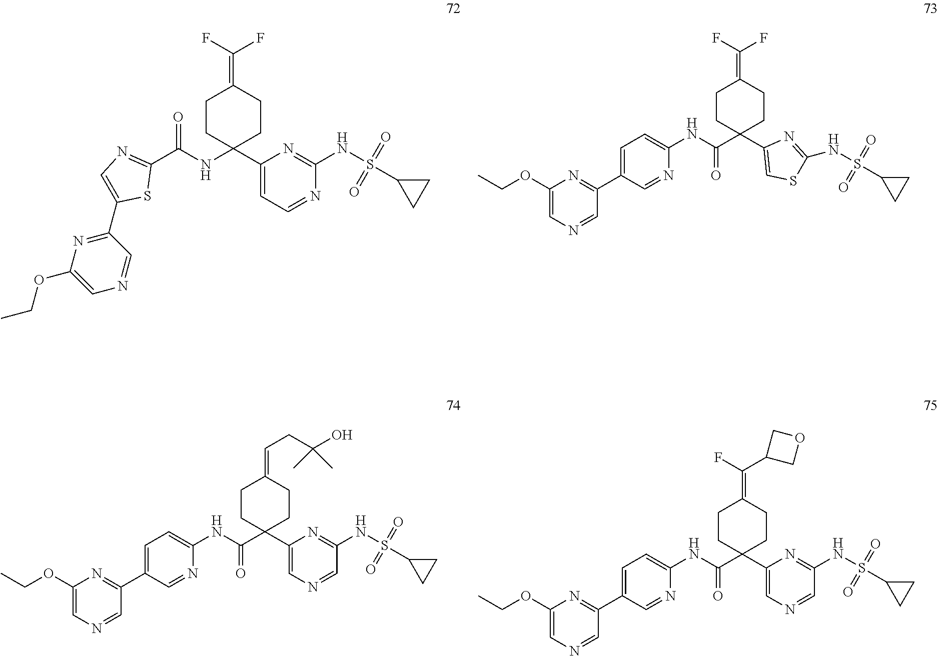 [SMILES: CCOc1cncc(-c2cnc(C(=O)NC3(c4ccnc(NS(=O)(=O)C5CC5)n4)CCC(=C(F)F)CC3)s2)n1 ; CCOc1cncc(-c2ccc(NC(=O)C3(c4csc(NS(=O)(=O)C5CC5)n4)CCC(=C(F)F)CC3)nc2)n1 ; CCOc1cncc(-c2ccc(NC(=O)C3(c4cncc(NS(=O)(=O)C5CC5)n4)CCC(=CCC(C)(C)O)CC3)nc2)n1 ; CCOc1cncc(-c2ccc(NC(=O)C3(c4cncc(NS(=O)(=O)C5CC5)n4)CCC(=C(F)C4COC4)CC3)nc2)n1]

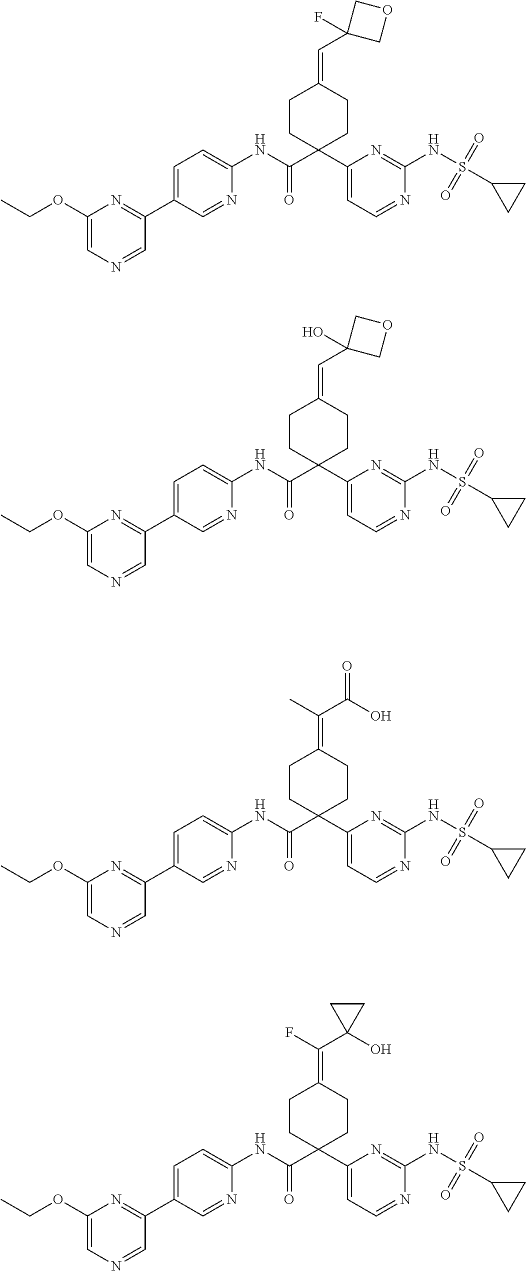 [SMILES: CCOc1cncc(-c2ccc(NC(=O)C3(c4ccnc(NS(=O)(=O)C5CC5)n4)CCC(=CC4(F)COC4)CC3)nc2)n1 ; CCOc1cncc(-c2ccc(NC(=O)C3(c4ccnc(NS(=O)(=O)C5CC5)n4)CCC(=CC4(O)COC4)CC3)nc2)n1 ; CCOc1cncc(-c2ccc(NC(=O)C3(c4ccnc(NS(=O)(=O)C5CC5)n4)CCC(=C(C)C(=O)O)CC3)nc2)n1 ; CCOc1cncc(-c2ccc(NC(=O)C3(c4ccnc(NS(=O)(=O)C5CC5)n4)CCC(=C(F)C4(O)CC4)CC3)nc2)n1]

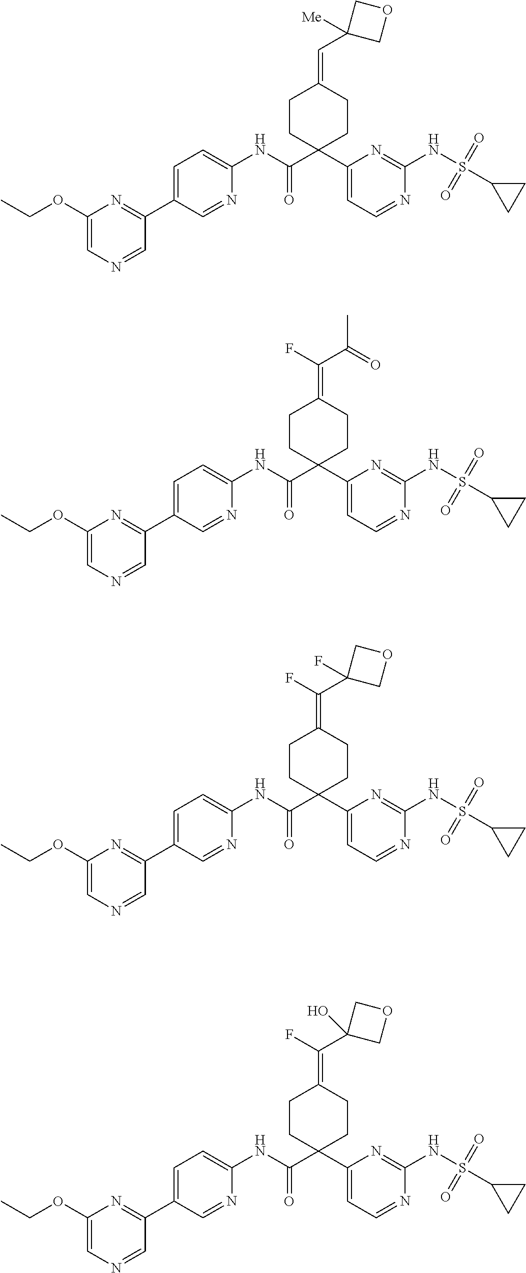 [SMILES: CCOc1cncc(-c2ccc(NC(=O)C3(c4ccnc(NS(=O)(=O)C5CC5)n4)CCC(=CC4(C)COC4)CC3)nc2)n1 ; CCOc1cncc(-c2ccc(NC(=O)C3(c4ccnc(NS(=O)(=O)C5CC5)n4)CCC(=C(F)C(C)=O)CC3)nc2)n1 ; CCOc1cncc(-c2ccc(NC(=O)C3(c4ccnc(NS(=O)(=O)C5CC5)n4)CCC(=C(F)C4(F)COC4)CC3)nc2)n1 ; CCOc1cncc(-c2ccc(NC(=O)C3(c4ccnc(NS(=O)(=O)C5CC5)n4)CCC(=C(F)C4(O)COC4)CC3)nc2)n1]

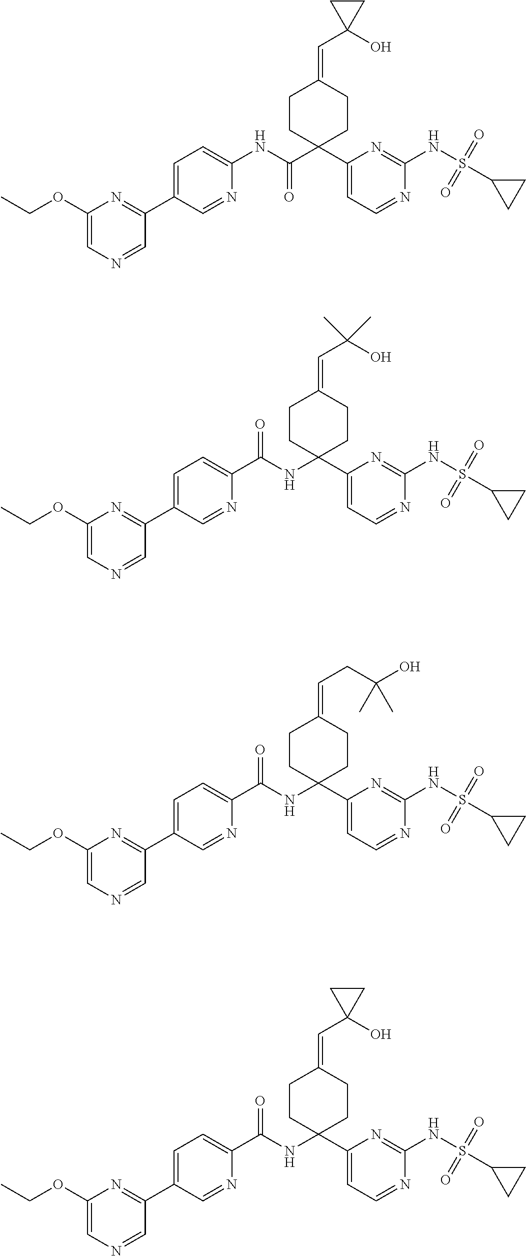 [SMILES: CCOc1cncc(-c2ccc(NC(=O)C3(c4ccnc(NS(=O)(=O)C5CC5)n4)CCC(=CC4(O)CC4)CC3)nc2)n1 ; CCOc1cncc(-c2ccc(C(=O)NC3(c4ccnc(NS(=O)(=O)C5CC5)n4)CCC(=CC(C)(C)O)CC3)nc2)n1 ; CCOc1cncc(-c2ccc(C(=O)NC3(c4ccnc(NS(=O)(=O)C5CC5)n4)CCC(=CCC(C)(C)O)CC3)nc2)n1 ; CCOc1cncc(-c2ccc(C(=O)NC3(c4ccnc(NS(=O)(=O)C5CC5)n4)CCC(=CC4(O)CC4)CC3)nc2)n1]

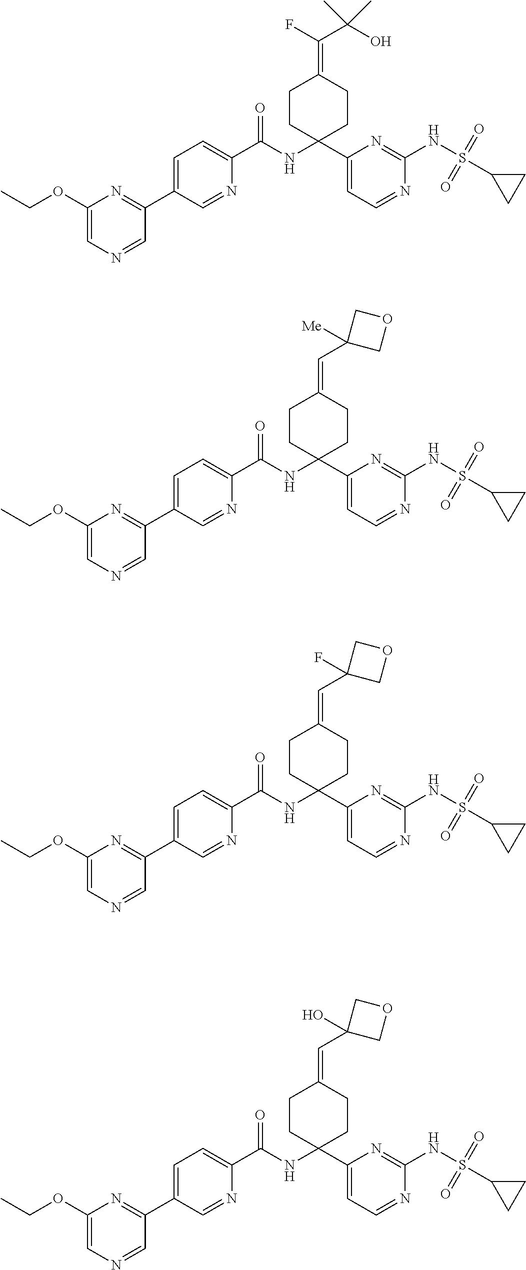 [SMILES: CCOc1cncc(-c2ccc(C(=O)NC3(c4ccnc(NS(=O)(=O)C5CC5)n4)CCC(=C(F)C(C)(C)O)CC3)nc2)n1 ; CCOc1cncc(-c2ccc(C(=O)NC3(c4ccnc(NS(=O)(=O)C5CC5)n4)CCC(=CC4(C)COC4)CC3)nc2)n1 ; CCOc1cncc(-c2ccc(C(=O)NC3(c4ccnc(NS(=O)(=O)C5CC5)n4)CCC(=CC4(F)COC4)CC3)nc2)n1 ; CCOc1cncc(-c2ccc(C(=O)NC3(c4ccnc(NS(=O)(=O)C5CC5)n4)CCC(=CC4(O)COC4)CC3)nc2)n1]

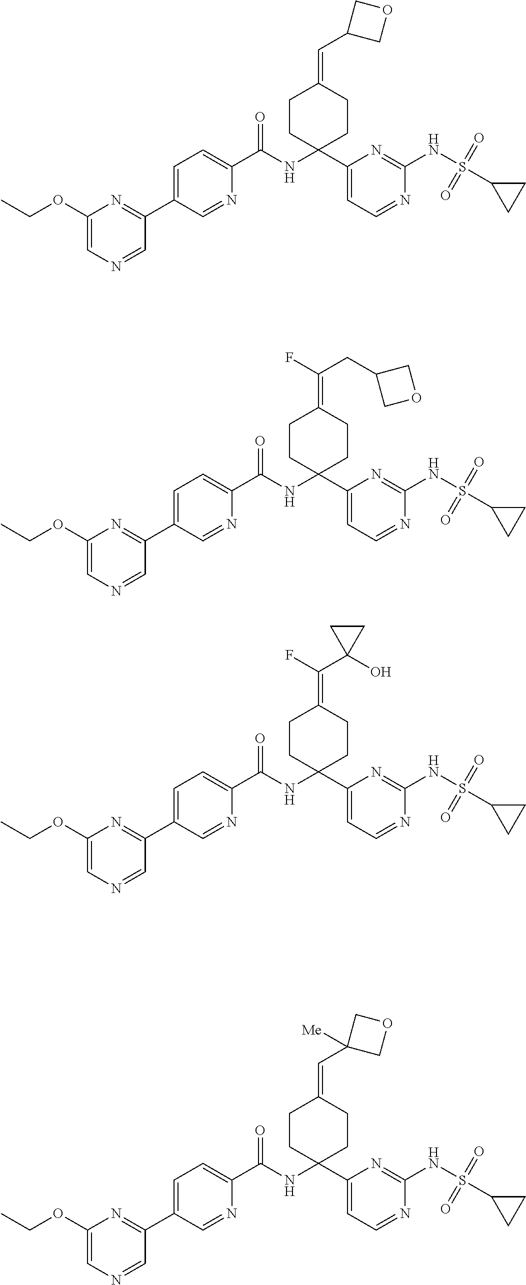 [SMILES: CCOc1cncc(-c2ccc(C(=O)NC3(c4ccnc(NS(=O)(=O)C5CC5)n4)CCC(=CC4COC4)CC3)nc2)n1 ; CCOc1cncc(-c2ccc(C(=O)NC3(c4ccnc(NS(=O)(=O)C5CC5)n4)CCC(=C(F)CC4COC4)CC3)nc2)n1 ; CCOc1cncc(-c2ccc(C(=O)NC3(c4ccnc(NS(=O)(=O)C5CC5)n4)CCC(=C(F)C4(O)CC4)CC3)nc2)n1 ; CCOc1cncc(-c2ccc(C(=O)NC3(c4ccnc(NS(=O)(=O)C5CC5)n4)CCC(=CC4(C)COC4)CC3)nc2)n1]

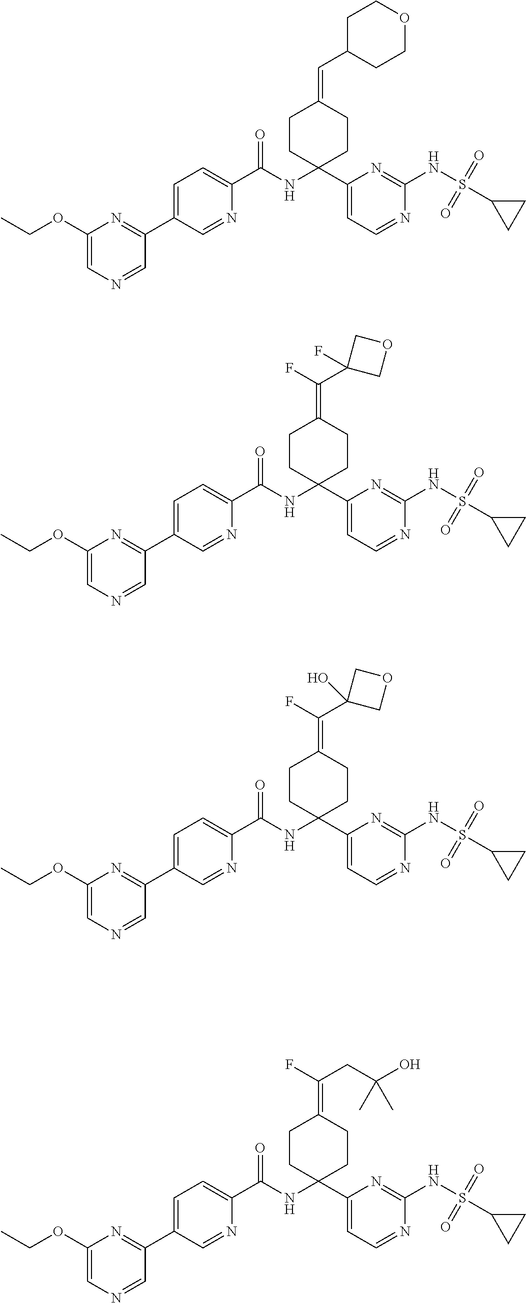 [SMILES: CCOc1cncc(-c2ccc(C(=O)NC3(c4ccnc(NS(=O)(=O)C5CC5)n4)CCC(=CC4CCOCC4)CC3)nc2)n1 ; CCOc1cncc(-c2ccc(C(=O)NC3(c4ccnc(NS(=O)(=O)C5CC5)n4)CCC(=C(F)C4(F)COC4)CC3)nc2)n1 ; CCOc1cncc(-c2ccc(C(=O)NC3(c4ccnc(NS(=O)(=O)C5CC5)n4)CCC(=C(F)C4(O)COC4)CC3)nc2)n1 ; CCOc1cncc(-c2ccc(C(=O)NC3(c4ccnc(NS(=O)(=O)C5CC5)n4)CCC(=C(F)CC(C)(C)O)CC3)nc2)n1]

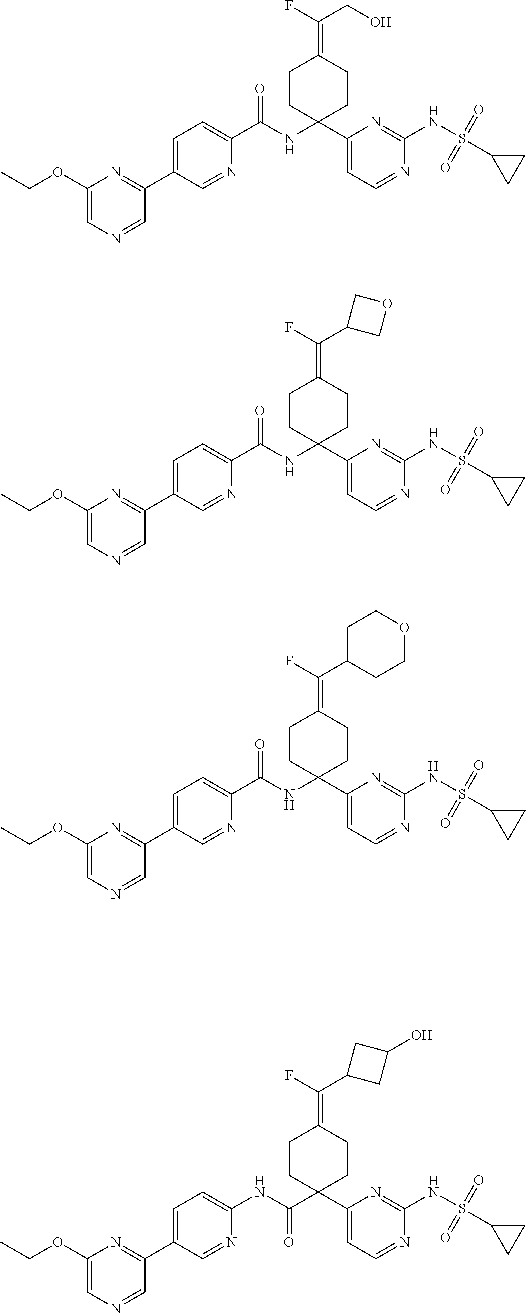 [SMILES: CCOc1cncc(-c2ccc(C(=O)NC3(c4ccnc(NS(=O)(=O)C5CC5)n4)CCC(=C(F)CO)CC3)nc2)n1 ; CCOc1cncc(-c2ccc(C(=O)NC3(c4ccnc(NS(=O)(=O)C5CC5)n4)CCC(=C(F)C4COC4)CC3)nc2)n1 ; CCOc1cncc(-c2ccc(C(=O)NC3(c4ccnc(NS(=O)(=O)C5CC5)n4)CCC(=C(F)C4CCOCC4)CC3)nc2)n1 ; CCOc1cncc(-c2ccc(NC(=O)C3(c4ccnc(NS(=O)(=O)C5CC5)n4)CCC(=C(F)C4CC(O)C4)CC3)nc2)n1]

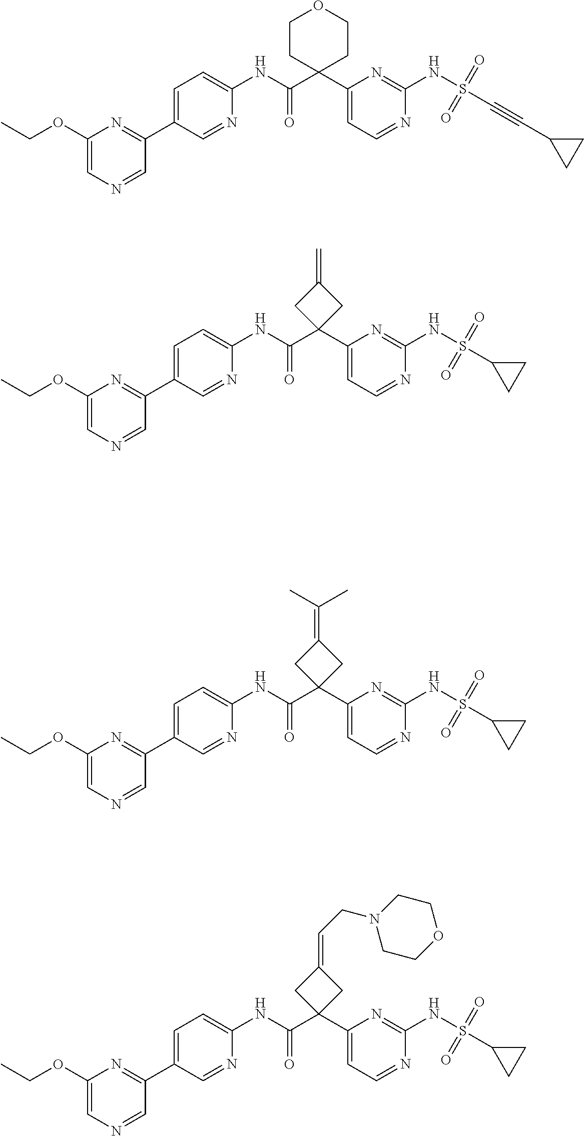 [SMILES: CCOc1cncc(-c2ccc(NC(=O)C3(c4ccnc(NS(=O)(=O)C#CC5CC5)n4)CCOCC3)nc2)n1 ; C=C1CC(C(=O)Nc2ccc(-c3cncc(OCC)n3)cn2)(c2ccnc(NS(=O)(=O)C3CC3)n2)C1 ; CCOc1cncc(-c2ccc(NC(=O)C3(c4ccnc(NS(=O)(=O)C5CC5)n4)CC(=C(C)C)C3)nc2)n1 ; CCOc1cncc(-c2ccc(NC(=O)C3(c4ccnc(NS(=O)(=O)C5CC5)n4)CC(=CCN4CCOCC4)C3)nc2)n1]

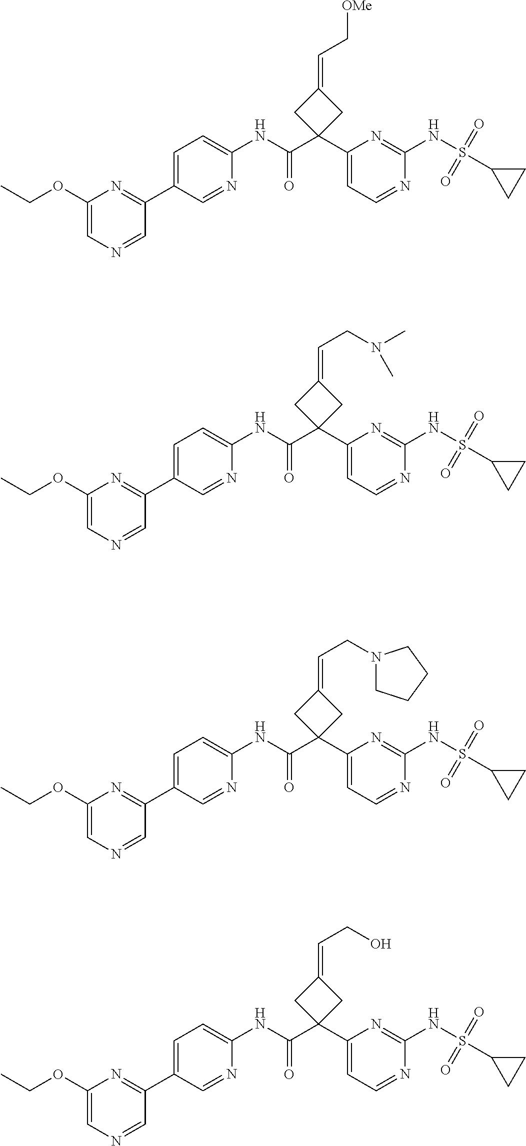 [SMILES: CCOc1cncc(-c2ccc(NC(=O)C3(c4ccnc(NS(=O)(=O)C5CC5)n4)CC(=CCOC)C3)nc2)n1 ; CCOc1cncc(-c2ccc(NC(=O)C3(c4ccnc(NS(=O)(=O)C5CC5)n4)CC(=CCN(C)C)C3)nc2)n1 ; CCOc1cncc(-c2ccc(NC(=O)C3(c4ccnc(NS(=O)(=O)C5CC5)n4)CC(=CCN4CCCC4)C3)nc2)n1 ; CCOc1cncc(-c2ccc(NC(=O)C3(c4ccnc(NS(=O)(=O)C5CC5)n4)CC(=CCO)C3)nc2)n1]

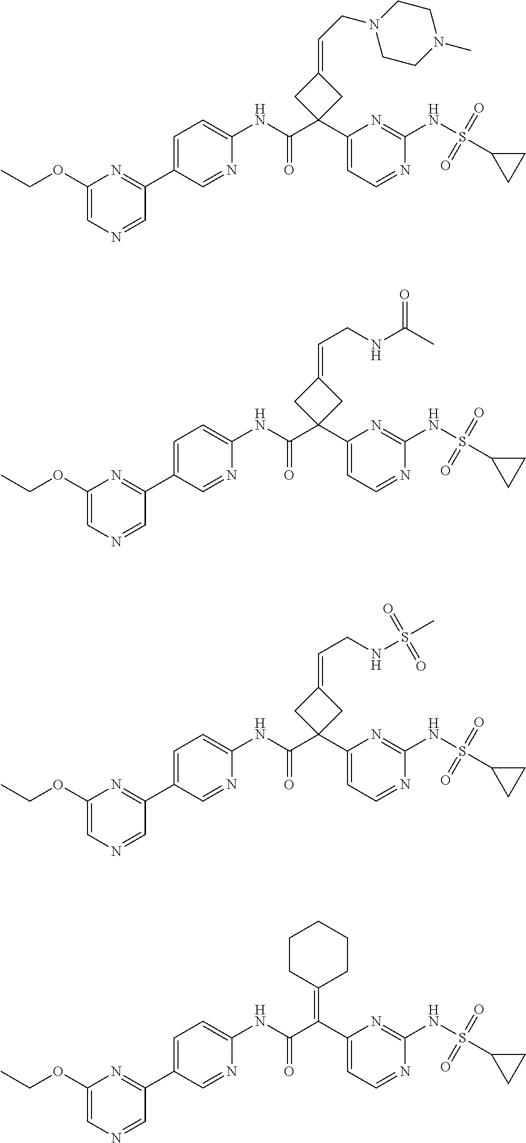 [SMILES: CCOc1cncc(-c2ccc(NC(=O)C3(c4ccnc(NS(=O)(=O)C5CC5)n4)CC(=CCN4CCN(C)CC4)C3)nc2)n1 ; CCOc1cncc(-c2ccc(NC(=O)C3(c4ccnc(NS(=O)(=O)C5CC5)n4)CC(=CCNC(C)=O)C3)nc2)n1 ; CCOc1cncc(-c2ccc(NC(=O)C3(c4ccnc(NS(=O)(=O)C5CC5)n4)CC(=CCNS(C)(=O)=O)C3)nc2)n1 ; CCOc1cncc(-c2ccc(NC(=O)C(=C3CCCCC3)c3ccnc(NS(=O)(=O)C4CC4)n3)nc2)n1]

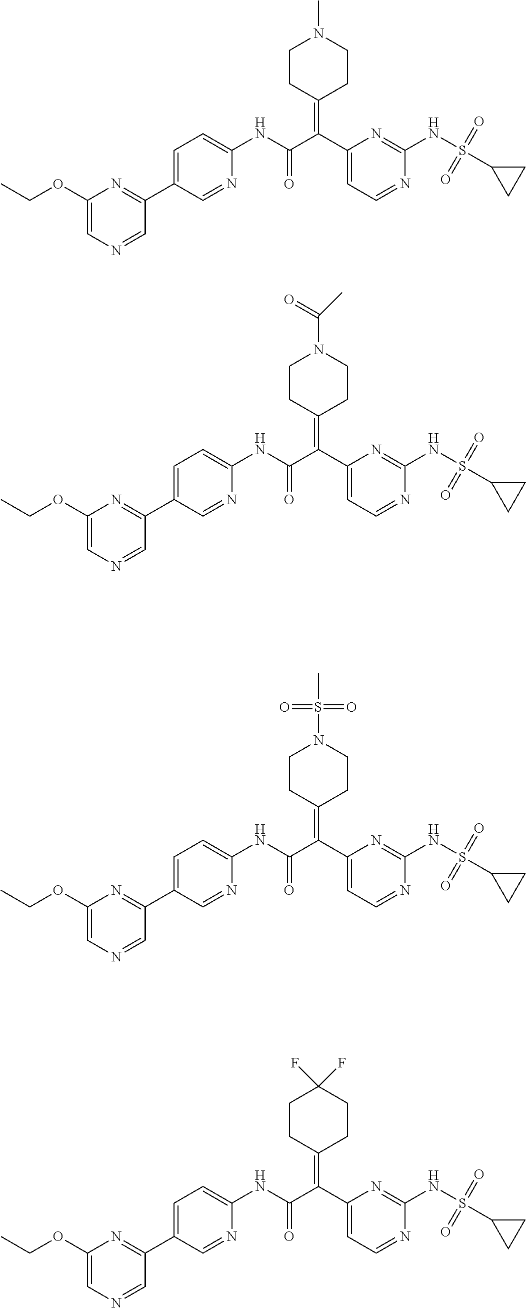 [SMILES: CCOc1cncc(-c2ccc(NC(=O)C(=C3CCN(C)CC3)c3ccnc(NS(=O)(=O)C4CC4)n3)nc2)n1 ; CCOc1cncc(-c2ccc(NC(=O)C(=C3CCN(C(C)=O)CC3)c3ccnc(NS(=O)(=O)C4CC4)n3)nc2)n1 ; CCOc1cncc(-c2ccc(NC(=O)C(=C3CCN(S(C)(=O)=O)CC3)c3ccnc(NS(=O)(=O)C4CC4)n3)nc2)n1 ; CCOc1cncc(-c2ccc(NC(=O)C(=C3CCC(F)(F)CC3)c3ccnc(NS(=O)(=O)C4CC4)n3)nc2)n1]

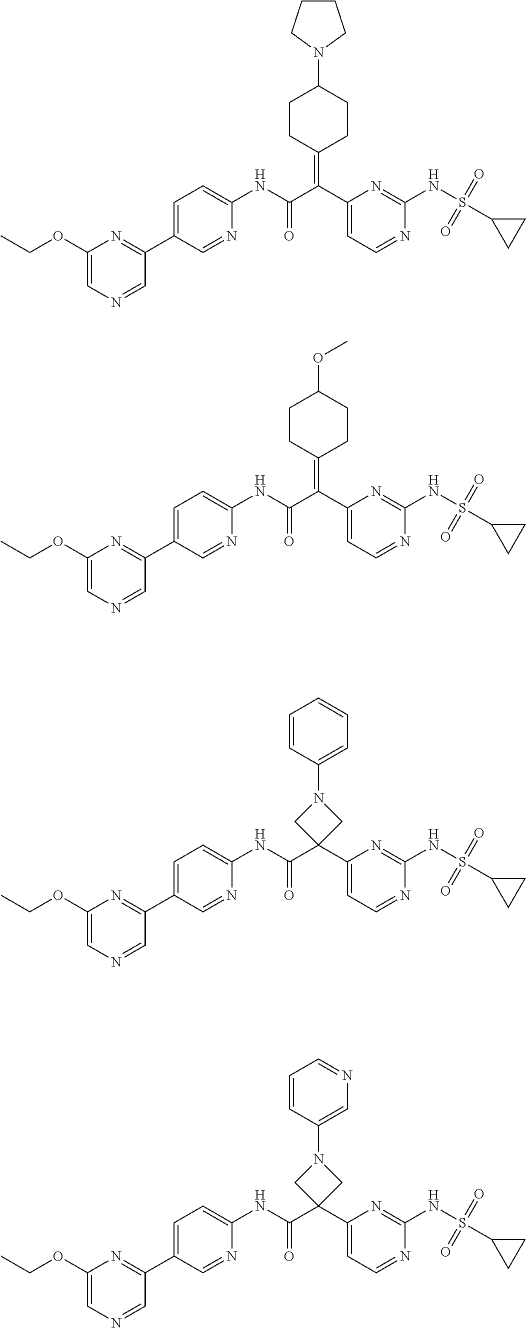 [SMILES: CCOc1cncc(-c2ccc(NC(=O)C(=C3CCC(N4CCCC4)CC3)c3ccnc(NS(=O)(=O)C4CC4)n3)nc2)n1 ; CCOc1cncc(-c2ccc(NC(=O)C(=C3CCC(OC)CC3)c3ccnc(NS(=O)(=O)C4CC4)n3)nc2)n1 ; CCOc1cncc(-c2ccc(NC(=O)C3(c4ccnc(NS(=O)(=O)C5CC5)n4)CN(c4ccccc4)C3)nc2)n1 ; CCOc1cncc(-c2ccc(NC(=O)C3(c4ccnc(NS(=O)(=O)C5CC5)n4)CN(c4cccnc4)C3)nc2)n1]

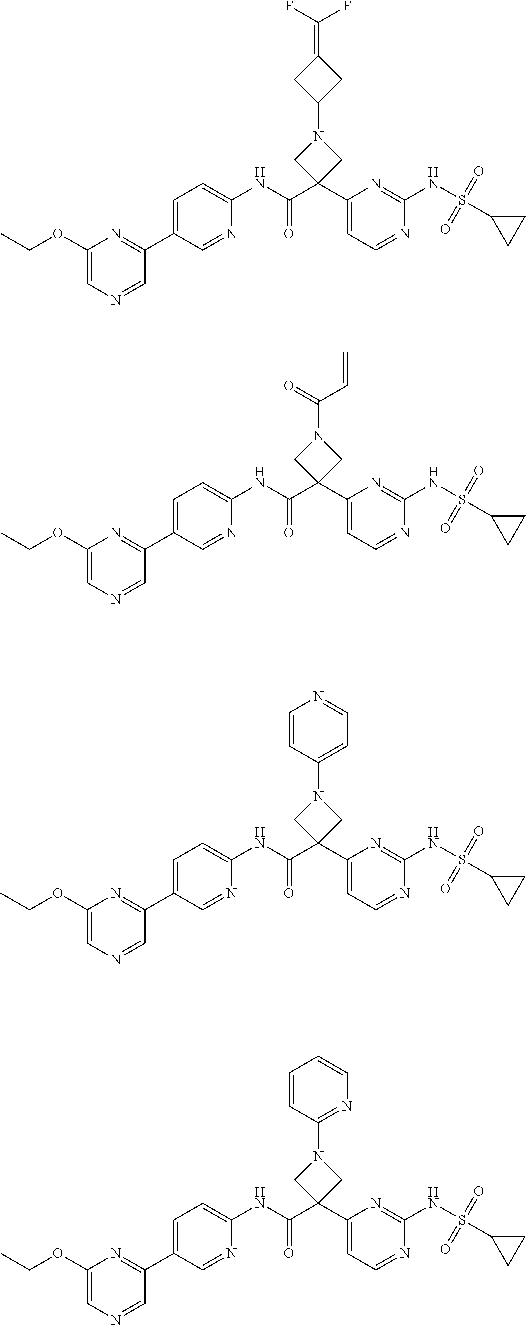 [SMILES: CCOc1cncc(-c2ccc(NC(=O)C3(c4ccnc(NS(=O)(=O)C5CC5)n4)CN(C4CC(=C(F)F)C4)C3)nc2)n1 ; C=CC(=O)N1CC(C(=O)Nc2ccc(-c3cncc(OCC)n3)cn2)(c2ccnc(NS(=O)(=O)C3CC3)n2)C1 ; CCOc1cncc(-c2ccc(NC(=O)C3(c4ccnc(NS(=O)(=O)C5CC5)n4)CN(c4ccncc4)C3)nc2)n1 ; CCOc1cncc(-c2ccc(NC(=O)C3(c4ccnc(NS(=O)(=O)C5CC5)n4)CN(c4ccccn4)C3)nc2)n1]

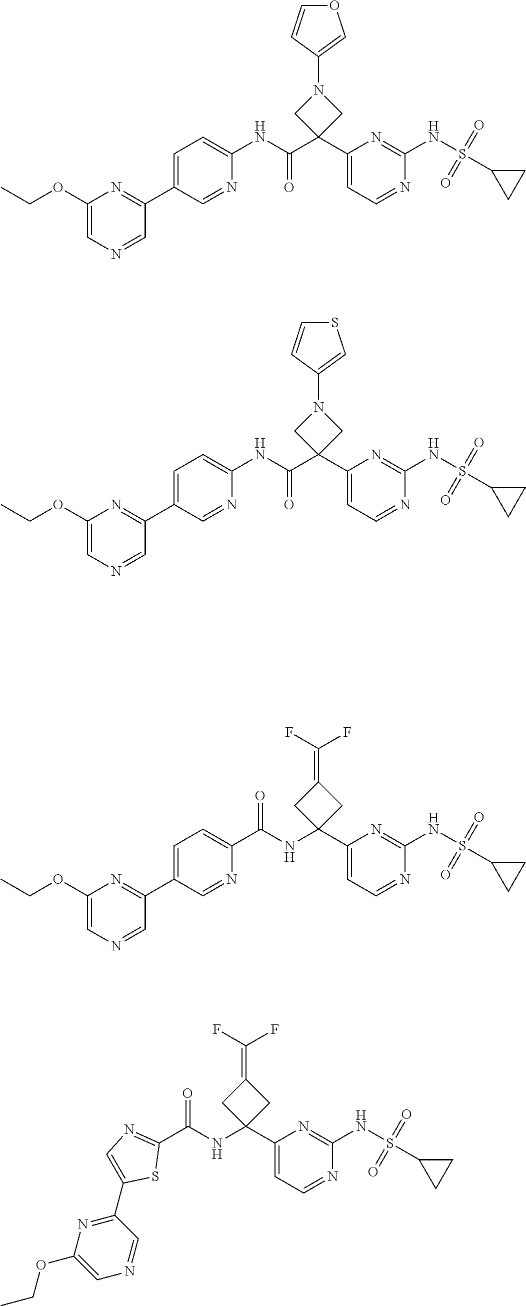 [SMILES: CCOc1cncc(-c2ccc(NC(=O)C3(c4ccnc(NS(=O)(=O)C5CC5)n4)CN(c4ccoc4)C3)nc2)n1 ; CCOc1cncc(-c2ccc(NC(=O)C3(c4ccnc(NS(=O)(=O)C5CC5)n4)CN(c4ccsc4)C3)nc2)n1 ; CCOc1cncc(-c2ccc(C(=O)NC3(c4ccnc(NS(=O)(=O)C5CC5)n4)CC(=C(F)F)C3)nc2)n1 ; CCOc1cncc(-c2cnc(C(=O)NC3(c4ccnc(NS(=O)(=O)C5CC5)n4)CC(=C(F)F)C3)s2)n1]

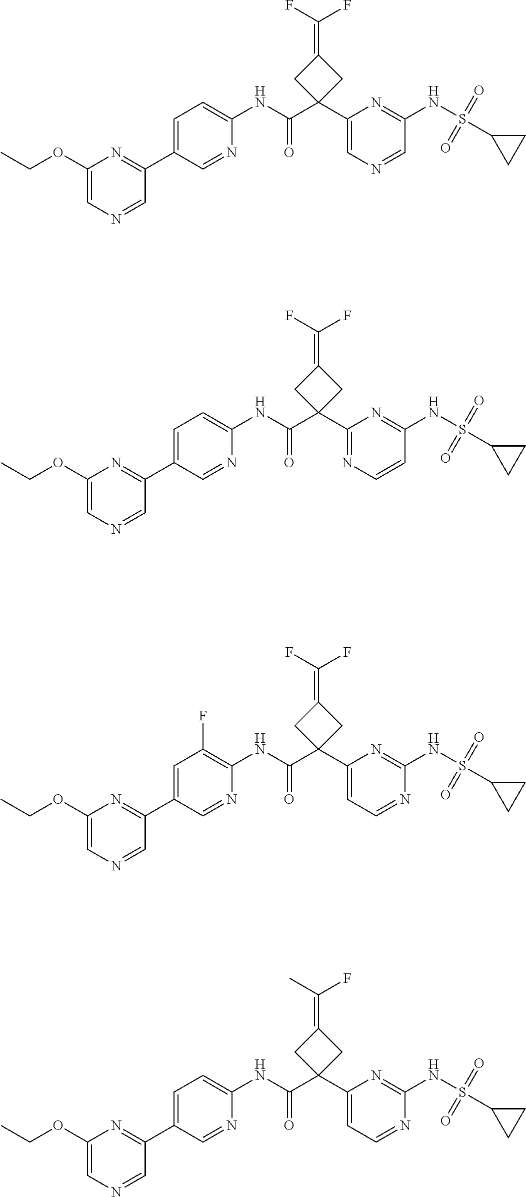 [SMILES: CCOc1cncc(-c2ccc(NC(=O)C3(c4cncc(NS(=O)(=O)C5CC5)n4)CC(=C(F)F)C3)nc2)n1 ; CCOc1cncc(-c2ccc(NC(=O)C3(c4nccc(NS(=O)(=O)C5CC5)n4)CC(=C(F)F)C3)nc2)n1 ; CCOc1cncc(-c2cnc(NC(=O)C3(c4ccnc(NS(=O)(=O)C5CC5)n4)CC(=C(F)F)C3)c(F)c2)n1 ; CCOc1cncc(-c2ccc(NC(=O)C3(c4ccnc(NS(=O)(=O)C5CC5)n4)CC(=C(C)F)C3)nc2)n1]

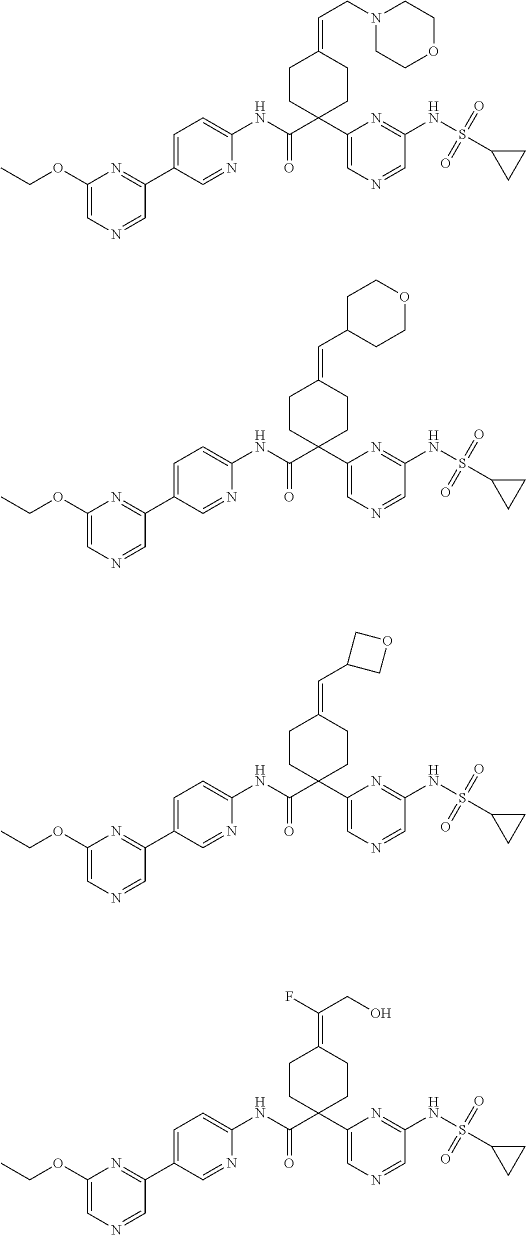 [SMILES: CCOc1cncc(-c2ccc(NC(=O)C3(c4cncc(NS(=O)(=O)C5CC5)n4)CCC(=CCN4CCOCC4)CC3)nc2)n1 ; CCOc1cncc(-c2ccc(NC(=O)C3(c4cncc(NS(=O)(=O)C5CC5)n4)CCC(=CC4CCOCC4)CC3)nc2)n1 ; CCOc1cncc(-c2ccc(NC(=O)C3(c4cncc(NS(=O)(=O)C5CC5)n4)CCC(=CC4COC4)CC3)nc2)n1 ; CCOc1cncc(-c2ccc(NC(=O)C3(c4cncc(NS(=O)(=O)C5CC5)n4)CCC(=C(F)CO)CC3)nc2)n1]

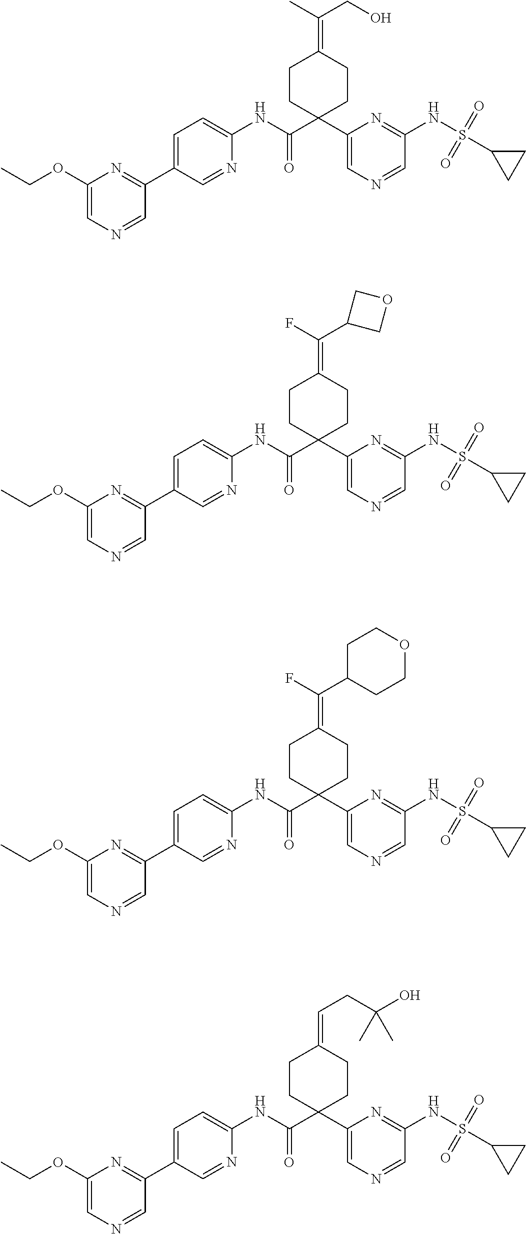 [SMILES: CCOc1cncc(-c2ccc(NC(=O)C3(c4cncc(NS(=O)(=O)C5CC5)n4)CCC(=C(C)CO)CC3)nc2)n1 ; CCOc1cncc(-c2ccc(NC(=O)C3(c4cncc(NS(=O)(=O)C5CC5)n4)CCC(=C(F)C4COC4)CC3)nc2)n1 ; CCOc1cncc(-c2ccc(NC(=O)C3(c4cncc(NS(=O)(=O)C5CC5)n4)CCC(=C(F)C4CCOCC4)CC3)nc2)n1 ; CCOc1cncc(-c2ccc(NC(=O)C3(c4cncc(NS(=O)(=O)C5CC5)n4)CCC(=CCC(C)(C)O)CC3)nc2)n1]

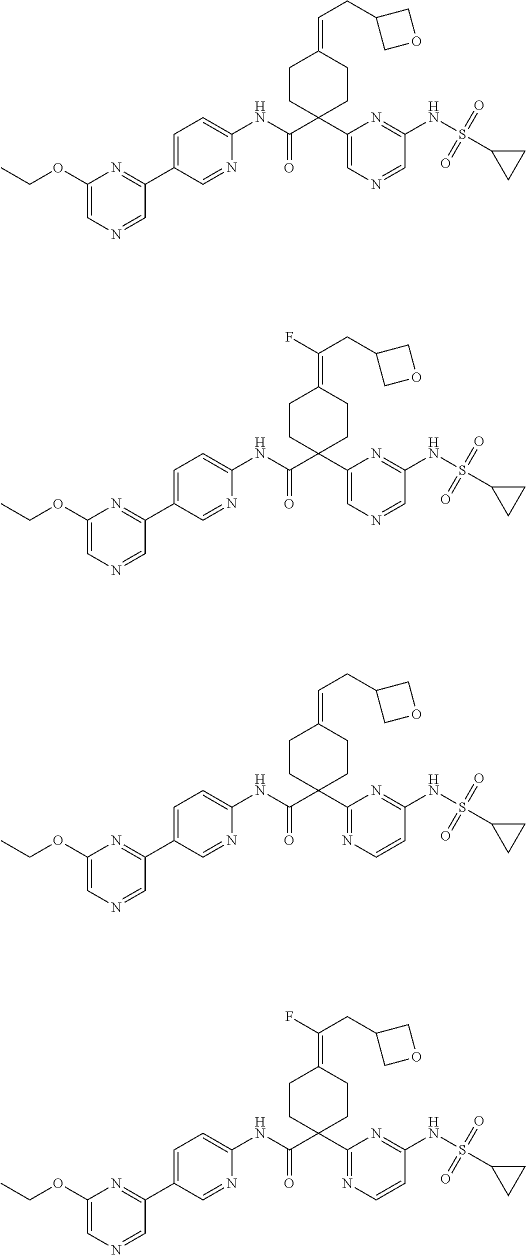 [SMILES: CCOc1cncc(-c2ccc(NC(=O)C3(c4cncc(NS(=O)(=O)C5CC5)n4)CCC(=CCC4COC4)CC3)nc2)n1 ; CCOc1cncc(-c2ccc(NC(=O)C3(c4cncc(NS(=O)(=O)C5CC5)n4)CCC(=C(F)CC4COC4)CC3)nc2)n1 ; CCOc1cncc(-c2ccc(NC(=O)C3(c4nccc(NS(=O)(=O)C5CC5)n4)CCC(=CCC4COC4)CC3)nc2)n1 ; CCOc1cncc(-c2ccc(NC(=O)C3(c4nccc(NS(=O)(=O)C5CC5)n4)CCC(=C(F)CC4COC4)CC3)nc2)n1]

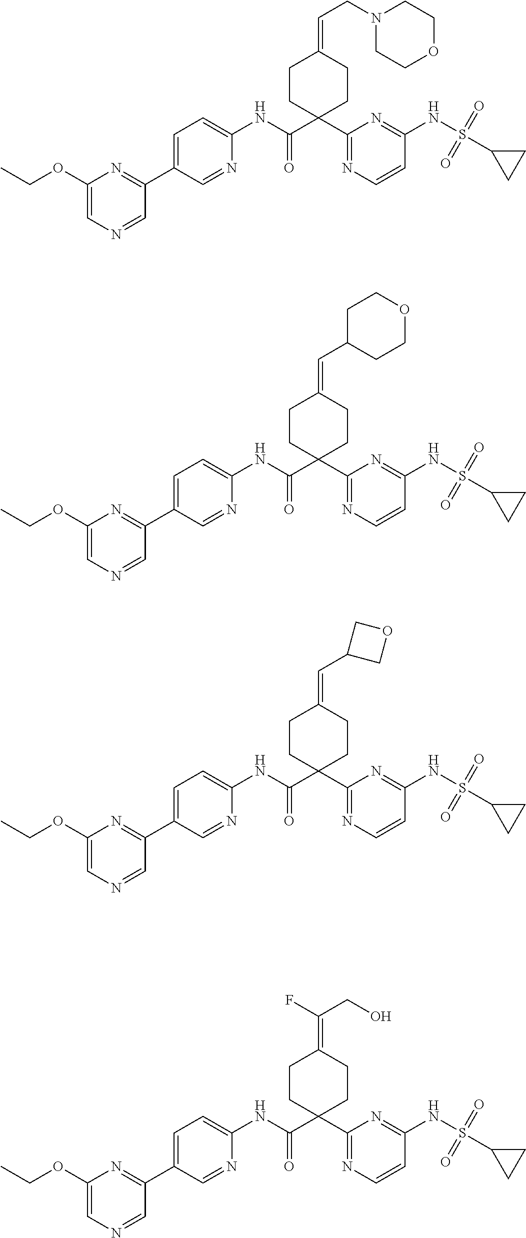 [SMILES: CCOc1cncc(-c2ccc(NC(=O)C3(c4nccc(NS(=O)(=O)C5CC5)n4)CCC(=CCN4CCOCC4)CC3)nc2)n1 ; CCOc1cncc(-c2ccc(NC(=O)C3(c4nccc(NS(=O)(=O)C5CC5)n4)CCC(=CC4CCOCC4)CC3)nc2)n1 ; CCOc1cncc(-c2ccc(NC(=O)C3(c4nccc(NS(=O)(=O)C5CC5)n4)CCC(=CC4COC4)CC3)nc2)n1 ; CCOc1cncc(-c2ccc(NC(=O)C3(c4nccc(NS(=O)(=O)C5CC5)n4)CCC(=C(F)CO)CC3)nc2)n1]

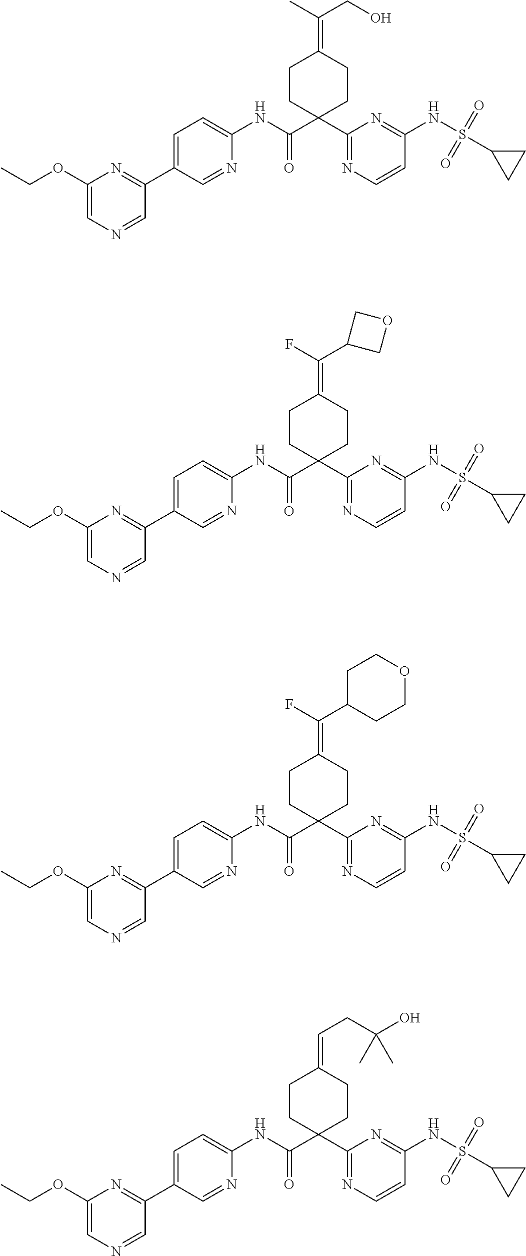 [SMILES: CCOc1cncc(-c2ccc(NC(=O)C3(c4nccc(NS(=O)(=O)C5CC5)n4)CCC(=C(C)CO)CC3)nc2)n1 ; CCOc1cncc(-c2ccc(NC(=O)C3(c4nccc(NS(=O)(=O)C5CC5)n4)CCC(=C(F)C4COC4)CC3)nc2)n1 ; CCOc1cncc(-c2ccc(NC(=O)C3(c4nccc(NS(=O)(=O)C5CC5)n4)CCC(=C(F)C4CCOCC4)CC3)nc2)n1 ; CCOc1cncc(-c2ccc(NC(=O)C3(c4nccc(NS(=O)(=O)C5CC5)n4)CCC(=CCC(C)(C)O)CC3)nc2)n1]

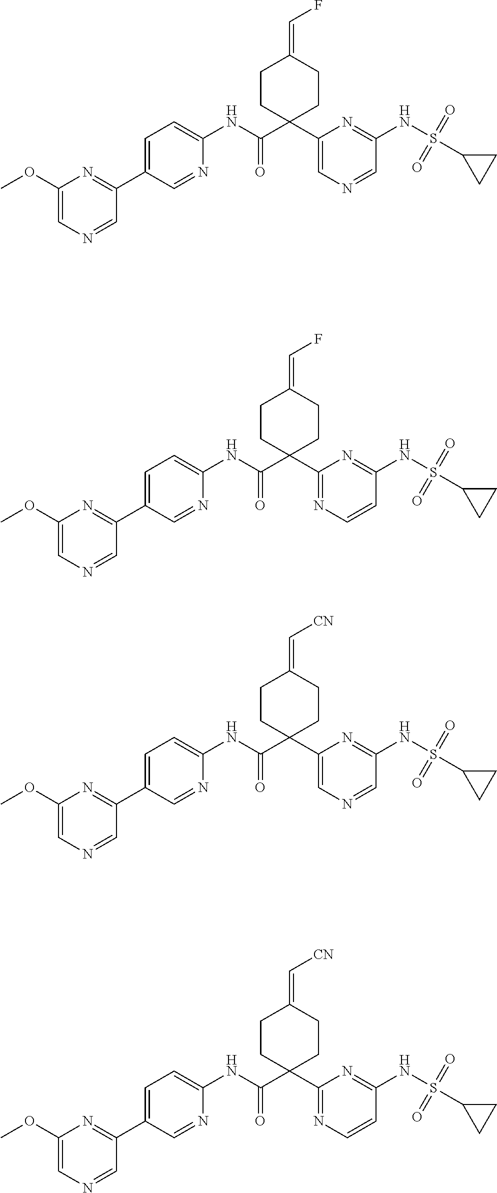 [SMILES: COc1cncc(-c2ccc(NC(=O)C3(c4cncc(NS(=O)(=O)C5CC5)n4)CCC(=CF)CC3)nc2)n1 ; COc1cncc(-c2ccc(NC(=O)C3(c4nccc(NS(=O)(=O)C5CC5)n4)CCC(=CF)CC3)nc2)n1 ; COc1cncc(-c2ccc(NC(=O)C3(c4cncc(NS(=O)(=O)C5CC5)n4)CCC(=CC#N)CC3)nc2)n1 ; COc1cncc(-c2ccc(NC(=O)C3(c4nccc(NS(=O)(=O)C5CC5)n4)CCC(=CC#N)CC3)nc2)n1]

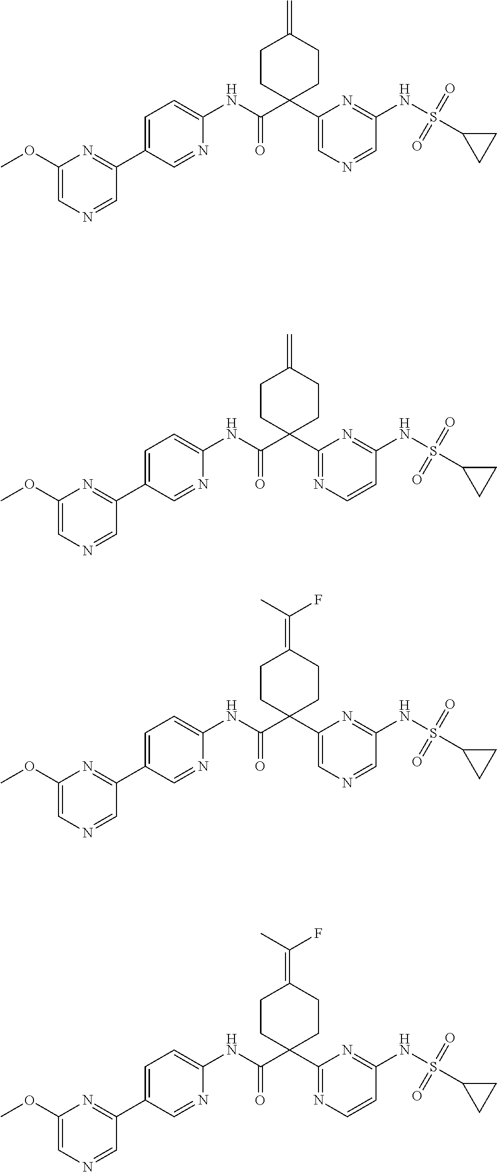 [SMILES: C=C1CCC(C(=O)Nc2ccc(-c3cncc(OC)n3)cn2)(c2cncc(NS(=O)(=O)C3CC3)n2)CC1 ; C=C1CCC(C(=O)Nc2ccc(-c3cncc(OC)n3)cn2)(c2nccc(NS(=O)(=O)C3CC3)n2)CC1 ; COc1cncc(-c2ccc(NC(=O)C3(c4cncc(NS(=O)(=O)C5CC5)n4)CCC(=C(C)F)CC3)nc2)n1 ; COc1cncc(-c2ccc(NC(=O)C3(c4nccc(NS(=O)(=O)C5CC5)n4)CCC(=C(C)F)CC3)nc2)n1]

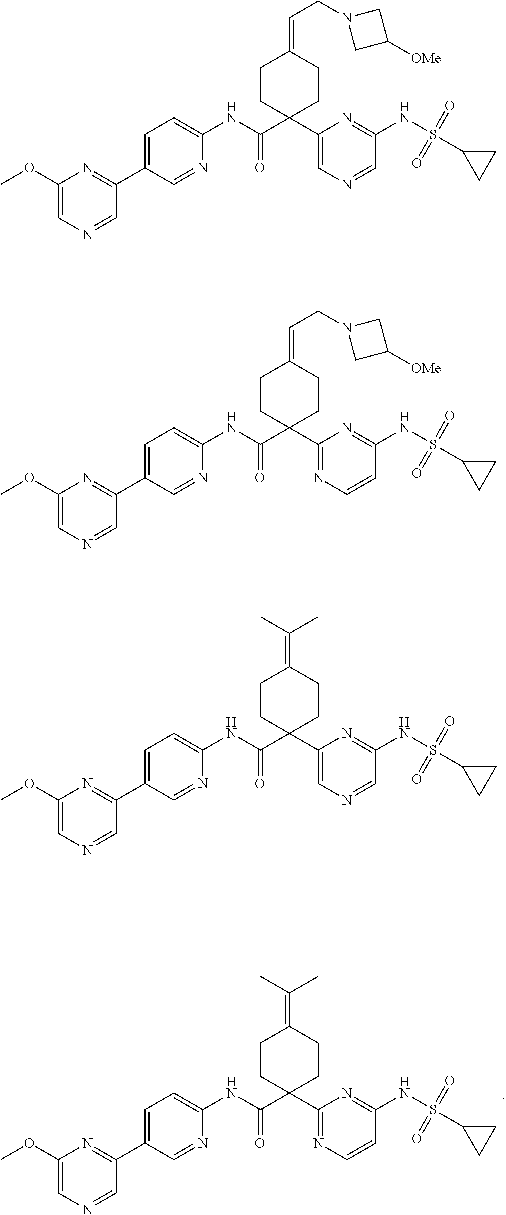 [SMILES: COc1cncc(-c2ccc(NC(=O)C3(c4cncc(NS(=O)(=O)C5CC5)n4)CCC(=CCN4CC(OC)C4)CC3)nc2)n1 ; COc1cncc(-c2ccc(NC(=O)C3(c4nccc(NS(=O)(=O)C5CC5)n4)CCC(=CCN4CC(OC)C4)CC3)nc2)n1 ; COc1cncc(-c2ccc(NC(=O)C3(c4cncc(NS(=O)(=O)C5CC5)n4)CCC(=C(C)C)CC3)nc2)n1 ; COc1cncc(-c2ccc(NC(=O)C3(c4nccc(NS(=O)(=O)C5CC5)n4)CCC(=C(C)C)CC3)nc2)n1]

15. A pharmaceutical composition, characterized in that it comprises a compound according to any one of claims 1 to 14, or an optical isomer, pharmaceutically acceptable salt, prodrug, deuterated derivative, hydrate, or solvate thereof, and a pharmaceutically acceptable carrier.

16. Use of a compound according to any one of claims 1 to 14, or an optical isomer, pharmaceutically acceptable salt, prodrug, deuterated derivative, hydrate, or solvate thereof, characterized in that it is for the preparation of a medicament for treating a disease, disorder, or condition associated with CTPS1 activity or expression level.

17. The use according to claim 16, characterized in that the disease, disorder, or condition is selected from the group consisting of psoriasis, rheumatoid arthritis, systemic lupus erythematosus, lupus nephritis or cutaneous lupus, myasthenia gravis, multiple sclerosis, scleroderma, alopecia areata, inflammatory bowel disease and other various autoimmune diseases; graft-versus-host disease; promotion of vascular smooth muscle cell proliferation and repair after vascular injury or surgery; T-cell lymphoma, B-cell lymphoma, acute myeloid leukemia, acute lymphoblastic leukemia, chronic lymphocytic leukemia, angioimmunoblastic T-cell lymphoma, B-cell acute lymphoblastic leukemia, Hodgkin lymphoma, T-cell non-Hodgkin lymphoma (including natural killer/T-cell lymphoma, enteropathy-associated T-cell lymphoma, adult T-cell leukemia/lymphoma, hepatosplenic T-cell lymphoma, and cutaneous T-cell lymphoma), T-cell acute lymphoblastic leukemia, B-cell non-Hodgkin lymphoma (including Burkitt lymphoma, diffuse large B-cell lymphoma, follicular lymphoma, mantle cell lymphoma, and marginal zone lymphoma), hairy cell leukemia, lymphoblastic lymphoma, lymphoplasmacytic lymphoma, mucosa-associated lymphoid tissue lymphoma, multiple myeloma, myelodysplastic syndrome, plasma cell myeloma, primary mediastinal large B-cell lymphoma, primary myelofibrosis, essential thrombocythemia, and polycythemia vera and other various hematological cancers.
